(19) United States
(12) Patent Application Publication
(10) Pub. No.: US 2025/0098529 A1
(43) Pub. Date: Mar. 20, 2025

(54) LIGHT EMITTING ELEMENT, AMINE COMPOUND FOR THE SAME, AND DISPLAY DEVICE INCLUDING THE SAME

(71) Applicant: Samsung Display Co., Ltd., Yongin-si (KR)

(72) Inventor: Naoya SAKAMOTO, Yokohama (JP)

(21) Appl. No.: 18/814,363

(22) Filed: Aug. 23, 2024

(30) Foreign Application Priority Data
Aug. 28, 2023 (KR) 10-2023-0112575

(51) Int. Cl. H10K 85/60; C07D 307/91; C07D 333/74; C07D 333/76; H10K 50/12; H10K 50/155

(52) U.S. Cl. CPC H10K 85/636; C07D 307/91; C07D 333/74; C07D 333/76; H10K 85/626; H10K 85/633; H10K 50/12; H10K 50/155; H10K 85/615; H10K 85/6574; H10K 85/6576

(57) ABSTRACT

A light emitting element includes a first electrode, a second electrode on the first electrode, an emission layer between the first electrode and the second electrode, and a hole transport region between the first electrode and the emission layer, wherein the hole transport region may include an amine compound represented by Formula 1:

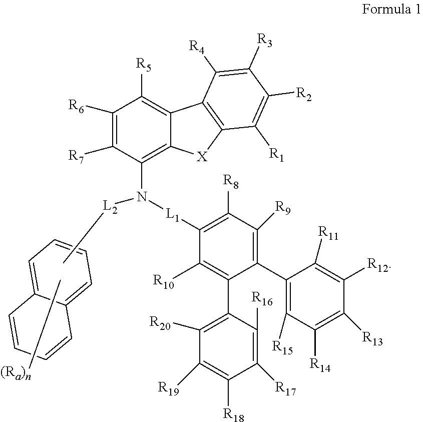

### CROSS-REFERENCE TO RELATED APPLICATION

The present application claims priority to and the benefit of Korean Patent Application No. 10-2023-0112575, filed on Aug. 28, 2023, in the Korean Intellectual Property Office, the entire content of which is incorporated herein by reference.

### BACKGROUND

### 1. Field

One or more embodiments of the present disclosure relate to a light emitting element, an amine compound utilized therein, and a display device including the light emitting element.

### 2. Description of the Related Art

Recently, the development of an organic electroluminescence display device as an image display device is being actively conducted. The organic electroluminescence display device includes a self-luminescent light emitting element in which holes and electrons injected respectively from a first electrode and a second electrode combine in an emission layer of the light emitting element, and cause a luminescent material of the emission layer to emit light to implement display of images.

In the application of a light emitting element to a display device, there is a demand and desire for a light emitting element having improved light efficiency and/or improved service life, and thus the development on materials for a light emitting element capable of stably attaining such characteristics is being continuously pursued or required.

For example, the development on materials for a hole transport region having improved charge transport properties and material stability is being actively carried out in order to implement a light emitting element having a relatively high efficiency and a relatively long service life.

### SUMMARY

One or more aspects of embodiments of the present disclosure are directed toward a light emitting element in which luminous efficiency and a service life are improved.

One or more aspects of embodiments of the present disclosure are directed toward an amine compound which is a material for a light emitting element having high efficiency and long service life characteristics.

One or more aspects of embodiments of the present disclosure are directed toward a display device including the light emitting element in which the luminous efficiency and service life are improved, thereby having excellent or suitable display quality.

Additional aspects will be set forth in part in the description which follows and, in part, will be apparent from the description, or may be learned by practice of the presented embodiments.

According to one or more embodiments of the present disclosure, an amine compound is represented by Formula 1:

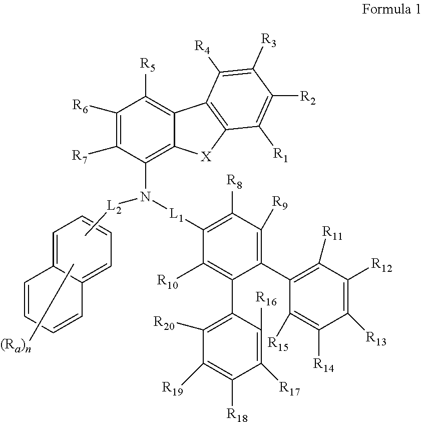

In Formula 1, X may be O or S, and R₁ to R₂₀ may each independently be hydrogen, deuterium, a halogen, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or may be independently bonded to an adjacent group to form (or provide) a ring. L₁ may be a direct linkage, a substituted or unsubstituted arylene group having 6 to 30 ring-forming carbon atoms, or a substituted or unsubstituted heteroarylene group having 2 to 30 ring-forming carbon atoms, and L₂ may be a substituted or unsubstituted arylene group having 6 to 30 ring-forming carbon atoms. n may be an integer of 1 to 7, and Rₐ may be hydrogen, deuterium, or a substituted or unsubstituted aryl group having 6 to 30 ring-forming carbon atoms.

In one or more embodiments, the amine compound represented by Formula 1 may be represented by Formula 1A or Formula 1B:

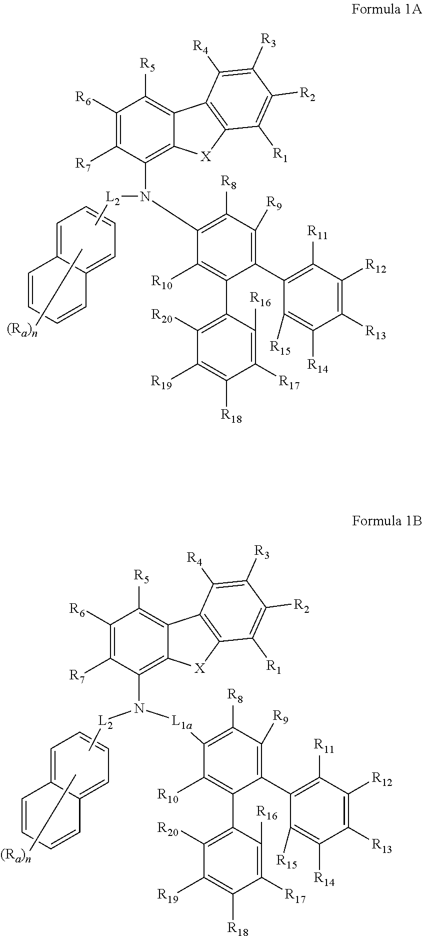

In Formula 1B, L₁ₐ may be a substituted or unsubstituted arylene group having 6 to 30 ring-forming carbon atoms or a substituted or unsubstituted heteroarylene group having 2 to 30 ring-forming carbon atoms, and R₈ and R₉ may not be bonded to an adjacent group to form (or provide) a ring, and in Formula 1A and Formula 1B, X, R₁ to R₂₀, n, Rₐ, and L₂ may each independently be the same as defined in Formula 1.

In one or more embodiments, the amine compound represented by Formula 1 may be represented by Formula 1-1 or Formula 1-2. In Formula 1-1 and Formula 1-2, R₁ to R₂₀, L₁, L₂, Rₐ, and n may each independently be the same as defined in Formula 1.

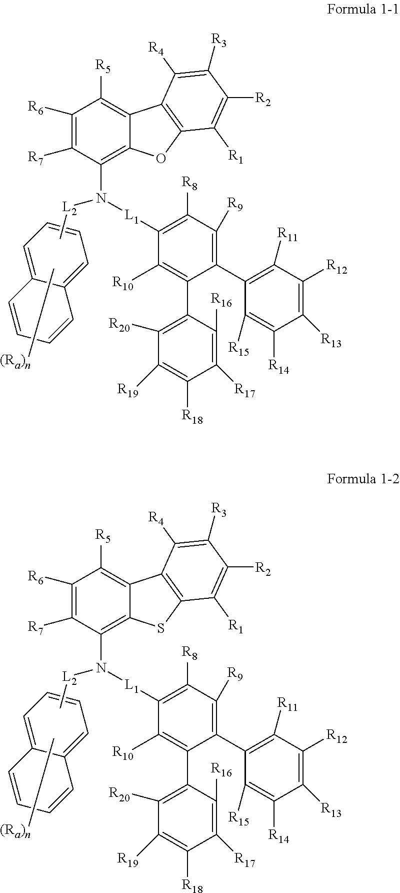

In one or more embodiments, L₂ may be a substituted or unsubstituted phenylene group or a substituted or unsubstituted divalent biphenyl group.

In one or more embodiments, n may be 1, and Rₐ may be a substituted or unsubstituted phenyl group or a substituted or unsubstituted naphthyl group.

In one or more embodiments, in Formula 1, at least one hydrogen may be substituted with deuterium.

In one or more embodiments, in Formula 1, R₁ to R₂₀, Rₐ, L₁, and L₂ may not include (e.g., may exclude) a substituted or unsubstituted amine group.

According to one or more embodiments of the present disclosure, a light emitting element includes: a first electrode; a second electrode on (e.g., arranged on) the first electrode; an emission layer between the first electrode and the second electrode; and a hole transport region between the first electrode and the emission layer and including the amine compound of one or more embodiments of the present disclosure.

In one or more embodiments, the hole transport region may include at least one of a hole injection layer, a hole transport layer, an electron blocking layer, or an emission-auxiliary layer, and at least one of the hole injection layer, the hole transport layer, the electron blocking layer, or the emission-auxiliary layer may include the amine compound of one or more embodiments of the present disclosure.

In one or more embodiments, the hole transport region may include a hole injection layer on (e.g., arranged on) the first electrode, and a hole transport layer on (e.g., arranged on) the hole injection layer, and the hole transport layer may include the amine compound of one or more embodiments of the present disclosure.

In one or more embodiments, the emission layer may include a compound represented by Formula E-1:

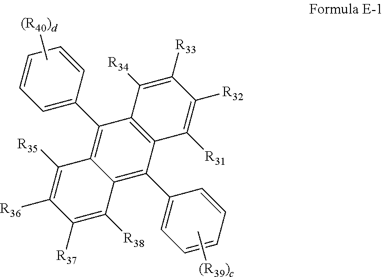

In Formula E-1, c and d may each independently be an integer of 0 to 5, and R₃₁ to R₄₀ may each independently be hydrogen, deuterium, a halogen, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or may independently be bonded to an adjacent group to form (or provide) a ring.

In one or more embodiments of the present disclosure, a display device includes a base layer; a circuit layer on (e.g., arranged on) the based layer; and a display element layer on e.g., arranged on) the circuit layer and includes a light emitting element, wherein the light emitting element includes a first electrode, a second electrode on (e.g., arranged on) the first electrode, an emission layer between the first electrode and the second electrode, and a hole transport region between the first electrode and the emission layer and including the amine compound of one or more embodiments of the present disclosure.

In one or more embodiments, the light emitting element may be to emit blue light.

In one or more embodiments, the display device may further include a light control layer including a quantum dot.

### BRIEF DESCRIPTION OF THE DRAWINGS

The accompanying drawings are included to provide a further understanding of the present disclosure, and are incorporated in and constitute a part of this disclosure. The drawings illustrate example embodiments of the present disclosure and, together with the description, serve to explain principles of the present disclosure. Above and/or other aspects of the present disclosure should become apparent and appreciated from the following description of embodiments taken in conjunction with the accompanying drawings. In the drawings:

FIG. 1 is a plan view illustrating a display device according to one or more embodiments of the present disclosure;

FIG. 2 is a cross-sectional view illustrating a part taken along the line I-I′ of FIG. 1;

FIG. 3 is a cross-sectional view schematically illustrating a light emitting element of one or more embodiments of the present disclosure;

FIG. 4 is a cross-sectional view schematically illustrating a light emitting element of one or more embodiments of the present disclosure;

FIG. 5 is a cross-sectional view schematically illustrating a light emitting element of one or more embodiments of the present disclosure;

FIG. 6 is a cross-sectional view schematically illustrating a light emitting element of one or more embodiments of the present disclosure;

FIG. 7 is a cross-sectional view schematically illustrating a light emitting element of one or more embodiments of the present disclosure;

FIG. 8 is a cross-sectional view illustrating a display device according to one or more embodiments of the present disclosure;

FIG. 9 is a cross-sectional view illustrating a display device according to one or more embodiments of the present disclosure;

FIG. 10 is a cross-sectional view illustrating a display device according to one or more embodiments of the present disclosure;

FIG. 11 is a cross-sectional view illustrating a display device according to one or more embodiments of the present disclosure; and

FIG. 12 is a view illustrating the inside of a vehicle in which display devices of embodiments are arranged according to one or more embodiments of the present disclosure.

### DETAILED DESCRIPTION

The present disclosure may be modified in one or more suitable manners and have many forms, and thus specific embodiments will be exemplified in the drawings and described in more detail in the detailed description of the present disclosure. It should be understood, however, that it is not intended to limit the present disclosure to the particular forms disclosed, but rather, is intended to cover all modifications, equivalents, and alternatives falling within the spirit and scope of the present disclosure.

When explaining each of drawings, like reference numbers are utilized for referring to like elements. In the accompanying drawings, the dimensions of each structure may be exaggeratingly illustrated for clarity of the present disclosure. It will be understood that, although the terms “first,” “second,” and/or the like, may be utilized herein to describe one or more suitable components, these components should not be limited by these terms. These terms are only utilized to distinguish one component from another. For example, a first component could be termed a second component, and, similarly, a second component could be termed a first component, without departing from the scope of example embodiments of the present disclosure. As utilized herein, the singular forms, “a,” “an,” “one,” and “the” are intended to include the plural forms as well, unless the context clearly indicates otherwise. Further, the utilization of “may” when describing embodiments of the present disclosure refers to “one or more embodiments of the present disclosure”.

In the present disclosure, it will be understood that the terms “comprise(s)/comprising,” “include(s)/including,” “have/has/having” and/or the like specify the presence of features, numbers, steps, operations, component, parts, or combinations thereof disclosed in the disclosure, but do not exclude the possibility of presence or addition of one or more other features, numbers, steps, operations, component, parts, or combinations thereof. As used herein, the terms “and,” “or,” and “and/or” may include any and all combinations of one or more of the associated listed items. Expressions such as “at least one of,” “one of,” and “selected from,” when preceding a list of elements, modify the entire list of elements and do not modify the individual elements of the list. For example, “at least one of a, b, or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

In the present disclosure, if (e.g., when) a layer, a film, a region, or a plate is referred to as being “on” or “in an upper portion of” another layer, film, region, or plate, it may be not only “directly on” the layer, film, region, or plate, but intervening layers, films, regions, or plates may also be present. Opposite this, if (e.g., when) a layer, a film, a region, or a plate is referred to as being “below”, “in a lower portion of” another layer, film, region, or plate, it may be not only directly under the layer, film, region, or plate, but intervening layers, films, regions, or plates may also be present. In one or more embodiments, it will be understood that if (e.g., when) a part is referred to as being “on” another part, it may be arranged above the other part, or arranged under the other part as well. In the present disclosure, “directly on” may refer to that there are no additional layers, films, regions, plates, etc., between a layer, a film, a region, a plate, and/or the like and the other part. For example, “directly on” may refer to two layers or two members are disposed without utilizing an additional member such as an adhesive member therebetween.

In the present disclosure, the term “substituted or unsubstituted” may refer to substituted or unsubstituted with at least one substituent selected from among the group consisting of deuterium, a halogen, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In one or more embodiments, each of the substituents exemplified above may be substituted or unsubstituted. For example, a biphenyl group may be interpreted as an aryl group or a phenyl group substituted with a phenyl group.

In the present disclosure, the phrase “bonded to an adjacent group to form (or provide) a ring” may refer to that a group is bonded to an adjacent group to form (or provide) a substituted or unsubstituted hydrocarbon ring or a substituted or unsubstituted heterocycle. The hydrocarbon ring may include an aliphatic hydrocarbon ring and/or an aromatic hydrocarbon ring. The heterocycle may include an aliphatic heterocycle and/or an aromatic heterocycle. The hydrocarbon ring and the heterocycle may each be monocyclic or polycyclic. In one or more embodiments, the rings formed by adjacent groups being bonded to each other may be connected to another ring to form (or provide) a spiro structure.

In the present disclosure, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other, and two ethyl groups in 1,1-diethylcyclopentane may be interpreted as “adjacent groups” to each other. In one or more embodiments, two methyl groups in 4,5-dimethylphenanthrene may be interpreted as “adjacent groups” to each other.

In the present disclosure, examples of a halogen may include fluorine, chlorine, bromine, or iodine.

In the present disclosure, an alkyl group may be linear or branched. The number of carbons in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-heneicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but embodiments of the present disclosure are not limited thereto.

In the present disclosure, a cycloalkyl group may refer to a cyclic alkyl group. The number of carbons in the cycloalkyl group may be 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but embodiments of the present disclosure are not limited thereto.

In the present disclosure, an alkenyl group refers to a hydrocarbon group including at least one carbon-carbon double bond in the middle or terminal of an alkyl group having 2 or more carbon atoms. The alkenyl group may be linear or branched. The number of carbon atoms in the alkenyl group is not specifically limited, for example, may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styryl vinyl group, and/or the like, but embodiments of the present disclosure are not limited thereto.

In the present disclosure, an alkynyl group refers to a hydrocarbon group including at least one carbon-carbon triple bond in the middle or terminal of an alkyl group having 2 or more carbon atoms. The alkynyl group may be linear or branched. Although the number of carbon atoms is not specifically limited, it may be 2 to 30, 2 to 20, or 2 to 10. Examples of the alkynyl group may include an ethynyl group, a propynyl group, and/or the like, but embodiments of the present disclosure are not limited thereto.

In the present disclosure, a hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the present disclosure, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and/or the like, but embodiments of the present disclosure are not limited thereto.

In the present disclosure, a fluorenyl group may be substituted, and two substituents may be bonded to each other to form (or provide) a spiro structure. Examples of the substituted fluorenyl group are as follows. However, embodiments of the present disclosure are not limited thereto.

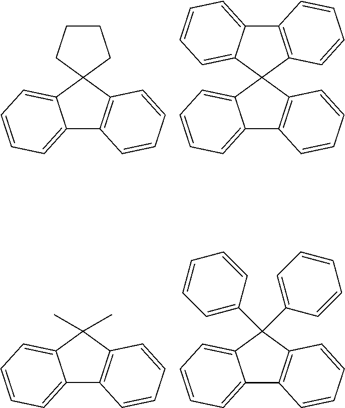

A heterocyclic group utilized herein refers to any functional group or substituent derived from a ring containing at least one of B, O, N, P, Si, or Se as a heteroatom. The heterocyclic group may include an aliphatic heterocyclic group and/or an aromatic heterocyclic group. The aromatic heterocyclic group may be a heteroaryl group. The aliphatic heterocycle and the aromatic heterocycle may each be monocyclic or polycyclic.

In the present disclosure, a heterocyclic group may contain at least one of B, O, N, P, Si or S as a heteroatom. If the heterocyclic group contains two or more heteroatoms, the two or more heteroatoms may be the same as or different from each other. The heterocyclic group may be a monocyclic heterocyclic group or a polycyclic heterocyclic group, and may include a heteroaryl group. The number of ring-forming carbon atoms in the heterocyclic group may be 2 to 30, 2 to 20, or 2 to 10.

In the present disclosure, an aliphatic heterocyclic group may include at least one of B, O, N, P, Si, or S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, but embodiments of the present disclosure are not limited thereto.

In the present disclosure, a heteroaryl group may contain at least one of B, O, N, P, Si, or S as a heteroatom. If the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and/or the like, but embodiments of the present disclosure are not limited thereto.

In the present disclosure, the above description of the aryl group may be applied to an arylene group except that the arylene group is a divalent group. The above description of the heteroaryl group may be applied to a heteroarylene group except that the heteroarylene group is a divalent group.

In the present disclosure, a silyl group may include an alkylsilyl group and/or an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, but embodiments of the present disclosure are not limited thereto.

In the present disclosure, the number of carbon atoms in a carbonyl group is not specifically limited, for example, may be 1 to 40, 1 to 30, or 1 to 20. For example, the carbonyl group may have the following structures, but embodiments of the present disclosure are not limited thereto.

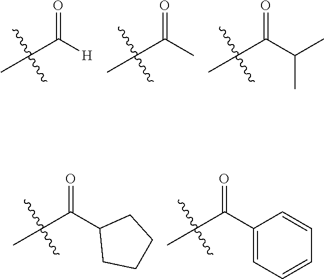

In the present disclosure, the number of carbon atoms in a sulfinyl group and/or a sulfonyl group is not particularly limited, for example, may be 1 to 30. The sulfinyl group may include an alkyl sulfinyl group and/or an aryl sulfinyl group. The sulfonyl group may include an alkyl sulfonyl group and/or an aryl sulfonyl group.

In the present disclosure, a thio group may include an alkylthio group and/or an arylthio group. The thio group may refer to that a sulfur atom is bonded to the alkyl group or the aryl group defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but embodiments of the present disclosure are not limited thereto.

In the present disclosure, an oxy group may refer to that an oxygen atom is bonded to the alkyl group or the aryl group defined above. The oxy group may include an alkoxy group and/or an aryl oxy group. The alkoxy group may be a linear chain, a branched chain, or a ring. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like, but embodiments of the present disclosure are not limited thereto.

A boron group utilized herein may refer to that a boron atom is bonded to the alkyl group or the aryl group defined above. The boron group may include an alkyl boron group and/or an aryl boron group. Examples of the boron group may include a dimethylboron group, a trimethylboron group, a t-butyldimethylboron group, a diphenylboron group, a phenylboron group, and/or the like, but embodiments of the present disclosure are not limited thereto.

In the present disclosure, the number of carbon atoms in an amine group is not specifically limited, for example, may be 1 to 30. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, and/or the like, but embodiments of the present disclosure are not limited thereto.

In the present disclosure, the alkyl group among an alkylthio group, an alkylsulfoxy group, an alkylaryl group, an alkylamino group, an alkyl boron group, an alkyl silyl group, and an alkyl amine group may be the same as the examples of the alkyl group described above.

In the present disclosure, the aryl group among an aryloxy group, an arylthio group, an arylsulfoxy group, an arylamino group, an arylboron group, an arylsilyl group, an arylamine group may be the same as the examples of the aryl group described above.

In the present disclosure, a direct linkage may refer to a single bond.

In the present disclosure,

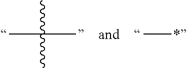

refer to a position to be connected.

Hereinafter, embodiments of the present disclosure will be described in more detail with reference to the accompanying drawings.

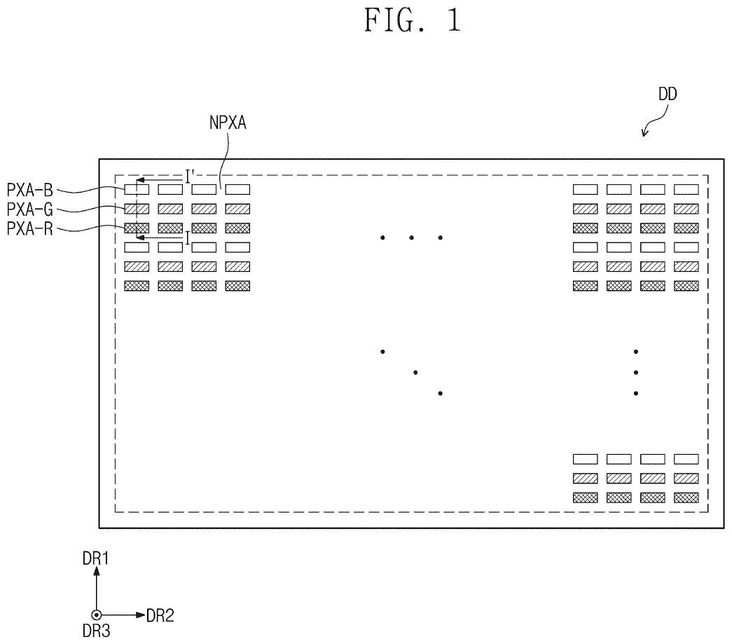

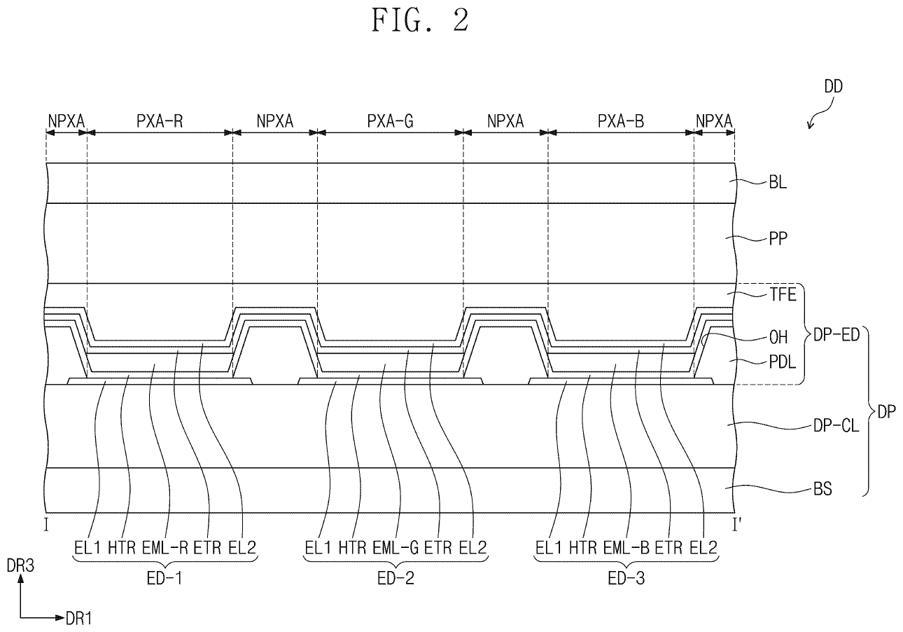

FIG. 1 is a plan view illustrating a display device DD according to one or more embodiments of the present disclosure. FIG. 2 is a cross-sectional view of the display device DD according to one or more embodiments. FIG. 2 is a cross-sectional view illustrating a part taken along the line I-I′ of FIG. 1.

The display device DD may include a display panel DP and an optical layer PP arranged on the display panel DP. The display panel DP may include light emitting elements ED-1, ED-2, and ED-3. The display device DD may include a plurality of light emitting elements ED-1, ED-2, and ED-3. The optical layer PP may be arranged on the display panel DP to control reflected light in the display panel DP due to external light. The optical layer PP may include, for example, a polarization layer and/or a color filter layer. In one or more embodiments, the optical layer PP may not be provided from the display device DD.

A base substrate BL may be arranged on the optical layer PP. The base substrate BL may be a member which provides a base surface on which the optical layer PP arranged. The base substrate BL may be a glass substrate, a metal substrate, a plastic substrate, and/or the like. However, embodiments of the present disclosure are not limited thereto, and the base substrate BL may be an inorganic layer, an organic layer, or a composite material layer. In one or more embodiments, the base substrate BL may not be provided.

The display device DD according to one or more embodiments may further include a filling layer. The filling layer may be arranged between a display element layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and a display element layer DP-ED. The display element layer DP-ED may include a pixel defining layer PDL, the light emitting elements ED-1, ED-2, and ED-3 arranged between respective portions of the pixel defining layer PDL, and an encapsulation layer TFE arranged on the light emitting elements ED-1, ED-2, and ED-3.

The base layer BS may be a member which provides a base surface on which the display element layer DP-ED is arranged. The base layer BS may be a glass substrate, a metal substrate, a plastic substrate, and/or the like. However, embodiments of the present disclosure are not limited thereto, and the base layer BS may be an inorganic layer, an organic layer, or a composite material layer.

In one or more embodiments, the circuit layer DP-CL may be arranged on the base layer BS, and the circuit layer DP-CL may include a plurality of transistors. Each of the transistors may include a control electrode, an input electrode, and an output electrode. For example, in some embodiments, the circuit layer DP-CL may include switching transistor(s) and driving transistor(s) for driving the light emitting elements ED-1, ED-2, and ED-3 of the display element layer DP-ED.

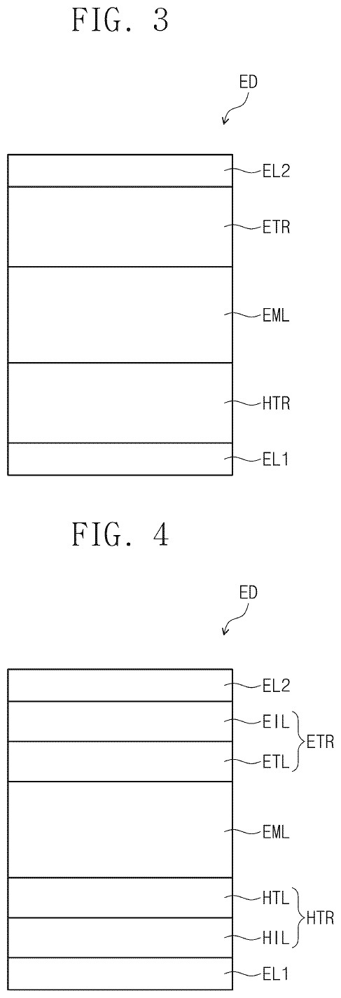

Each of the light emitting elements ED-1, ED-2, and ED-3 may have a structure of one of light emitting elements ED of embodiments according to FIGS. 3 to 7, which will be described later. Each of the light emitting elements ED-1, ED-2, and ED-3 may include a first electrode EL1, a hole transport region HTR, respective emission layers EML-R, EML-G, and EML-B, an electron transport region ETR, and a second electrode EL2.

FIG. 2 illustrates an embodiment in which the respective emission layers EML-R, EML-G, and EML-B of the light emitting elements ED-1, ED-2, and ED-3 are arranged in openings OH defined in the pixel defining layer PDL, and the hole transport region HTR, the electron transport region ETR, and the second electrode EL2 are provided as a common layer in the entire light emitting elements ED-1, ED-2, and ED-3. However, embodiments of the present disclosure are not limited thereto, for example, the hole transport region HTR and the electron transport region ETR in one or more embodiments may be provided by being patterned inside the openings OH defined in the pixel defining layer PDL. For example, the hole transport region HTR, the respective emission layers EML-R, EML-G, and EML-B, and the electron transport region ETR of the light emitting elements ED-1, ED-2, and ED-3 in one or more embodiments may be provided by being patterned in an inkjet printing method.

The encapsulation layer TFE may cover the light emitting elements ED-1, ED-2, and ED-3. The encapsulation layer TFE may seal the display element layer DP-ED. The encapsulation layer TFE may be a thin film encapsulation layer. The encapsulation layer TFE may be formed by laminating one layer or a plurality of layers. The encapsulation layer TFE may include at least one insulation layer. In some embodiments, the encapsulation layer TFE may include at least one inorganic film (hereinafter, an encapsulation-inorganic film). In some embodiments, the encapsulation layer TFE may include at least one organic film (hereinafter, an encapsulation-organic film) and at least one encapsulation-inorganic film.

The encapsulation-inorganic film protects the display element layer DP-ED from moisture/oxygen, and the encapsulation-organic film protects the display element layer DP-ED from foreign substances such as dust particles. The encapsulation-inorganic film may include silicon nitride, silicon oxynitride, silicon oxide, titanium oxide, aluminum oxide, and/or the like, but embodiments of the present disclosure are not particularly limited thereto. In some embodiments, the encapsulation-organic film may include an acrylic-based compound, an epoxy-based compound, and/or the like. In some embodiments, the encapsulation-organic film may include a photopolymerizable organic material, but embodiments of the present disclosure are not particularly limited thereto.

The encapsulation layer TFE may be arranged on the second electrode EL2 and may be arranged filling the opening OH.

Referring to FIG. 1 and FIG. 2, the display device DD may include a non-light emitting region NPXA and light emitting regions PXA-R, PXA-G, and PXA-B. The light emitting regions PXA-R, PXA-G, and PXA-B may be regions in which light generated by the respective light emitting elements ED-1, ED-2, and ED-3 is emitted. The light emitting regions PXA-R, PXA-G, and PXA-B may be spaced and/or apart from each other on a plane.

Each of the light emitting regions PXA-R, PXA-G, and PXA-B may be a region divided by the pixel defining layer PDL. The non-light emitting regions NPXA may be areas between adjacent light emitting regions PXA-R, PXA-G, and PXA-B, which correspond to the pixel defining layer PDL. In one or more embodiments, the light emitting regions PXA-R, PXA-G, and PXA-B may respectively correspond to pixels. The pixel defining layer PDL may divide the light emitting elements ED-1, ED-2, and ED-3. The respective emission layers EML-R, EML-G, and EML-B of the light emitting elements ED-1, ED-2, and ED-3 may be arranged in openings OH defined in the pixel defining layer PDL and separated from each other.

The light emitting regions PXA-R, PXA-G, and PXA-B may be divided into a plurality of groups according to the color of light generated from the light emitting elements ED-1, ED-2, and ED-3. In the display device DD of one or more embodiments illustrated in FIG. 1 and FIG. 2, three light emitting regions PXA-R, PXA-G, and PXA-B, which emit red light, green light, and blue light, respectively, are exemplarily illustrated. For example, in one or more embodiments, the display device DD may include the red light emitting region PXA-R, the green light emitting region PXA-G, and the blue light emitting region PXA-B that are separated from each other. In the display device DD according to one or more embodiments, the plurality of light emitting elements ED-1, ED-2, and ED-3 may be to emit light beams having wavelengths different from each other. For example, in some embodiments, the display device DD may include a first light emitting element ED-1 that emits red light, a second light emitting element ED-2 that emits green light, and a third light emitting element ED-3 that emits blue light. For example, in one or more embodiments, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, embodiments of the present disclosure are not limited thereto, and the first to third light emitting elements ED-1, ED-2, and ED-3 may be to emit light beams in substantially the same wavelength range or at least one light emitting element may be to emit a light beam in a wavelength range different from the others. For example, in some embodiments, the first to third light emitting elements ED-1, ED-2, and ED-3 may all emit blue light.

The light emitting regions PXA-R, PXA-G, and PXA-B in the display device DD according to one or more embodiments may be arranged in a stripe form. Referring to FIG. 1, a plurality of red light emitting regions PXA-R may be arranged with each other along a second direction axis DR2, a plurality of green light emitting regions PXA-G may be arranged with each other along the second direction axis DR2, and a plurality of blue light emitting regions PXA-B may be arranged with each other along the second direction axis DR2. In one or more embodiments, the red light emitting region PXA-R, the green light emitting region PXA-G, and the blue light emitting region PXA-B may be alternately arranged in the stated order along a first direction axis DR1.

FIG. 1 and FIG. 2 illustrate that all the light emitting regions PXA-R, PXA-G, and PXA-B have similar area, but embodiments of the present disclosure are not limited thereto. Thus, the light emitting regions PXA-R, PXA-G, and PXA-B may have different areas from each other according to the wavelength range of the emitted light. The areas of the light emitting regions PXA-R, PXA-G, and PXA-B may refer to areas if (e.g., when) viewed on a plane defined by the first direction axis DR1 and the second direction axis DR2 (e.g., the areas in a plan view).

In one or more embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality desired or required in the display device DD. For example, in one or more embodiments, the arrangement form of the light emitting regions PXA-R, PXA-G, and PXA-B may be a pentile (PENTILE®) arrangement form (for example, an RGBG matrix, an RGBG structure, or an RGBG matrix structure), or a diamond (Diamond Pixel™) arrangement form (e.g., a display (e.g., an OLED display) containing red, blue, and green (RGB) light-emitting regions arranged in the shape of diamonds). PENTILE® is a duly registered trademark of Samsung Display Co., Ltd. Diamond Pixel™ is a trademark of Samsung Display Co., Ltd.

In one or more embodiments, the areas of the light emitting regions PXA-R, PXA-G, and PXA-B may be different from each other. For example, in an embodiment, the area of the green light emitting region PXA-G may be smaller than that of the blue light emitting region PXA-B, but embodiments of the present disclosure are not limited thereto.

Hereinafter, FIGS. 3 to 7 are each a cross-sectional view schematically illustrating a light emitting element according to one or more embodiments. Each of the light emitting elements ED according to one or more embodiments may include a first electrode EL1, a hole transport region HTR, an emission layer EML, an electron transport region ETR, and a second electrode EL2 that are sequentially stacked.

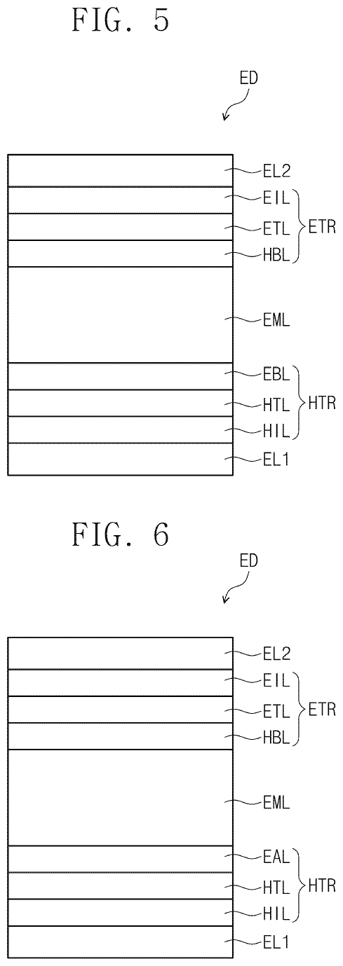

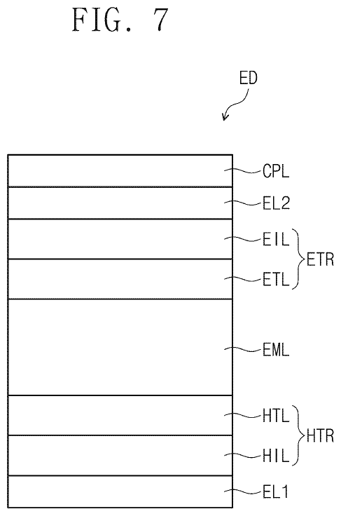

Compared with FIG. 3, FIG. 4 illustrates a cross-sectional view of a light emitting element ED of one or more embodiments, in which a hole transport region HTR includes a hole injection layer HIL and a hole transport layer HTL, and an electron transport region ETR includes an electron injection layer EIL and an electron transport layer ETL. Compared with FIG. 3, FIG. 5 illustrates a cross-sectional view of a light emitting element ED of one or more embodiments, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. As compared with FIG. 3, FIG. 6 illustrates a cross-sectional view of a light emitting element ED of one or more embodiments, in which a hole transport region HTR includes a hole injection layer HIL, a hole transport layer HTL, and an emission-auxiliary layer EAL, and an electron transport region ETR includes an electron injection layer EIL, an electron transport layer ETL, and a hole blocking layer HBL. Compared with FIG. 4, FIG. 7 illustrates a cross-sectional view of a light emitting element ED of one or more embodiments including a capping layer CPL arranged on a second electrode EL2.

The first electrode EL1 has conductivity (e.g., is a conductor). The first electrode EL1 may be formed of a metal material, a metal alloy, or a conductive compound. The first electrode EL1 may be an anode or a cathode. However, embodiments of the present disclosure are not limited thereto. In one or more embodiments, the first electrode EL1 may be a pixel electrode. The first electrode EL1 may be a transmissive electrode, a transflective electrode, or a reflective electrode. The first electrode EL1 may include at least one selected from among silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), lithium fluoride (LiF), molybdenum (Mo), titanium (Ti), tungsten (W,) indium (In), tin (Sn), and zinc (Zn), a compound of two or more selected therefrom (thereof), a mixture of two or more selected therefrom, or an oxide thereof.

If the first electrode EL1 is a transmissive electrode, the first electrode EL1 may include a transparent metal oxide such as indium tin oxide (ITO), indium zinc oxide (IZO), zinc oxide (ZnO), and/or indium tin zinc oxide (ITZO). If the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound thereof, or a mixture thereof (e.g., a mixture of Ag and Mg). In one or more embodiments, the first electrode EL1 may have a multilayer structure including a reflective film or a transflective film formed of one or more of the above-described materials, and a transparent conductive film formed of ITO, IZO, ZnO, ITZO, and/or the like. For example, in some embodiments, the first electrode EL1 may have a three-layer structure of ITO/Ag/ITO, but embodiments of the present disclosure are not limited thereto. In one or more embodiments, the first electrode EL1 may include one of the above-described metal materials, combinations of at least two metal materials of the above-described metal materials, oxides of the above-described metal materials, and/or the like. A thickness of the first electrode EL1 may be from about 700 angstrom (Å) to about 10,000 Å. For example, in one or more embodiments, the thickness of the first electrode EL1 may be from about 1,000 Å to about 3,000 Å.

The hole transport region HTR may be provided on the first electrode EL1. The hole transport region HTR may be arranged between the first electrode EL1 and the emission layer EML.

The hole transport region HTR may include at least one of a hole injection layer HIL, a hole transport layer HTL, an emission-auxiliary layer EAL, or an electron blocking layer EBL. The emission-auxiliary layer EAL may be referred to as a buffer layer. A thickness of the hole transport region HTR may be, for example, from about 50 Å to about 15,000 Å.

The hole transport region HTR may have a single layer formed of a single material, a single layer formed of a plurality of different materials, or a multilayer structure including a plurality of layers formed of a plurality of different materials.

For example, in one or more embodiments, the hole transport region HTR may have a single layer structure of a hole injection layer HIL or a hole transport layer HTL, or may have a single layer structure formed of a hole injection material and a hole transport material. In one or more embodiments, the hole transport region HTR may have a single layer structure formed of a plurality of different materials, or a structure in which a hole injection layer HIL/hole transport layer HTL, a hole injection layer HIL/hole transport layer HTL/emission-auxiliary layer EAL, a hole injection layer HIL/emission-auxiliary layer EAL, a hole transport layer HTL/emission-auxiliary layer EAL, or a hole injection layer HIL/hole transport layer HTL/electron blocking layer EBL are stacked in order (e.g., in the stated order) from the first electrode EL1, but embodiments of the present disclosure are not limited thereto. In one or more embodiments, the hole transport layer HTL may have a single layer or a multilayer structure having a plurality of layers.

The hole transport region HTR may be formed utilizing one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, the light emitting element ED may include the amine compound represented by Formula 1 of one or more embodiments in the hole transport region HTR. In one or more embodiments, at least one of the hole injection layer HIL, the hole transport layer HTL, the electron blocking layer EBL, or the emission-auxiliary layer EAL may include the amine compound represented by Formula 1 of one or more embodiments. For example, the hole transport layer HTL in the light emitting element ED of one or more embodiments may include the amine compound of one or more embodiments.

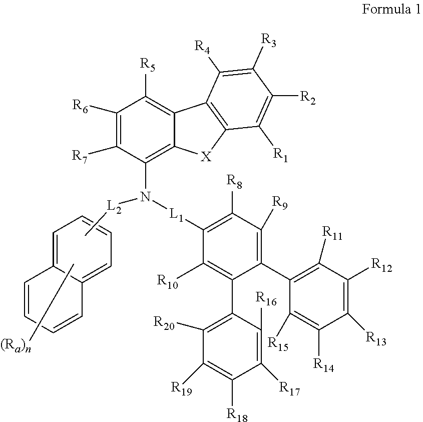

The amine compound of one or more embodiments may include all of a dibenzoheterole group, a naphthyl group, and a terphenyl group bonded to the nitrogen atom (N) of the amine compound. The amine compound of one or more embodiments may include a substituted or unsubstituted dibenzoheterole group directly bonded to the nitrogen atom (N), a substituted or unsubstituted naphthyl group bonded to the nitrogen atom via a linker, and a substituted or unsubstituted ortho-terphenyl group directly bonded to the nitrogen atom or bonded via a linker. In one or more embodiments, the amine compound may be a monoamine compound that does not contain an additional amine substituent.

The substituted or unsubstituted dibenzoheterole group in the amine compound of one or more embodiments may be directly bonded to the nitrogen atom at position c1 adjacent to a heteroatom X as indicated herein. In the present disclosure, the dibenzoheterole group, which is a configuration bonded to the nitrogen atom of the amine compound, may be referred to as a 4-dibenzoheterole group.

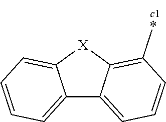

In one or more embodiments, the substituted or unsubstituted ortho-terphenyl group in the amine compound may be directly bonded to the nitrogen atom at position c2 as indicated herein or bonded to the nitrogen atom via a linker. In the present disclosure, the ortho-terphenyl group, which is a configuration bonded to the nitrogen atom of the amine compound, may be referred to as a 3,4-substituted phenyl group.

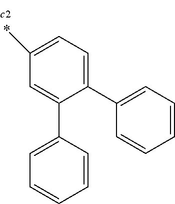 [SMILES: *c1ccc(-c2ccccc2)c(-c2ccccc2)c1]

The amine compound of one or more embodiments may include the 4-dibenzoheterole group and the naphthyl group, and thus may exhibit the effects of excellent or suitable charge transport properties and excellent or suitable electron resistance. In addition, the amine compound according to one or more embodiments may include the 3,4-substituted phenyl group to adjust charge balance by a steric effect, thereby exhibiting excellent or suitable charge transport properties. For example, the amine compound of one or more embodiments includes the dibenzoheterole group and the ortho-terphenyl group that are bonded to the nitrogen atom at a specified position, and further includes the naphthyl group that is bonded to the nitrogen atom, thereby having excellent or suitable charge transport properties and material stability, and thus contributing to high efficiency and long service life of the light emitting element.

In Formula 1, X may be O or S. For example, in the amine compound of one or more embodiments, the dibenzoheterole group bonded to the nitrogen atom of the amine compound may be a substituted or unsubstituted dibenzofuran group or a substituted or unsubstituted dibenzothiophene group.

In Formula 1, R₁ to R₂₀ may each independently be hydrogen, deuterium, a halogen, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or may be independently bonded to an adjacent group to form (or provide) a ring.

When one or more of R₁ to R₂₀ are bonded to an adjacent group to form (or provide) a ring, the one or more of R₁ to R₂₀ may be bonded to an adjacent group to form (or provide) a saturated hydrocarbon ring or an unsaturated hydrocarbon ring. For example, one or more of R₁ to R₂₀ may be bonded to the substituted ring to form (or provide) a fused ring.

In one or more embodiments, R₁ to R₇ may all be hydrogens, or at least one thereof may be a substituted or unsubstituted aryl group, and/or may be bonded to an adjacent group to form (or provide) a ring. For example, in one or more embodiments, R₁ to R₇ may all be hydrogen atoms, or at least one thereof may be a substituted or unsubstituted phenyl group, and/or may be bonded to a benzene ring of an adjacent dibenzoheterole group to form (or provide) a fused ring. However, embodiments of the present disclosure are not limited thereto.

In one or more embodiments, R₈ to R₂₀ may all be hydrogens, or at least one thereof may be a substituted or unsubstituted alkyl group, a substituted or unsubstituted aryl group, or a halogen, and/or may be bonded to an adjacent group to form (or provide) a ring. For example, in one or more embodiments, R₈ to R₂₀ may all be hydrogens, or at least one thereof may be a substituted or unsubstituted phenyl group, or at least one thereof may be an alkyl group having 1 to 5 carbon atoms, or at least one thereof may be a halogen, and/or may be bonded to an adjacent benzene ring to form (or provide) a fused ring. However, embodiments of the present disclosure are not limited thereto.

In Formula 1, L₁ may be a direct linkage, a substituted or unsubstituted arylene group having 6 to 30 ring-forming carbon atoms, or a substituted or unsubstituted heteroarylene group having 2 to 30 ring-forming carbon atoms. For example, in one or more embodiments, in Formula 1, L₁ may be a direct linkage or a substituted or unsubstituted arylene group.

In Formula 1, L₂ may be a substituted or unsubstituted arylene group having 6 to 30 ring-forming carbon atoms. In one or more embodiments, L₂ may be a substituted or unsubstituted phenylene group or a substituted or unsubstituted divalent biphenyl group. However, embodiments of the present disclosure are not limited thereto.

In Formula 1, n may be an integer of 1 to 7, and Rₐ may be hydrogen, deuterium, or a substituted or unsubstituted aryl group having 6 to 30 ring-forming carbon atoms. For example, in one or more embodiments, n may be 1 and Rₐ may be a substituted or unsubstituted phenyl group or a substituted or unsubstituted naphthyl group. However, embodiments of the present disclosure are not limited thereto.

In one or more embodiments, in the amine compound represented by Formula 1, at least one hydrogen may be substituted with deuterium. In Formula 1, R₁ to R₂₀ may each independently be deuterium or include deuterium as a substituent. In one or more embodiments, L₁ and L₂ may each independently include deuterium as a substituent.

In Formula 1, R₁ to R₂₀, Rₐ, L₁, and L₂ may not include (e.g., may exclude) a substituted or unsubstituted amine group. For example, in one or more embodiments, the amine compound represented by Formula 1 may be a monoamine compound that does not further include an amine group.

In one or more embodiments, the amine compound represented by Formula 1 may be represented by Formula 1-1 or Formula 1-2. Formula 1-1 represents embodiments of the amine compound including dibenzofuran as the dibenzoheterole group, and Formula 1-2 represents embodiments of the amine compound including dibenzothiophene as the dibenzoheterole group.

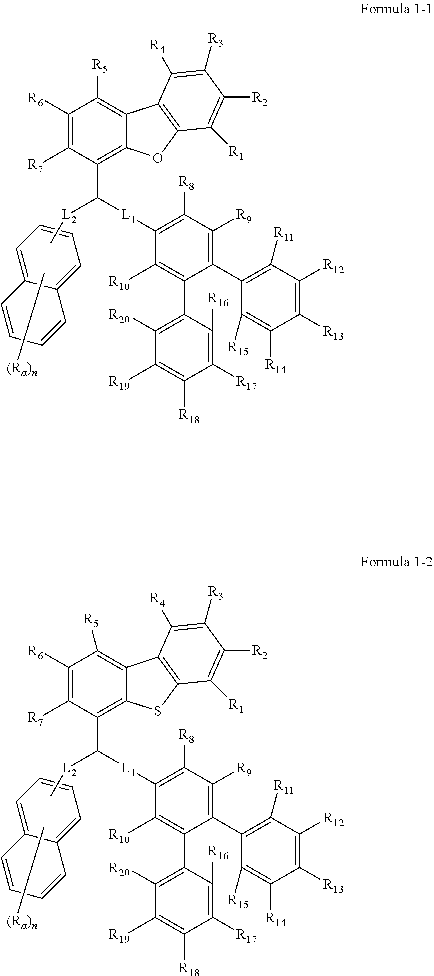

In Formula 1-1 and Formula 1-2, the same as described in Formula 1 may be applied to R₁ to R₂₀, L₁, L₂, Rₐ, and n.

In the amine compound represented by Formula 1 of one or more embodiments, the ortho-terphenyl group may be directly bonded to the nitrogen atom of the amine or bonded to the nitrogen atom via a linker.

In one or more embodiments, the amine compound represented by Formula 1 may be represented by Formula 1A or Formula 1B. Formula 1A shows the amine compound of one or more embodiments in which the ortho-terphenyl group is directly bonded to the nitrogen atom of the amine compound, and Formula 1B shows the amine compound of one or more embodiments in which the ortho-terphenyl group is bonded to the nitrogen atom via L₁ₐ, which is a linker.

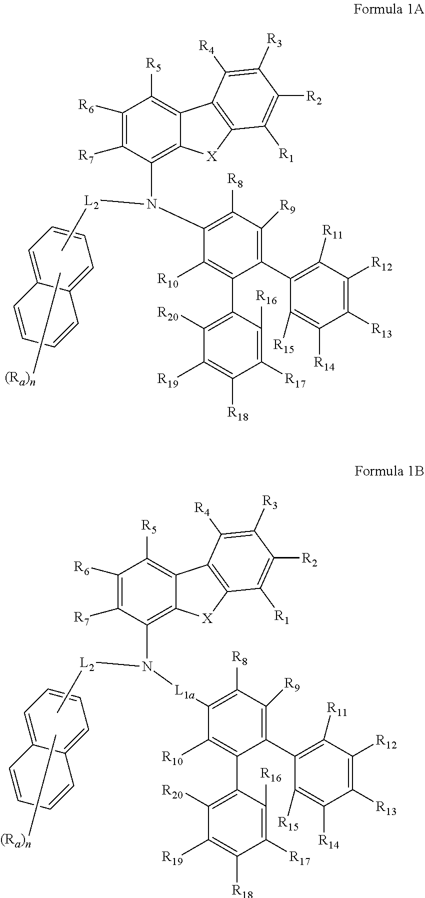

In Formula 1B, L₁ₐ may be a substituted or unsubstituted arylene group having 6 to 30 ring-forming carbon atoms or a substituted or unsubstituted heteroarylene group having 2 to 30 ring-forming carbon atoms, and R₈ and R₉ may not be bonded to an adjacent group to form (or provide) a ring.

In one or more embodiments, in Formula 1A and Formula 1B, the same as described in Formula 1 may be applied to X, R₁ to R₂₀, n, Rₐ, and L₂.

The amine compound represented by Formula 1 of one or more embodiments may be one selected from among compounds of Compound Group 1. The hole transport region HTR of the light emitting element ED of one or more embodiments may include at least one selected from among the amine compounds disclosed in Compound Group 1. D in Compound Group 1 is deuterium.

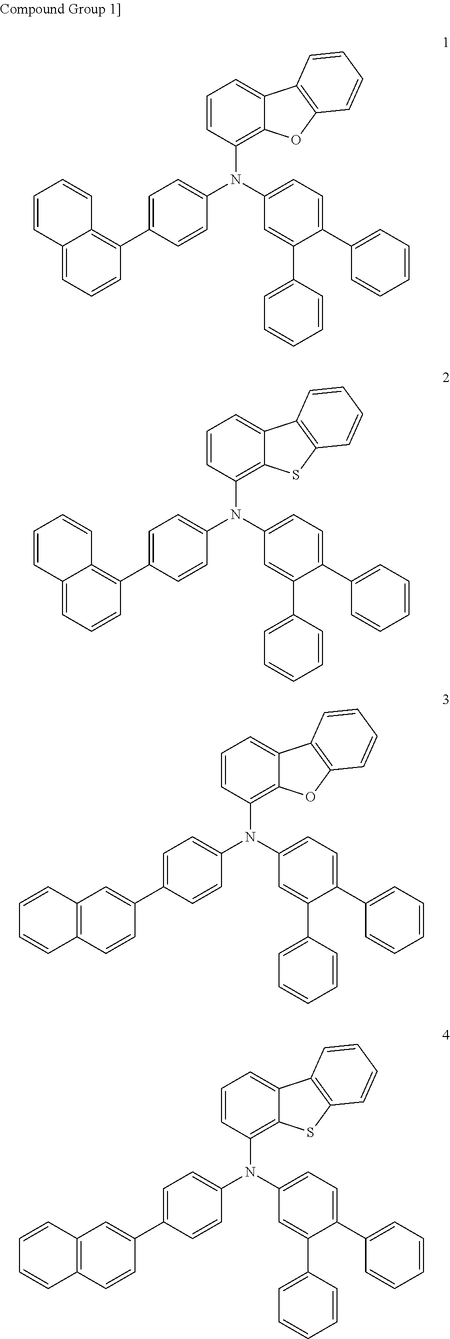

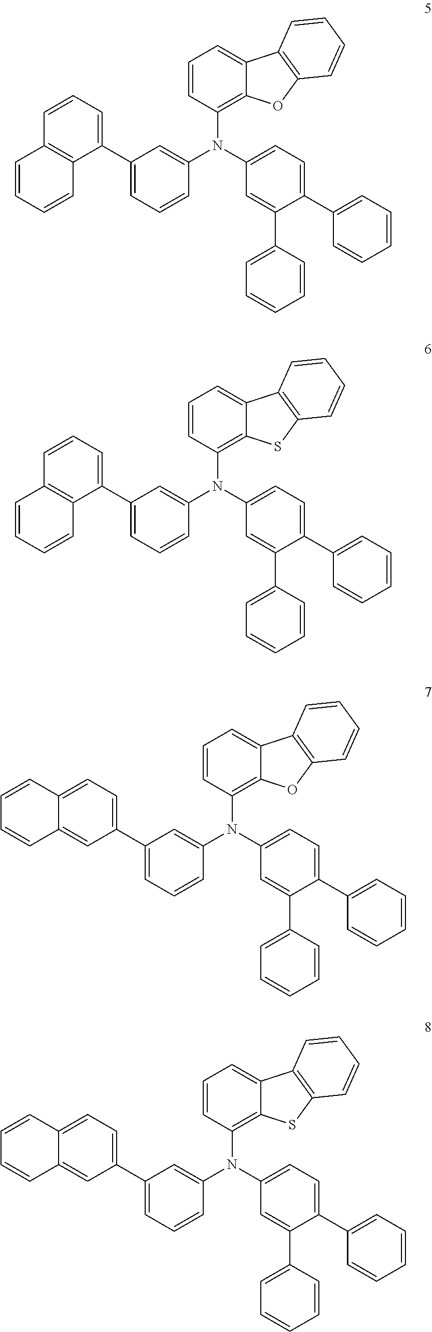 [SMILES: c1ccc(-c2ccc(N(c3cccc(-c4cccc5ccccc45)c3)c3cccc4c3oc3ccccc34)cc2-c2ccccc2)cc1 ; c1ccc(-c2ccc(N(c3cccc(-c4cccc5ccccc45)c3)c3cccc4c3sc3ccccc34)cc2-c2ccccc2)cc1 ; c1ccc(-c2ccc(N(c3cccc(-c4ccc5ccccc5c4)c3)c3cccc4c3oc3ccccc34)cc2-c2ccccc2)cc1 ; c1ccc(-c2ccc(N(c3cccc(-c4ccc5ccccc5c4)c3)c3cccc4c3sc3ccccc34)cc2-c2ccccc2)cc1]

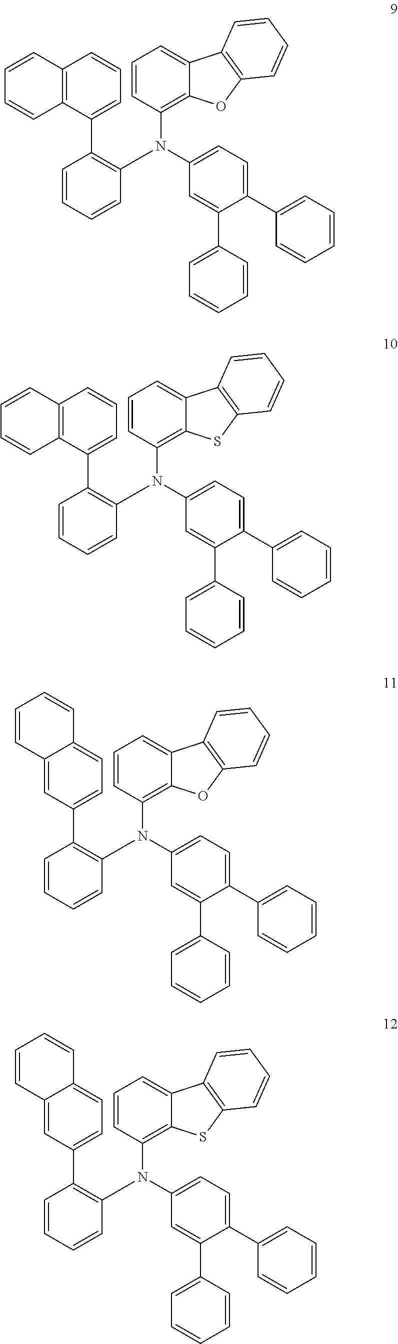

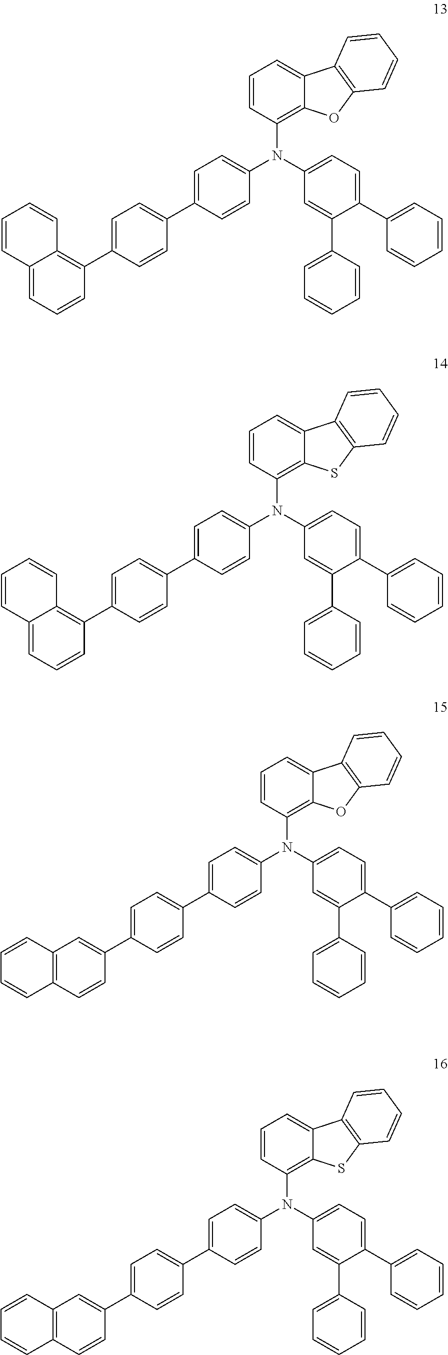 [SMILES: c1ccc(-c2ccc(N(c3ccc(-c4ccc(-c5cccc6ccccc56)cc4)cc3)c3cccc4c3oc3ccccc34)cc2-c2ccccc2)cc1 ; c1ccc(-c2ccc(N(c3ccc(-c4ccc(-c5cccc6ccccc56)cc4)cc3)c3cccc4c3sc3ccccc34)cc2-c2ccccc2)cc1 ; c1ccc(-c2ccc(N(c3ccc(-c4ccc(-c5ccc6ccccc6c5)cc4)cc3)c3cccc4c3oc3ccccc34)cc2-c2ccccc2)cc1 ; c1ccc(-c2ccc(N(c3ccc(-c4ccc(-c5ccc6ccccc6c5)cc4)cc3)c3cccc4c3sc3ccccc34)cc2-c2ccccc2)cc1]

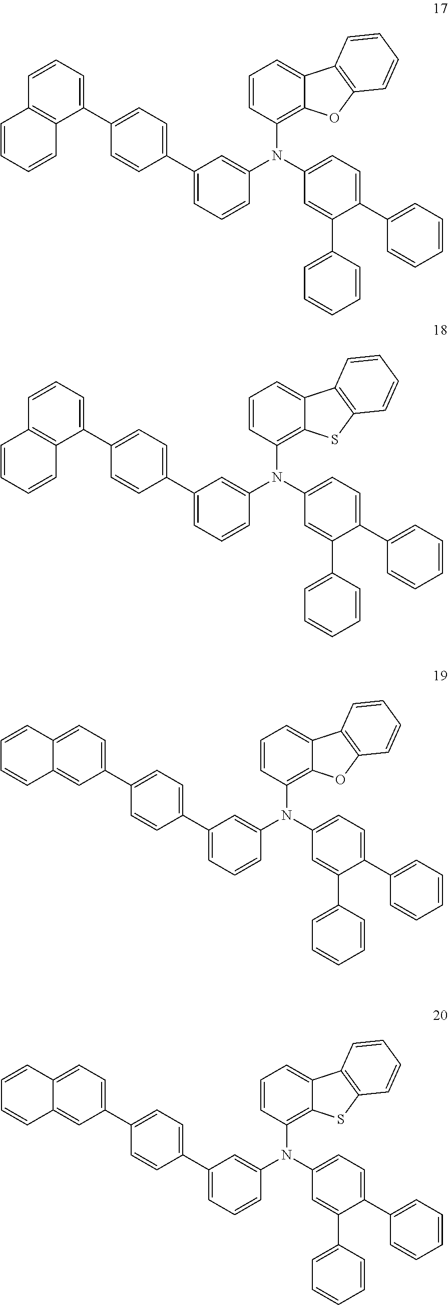 [SMILES: c1ccc(-c2ccc(N(c3cccc(-c4ccc(-c5cccc6ccccc56)cc4)c3)c3cccc4c3oc3ccccc34)cc2-c2ccccc2)cc1 ; c1ccc(-c2ccc(N(c3cccc(-c4ccc(-c5cccc6ccccc56)cc4)c3)c3cccc4c3sc3ccccc34)cc2-c2ccccc2)cc1 ; c1ccc(-c2ccc(N(c3cccc(-c4ccc(-c5ccc6ccccc6c5)cc4)c3)c3cccc4c3oc3ccccc34)cc2-c2ccccc2)cc1 ; c1ccc(-c2ccc(N(c3cccc(-c4ccc(-c5ccc6ccccc6c5)cc4)c3)c3cccc4c3sc3ccccc34)cc2-c2ccccc2)cc1]

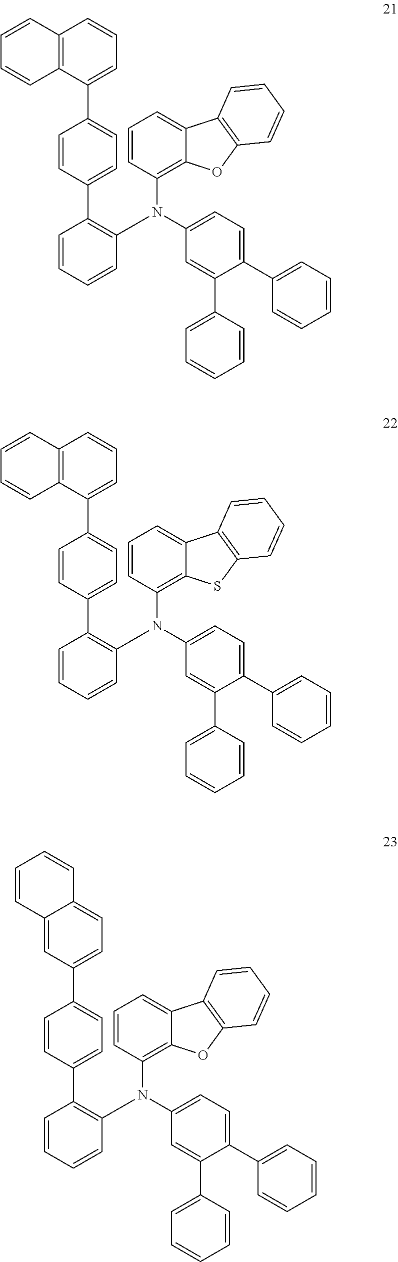 [SMILES: c1ccc(-c2ccc(N(c3ccccc3-c3ccc(-c4cccc5ccccc45)cc3)c3cccc4c3oc3ccccc34)cc2-c2ccccc2)cc1 ; c1ccc(-c2ccc(N(c3ccccc3-c3ccc(-c4cccc5ccccc45)cc3)c3cccc4c3sc3ccccc34)cc2-c2ccccc2)cc1 ; c1ccc(-c2ccc(N(c3ccccc3-c3ccc(-c4ccc5ccccc5c4)cc3)c3cccc4c3oc3ccccc34)cc2-c2ccccc2)cc1]

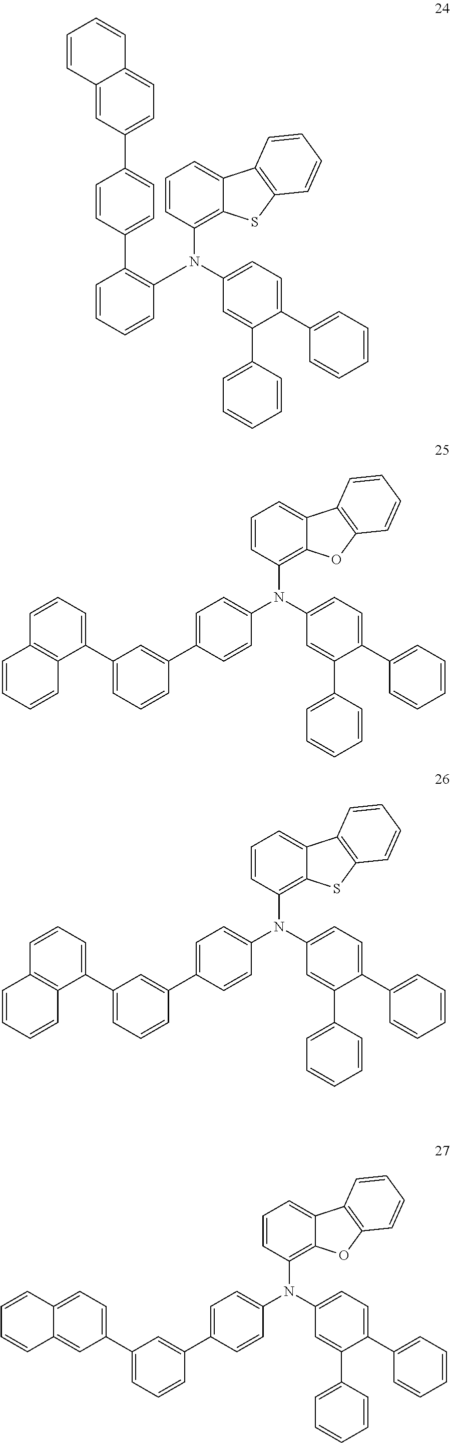 [SMILES: c1ccc(-c2ccc(N(c3ccccc3-c3ccc(-c4ccc5ccccc5c4)cc3)c3cccc4c3sc3ccccc34)cc2-c2ccccc2)cc1 ; c1ccc(-c2ccc(N(c3ccc(-c4cccc(-c5cccc6ccccc56)c4)cc3)c3cccc4c3oc3ccccc34)cc2-c2ccccc2)cc1 ; c1ccc(-c2ccc(N(c3ccc(-c4cccc(-c5cccc6ccccc56)c4)cc3)c3cccc4c3sc3ccccc34)cc2-c2ccccc2)cc1 ; c1ccc(-c2ccc(N(c3ccc(-c4cccc(-c5ccc6ccccc6c5)c4)cc3)c3cccc4c3oc3ccccc34)cc2-c2ccccc2)cc1]

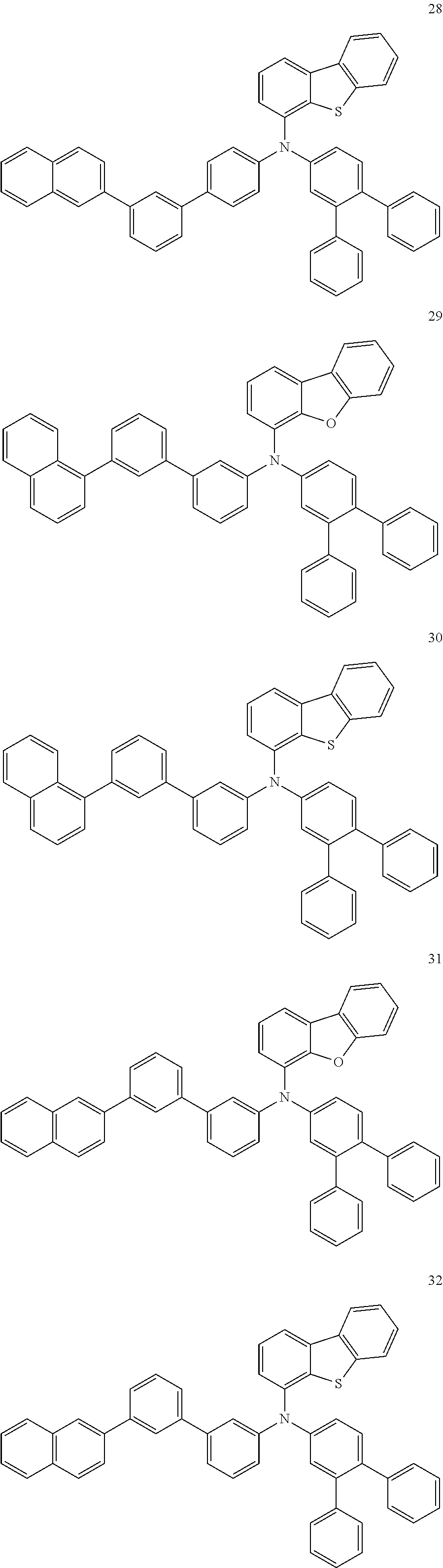 [SMILES: c1ccc(-c2ccc(N(c3ccc(-c4cccc(-c5ccc6ccccc6c5)c4)cc3)c3cccc4c3sc3ccccc34)cc2-c2ccccc2)cc1 ; c1ccc(-c2ccc(N(c3cccc(-c4cccc(-c5cccc6ccccc56)c4)c3)c3cccc4c3oc3ccccc34)cc2-c2ccccc2)cc1 ; c1ccc(-c2ccc(N(c3cccc(-c4cccc(-c5cccc6ccccc56)c4)c3)c3cccc4c3sc3ccccc34)cc2-c2ccccc2)cc1 ; c1ccc(-c2ccc(N(c3cccc(-c4cccc(-c5ccc6ccccc6c5)c4)c3)c3cccc4c3oc3ccccc34)cc2-c2ccccc2)cc1 ; c1ccc(-c2ccc(N(c3cccc(-c4cccc(-c5ccc6ccccc6c5)c4)c3)c3cccc4c3sc3ccccc34)cc2-c2ccccc2)cc1]

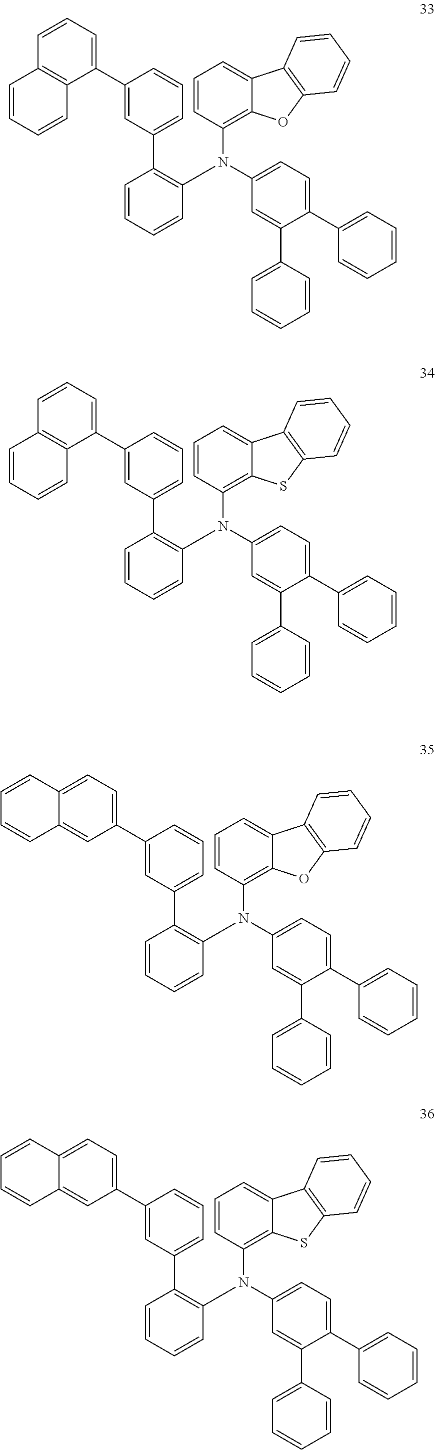 [SMILES: c1ccc(-c2ccc(N(c3ccccc3-c3cccc(-c4cccc5ccccc45)c3)c3cccc4c3oc3ccccc34)cc2-c2ccccc2)cc1 ; c1ccc(-c2ccc(N(c3ccccc3-c3cccc(-c4cccc5ccccc45)c3)c3cccc4c3sc3ccccc34)cc2-c2ccccc2)cc1 ; c1ccc(-c2ccc(N(c3ccccc3-c3cccc(-c4ccc5ccccc5c4)c3)c3cccc4c3oc3ccccc34)cc2-c2ccccc2)cc1 ; c1ccc(-c2ccc(N(c3ccccc3-c3cccc(-c4ccc5ccccc5c4)c3)c3cccc4c3sc3ccccc34)cc2-c2ccccc2)cc1]

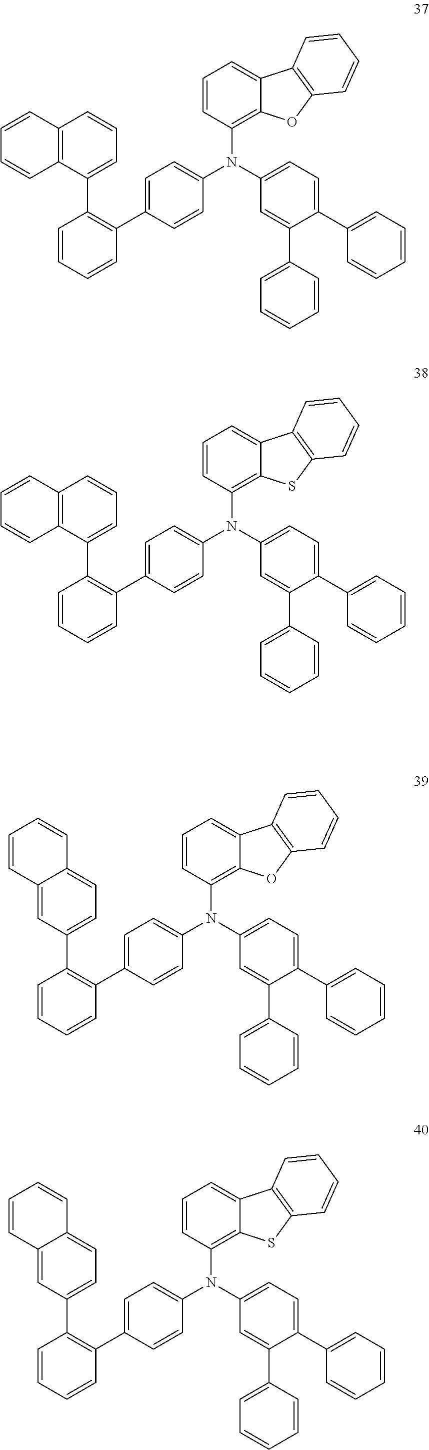 [SMILES: c1ccc(-c2ccc(N(c3ccc(-c4ccccc4-c4cccc5ccccc45)cc3)c3cccc4c3oc3ccccc34)cc2-c2ccccc2)cc1 ; c1ccc(-c2ccc(N(c3ccc(-c4ccccc4-c4cccc5ccccc45)cc3)c3cccc4c3sc3ccccc34)cc2-c2ccccc2)cc1 ; c1ccc(-c2ccc(N(c3ccc(-c4ccccc4-c4ccc5ccccc5c4)cc3)c3cccc4c3oc3ccccc34)cc2-c2ccccc2)cc1 ; c1ccc(-c2ccc(N(c3ccc(-c4ccccc4-c4ccc5ccccc5c4)cc3)c3cccc4c3sc3ccccc34)cc2-c2ccccc2)cc1]

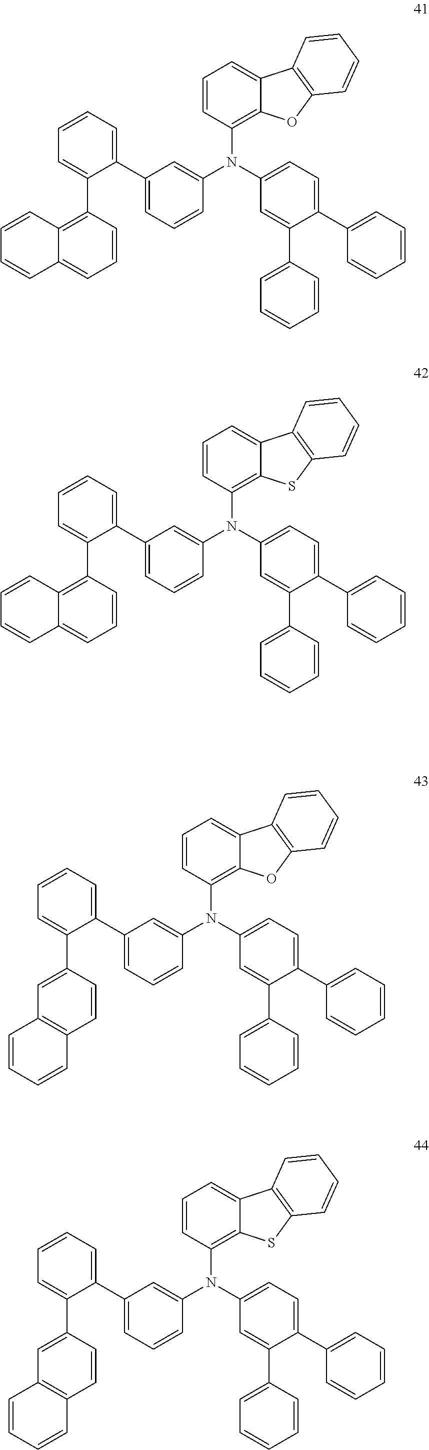 [SMILES: c1ccc(-c2ccc(N(c3cccc(-c4ccccc4-c4cccc5ccccc45)c3)c3cccc4c3oc3ccccc34)cc2-c2ccccc2)cc1 ; c1ccc(-c2ccc(N(c3cccc(-c4ccccc4-c4cccc5ccccc45)c3)c3cccc4c3sc3ccccc34)cc2-c2ccccc2)cc1 ; c1ccc(-c2ccc(N(c3cccc(-c4ccccc4-c4ccc5ccccc5c4)c3)c3cccc4c3oc3ccccc34)cc2-c2ccccc2)cc1 ; c1ccc(-c2ccc(N(c3cccc(-c4ccccc4-c4ccc5ccccc5c4)c3)c3cccc4c3sc3ccccc34)cc2-c2ccccc2)cc1]

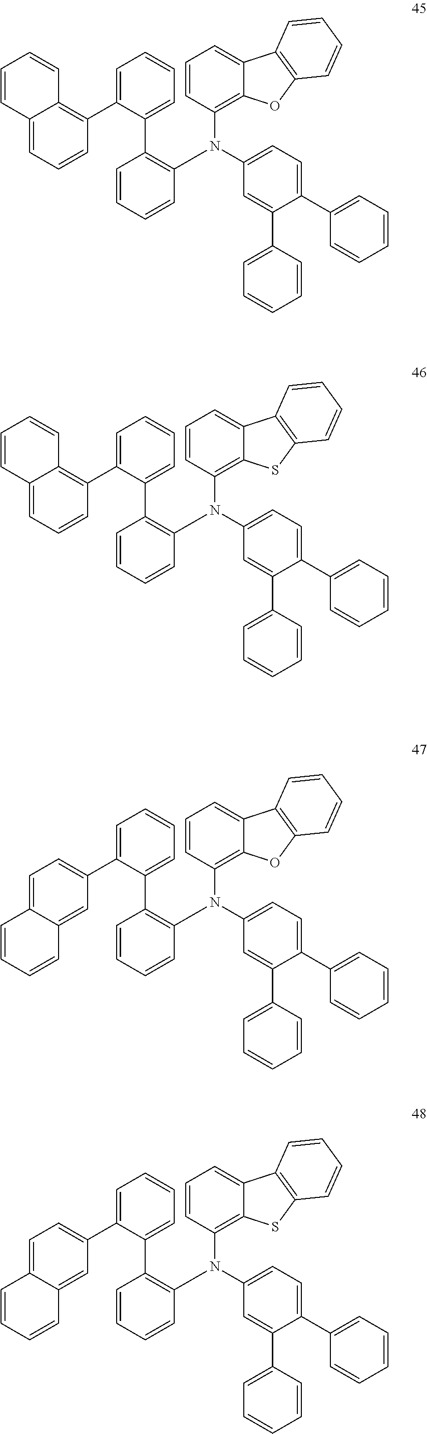 [SMILES: c1ccc(-c2ccc(N(c3ccccc3-c3ccccc3-c3cccc4ccccc34)c3cccc4c3oc3ccccc34)cc2-c2ccccc2)cc1 ; c1ccc(-c2ccc(N(c3ccccc3-c3ccccc3-c3cccc4ccccc34)c3cccc4c3sc3ccccc34)cc2-c2ccccc2)cc1 ; c1ccc(-c2ccc(N(c3ccccc3-c3ccccc3-c3ccc4ccccc4c3)c3cccc4c3oc3ccccc34)cc2-c2ccccc2)cc1 ; c1ccc(-c2ccc(N(c3ccccc3-c3ccccc3-c3ccc4ccccc4c3)c3cccc4c3sc3ccccc34)cc2-c2ccccc2)cc1]

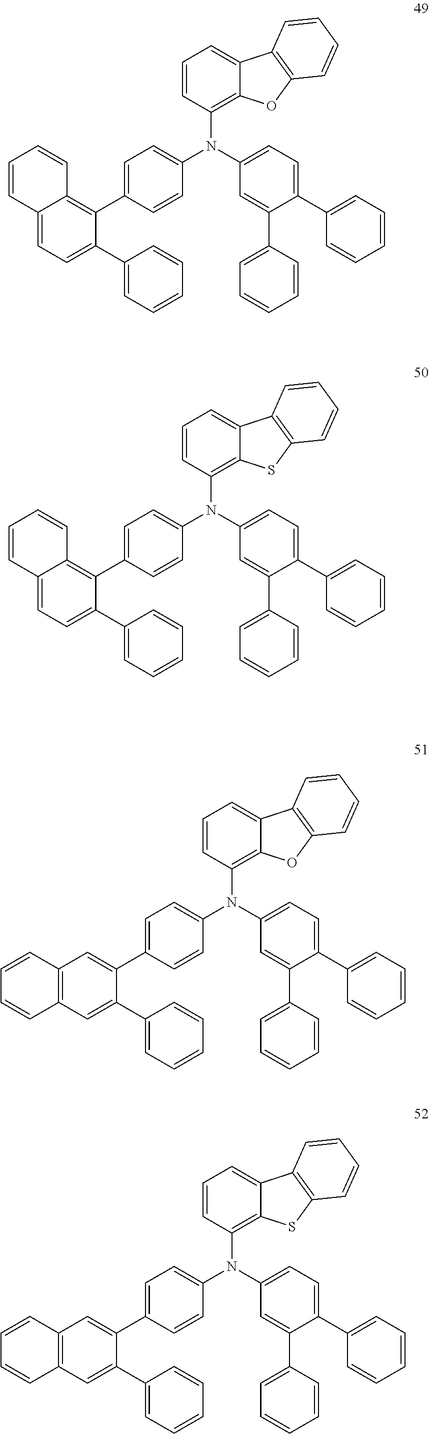 [SMILES: c1ccc(-c2ccc(N(c3ccc(-c4c(-c5ccccc5)ccc5ccccc45)cc3)c3cccc4c3oc3ccccc34)cc2-c2ccccc2)cc1 ; c1ccc(-c2ccc(N(c3ccc(-c4c(-c5ccccc5)ccc5ccccc45)cc3)c3cccc4c3sc3ccccc34)cc2-c2ccccc2)cc1 ; c1ccc(-c2ccc(N(c3ccc(-c4cc5ccccc5cc4-c4ccccc4)cc3)c3cccc4c3oc3ccccc34)cc2-c2ccccc2)cc1 ; c1ccc(-c2ccc(N(c3ccc(-c4cc5ccccc5cc4-c4ccccc4)cc3)c3cccc4c3sc3ccccc34)cc2-c2ccccc2)cc1]

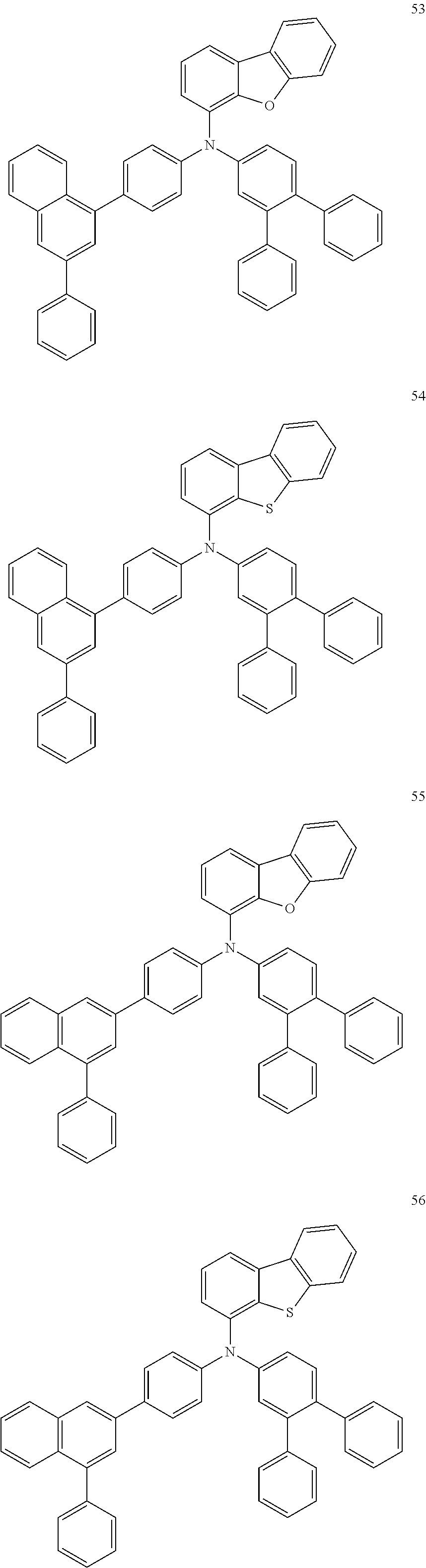 [SMILES: c1ccc(-c2cc(-c3ccc(N(c4ccc(-c5ccccc5)c(-c5ccccc5)c4)c4cccc5c4oc4ccccc45)cc3)c3ccccc3c2)cc1 ; c1ccc(-c2cc(-c3ccc(N(c4ccc(-c5ccccc5)c(-c5ccccc5)c4)c4cccc5c4sc4ccccc45)cc3)c3ccccc3c2)cc1 ; c1ccc(-c2ccc(N(c3ccc(-c4cc(-c5ccccc5)c5ccccc5c4)cc3)c3cccc4c3oc3ccccc34)cc2-c2ccccc2)cc1 ; c1ccc(-c2ccc(N(c3ccc(-c4cc(-c5ccccc5)c5ccccc5c4)cc3)c3cccc4c3sc3ccccc34)cc2-c2ccccc2)cc1]

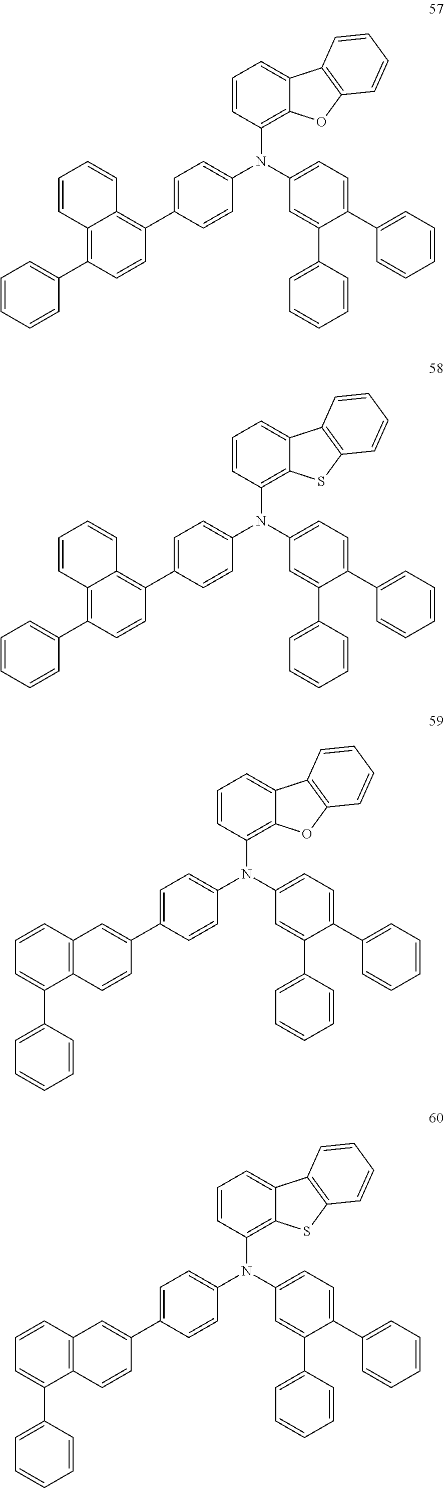 [SMILES: c1ccc(-c2ccc(N(c3ccc(-c4ccc(-c5ccccc5)c5ccccc45)cc3)c3cccc4c3oc3ccccc34)cc2-c2ccccc2)cc1 ; c1ccc(-c2ccc(N(c3ccc(-c4ccc(-c5ccccc5)c5ccccc45)cc3)c3cccc4c3sc3ccccc34)cc2-c2ccccc2)cc1 ; c1ccc(-c2ccc(N(c3ccc(-c4ccc5c(-c6ccccc6)cccc5c4)cc3)c3cccc4c3oc3ccccc34)cc2-c2ccccc2)cc1 ; c1ccc(-c2ccc(N(c3ccc(-c4ccc5c(-c6ccccc6)cccc5c4)cc3)c3cccc4c3sc3ccccc34)cc2-c2ccccc2)cc1]

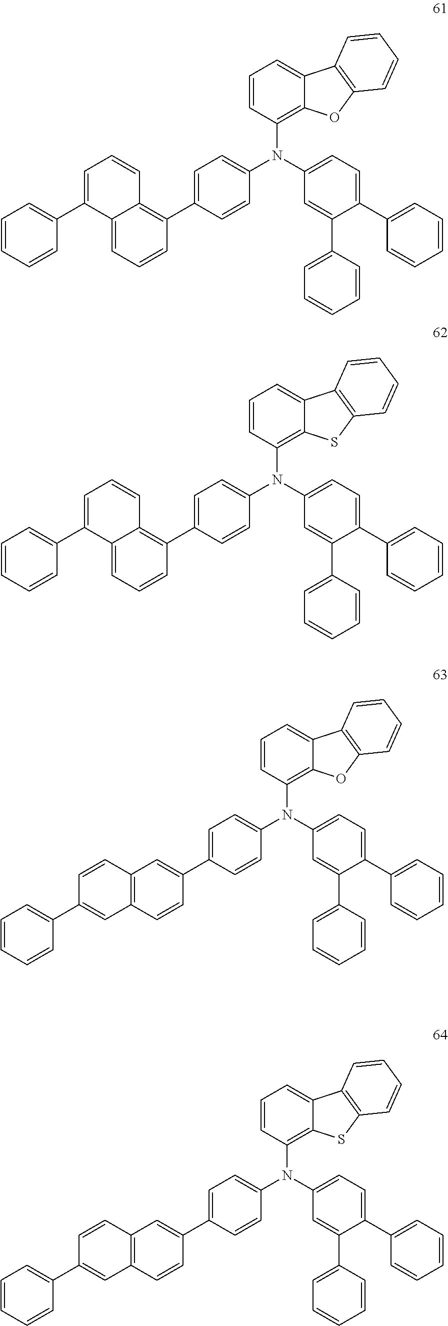 [SMILES: c1ccc(-c2ccc(N(c3ccc(-c4cccc5c(-c6ccccc6)cccc45)cc3)c3cccc4c3oc3ccccc34)cc2-c2ccccc2)cc1 ; c1ccc(-c2ccc(N(c3ccc(-c4cccc5c(-c6ccccc6)cccc45)cc3)c3cccc4c3sc3ccccc34)cc2-c2ccccc2)cc1 ; c1ccc(-c2ccc3cc(-c4ccc(N(c5ccc(-c6ccccc6)c(-c6ccccc6)c5)c5cccc6c5oc5ccccc56)cc4)ccc3c2)cc1 ; c1ccc(-c2ccc3cc(-c4ccc(N(c5ccc(-c6ccccc6)c(-c6ccccc6)c5)c5cccc6c5sc5ccccc56)cc4)ccc3c2)cc1]

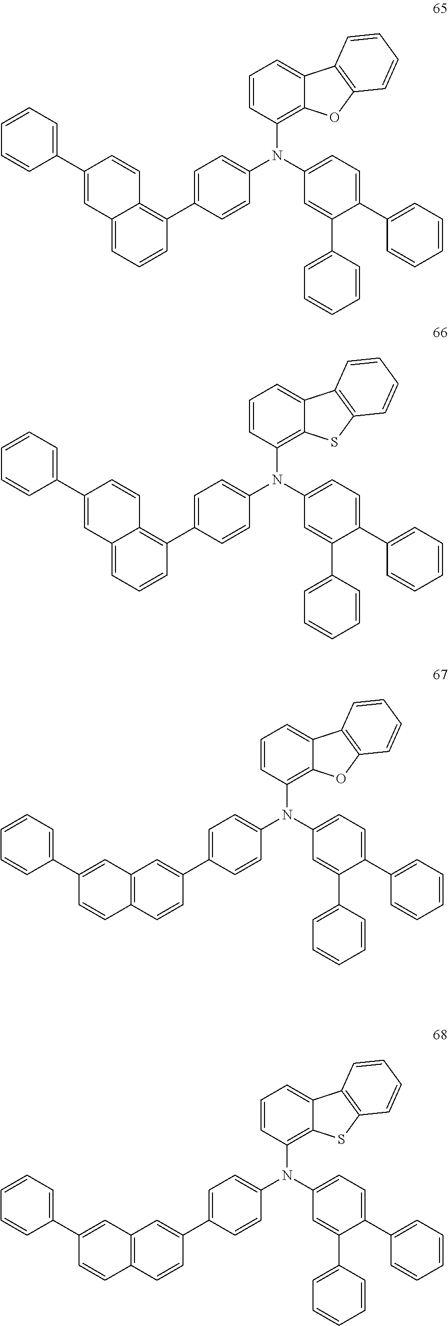 [SMILES: c1ccc(-c2ccc3c(-c4ccc(N(c5ccc(-c6ccccc6)c(-c6ccccc6)c5)c5cccc6c5oc5ccccc56)cc4)cccc3c2)cc1 ; c1ccc(-c2ccc3c(-c4ccc(N(c5ccc(-c6ccccc6)c(-c6ccccc6)c5)c5cccc6c5sc5ccccc56)cc4)cccc3c2)cc1 ; c1ccc(-c2ccc3ccc(-c4ccc(N(c5ccc(-c6ccccc6)c(-c6ccccc6)c5)c5cccc6c5oc5ccccc56)cc4)cc3c2)cc1 ; c1ccc(-c2ccc3ccc(-c4ccc(N(c5ccc(-c6ccccc6)c(-c6ccccc6)c5)c5cccc6c5sc5ccccc56)cc4)cc3c2)cc1]

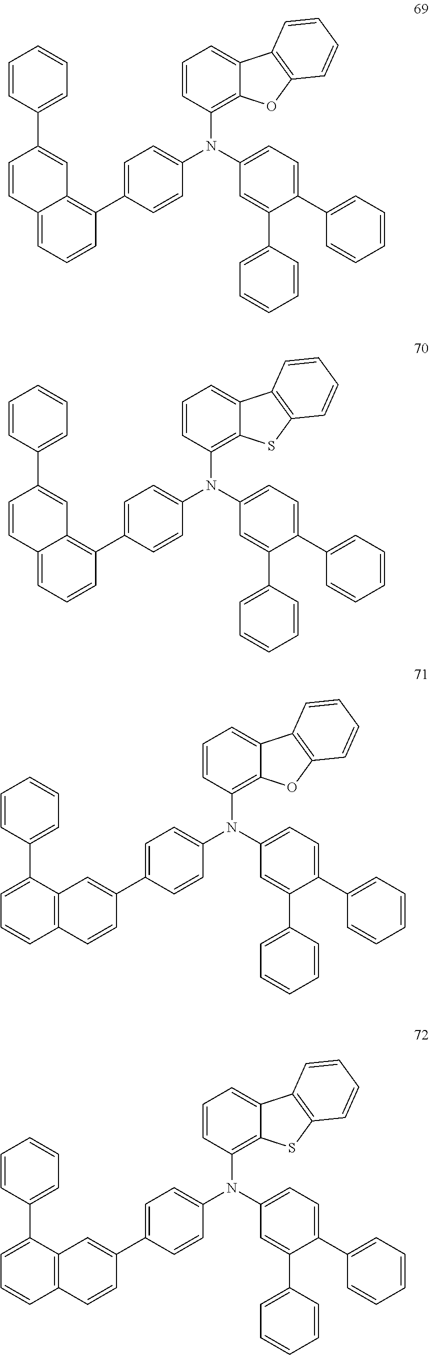 [SMILES: c1ccc(-c2ccc3cccc(-c4ccc(N(c5ccc(-c6ccccc6)c(-c6ccccc6)c5)c5cccc6c5oc5ccccc56)cc4)c3c2)cc1 ; c1ccc(-c2ccc3cccc(-c4ccc(N(c5ccc(-c6ccccc6)c(-c6ccccc6)c5)c5cccc6c5sc5ccccc56)cc4)c3c2)cc1 ; c1ccc(-c2ccc(N(c3ccc(-c4ccc5cccc(-c6ccccc6)c5c4)cc3)c3cccc4c3oc3ccccc34)cc2-c2ccccc2)cc1 ; c1ccc(-c2ccc(N(c3ccc(-c4ccc5cccc(-c6ccccc6)c5c4)cc3)c3cccc4c3sc3ccccc34)cc2-c2ccccc2)cc1]

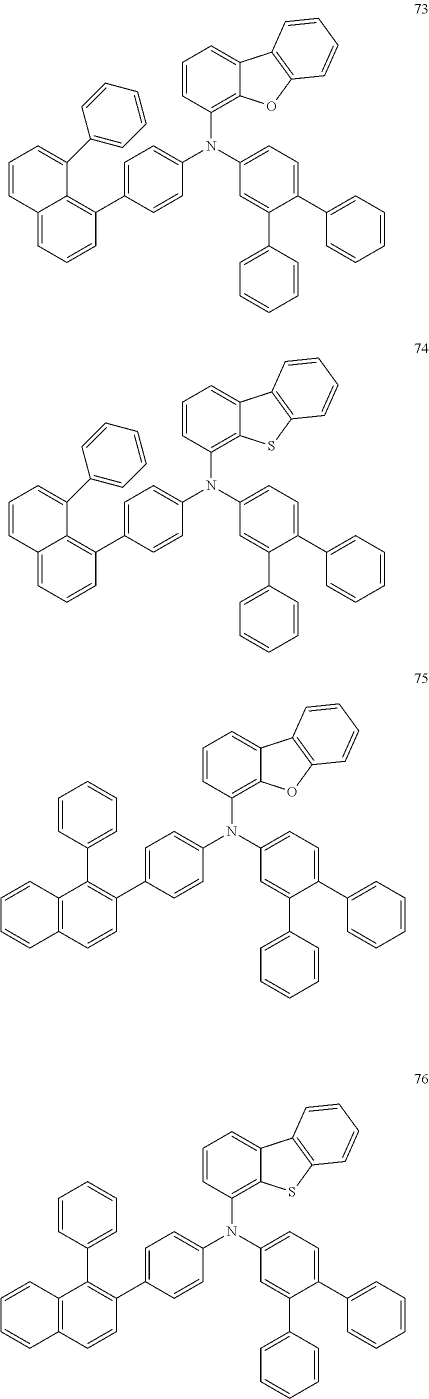 [SMILES: c1ccc(-c2ccc(N(c3ccc(-c4cccc5cccc(-c6ccccc6)c45)cc3)c3cccc4c3oc3ccccc34)cc2-c2ccccc2)cc1 ; c1ccc(-c2ccc(N(c3ccc(-c4cccc5cccc(-c6ccccc6)c45)cc3)c3cccc4c3sc3ccccc34)cc2-c2ccccc2)cc1 ; c1ccc(-c2ccc(N(c3ccc(-c4ccc5ccccc5c4-c4ccccc4)cc3)c3cccc4c3oc3ccccc34)cc2-c2ccccc2)cc1 ; c1ccc(-c2ccc(N(c3ccc(-c4ccc5ccccc5c4-c4ccccc4)cc3)c3cccc4c3sc3ccccc34)cc2-c2ccccc2)cc1]

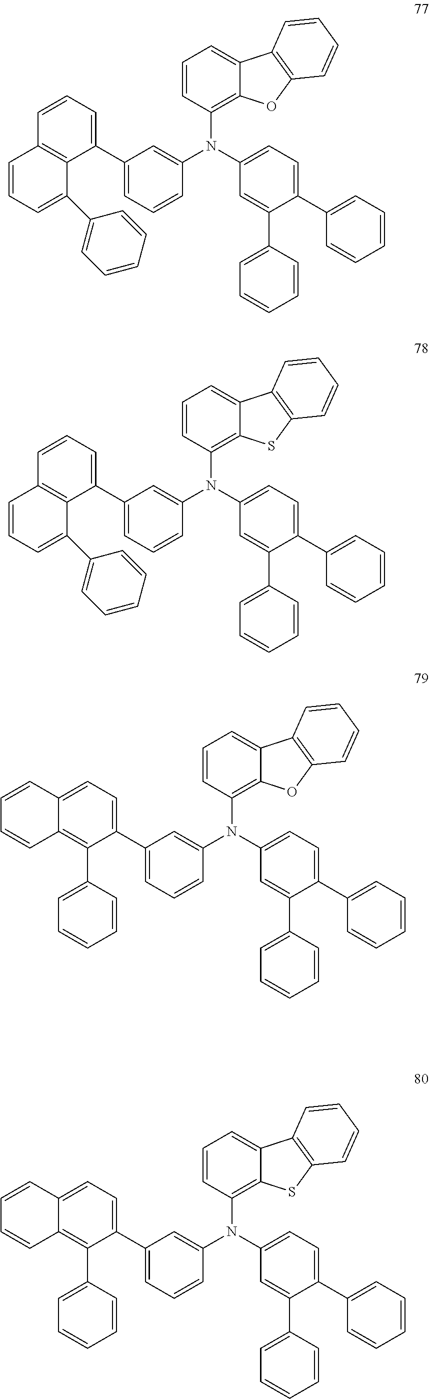 [SMILES: c1ccc(-c2ccc(N(c3cccc(-c4cccc5cccc(-c6ccccc6)c45)c3)c3cccc4c3oc3ccccc34)cc2-c2ccccc2)cc1 ; c1ccc(-c2ccc(N(c3cccc(-c4cccc5cccc(-c6ccccc6)c45)c3)c3cccc4c3sc3ccccc34)cc2-c2ccccc2)cc1 ; c1ccc(-c2ccc(N(c3cccc(-c4ccc5ccccc5c4-c4ccccc4)c3)c3cccc4c3oc3ccccc34)cc2-c2ccccc2)cc1 ; c1ccc(-c2ccc(N(c3cccc(-c4ccc5ccccc5c4-c4ccccc4)c3)c3cccc4c3sc3ccccc34)cc2-c2ccccc2)cc1]

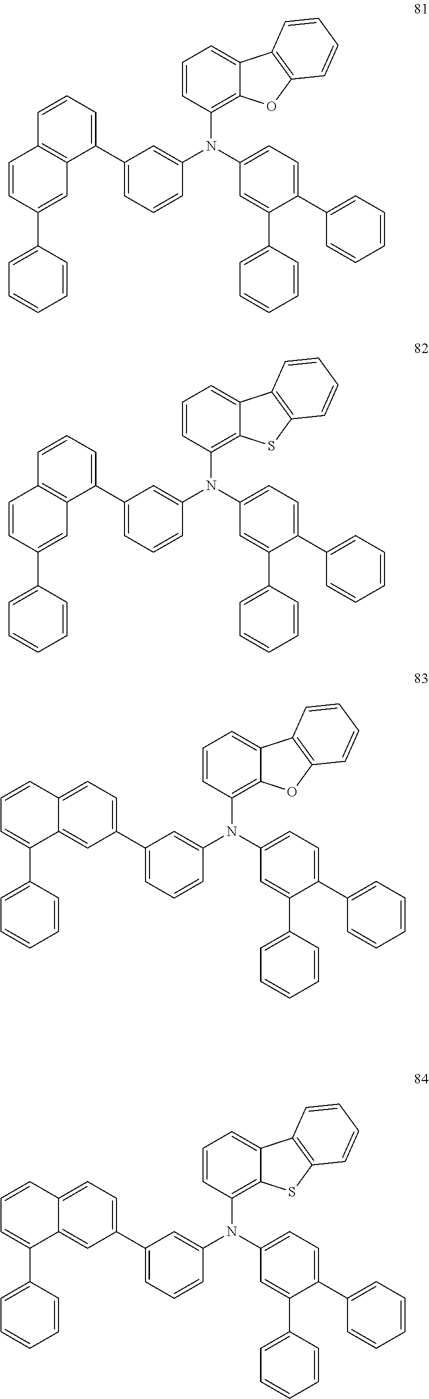 [SMILES: c1ccc(-c2ccc3cccc(-c4cccc(N(c5ccc(-c6ccccc6)c(-c6ccccc6)c5)c5cccc6c5oc5ccccc56)c4)c3c2)cc1 ; c1ccc(-c2ccc3cccc(-c4cccc(N(c5ccc(-c6ccccc6)c(-c6ccccc6)c5)c5cccc6c5sc5ccccc56)c4)c3c2)cc1 ; c1ccc(-c2ccc(N(c3cccc(-c4ccc5cccc(-c6ccccc6)c5c4)c3)c3cccc4c3oc3ccccc34)cc2-c2ccccc2)cc1 ; c1ccc(-c2ccc(N(c3cccc(-c4ccc5cccc(-c6ccccc6)c5c4)c3)c3cccc4c3sc3ccccc34)cc2-c2ccccc2)cc1]

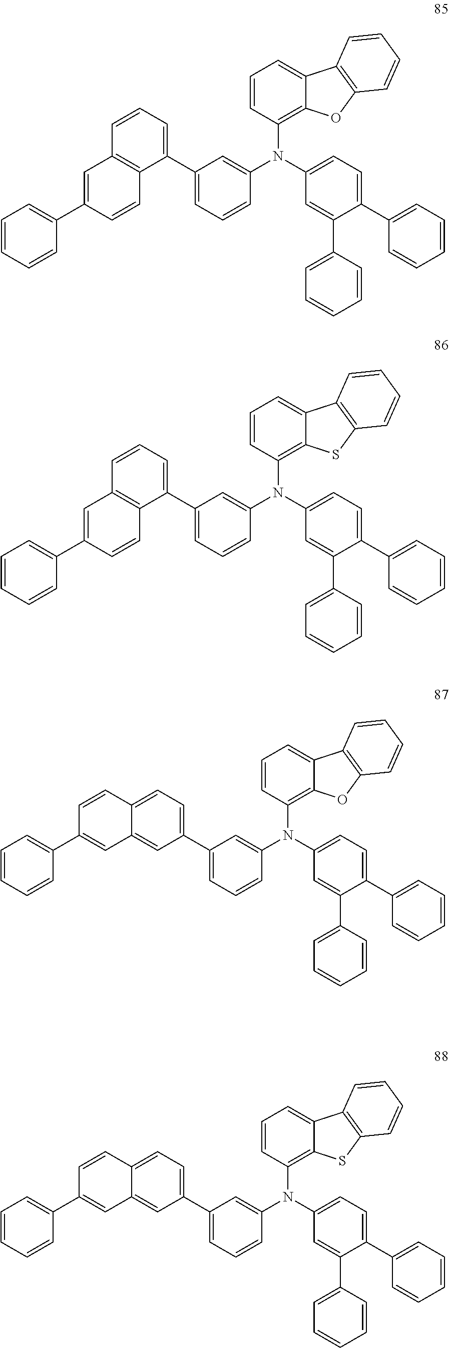 [SMILES: c1ccc(-c2ccc3c(-c4cccc(N(c5ccc(-c6ccccc6)c(-c6ccccc6)c5)c5cccc6c5oc5ccccc56)c4)cccc3c2)cc1 ; c1ccc(-c2ccc3c(-c4cccc(N(c5ccc(-c6ccccc6)c(-c6ccccc6)c5)c5cccc6c5sc5ccccc56)c4)cccc3c2)cc1 ; c1ccc(-c2ccc3ccc(-c4cccc(N(c5ccc(-c6ccccc6)c(-c6ccccc6)c5)c5cccc6c5oc5ccccc56)c4)cc3c2)cc1 ; c1ccc(-c2ccc3ccc(-c4cccc(N(c5ccc(-c6ccccc6)c(-c6ccccc6)c5)c5cccc6c5sc5ccccc56)c4)cc3c2)cc1]

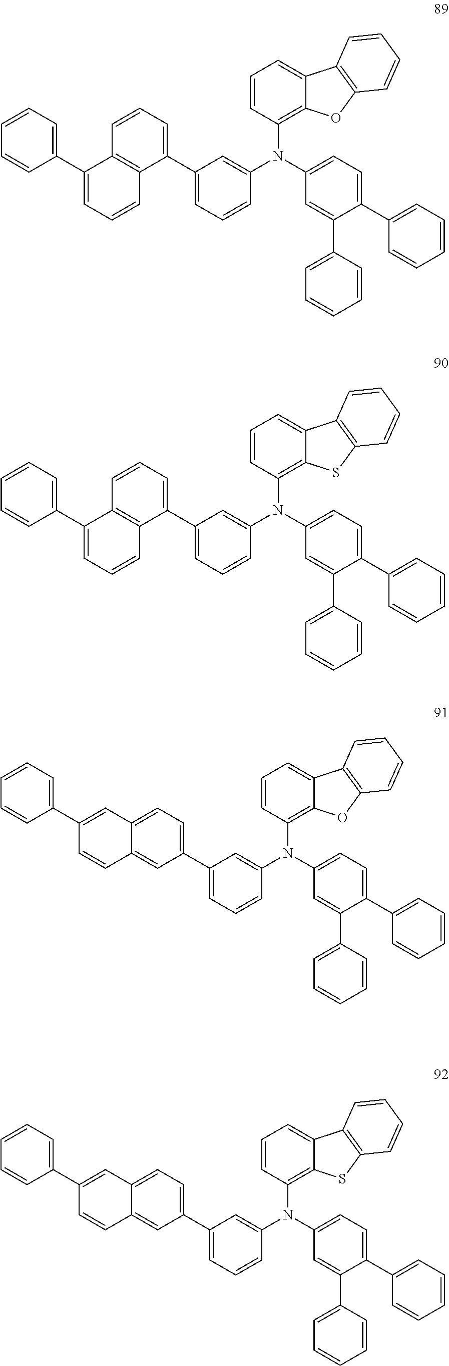 [SMILES: c1ccc(-c2ccc(N(c3cccc(-c4cccc5c(-c6ccccc6)cccc45)c3)c3cccc4c3oc3ccccc34)cc2-c2ccccc2)cc1 ; c1ccc(-c2ccc(N(c3cccc(-c4cccc5c(-c6ccccc6)cccc45)c3)c3cccc4c3sc3ccccc34)cc2-c2ccccc2)cc1 ; c1ccc(-c2ccc3cc(-c4cccc(N(c5ccc(-c6ccccc6)c(-c6ccccc6)c5)c5cccc6c5oc5ccccc56)c4)ccc3c2)cc1 ; c1ccc(-c2ccc3cc(-c4cccc(N(c5ccc(-c6ccccc6)c(-c6ccccc6)c5)c5cccc6c5sc5ccccc56)c4)ccc3c2)cc1]

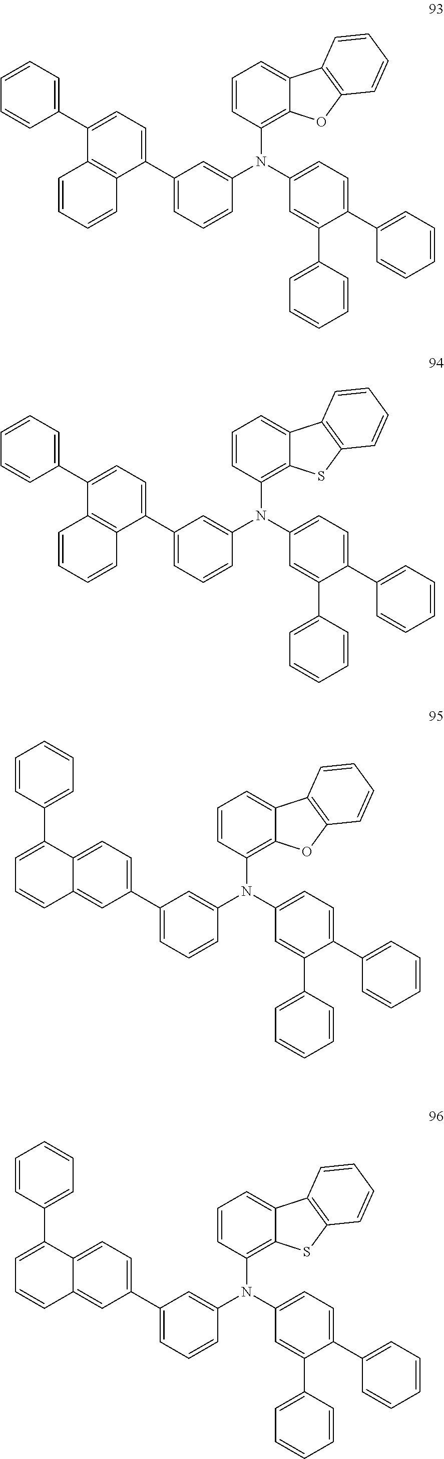 [SMILES: c1ccc(-c2ccc(N(c3cccc(-c4ccc(-c5ccccc5)c5ccccc45)c3)c3cccc4c3oc3ccccc34)cc2-c2ccccc2)cc1 ; c1ccc(-c2ccc(N(c3cccc(-c4ccc(-c5ccccc5)c5ccccc45)c3)c3cccc4c3sc3ccccc34)cc2-c2ccccc2)cc1 ; c1ccc(-c2ccc(N(c3cccc(-c4ccc5c(-c6ccccc6)cccc5c4)c3)c3cccc4c3oc3ccccc34)cc2-c2ccccc2)cc1 ; c1ccc(-c2ccc(N(c3cccc(-c4ccc5c(-c6ccccc6)cccc5c4)c3)c3cccc4c3sc3ccccc34)cc2-c2ccccc2)cc1]

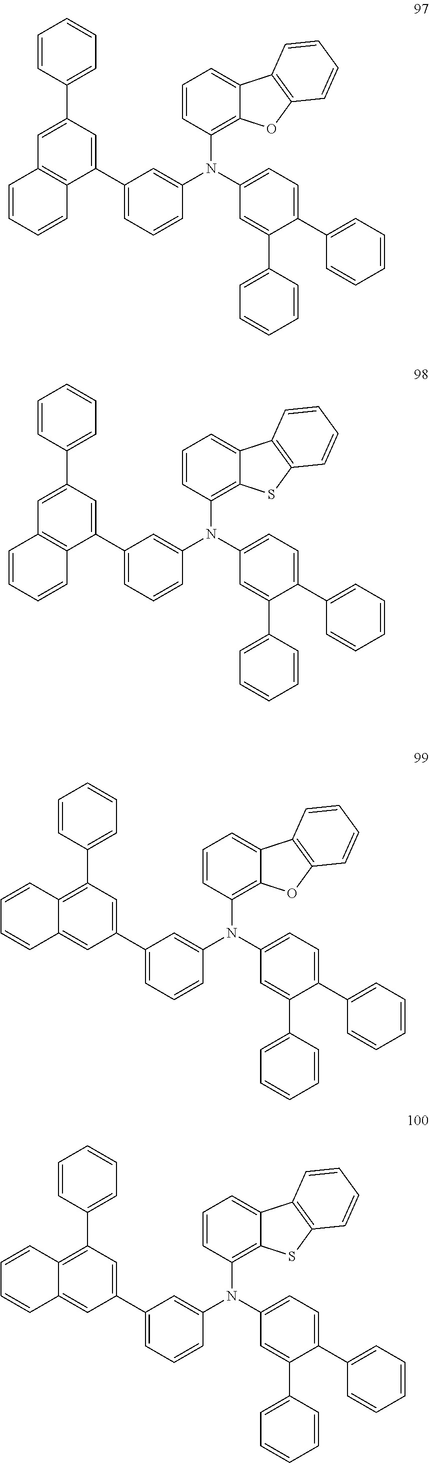 [SMILES: c1ccc(-c2cc(-c3cccc(N(c4ccc(-c5ccccc5)c(-c5ccccc5)c4)c4cccc5c4oc4ccccc45)c3)c3ccccc3c2)cc1 ; c1ccc(-c2cc(-c3cccc(N(c4ccc(-c5ccccc5)c(-c5ccccc5)c4)c4cccc5c4sc4ccccc45)c3)c3ccccc3c2)cc1 ; c1ccc(-c2ccc(N(c3cccc(-c4cc(-c5ccccc5)c5ccccc5c4)c3)c3cccc4c3oc3ccccc34)cc2-c2ccccc2)cc1 ; c1ccc(-c2ccc(N(c3cccc(-c4cc(-c5ccccc5)c5ccccc5c4)c3)c3cccc4c3sc3ccccc34)cc2-c2ccccc2)cc1]

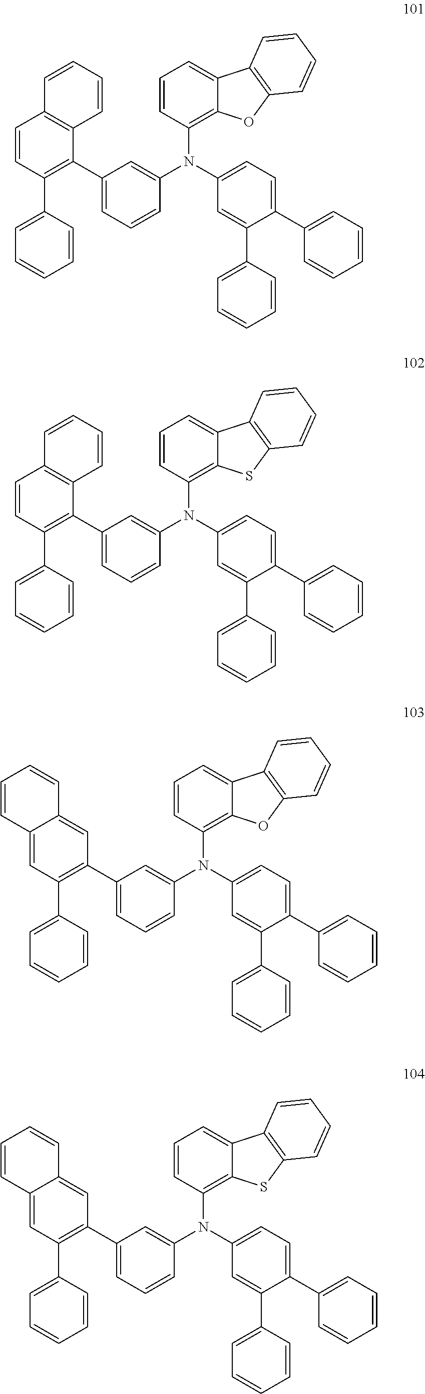 [SMILES: c1ccc(-c2ccc(N(c3cccc(-c4c(-c5ccccc5)ccc5ccccc45)c3)c3cccc4c3oc3ccccc34)cc2-c2ccccc2)cc1 ; c1ccc(-c2ccc(N(c3cccc(-c4c(-c5ccccc5)ccc5ccccc45)c3)c3cccc4c3sc3ccccc34)cc2-c2ccccc2)cc1 ; c1ccc(-c2ccc(N(c3cccc(-c4cc5ccccc5cc4-c4ccccc4)c3)c3cccc4c3oc3ccccc34)cc2-c2ccccc2)cc1 ; c1ccc(-c2ccc(N(c3cccc(-c4cc5ccccc5cc4-c4ccccc4)c3)c3cccc4c3sc3ccccc34)cc2-c2ccccc2)cc1]

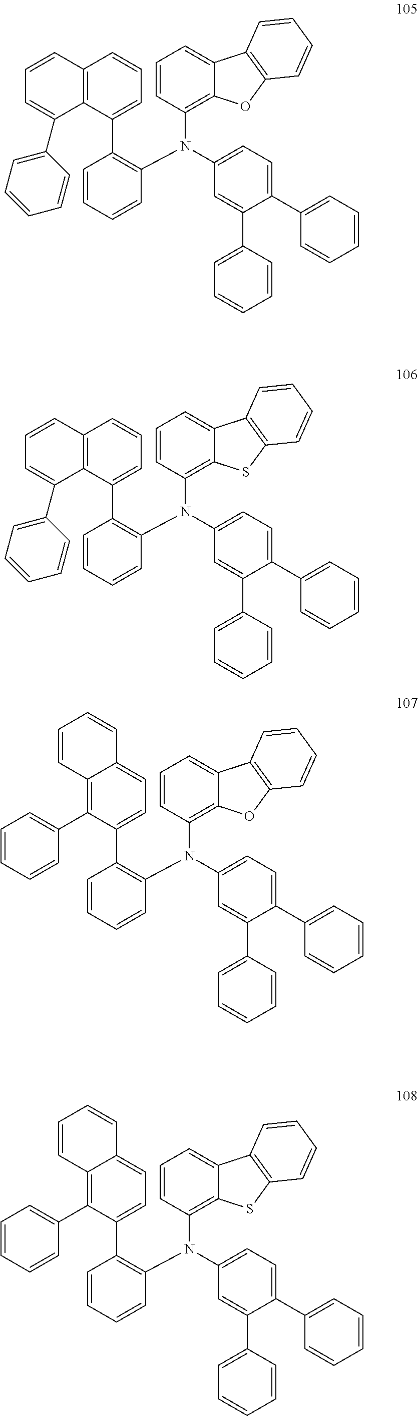 [SMILES: c1ccc(-c2ccc(N(c3ccccc3-c3cccc4cccc(-c5ccccc5)c34)c3cccc4c3oc3ccccc34)cc2-c2ccccc2)cc1 ; c1ccc(-c2ccc(N(c3ccccc3-c3cccc4cccc(-c5ccccc5)c34)c3cccc4c3sc3ccccc34)cc2-c2ccccc2)cc1 ; c1ccc(-c2ccc(N(c3ccccc3-c3ccc4ccccc4c3-c3ccccc3)c3cccc4c3oc3ccccc34)cc2-c2ccccc2)cc1 ; c1ccc(-c2ccc(N(c3ccccc3-c3ccc4ccccc4c3-c3ccccc3)c3cccc4c3sc3ccccc34)cc2-c2ccccc2)cc1]

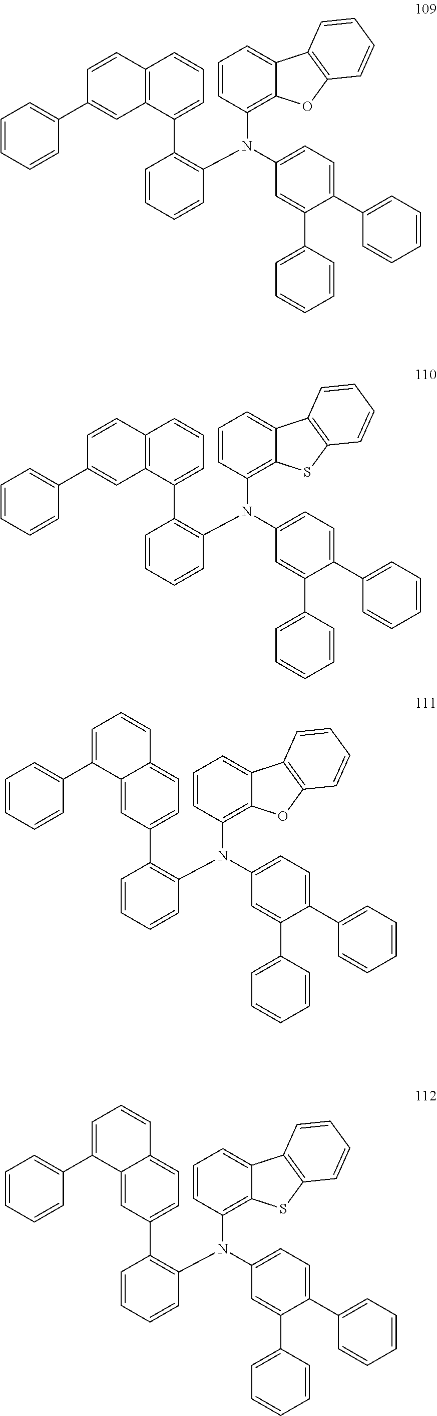 [SMILES: c1ccc(-c2ccc3cccc(-c4ccccc4N(c4ccc(-c5ccccc5)c(-c5ccccc5)c4)c4cccc5c4oc4ccccc45)c3c2)cc1 ; c1ccc(-c2ccc3cccc(-c4ccccc4N(c4ccc(-c5ccccc5)c(-c5ccccc5)c4)c4cccc5c4sc4ccccc45)c3c2)cc1 ; c1ccc(-c2ccc(N(c3ccccc3-c3ccc4cccc(-c5ccccc5)c4c3)c3cccc4c3oc3ccccc34)cc2-c2ccccc2)cc1 ; c1ccc(-c2ccc(N(c3ccccc3-c3ccc4cccc(-c5ccccc5)c4c3)c3cccc4c3sc3ccccc34)cc2-c2ccccc2)cc1]

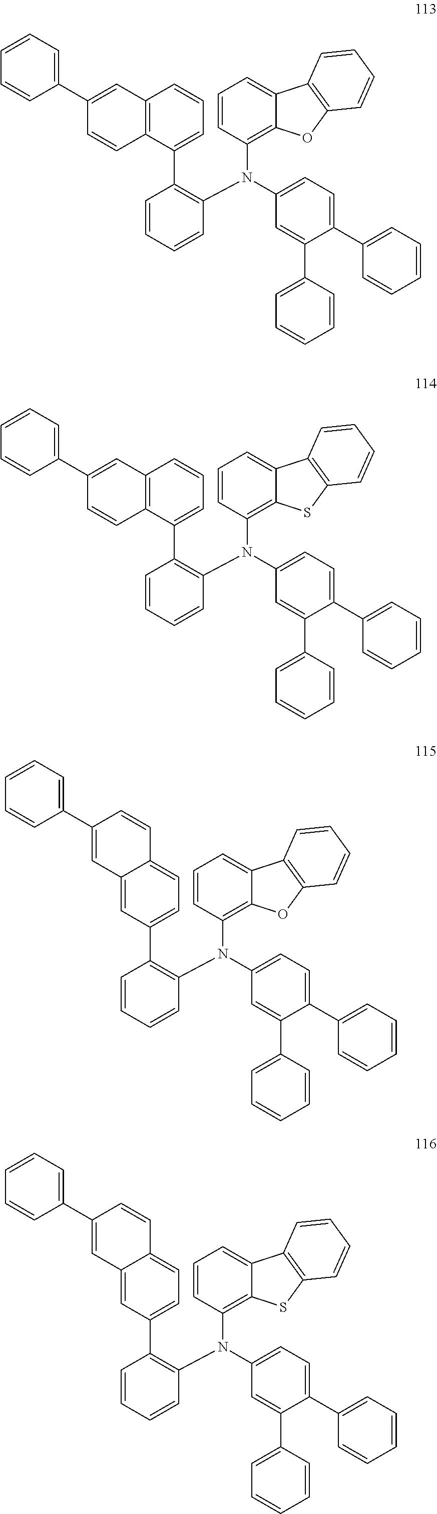 [SMILES: c1ccc(-c2ccc3c(-c4ccccc4N(c4ccc(-c5ccccc5)c(-c5ccccc5)c4)c4cccc5c4oc4ccccc45)cccc3c2)cc1 ; c1ccc(-c2ccc3c(-c4ccccc4N(c4ccc(-c5ccccc5)c(-c5ccccc5)c4)c4cccc5c4sc4ccccc45)cccc3c2)cc1 ; c1ccc(-c2ccc3ccc(-c4ccccc4N(c4ccc(-c5ccccc5)c(-c5ccccc5)c4)c4cccc5c4oc4ccccc45)cc3c2)cc1 ; c1ccc(-c2ccc3ccc(-c4ccccc4N(c4ccc(-c5ccccc5)c(-c5ccccc5)c4)c4cccc5c4sc4ccccc45)cc3c2)cc1]

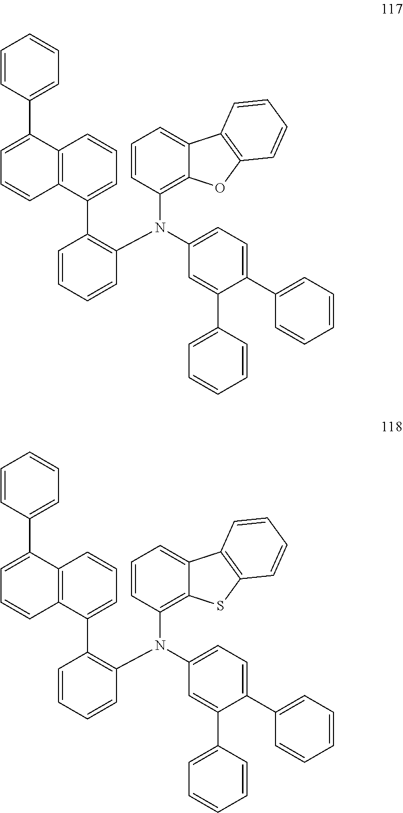 [SMILES: c1ccc(-c2ccc(N(c3ccccc3-c3cccc4c(-c5ccccc5)cccc34)c3cccc4c3oc3ccccc34)cc2-c2ccccc2)cc1 ; c1ccc(-c2ccc(N(c3ccccc3-c3cccc4c(-c5ccccc5)cccc34)c3cccc4c3sc3ccccc34)cc2-c2ccccc2)cc1]

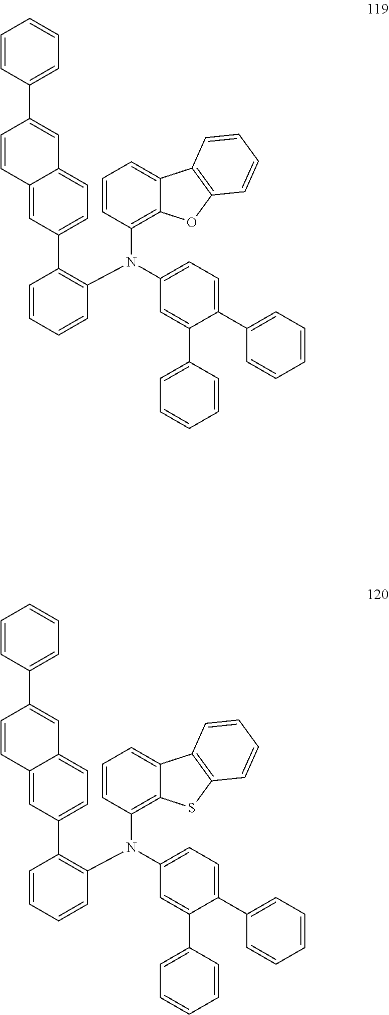 [SMILES: c1ccc(-c2ccc3cc(-c4ccccc4N(c4ccc(-c5ccccc5)c(-c5ccccc5)c4)c4cccc5c4oc4ccccc45)ccc3c2)cc1 ; c1ccc(-c2ccc3cc(-c4ccccc4N(c4ccc(-c5ccccc5)c(-c5ccccc5)c4)c4cccc5c4sc4ccccc45)ccc3c2)cc1]

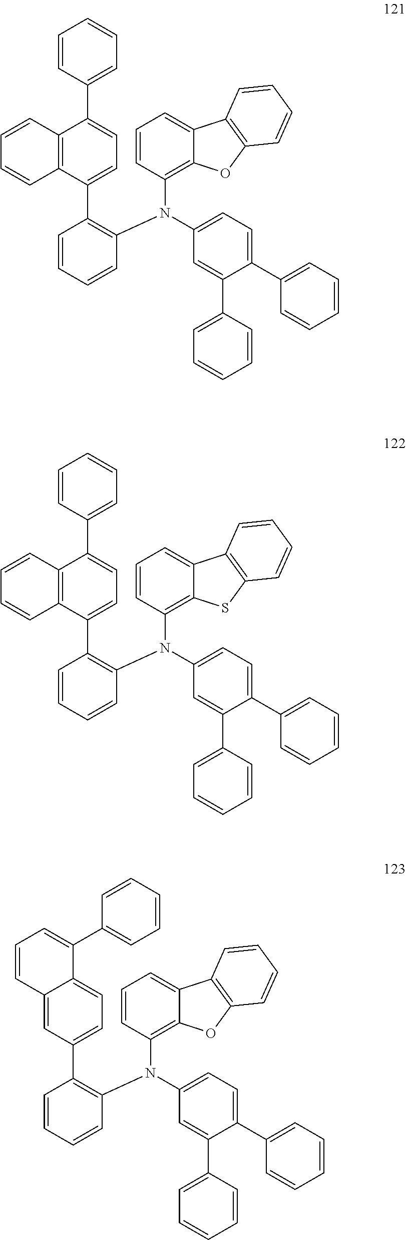 [SMILES: c1ccc(-c2ccc(N(c3ccccc3-c3ccc(-c4ccccc4)c4ccccc34)c3cccc4c3oc3ccccc34)cc2-c2ccccc2)cc1 ; c1ccc(-c2ccc(N(c3ccccc3-c3ccc(-c4ccccc4)c4ccccc34)c3cccc4c3sc3ccccc34)cc2-c2ccccc2)cc1 ; c1ccc(-c2ccc(N(c3ccccc3-c3ccc4c(-c5ccccc5)cccc4c3)c3cccc4c3oc3ccccc34)cc2-c2ccccc2)cc1]

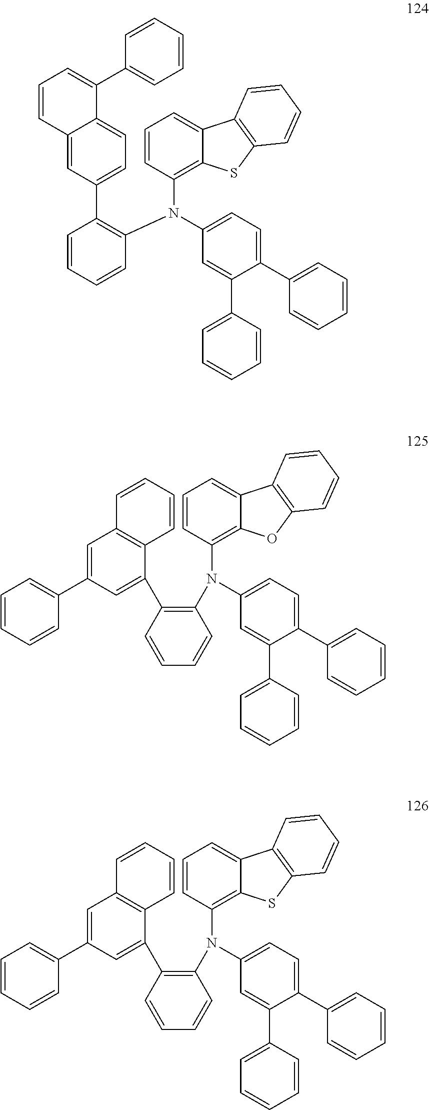 [SMILES: c1ccc(-c2ccc(N(c3ccccc3-c3ccc4c(-c5ccccc5)cccc4c3)c3cccc4c3sc3ccccc34)cc2-c2ccccc2)cc1 ; c1ccc(-c2cc(-c3ccccc3N(c3ccc(-c4ccccc4)c(-c4ccccc4)c3)c3cccc4c3oc3ccccc34)c3ccccc3c2)cc1 ; c1ccc(-c2cc(-c3ccccc3N(c3ccc(-c4ccccc4)c(-c4ccccc4)c3)c3cccc4c3sc3ccccc34)c3ccccc3c2)cc1]

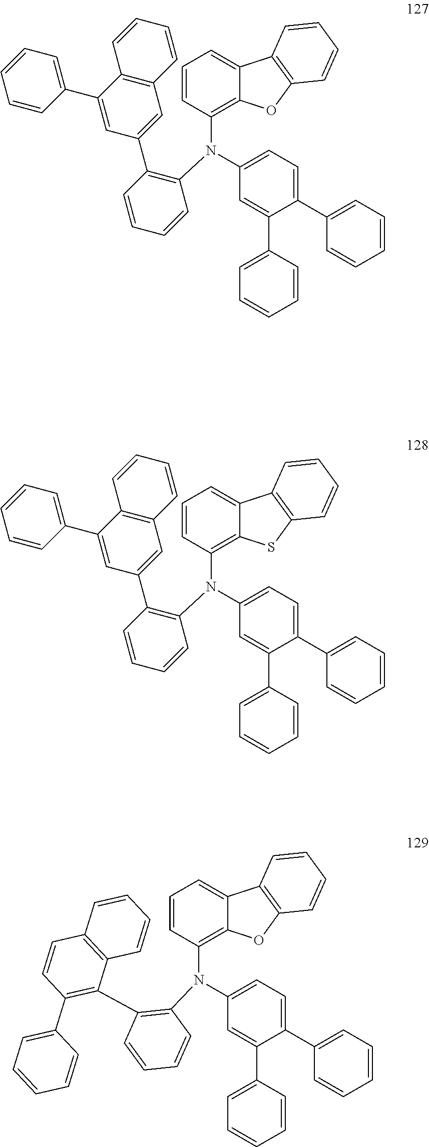 [SMILES: c1ccc(-c2ccc(N(c3ccccc3-c3cc(-c4ccccc4)c4ccccc4c3)c3cccc4c3oc3ccccc34)cc2-c2ccccc2)cc1 ; c1ccc(-c2ccc(N(c3ccccc3-c3cc(-c4ccccc4)c4ccccc4c3)c3cccc4c3sc3ccccc34)cc2-c2ccccc2)cc1 ; c1ccc(-c2ccc(N(c3ccccc3-c3c(-c4ccccc4)ccc4ccccc34)c3cccc4c3oc3ccccc34)cc2-c2ccccc2)cc1]

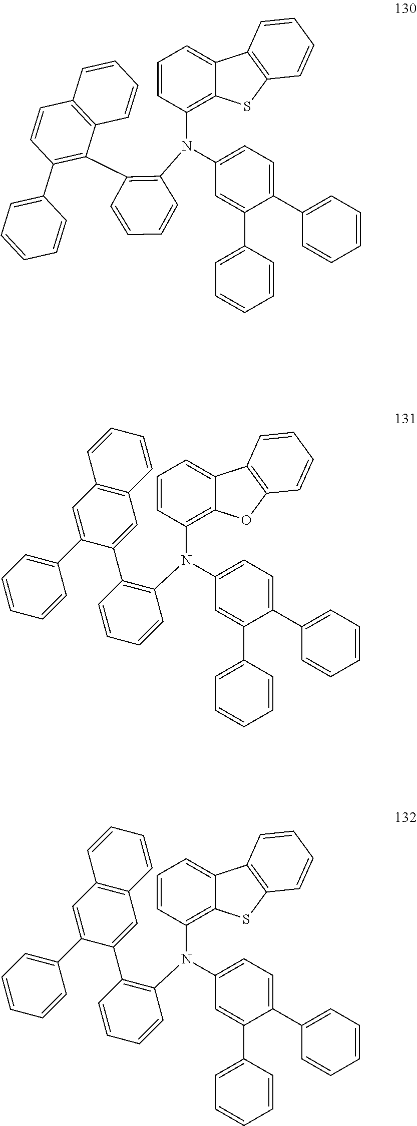 [SMILES: c1ccc(-c2ccc(N(c3ccccc3-c3c(-c4ccccc4)ccc4ccccc34)c3cccc4c3sc3ccccc34)cc2-c2ccccc2)cc1 ; c1ccc(-c2ccc(N(c3ccccc3-c3cc4ccccc4cc3-c3ccccc3)c3cccc4c3oc3ccccc34)cc2-c2ccccc2)cc1 ; c1ccc(-c2ccc(N(c3ccccc3-c3cc4ccccc4cc3-c3ccccc3)c3cccc4c3sc3ccccc34)cc2-c2ccccc2)cc1]

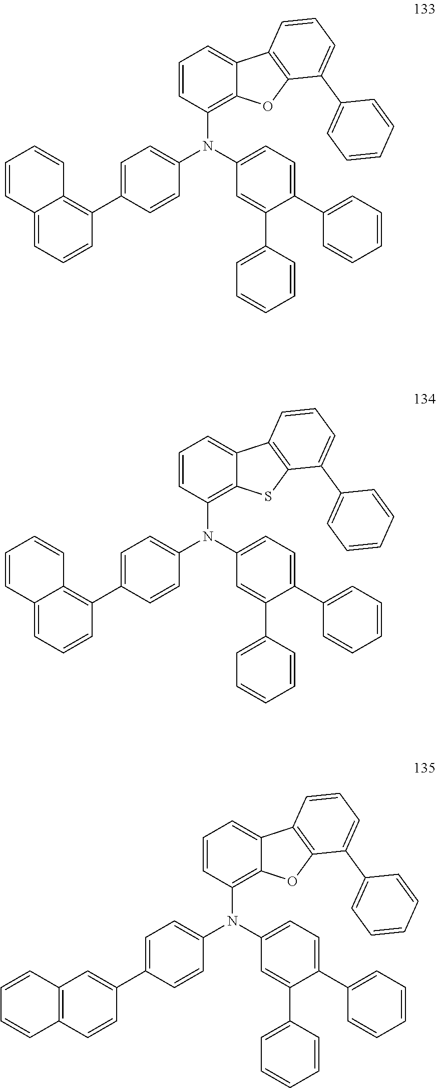 [SMILES: c1ccc(-c2ccc(N(c3ccc(-c4cccc5ccccc45)cc3)c3cccc4c3oc3c(-c5ccccc5)cccc34)cc2-c2ccccc2)cc1 ; c1ccc(-c2ccc(N(c3ccc(-c4cccc5ccccc45)cc3)c3cccc4c3sc3c(-c5ccccc5)cccc34)cc2-c2ccccc2)cc1 ; c1ccc(-c2ccc(N(c3ccc(-c4ccc5ccccc5c4)cc3)c3cccc4c3oc3c(-c5ccccc5)cccc34)cc2-c2ccccc2)cc1]

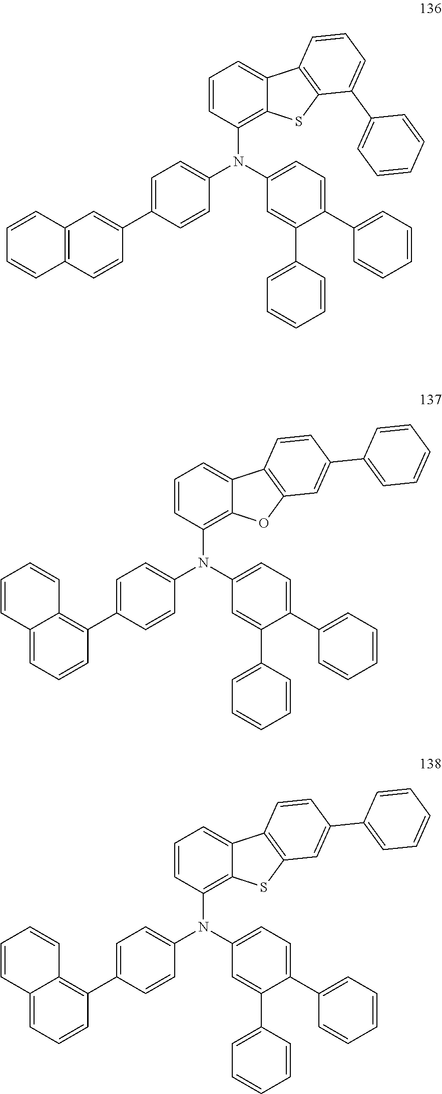 [SMILES: c1ccc(-c2ccc(N(c3ccc(-c4ccc5ccccc5c4)cc3)c3cccc4c3sc3c(-c5ccccc5)cccc34)cc2-c2ccccc2)cc1 ; c1ccc(-c2ccc3c(c2)oc2c(N(c4ccc(-c5cccc6ccccc56)cc4)c4ccc(-c5ccccc5)c(-c5ccccc5)c4)cccc23)cc1 ; c1ccc(-c2ccc3c(c2)sc2c(N(c4ccc(-c5cccc6ccccc56)cc4)c4ccc(-c5ccccc5)c(-c5ccccc5)c4)cccc23)cc1]

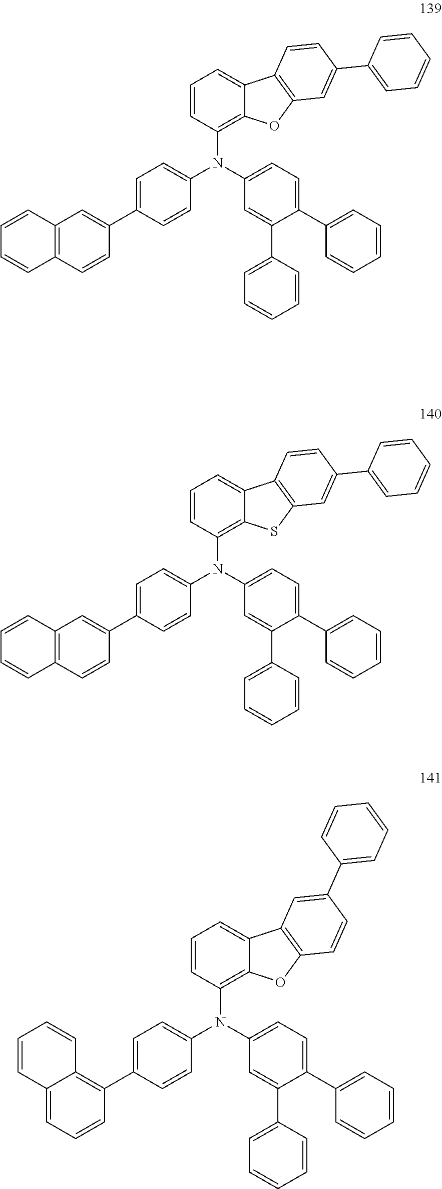 [SMILES: c1ccc(-c2ccc3c(c2)oc2c(N(c4ccc(-c5ccc6ccccc6c5)cc4)c4ccc(-c5ccccc5)c(-c5ccccc5)c4)cccc23)cc1 ; c1ccc(-c2ccc3c(c2)sc2c(N(c4ccc(-c5ccc6ccccc6c5)cc4)c4ccc(-c5ccccc5)c(-c5ccccc5)c4)cccc23)cc1 ; c1ccc(-c2ccc3oc4c(N(c5ccc(-c6cccc7ccccc67)cc5)c5ccc(-c6ccccc6)c(-c6ccccc6)c5)cccc4c3c2)cc1]

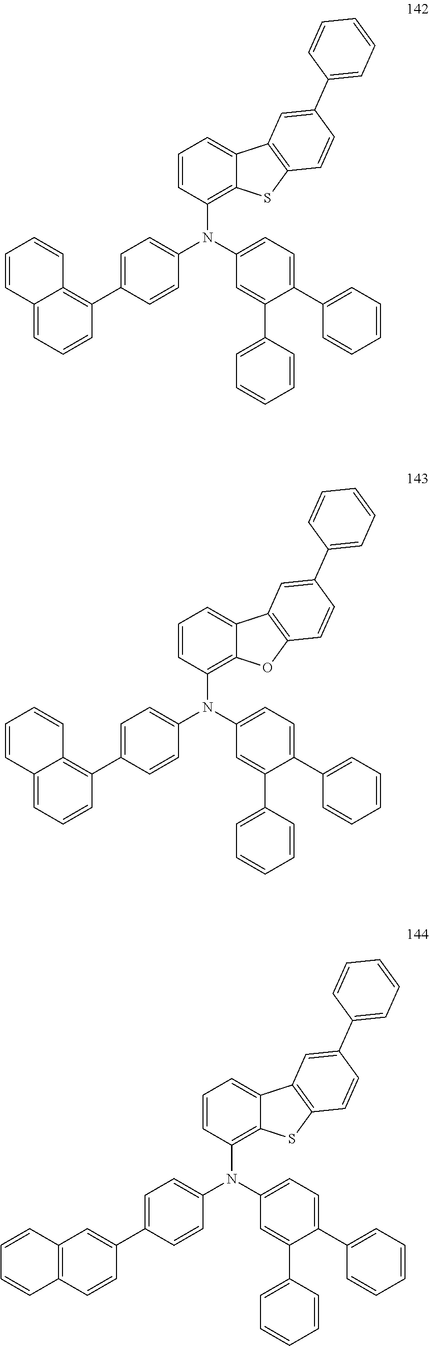 [SMILES: c1ccc(-c2ccc3sc4c(N(c5ccc(-c6cccc7ccccc67)cc5)c5ccc(-c6ccccc6)c(-c6ccccc6)c5)cccc4c3c2)cc1 ; c1ccc(-c2ccc3oc4c(N(c5ccc(-c6cccc7ccccc67)cc5)c5ccc(-c6ccccc6)c(-c6ccccc6)c5)cccc4c3c2)cc1 ; c1ccc(-c2ccc3sc4c(N(c5ccc(-c6ccc7ccccc7c6)cc5)c5ccc(-c6ccccc6)c(-c6ccccc6)c5)cccc4c3c2)cc1]

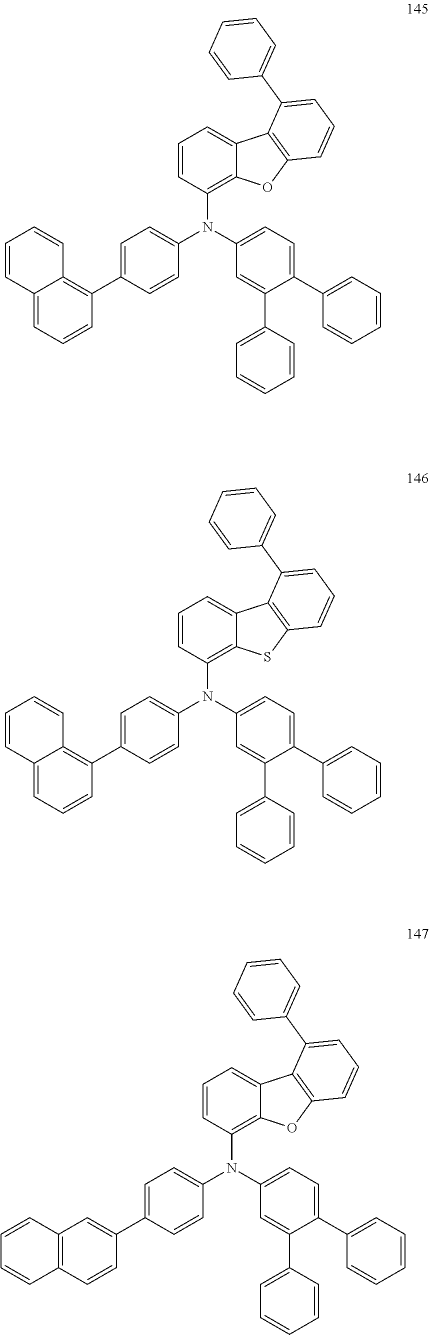 [SMILES: c1ccc(-c2ccc(N(c3ccc(-c4cccc5ccccc45)cc3)c3cccc4c3oc3cccc(-c5ccccc5)c34)cc2-c2ccccc2)cc1 ; c1ccc(-c2ccc(N(c3ccc(-c4cccc5ccccc45)cc3)c3cccc4c3sc3cccc(-c5ccccc5)c34)cc2-c2ccccc2)cc1 ; c1ccc(-c2ccc(N(c3ccc(-c4ccc5ccccc5c4)cc3)c3cccc4c3oc3cccc(-c5ccccc5)c34)cc2-c2ccccc2)cc1]

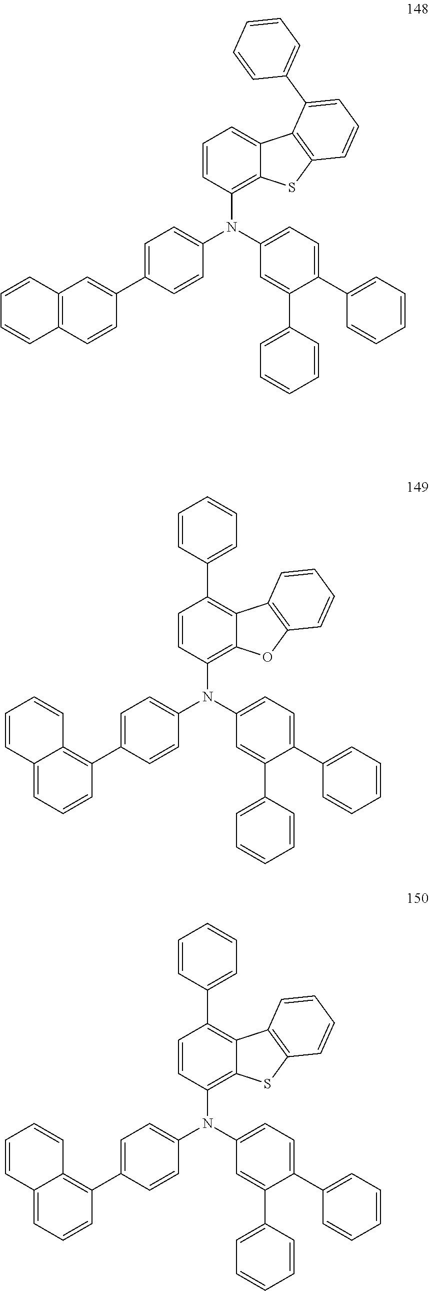 [SMILES: c1ccc(-c2ccc(N(c3ccc(-c4ccc5ccccc5c4)cc3)c3cccc4c3sc3cccc(-c5ccccc5)c34)cc2-c2ccccc2)cc1 ; c1ccc(-c2ccc(N(c3ccc(-c4cccc5ccccc45)cc3)c3ccc(-c4ccccc4)c4c3oc3ccccc34)cc2-c2ccccc2)cc1 ; c1ccc(-c2ccc(N(c3ccc(-c4cccc5ccccc45)cc3)c3ccc(-c4ccccc4)c4c3sc3ccccc34)cc2-c2ccccc2)cc1]

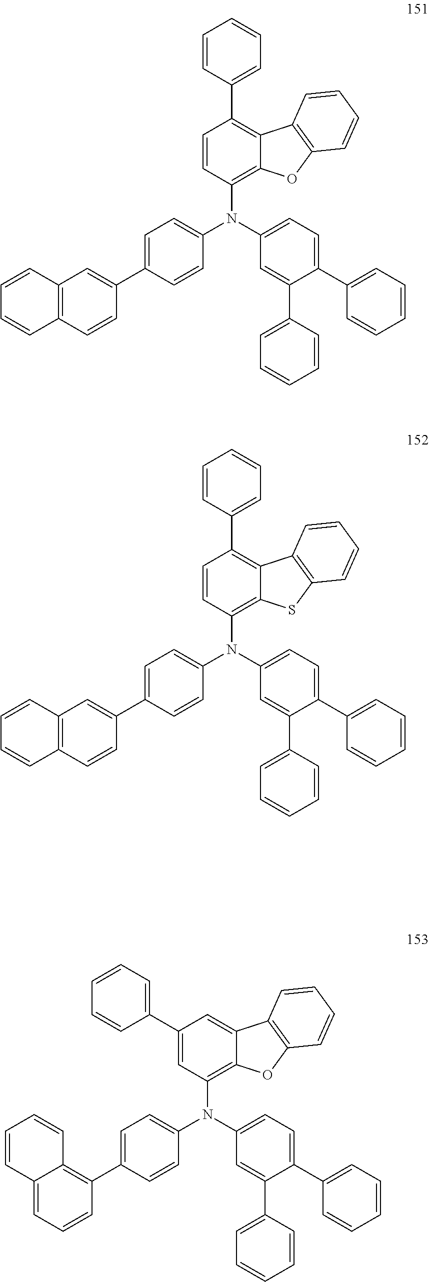 [SMILES: c1ccc(-c2ccc(N(c3ccc(-c4ccc5ccccc5c4)cc3)c3ccc(-c4ccccc4)c4c3oc3ccccc34)cc2-c2ccccc2)cc1 ; c1ccc(-c2ccc(N(c3ccc(-c4ccc5ccccc5c4)cc3)c3ccc(-c4ccccc4)c4c3sc3ccccc34)cc2-c2ccccc2)cc1 ; c1ccc(-c2cc(N(c3ccc(-c4cccc5ccccc45)cc3)c3ccc(-c4ccccc4)c(-c4ccccc4)c3)c3oc4ccccc4c3c2)cc1]

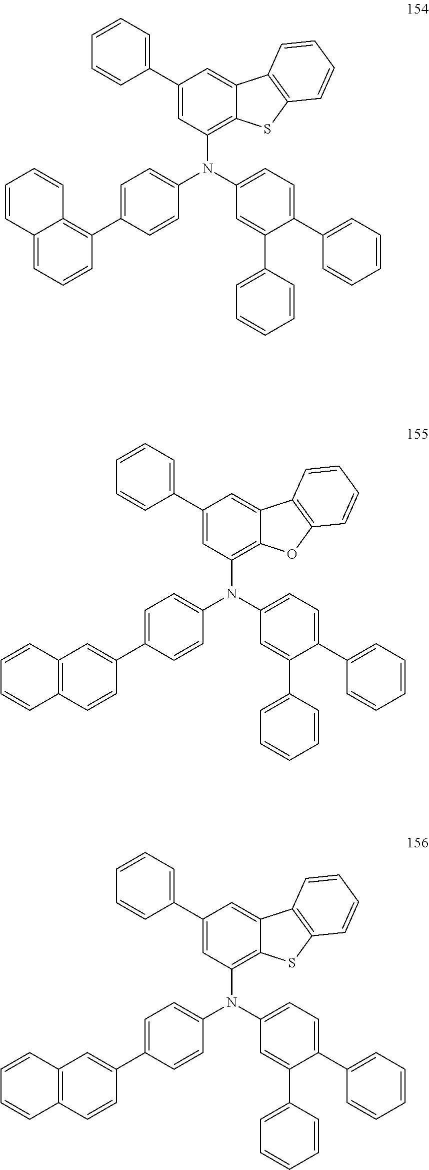 [SMILES: c1ccc(-c2cc(N(c3ccc(-c4cccc5ccccc45)cc3)c3ccc(-c4ccccc4)c(-c4ccccc4)c3)c3sc4ccccc4c3c2)cc1 ; c1ccc(-c2cc(N(c3ccc(-c4ccc5ccccc5c4)cc3)c3ccc(-c4ccccc4)c(-c4ccccc4)c3)c3oc4ccccc4c3c2)cc1 ; c1ccc(-c2cc(N(c3ccc(-c4ccc5ccccc5c4)cc3)c3ccc(-c4ccccc4)c(-c4ccccc4)c3)c3sc4ccccc4c3c2)cc1]

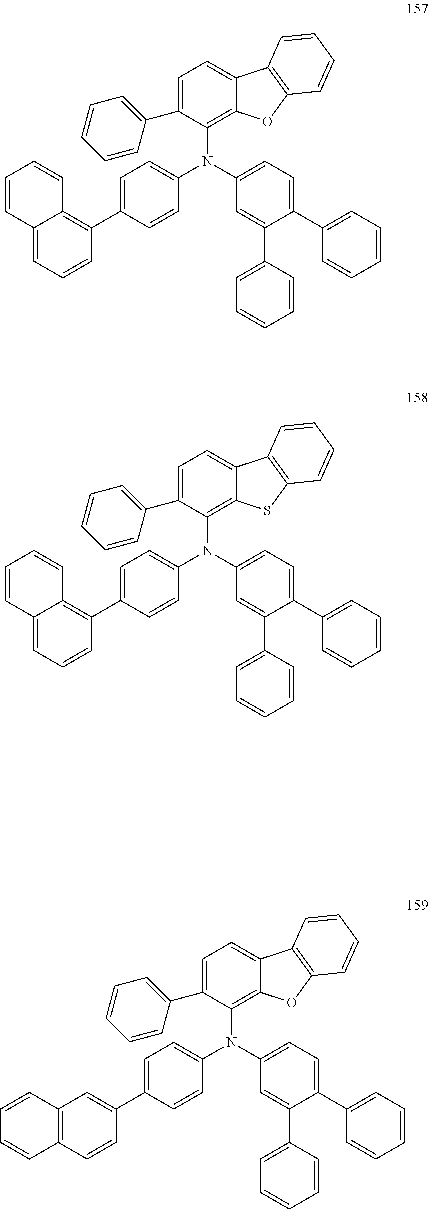 [SMILES: c1ccc(-c2ccc(N(c3ccc(-c4cccc5ccccc45)cc3)c3c(-c4ccccc4)ccc4c3oc3ccccc34)cc2-c2ccccc2)cc1 ; c1ccc(-c2ccc(N(c3ccc(-c4cccc5ccccc45)cc3)c3c(-c4ccccc4)ccc4c3sc3ccccc34)cc2-c2ccccc2)cc1 ; c1ccc(-c2ccc(N(c3ccc(-c4ccc5ccccc5c4)cc3)c3c(-c4ccccc4)ccc4c3oc3ccccc34)cc2-c2ccccc2)cc1]

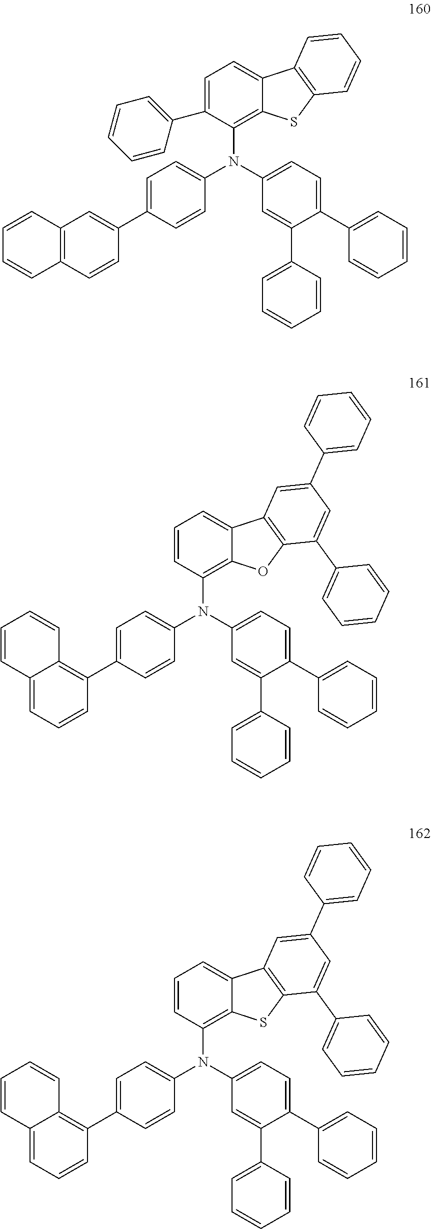 [SMILES: c1ccc(-c2ccc(N(c3ccc(-c4ccc5ccccc5c4)cc3)c3c(-c4ccccc4)ccc4c3sc3ccccc34)cc2-c2ccccc2)cc1 ; c1ccc(-c2cc(-c3ccccc3)c3oc4c(N(c5ccc(-c6cccc7ccccc67)cc5)c5ccc(-c6ccccc6)c(-c6ccccc6)c5)cccc4c3c2)cc1 ; c1ccc(-c2cc(-c3ccccc3)c3sc4c(N(c5ccc(-c6cccc7ccccc67)cc5)c5ccc(-c6ccccc6)c(-c6ccccc6)c5)cccc4c3c2)cc1]

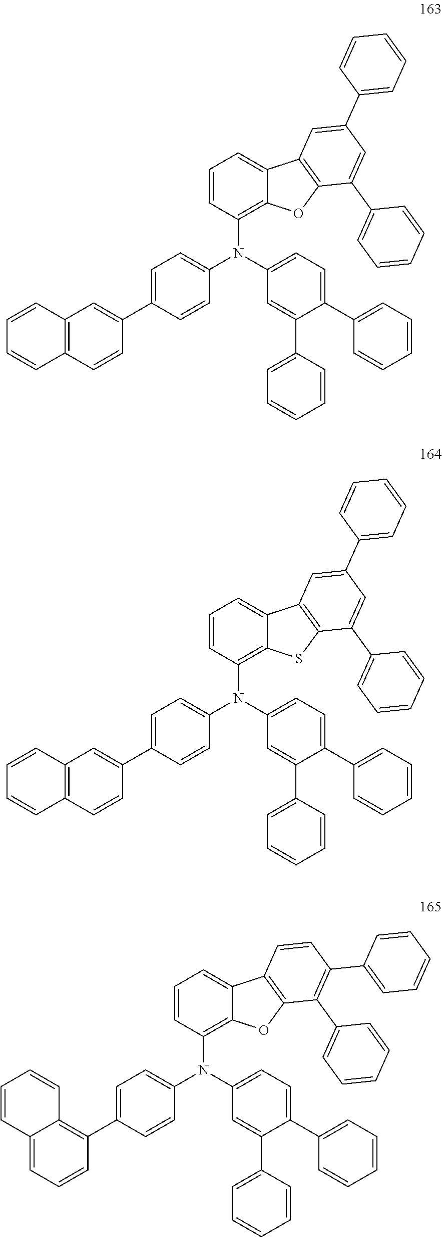 [SMILES: c1ccc(-c2cc(-c3ccccc3)c3oc4c(N(c5ccc(-c6ccc7ccccc7c6)cc5)c5ccc(-c6ccccc6)c(-c6ccccc6)c5)cccc4c3c2)cc1 ; c1ccc(-c2cc(-c3ccccc3)c3sc4c(N(c5ccc(-c6ccc7ccccc7c6)cc5)c5ccc(-c6ccccc6)c(-c6ccccc6)c5)cccc4c3c2)cc1 ; c1ccc(-c2ccc(N(c3ccc(-c4cccc5ccccc45)cc3)c3cccc4c3oc3c(-c5ccccc5)c(-c5ccccc5)ccc34)cc2-c2ccccc2)cc1]

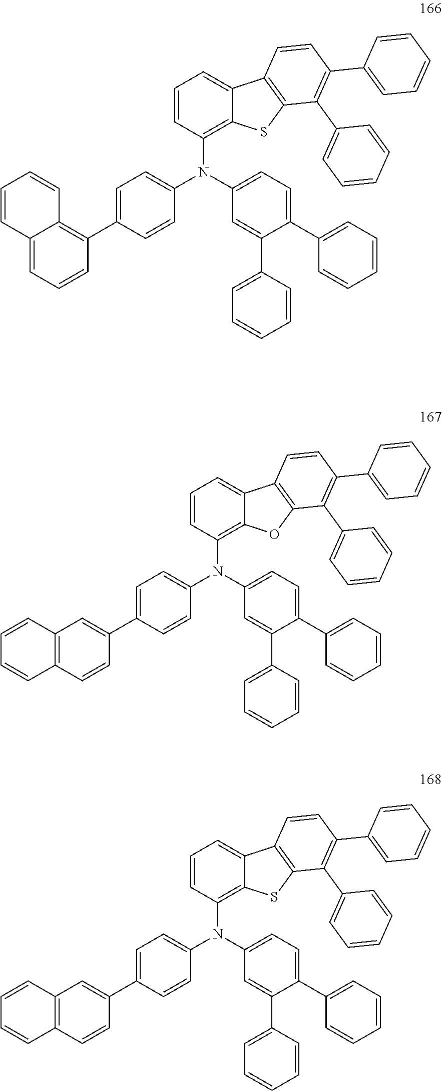 [SMILES: c1ccc(-c2ccc(N(c3ccc(-c4cccc5ccccc45)cc3)c3cccc4c3sc3c(-c5ccccc5)c(-c5ccccc5)ccc34)cc2-c2ccccc2)cc1 ; c1ccc(-c2ccc(N(c3ccc(-c4ccc5ccccc5c4)cc3)c3cccc4c3oc3c(-c5ccccc5)c(-c5ccccc5)ccc34)cc2-c2ccccc2)cc1 ; c1ccc(-c2ccc(N(c3ccc(-c4ccc5ccccc5c4)cc3)c3cccc4c3sc3c(-c5ccccc5)c(-c5ccccc5)ccc34)cc2-c2ccccc2)cc1]

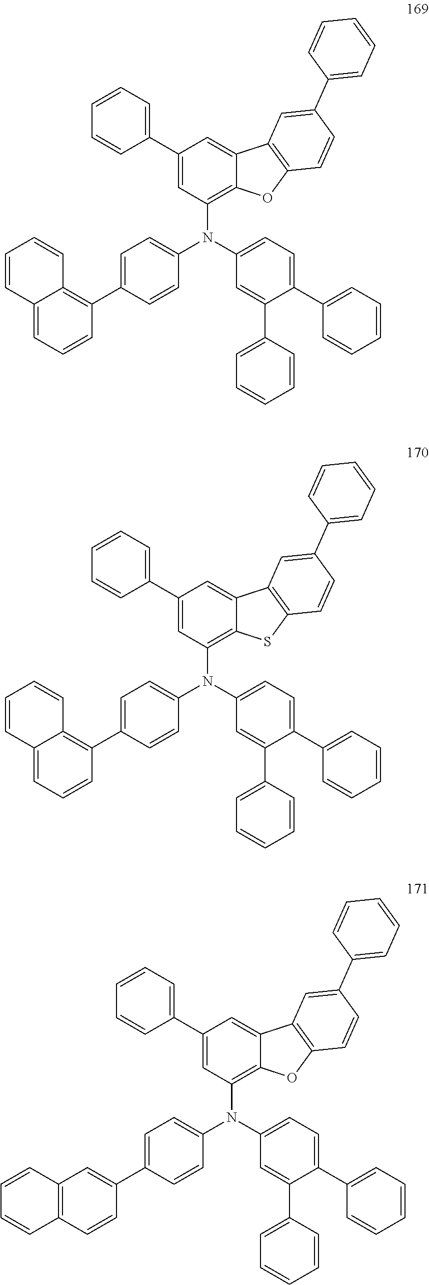 [SMILES: c1ccc(-c2ccc3oc4c(N(c5ccc(-c6cccc7ccccc67)cc5)c5ccc(-c6ccccc6)c(-c6ccccc6)c5)cc(-c5ccccc5)cc4c3c2)cc1 ; c1ccc(-c2ccc3sc4c(N(c5ccc(-c6cccc7ccccc67)cc5)c5ccc(-c6ccccc6)c(-c6ccccc6)c5)cc(-c5ccccc5)cc4c3c2)cc1 ; c1ccc(-c2ccc3oc4c(N(c5ccc(-c6ccc7ccccc7c6)cc5)c5ccc(-c6ccccc6)c(-c6ccccc6)c5)cc(-c5ccccc5)cc4c3c2)cc1]

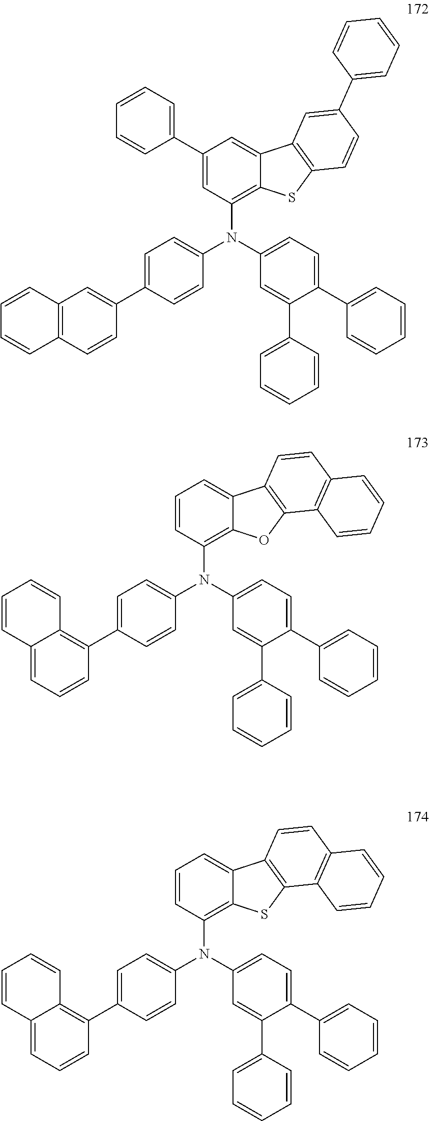 [SMILES: c1ccc(-c2ccc3sc4c(N(c5ccc(-c6ccc7ccccc7c6)cc5)c5ccc(-c6ccccc6)c(-c6ccccc6)c5)cc(-c5ccccc5)cc4c3c2)cc1 ; c1ccc(-c2ccc(N(c3ccc(-c4cccc5ccccc45)cc3)c3cccc4c3oc3c5ccccc5ccc43)cc2-c2ccccc2)cc1 ; c1ccc(-c2ccc(N(c3ccc(-c4cccc5ccccc45)cc3)c3cccc4c3sc3c5ccccc5ccc43)cc2-c2ccccc2)cc1]

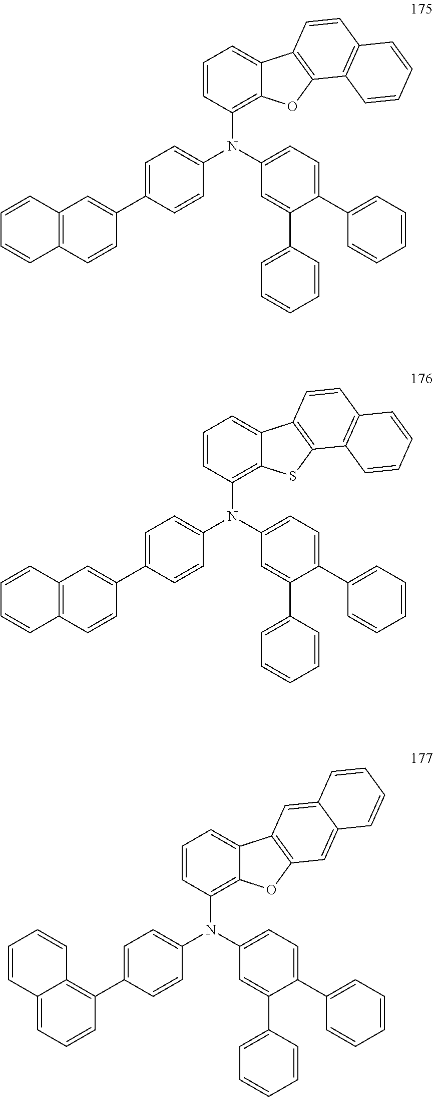 [SMILES: c1ccc(-c2ccc(N(c3ccc(-c4ccc5ccccc5c4)cc3)c3cccc4c3oc3c5ccccc5ccc43)cc2-c2ccccc2)cc1 ; c1ccc(-c2ccc(N(c3ccc(-c4ccc5ccccc5c4)cc3)c3cccc4c3sc3c5ccccc5ccc43)cc2-c2ccccc2)cc1 ; c1ccc(-c2ccc(N(c3ccc(-c4cccc5ccccc45)cc3)c3cccc4c3oc3cc5ccccc5cc34)cc2-c2ccccc2)cc1]

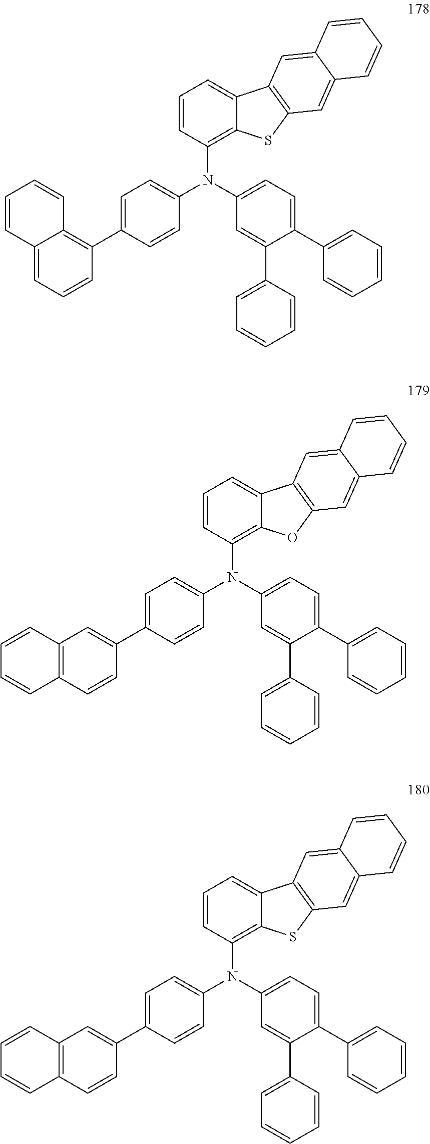 [SMILES: c1ccc(-c2ccc(N(c3ccc(-c4cccc5ccccc45)cc3)c3cccc4c3sc3cc5ccccc5cc34)cc2-c2ccccc2)cc1 ; c1ccc(-c2ccc(N(c3ccc(-c4ccc5ccccc5c4)cc3)c3cccc4c3oc3cc5ccccc5cc34)cc2-c2ccccc2)cc1 ; c1ccc(-c2ccc(N(c3ccc(-c4ccc5ccccc5c4)cc3)c3cccc4c3sc3cc5ccccc5cc34)cc2-c2ccccc2)cc1]

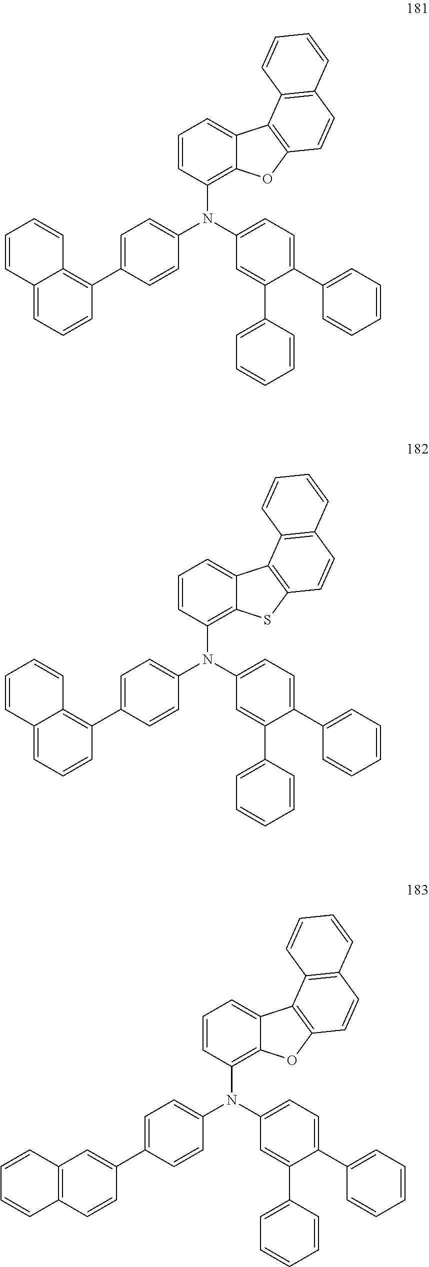 [SMILES: c1ccc(-c2ccc(N(c3ccc(-c4cccc5ccccc45)cc3)c3cccc4c3oc3ccc5ccccc5c34)cc2-c2ccccc2)cc1 ; c1ccc(-c2ccc(N(c3ccc(-c4cccc5ccccc45)cc3)c3cccc4c3sc3ccc5ccccc5c34)cc2-c2ccccc2)cc1 ; c1ccc(-c2ccc(N(c3ccc(-c4ccc5ccccc5c4)cc3)c3cccc4c3oc3ccc5ccccc5c34)cc2-c2ccccc2)cc1]

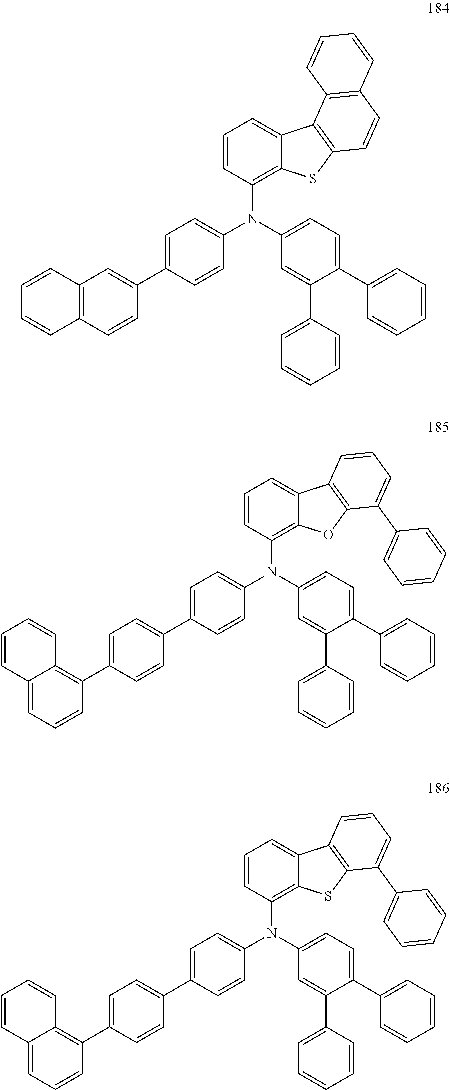 [SMILES: c1ccc(-c2ccc(N(c3ccc(-c4ccc5ccccc5c4)cc3)c3cccc4c3sc3ccc5ccccc5c34)cc2-c2ccccc2)cc1 ; c1ccc(-c2ccc(N(c3ccc(-c4ccc(-c5cccc6ccccc56)cc4)cc3)c3cccc4c3oc3c(-c5ccccc5)cccc34)cc2-c2ccccc2)cc1 ; c1ccc(-c2ccc(N(c3ccc(-c4ccc(-c5cccc6ccccc56)cc4)cc3)c3cccc4c3sc3c(-c5ccccc5)cccc34)cc2-c2ccccc2)cc1]

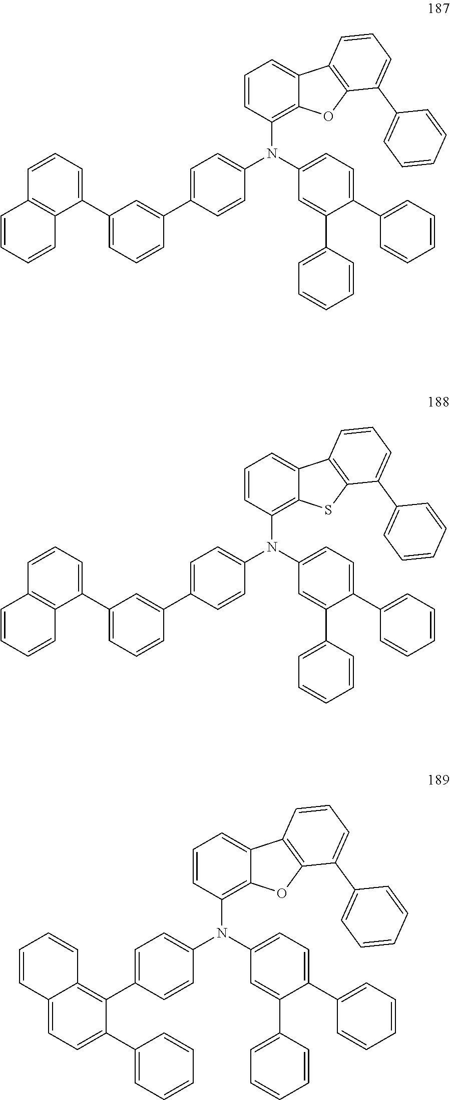 [SMILES: c1ccc(-c2ccc(N(c3ccc(-c4cccc(-c5cccc6ccccc56)c4)cc3)c3cccc4c3oc3c(-c5ccccc5)cccc34)cc2-c2ccccc2)cc1 ; c1ccc(-c2ccc(N(c3ccc(-c4cccc(-c5cccc6ccccc56)c4)cc3)c3cccc4c3sc3c(-c5ccccc5)cccc34)cc2-c2ccccc2)cc1 ; c1ccc(-c2ccc(N(c3ccc(-c4c(-c5ccccc5)ccc5ccccc45)cc3)c3cccc4c3oc3c(-c5ccccc5)cccc34)cc2-c2ccccc2)cc1]

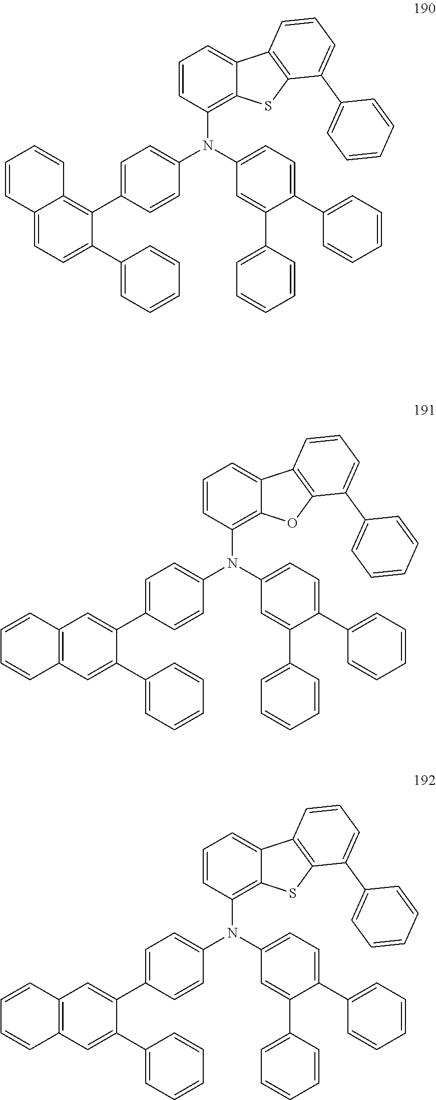 [SMILES: c1ccc(-c2ccc(N(c3ccc(-c4c(-c5ccccc5)ccc5ccccc45)cc3)c3cccc4c3sc3c(-c5ccccc5)cccc34)cc2-c2ccccc2)cc1 ; c1ccc(-c2ccc(N(c3ccc(-c4cc5ccccc5cc4-c4ccccc4)cc3)c3cccc4c3oc3c(-c5ccccc5)cccc34)cc2-c2ccccc2)cc1 ; c1ccc(-c2ccc(N(c3ccc(-c4cc5ccccc5cc4-c4ccccc4)cc3)c3cccc4c3sc3c(-c5ccccc5)cccc34)cc2-c2ccccc2)cc1]

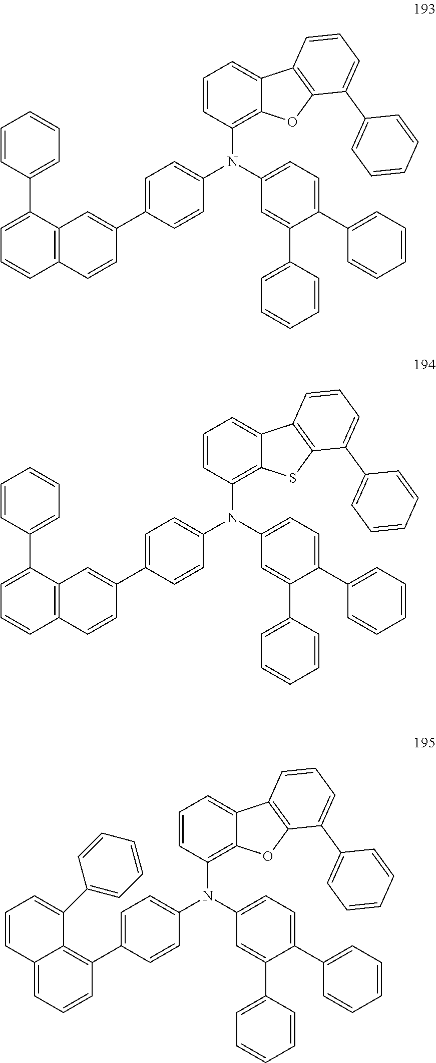 [SMILES: c1ccc(-c2ccc(N(c3ccc(-c4ccc5cccc(-c6ccccc6)c5c4)cc3)c3cccc4c3oc3c(-c5ccccc5)cccc34)cc2-c2ccccc2)cc1 ; c1ccc(-c2ccc(N(c3ccc(-c4ccc5cccc(-c6ccccc6)c5c4)cc3)c3cccc4c3sc3c(-c5ccccc5)cccc34)cc2-c2ccccc2)cc1 ; c1ccc(-c2ccc(N(c3ccc(-c4cccc5cccc(-c6ccccc6)c45)cc3)c3cccc4c3oc3c(-c5ccccc5)cccc34)cc2-c2ccccc2)cc1]

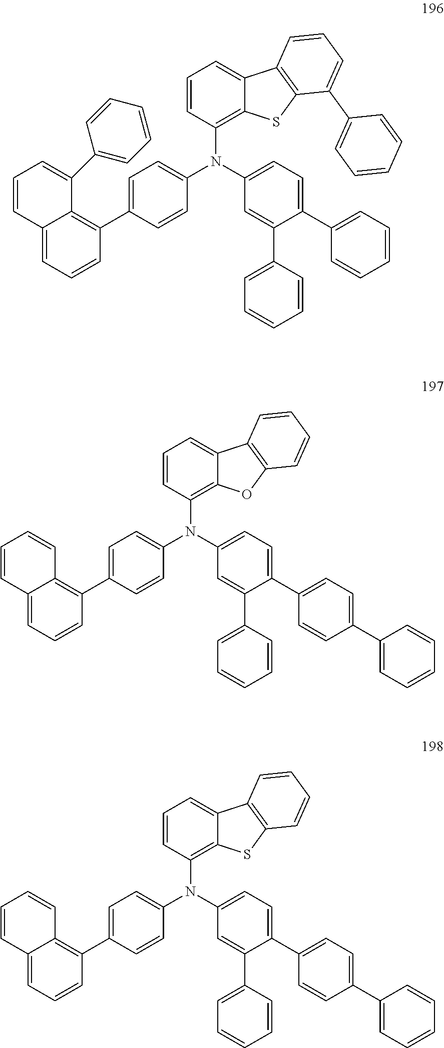 [SMILES: c1ccc(-c2ccc(N(c3ccc(-c4cccc5cccc(-c6ccccc6)c45)cc3)c3cccc4c3sc3c(-c5ccccc5)cccc34)cc2-c2ccccc2)cc1 ; c1ccc(-c2ccc(-c3ccc(N(c4ccc(-c5cccc6ccccc56)cc4)c4cccc5c4oc4ccccc45)cc3-c3ccccc3)cc2)cc1 ; c1ccc(-c2ccc(-c3ccc(N(c4ccc(-c5cccc6ccccc56)cc4)c4cccc5c4sc4ccccc45)cc3-c3ccccc3)cc2)cc1]

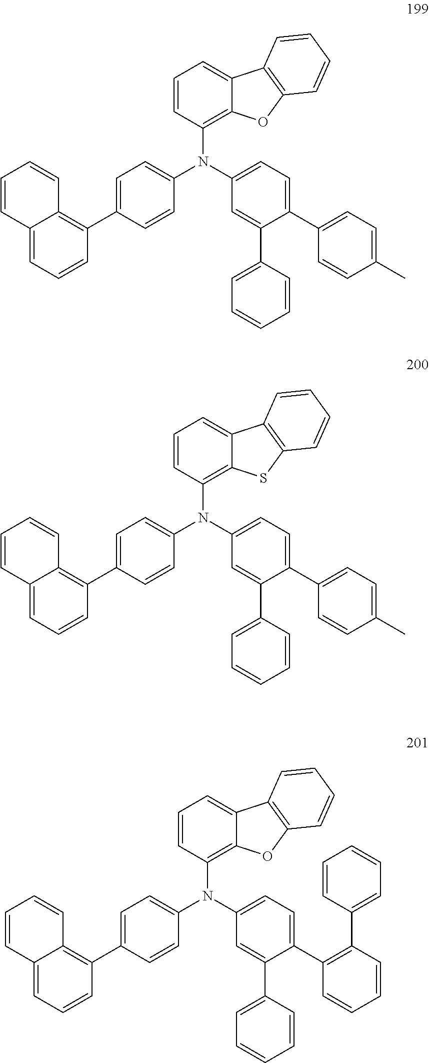 [SMILES: Cc1ccc(-c2ccc(N(c3ccc(-c4cccc5ccccc45)cc3)c3cccc4c3oc3ccccc34)cc2-c2ccccc2)cc1 ; Cc1ccc(-c2ccc(N(c3ccc(-c4cccc5ccccc45)cc3)c3cccc4c3sc3ccccc34)cc2-c2ccccc2)cc1 ; c1ccc(-c2ccccc2-c2ccc(N(c3ccc(-c4cccc5ccccc45)cc3)c3cccc4c3oc3ccccc34)cc2-c2ccccc2)cc1]

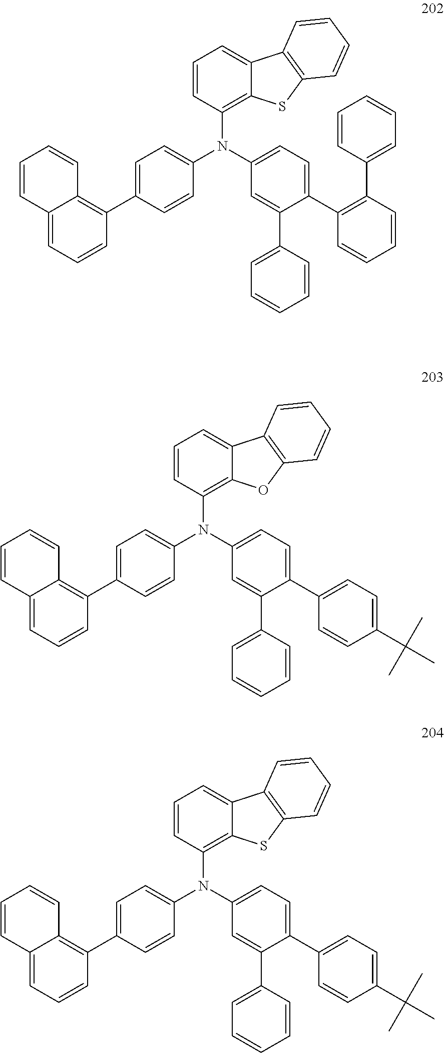 [SMILES: c1ccc(-c2ccccc2-c2ccc(N(c3ccc(-c4cccc5ccccc45)cc3)c3cccc4c3sc3ccccc34)cc2-c2ccccc2)cc1 ; CC(C)(C)c1ccc(-c2ccc(N(c3ccc(-c4cccc5ccccc45)cc3)c3cccc4c3oc3ccccc34)cc2-c2ccccc2)cc1 ; CC(C)(C)c1ccc(-c2ccc(N(c3ccc(-c4cccc5ccccc45)cc3)c3cccc4c3sc3ccccc34)cc2-c2ccccc2)cc1]

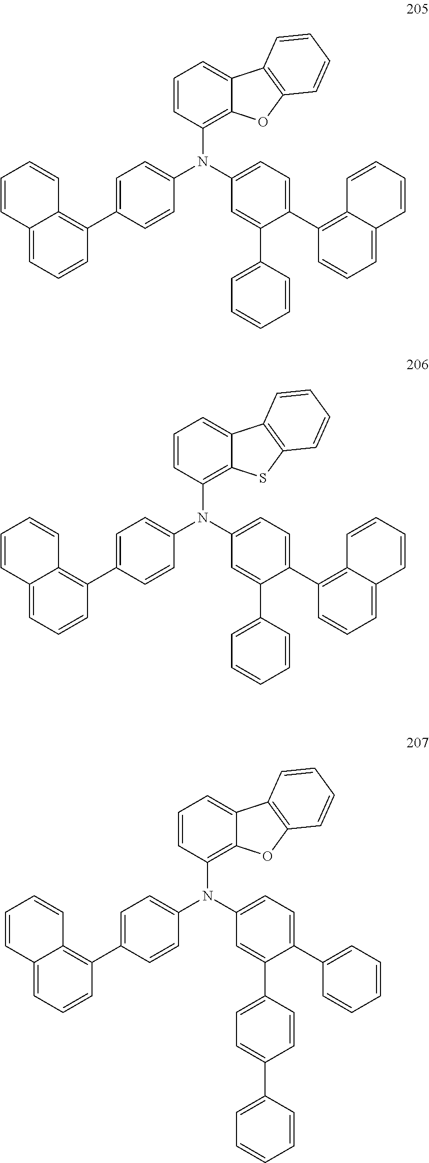 [SMILES: c1ccc(-c2cc(N(c3ccc(-c4cccc5ccccc45)cc3)c3cccc4c3oc3ccccc34)ccc2-c2cccc3ccccc23)cc1 ; c1ccc(-c2cc(N(c3ccc(-c4cccc5ccccc45)cc3)c3cccc4c3sc3ccccc34)ccc2-c2cccc3ccccc23)cc1 ; c1ccc(-c2ccc(-c3cc(N(c4ccc(-c5cccc6ccccc56)cc4)c4cccc5c4oc4ccccc45)ccc3-c3ccccc3)cc2)cc1]

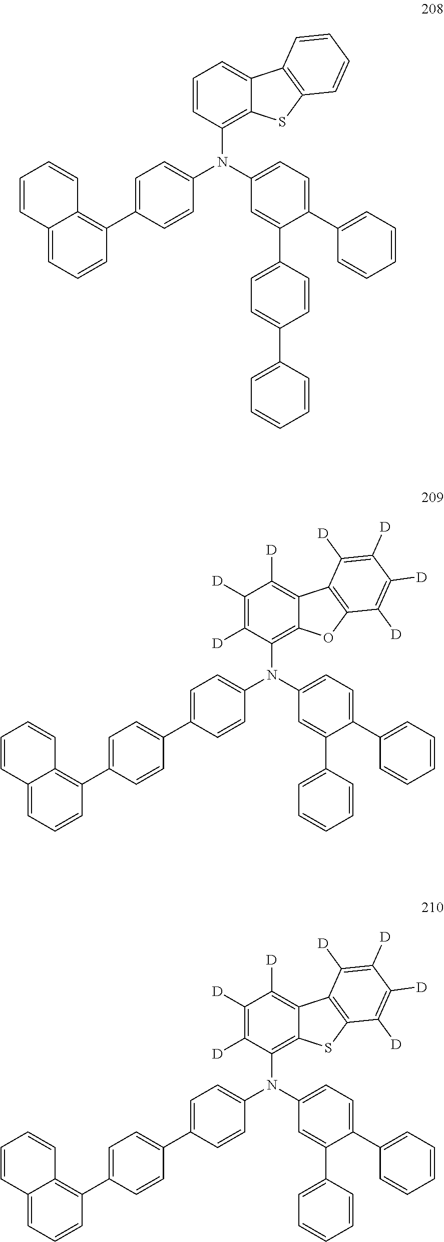 [SMILES: c1ccc(-c2ccc(-c3cc(N(c4ccc(-c5cccc6ccccc56)cc4)c4cccc5c4sc4ccccc45)ccc3-c3ccccc3)cc2)cc1 ; [2H]c1c([2H])c([2H])c2c(oc3c(N(c4ccc(-c5ccc(-c6cccc7ccccc67)cc5)cc4)c4ccc(-c5ccccc5)c(-c5ccccc5)c4)c([2H])c([2H])c([2H])c32)c1[2H] ; [2H]c1c([2H])c([2H])c2c(sc3c(N(c4ccc(-c5ccc(-c6cccc7ccccc67)cc5)cc4)c4ccc(-c5ccccc5)c(-c5ccccc5)c4)c([2H])c([2H])c([2H])c32)c1[2H]]

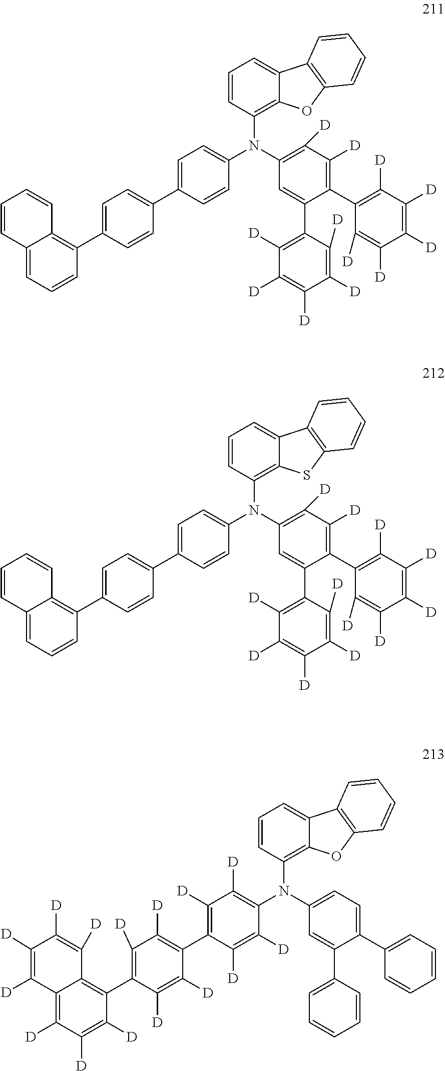 [SMILES: [2H]c1c(N(c2ccc(-c3ccc(-c4cccc5ccccc45)cc3)cc2)c2cccc3c2oc2ccccc23)cc(-c2c([2H])c([2H])c([2H])c([2H])c2[2H])c(-c2c([2H])c([2H])c([2H])c([2H])c2[2H])c1[2H] ; [2H]c1c(N(c2ccc(-c3ccc(-c4cccc5ccccc45)cc3)cc2)c2cccc3c2sc2ccccc23)cc(-c2c([2H])c([2H])c([2H])c([2H])c2[2H])c(-c2c([2H])c([2H])c([2H])c([2H])c2[2H])c1[2H] ; [2H]c1c([2H])c(-c2c([2H])c([2H])c([2H])c3c([2H])c([2H])c([2H])c([2H])c23)c([2H])c([2H])c1-c1c([2H])c([2H])c(N(c2ccc(-c3ccccc3)c(-c3ccccc3)c2)c2cccc3c2oc2ccccc23)c([2H])c1[2H]]

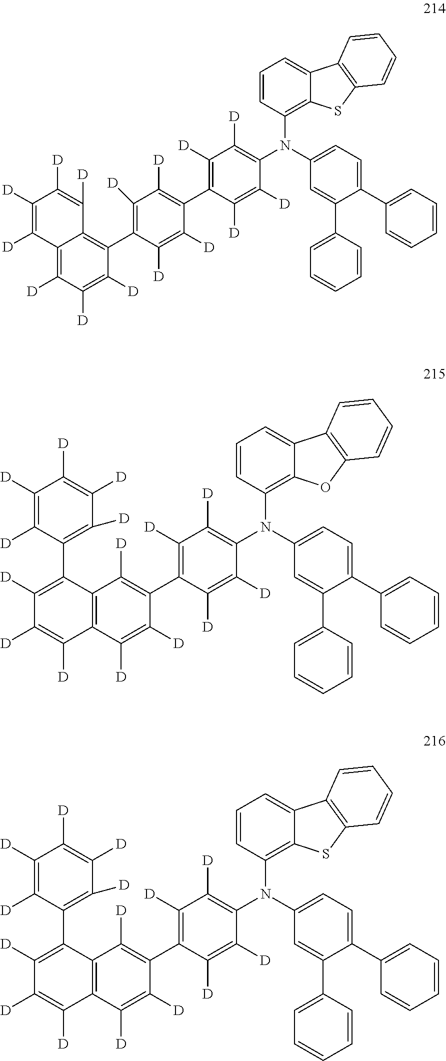

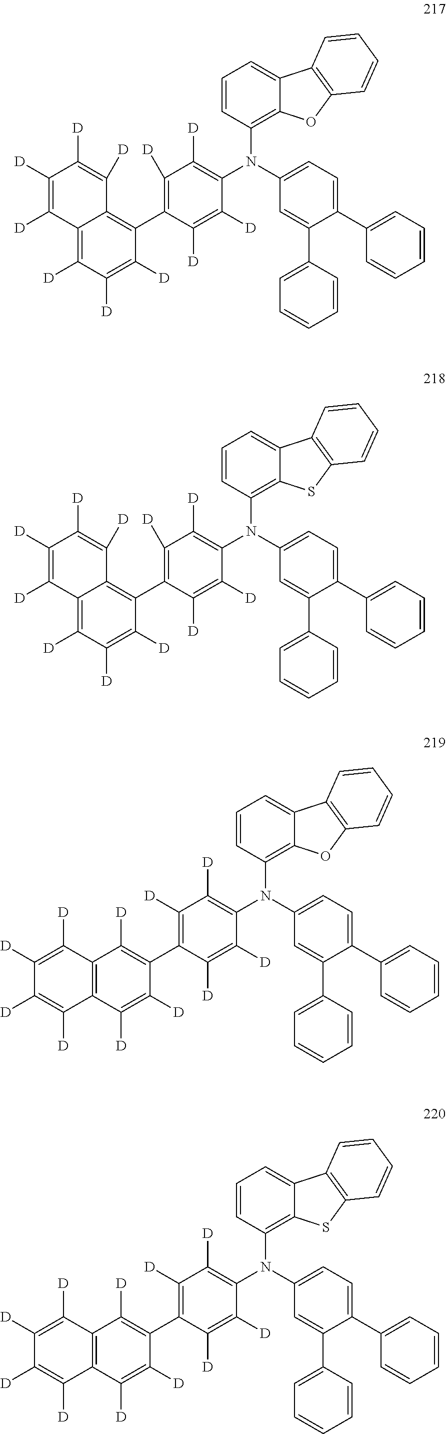

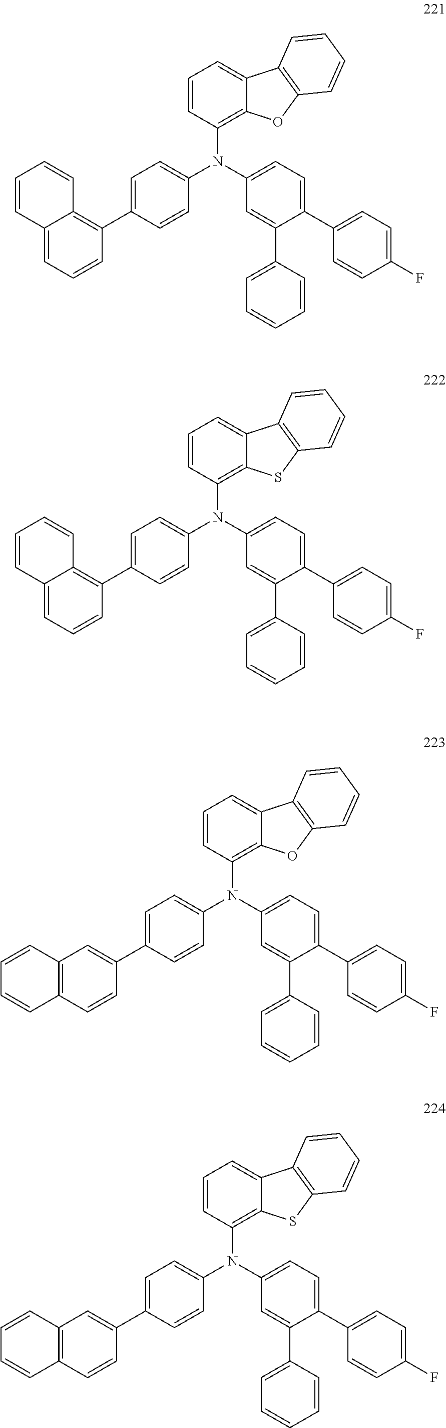 [SMILES: Fc1ccc(-c2ccc(N(c3ccc(-c4cccc5ccccc45)cc3)c3cccc4c3oc3ccccc34)cc2-c2ccccc2)cc1 ; Fc1ccc(-c2ccc(N(c3ccc(-c4cccc5ccccc45)cc3)c3cccc4c3sc3ccccc34)cc2-c2ccccc2)cc1 ; Fc1ccc(-c2ccc(N(c3ccc(-c4ccc5ccccc5c4)cc3)c3cccc4c3oc3ccccc34)cc2-c2ccccc2)cc1 ; Fc1ccc(-c2ccc(N(c3ccc(-c4ccc5ccccc5c4)cc3)c3cccc4c3sc3ccccc34)cc2-c2ccccc2)cc1]

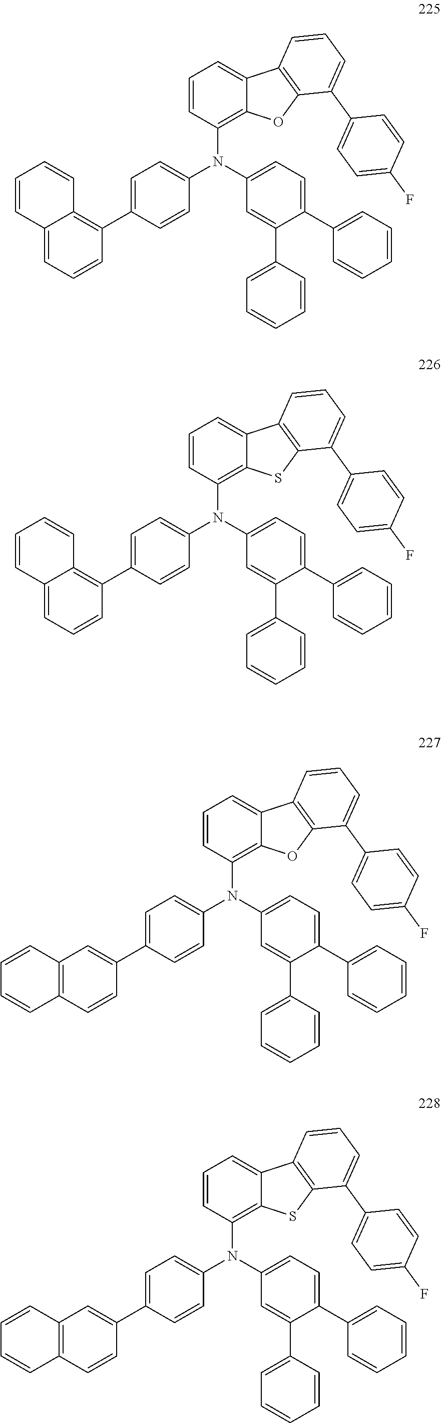 [SMILES: Fc1ccc(-c2cccc3c2oc2c(N(c4ccc(-c5cccc6ccccc56)cc4)c4ccc(-c5ccccc5)c(-c5ccccc5)c4)cccc23)cc1 ; Fc1ccc(-c2cccc3c2sc2c(N(c4ccc(-c5cccc6ccccc56)cc4)c4ccc(-c5ccccc5)c(-c5ccccc5)c4)cccc23)cc1 ; Fc1ccc(-c2cccc3c2oc2c(N(c4ccc(-c5ccc6ccccc6c5)cc4)c4ccc(-c5ccccc5)c(-c5ccccc5)c4)cccc23)cc1 ; Fc1ccc(-c2cccc3c2sc2c(N(c4ccc(-c5ccc6ccccc6c5)cc4)c4ccc(-c5ccccc5)c(-c5ccccc5)c4)cccc23)cc1]

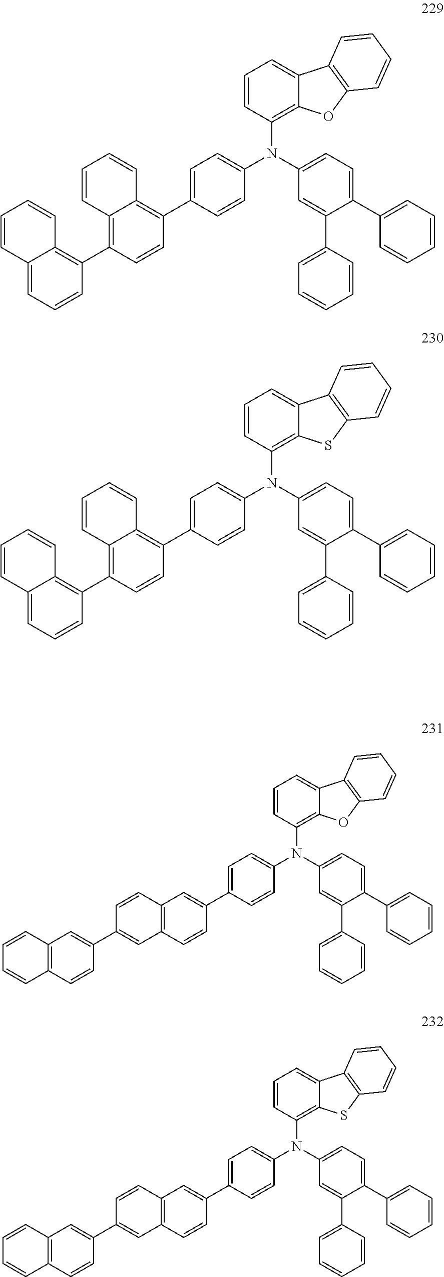 [SMILES: c1ccc(-c2ccc(N(c3ccc(-c4ccc(-c5cccc6ccccc56)c5ccccc45)cc3)c3cccc4c3oc3ccccc34)cc2-c2ccccc2)cc1 ; c1ccc(-c2ccc(N(c3ccc(-c4ccc(-c5cccc6ccccc56)c5ccccc45)cc3)c3cccc4c3sc3ccccc34)cc2-c2ccccc2)cc1 ; c1ccc(-c2ccc(N(c3ccc(-c4ccc5cc(-c6ccc7ccccc7c6)ccc5c4)cc3)c3cccc4c3oc3ccccc34)cc2-c2ccccc2)cc1 ; c1ccc(-c2ccc(N(c3ccc(-c4ccc5cc(-c6ccc7ccccc7c6)ccc5c4)cc3)c3cccc4c3sc3ccccc34)cc2-c2ccccc2)cc1]

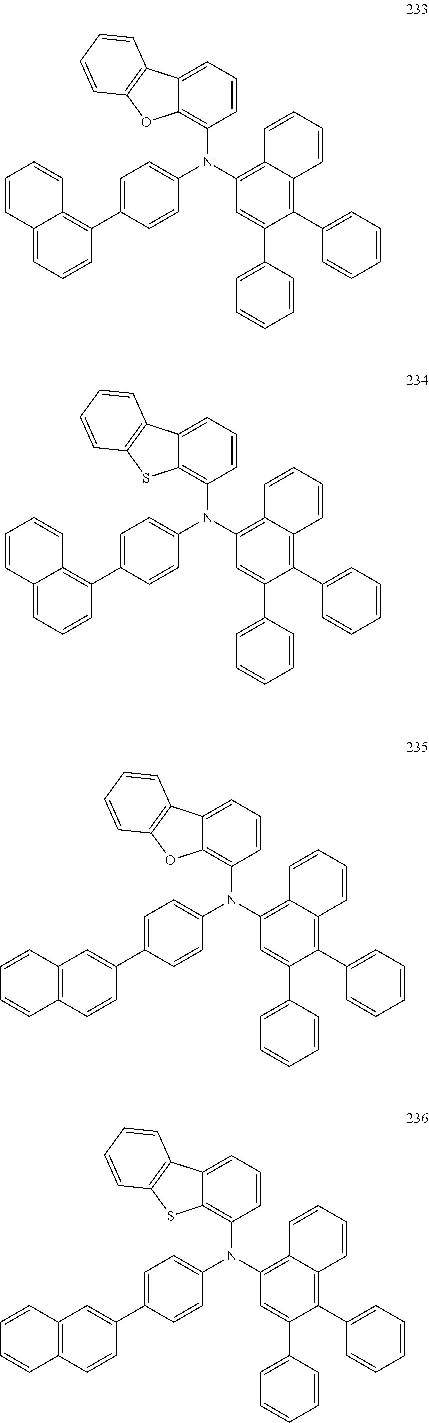 [SMILES: c1ccc(-c2cc(N(c3ccc(-c4cccc5ccccc45)cc3)c3cccc4c3oc3ccccc34)c3ccccc3c2-c2ccccc2)cc1 ; c1ccc(-c2cc(N(c3ccc(-c4cccc5ccccc45)cc3)c3cccc4c3sc3ccccc34)c3ccccc3c2-c2ccccc2)cc1 ; c1ccc(-c2cc(N(c3ccc(-c4ccc5ccccc5c4)cc3)c3cccc4c3oc3ccccc34)c3ccccc3c2-c2ccccc2)cc1 ; c1ccc(-c2cc(N(c3ccc(-c4ccc5ccccc5c4)cc3)c3cccc4c3sc3ccccc34)c3ccccc3c2-c2ccccc2)cc1]

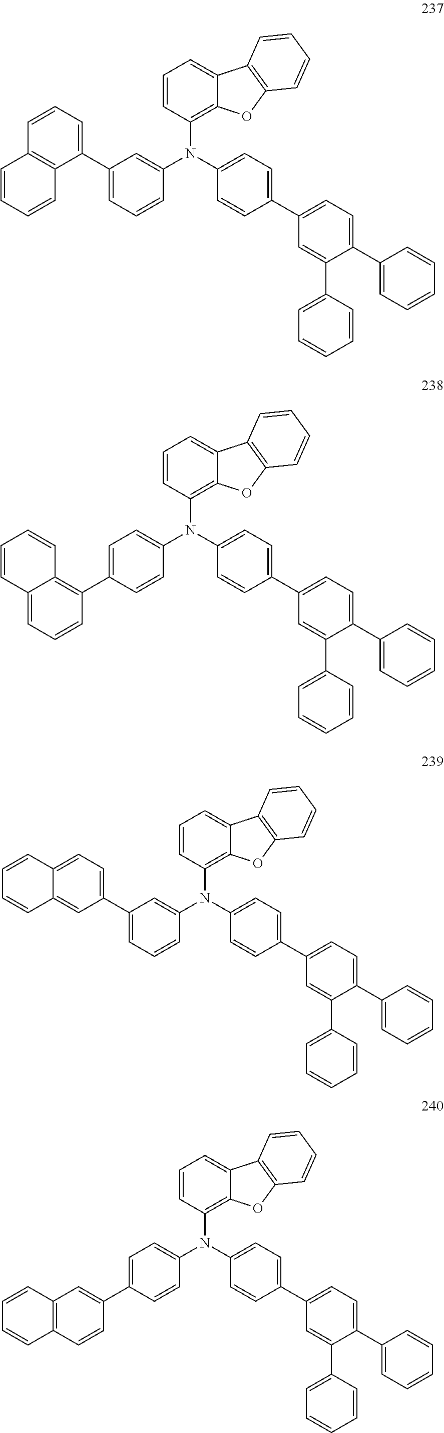 [SMILES: c1ccc(-c2ccc(-c3ccc(N(c4cccc(-c5cccc6ccccc56)c4)c4cccc5c4oc4ccccc45)cc3)cc2-c2ccccc2)cc1 ; c1ccc(-c2ccc(-c3ccc(N(c4ccc(-c5cccc6ccccc56)cc4)c4cccc5c4oc4ccccc45)cc3)cc2-c2ccccc2)cc1 ; c1ccc(-c2ccc(-c3ccc(N(c4cccc(-c5ccc6ccccc6c5)c4)c4cccc5c4oc4ccccc45)cc3)cc2-c2ccccc2)cc1 ; c1ccc(-c2ccc(-c3ccc(N(c4ccc(-c5ccc6ccccc6c5)cc4)c4cccc5c4oc4ccccc45)cc3)cc2-c2ccccc2)cc1]

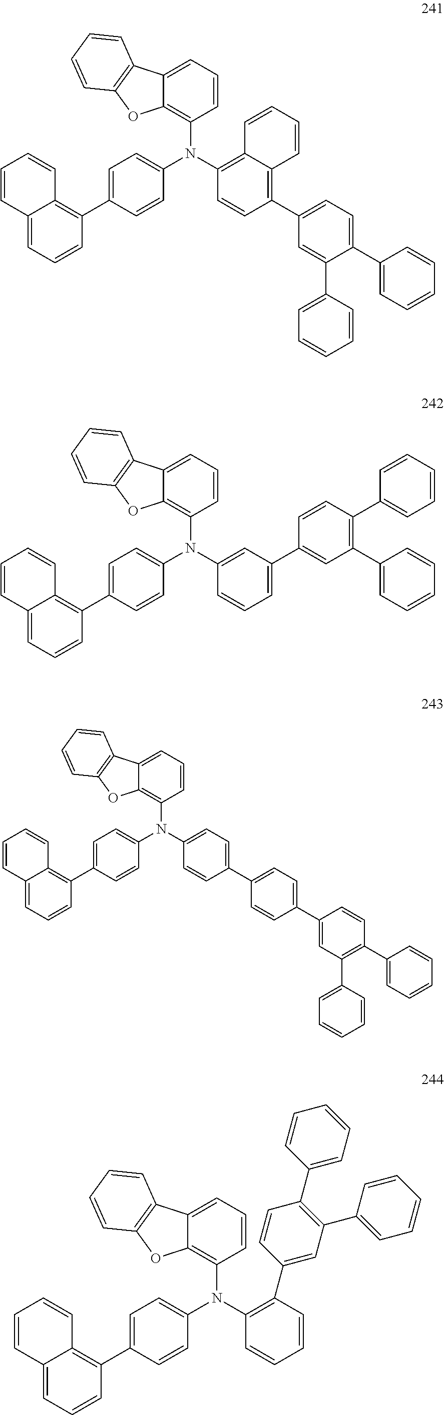 [SMILES: c1ccc(-c2ccc(-c3ccc(N(c4ccc(-c5cccc6ccccc56)cc4)c4cccc5c4oc4ccccc45)c4ccccc34)cc2-c2ccccc2)cc1 ; c1ccc(-c2ccc(-c3cccc(N(c4ccc(-c5cccc6ccccc56)cc4)c4cccc5c4oc4ccccc45)c3)cc2-c2ccccc2)cc1 ; c1ccc(-c2ccc(-c3ccc(-c4ccc(N(c5ccc(-c6cccc7ccccc67)cc5)c5cccc6c5oc5ccccc56)cc4)cc3)cc2-c2ccccc2)cc1 ; c1ccc(-c2ccc(-c3ccccc3N(c3ccc(-c4cccc5ccccc45)cc3)c3cccc4c3oc3ccccc34)cc2-c2ccccc2)cc1]

The amine compound represented by Formula 1 of one or more embodiments may include a dibenzoheterole (dibenzofuran or dibenzothiophene) moiety, a naphthyl group, and a 3,4-substituted phenyl group (ortho-terphenyl group), and in particular, the dibenzoheterole group and the 3,4-substituted phenyl group may each be characterized by being bonded to the nitrogen atom of the amine at a specific position. The amine compound of one or more embodiments may have excellent or suitable electrical stability and high charge transport ability due to the introduction of such a substituent and the specification of the substitution position. Accordingly, the service life of the amine compound of one or more embodiments may be improved. In addition, the light emitting element of one or more embodiments including the amine compound of one or more embodiments may have an improvement in luminous efficiency and service life.

In one or more embodiments, the hole transport region HTR in the light emitting element ED may further include a compound represented by Formula H-1. For example, the light emitting element ED of one or more embodiments may include a compound represented by Formula H-1 in another layer of the hole transport region HTR in which the amine compound of one or more embodiments of Formula 1 is not included. However, embodiments of the present disclosure are not limited thereto.

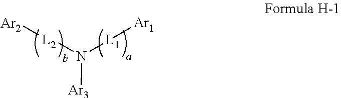

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In one or more embodiments, if (e.g., when) a or b is an integer of 2 or greater, a plurality of L₁'s and L₂'s may each independently be a substituted or unsubstituted arylene group having 6 to 30 ring-forming carbon atoms or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms or a substituted or unsubstituted heteroaryl group having 2 to 30 ring-forming carbon atoms. In one or more embodiments, in Formula H-1, Ar₃ may be a substituted or unsubstituted aryl group having 6 to 30 ring-forming carbon atoms or a substituted or unsubstituted heteroaryl group having 2 to 30 ring-forming carbon atoms.

The compound represented by Formula H-1 may be a monoamine compound. In one or more embodiments, the compound represented by Formula H-1 may be a diamine compound in which at least one selected from among Ar₁ to Ar₃ includes an amine group as a substituent. In one or more embodiments, the compound represented by Formula H-1 may be a carbazole-based compound including a substituted or unsubstituted carbazole group in at least one of Ar₁ or Ar₂, or a fluorene-based compound including a substituted or unsubstituted fluorene group in at least one of Ar₁ or Ar₂.

The compound represented by Formula H-1 may be any one selected from among compounds in Compound Group H. However, the compounds listed in Compound Group H are mere examples, and the compounds represented by Formula H-1 are not limited to those represented in Compound Group H:

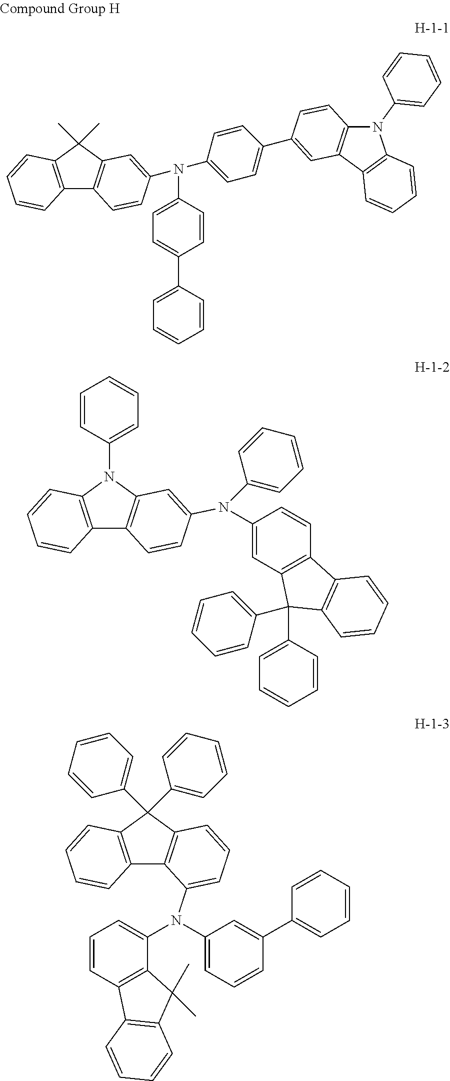 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

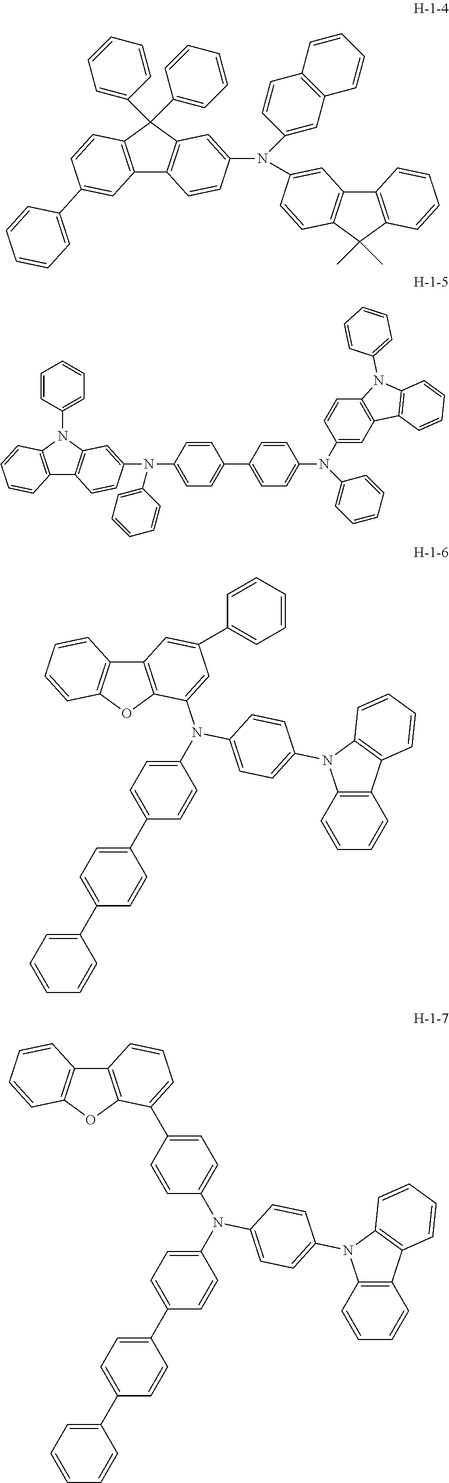 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

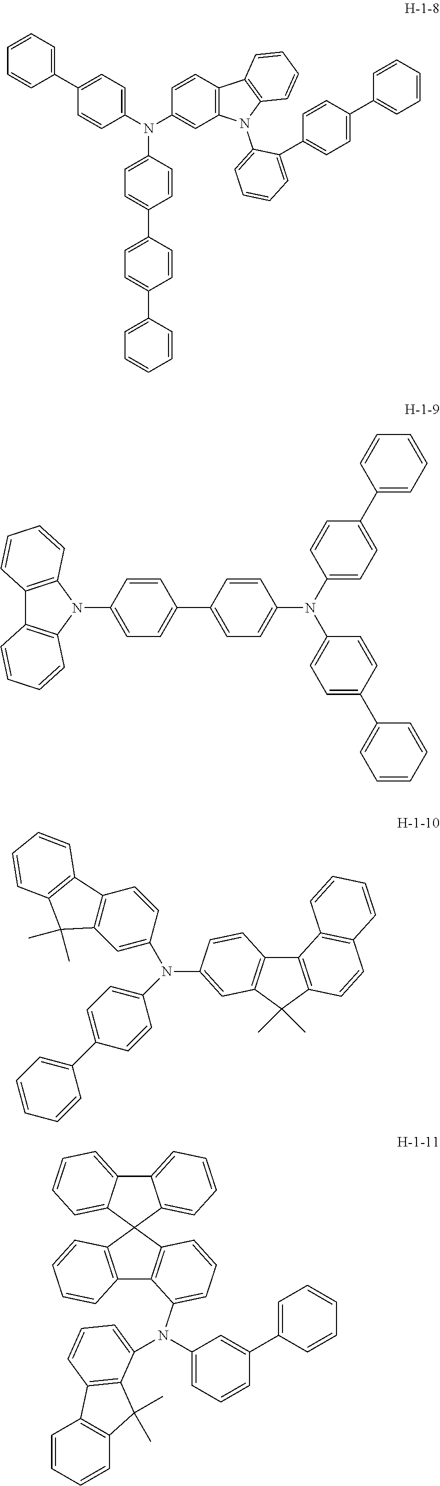

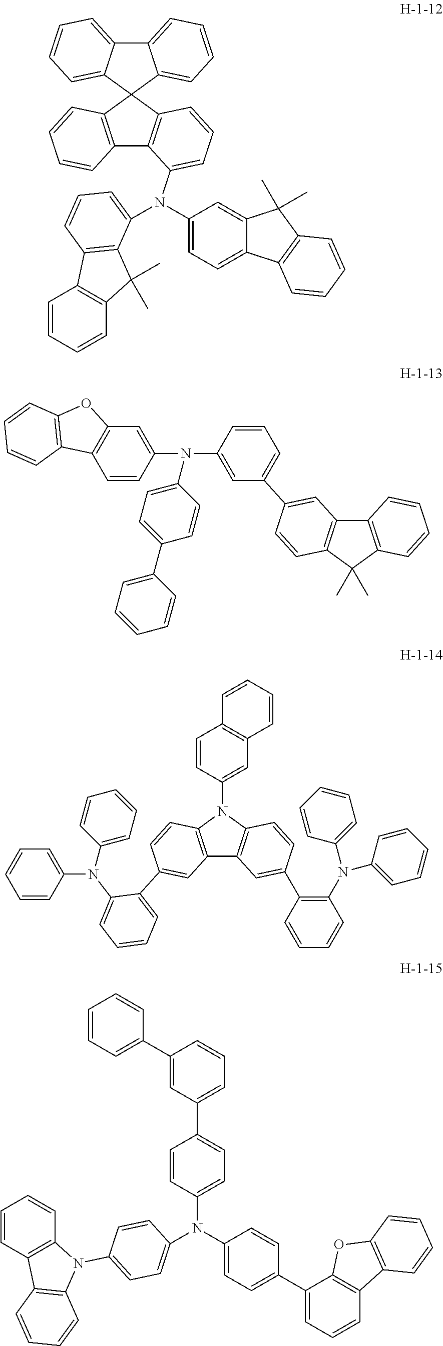 [SMILES: CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1]

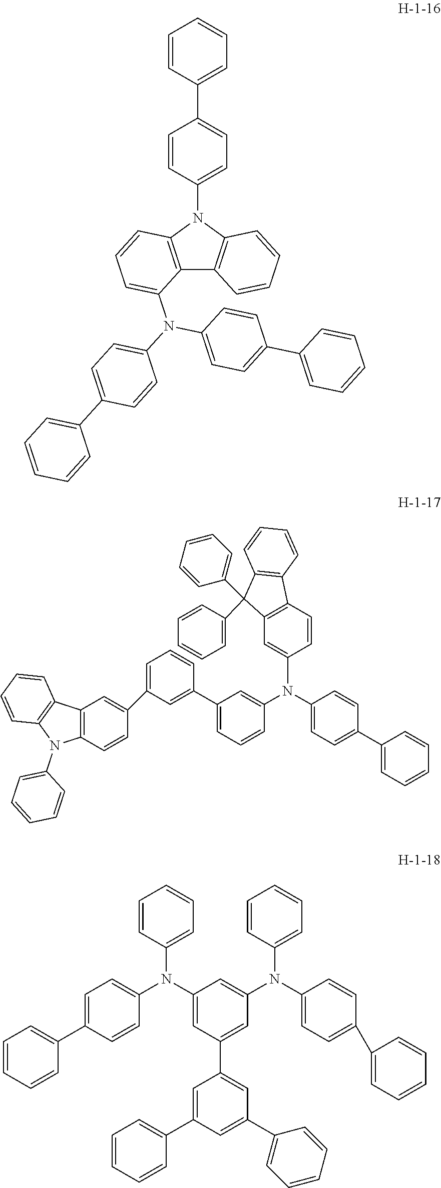 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

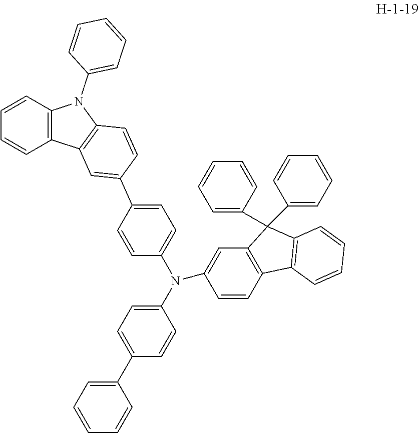 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In one or more embodiments, the hole transport region HTR may further include a phthalocyanine compound such as copper phthalocyanine; N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′4″-tris(N,N-diphenylamino)triphenylamine (TDATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-1-yl)-N,N′-diphenyl-benzidine (NPB), triphenylamine-containing polyetherketone (TPAPEK), 4-isopropyl-4′-methyldiphenyliodonium[tetrakis(pentafluorophenyl)borate], dipyrazino[2,3-f:2′,3′-h]quinoxaline-2,3,6,7,10,11-hexacarbonitrile (HATCN), and/or the like.

In one or more embodiments, the hole transport region HTR may further include a carbazole-based derivative such as N-phenyl carbazole and/or polyvinyl carbazole, a fluorene-based derivative, N,N′-bis(3-methylphenyl)-N,N′-diphenyl-[1,1-biphenyl]-4,4′-diamine (TPD), a triphenylamine-based derivative such as 4,4′,4″-tris(N-carbazolyl)triphenylamine (TCTA), N,N′-di(naphthalen-1-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 1,3-bis(N-carbazolyl)benzene (mCP), and/or the like.

In one or more embodiments, the hole transport region HTR may further include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include one or more of the above-described compounds of the hole transport region in at least one of the hole injection layer HIL, the hole transport layer HTL, or the electron blocking layer EBL.

A thickness of the hole transport region HTR may be from about 100 Å to about 10,000 Å, for example, from about 100 Å to about 5,000 Å. When the hole transport region HTR includes a hole injection layer HIL, the hole injection layer HIL may have, for example, a thickness of about 30 Å to about 1,000 Å. When the hole transport region HTR includes a hole transport layer HTL, the hole transport layer HTL may have a thickness of about 250 Å to about 1,000 Å. For example, if (e.g., when) the hole transport region HTR includes an electron blocking layer EBL, the electron blocking layer EBL may have a thickness of about 10 Å to about 1,000 Å. If the thicknesses of the hole transport region HTR, the hole injection layer HIL, the hole transport layer HTL, and the electron blocking layer EBL satisfy the above-described ranges, satisfactory hole transport properties may be achieved without a substantial increase in driving voltage.

In one or more embodiments, the hole transport region HTR may further include a charge generating material to increase conductivity in addition to the above-described materials. The charge generating material may be dispersed uniformly (substantially uniformly) or non-uniformly in the hole transport region HTR. The charge generating material may be, for example, a p-dopant. The p-dopant may include at least one of a halogenated metal compound, a quinone derivative, a metal oxide, or a cyano group-containing compound, but embodiments of the present disclosure are not limited thereto. For example, in some embodiments, the p-dopant may include a metal halide compound such as CuI and/or RbI, a quinone derivative such as tetracyanoquinodimethane (TCNQ) and/or 2,3,5,6-tetrafluoro-7,7′8,8-tetracyanoquinodimethane (F4-TCNQ), a metal oxide such as tungsten oxide and/or molybdenum oxide, a cyano group-containing compound such as dipyrazino[2,3-f:2′,3′-h]quinoxaline-2,3,6,7,10,11-hexacarbonitrile (HATCN) and/or 4-[2,3-bis[cyano-(4-cyano-2,3,5,6-tetrafluorophenyl)methylidene] cyclopropylidene]-cyanomethyl]-2,3,5,6-tetrafluorobenzonitrile (NDP9), and/or the like, but embodiments of the present disclosure are not limited thereto.

As described above, in some embodiments, the hole transport region HTR may further include at least one of the emission-auxiliary layer EAL or the electron blocking layer EBL in addition to the hole injection layer HIL and the hole transport layer HTL. The emission-auxiliary layer EAL may compensate for a resonance distance according to a wavelength of light emitted from the emission layer EML, and may increase light emission efficiency by adjusting a hole charge balance. In one or more embodiments, the emission-auxiliary layer EAL may also serve to prevent or reduce electron injection into the hole transport region HTR. Materials which may be included in the hole transport region HTR may be included in the emission-auxiliary layer EAL. The electron blocking layer EBL is a layer that serves to prevent or reduce the electron injection from the electron transport region ETR to the hole transport region HTR.

In the light-emitting element according to one or more embodiments, the emission layer EML may be provided on the hole transport region HTR. The emission layer EML may have, for example, a thickness of about 100 Å to about 1000 Å, or about 100 Å to about 300 Å. The emission layer EML may have a single layer formed of a single material, a single layer formed of a plurality of different materials, or a multilayer structure having a plurality of layers formed of a plurality of different materials.

In the light emitting element ED of one or more embodiments, the emission layer EML may be to emit blue light. The light emitting element ED of one or more embodiments may include the amine compound of one or more embodiments in a hole transport region HTR and may show high efficiency and long-life characteristics in a blue emission region. In one or more embodiments, the light emitting element ED may include the amine compound of one or more embodiments in the hole transport region HTR, and the emission layer EML may be to emit blue fluorescence. However, embodiments of the present disclosure are not limited thereto.

In the light emitting element ED of one or more embodiments, the emission layer EML may include at least one of anthracene derivatives, pyrene derivatives, fluoranthene derivatives, chrysene derivatives, dihydrobenzanthracene derivatives, or triphenylene derivatives. For example, in one or more embodiments, the emission layer EML may include one or more anthracene derivatives and/or one or more pyrene derivatives.

In the light emitting elements ED one or more embodiments, shown in FIG. 3 to FIG. 7, the emission layer EML may include a host and a dopant, and the emission layer EML may include a compound represented by Formula E-1. The compound represented by Formula E-1 may be utilized as a fluorescence host material.

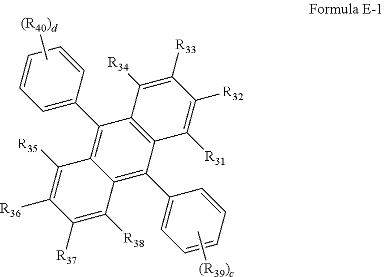

In Formula E-1, R₃₁ to R₄₀ may each independently be hydrogen, deuterium, a halogen, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form (or provide) a ring. In one or more embodiments, one or more selected from among R₃₁ to R₄₀ may be combined with an adjacent group to form (or provide) a saturated hydrocarbon ring, an unsaturated hydrocarbon ring, a saturated heterocycle, or an unsaturated heterocycle.

In Formula E-1, “c” and “d” may each independently be an integer of 0 to 5.

The compound represented by Formula E-1 may be any one selected from among Compound E1 to Compound E19.

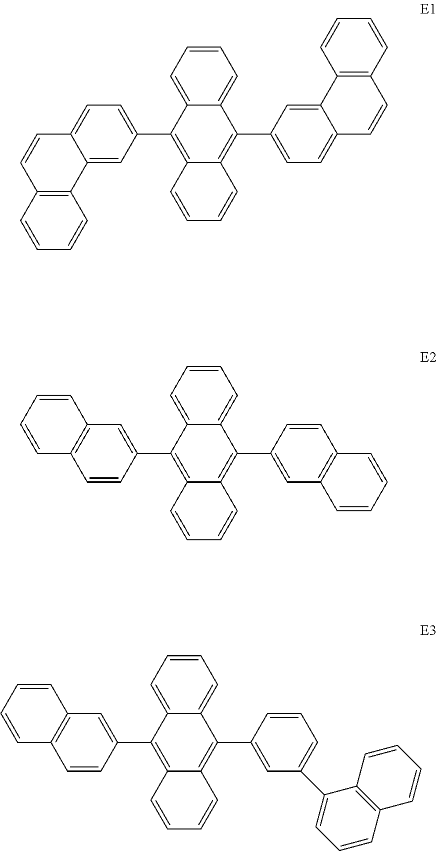

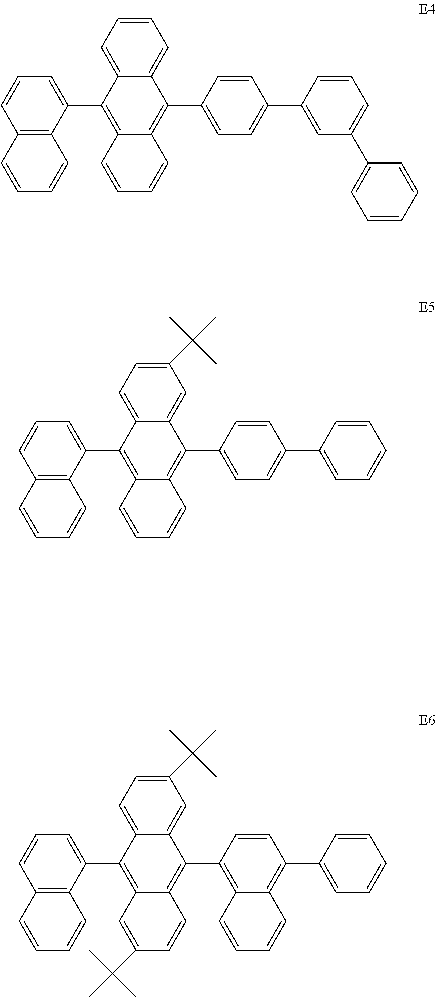

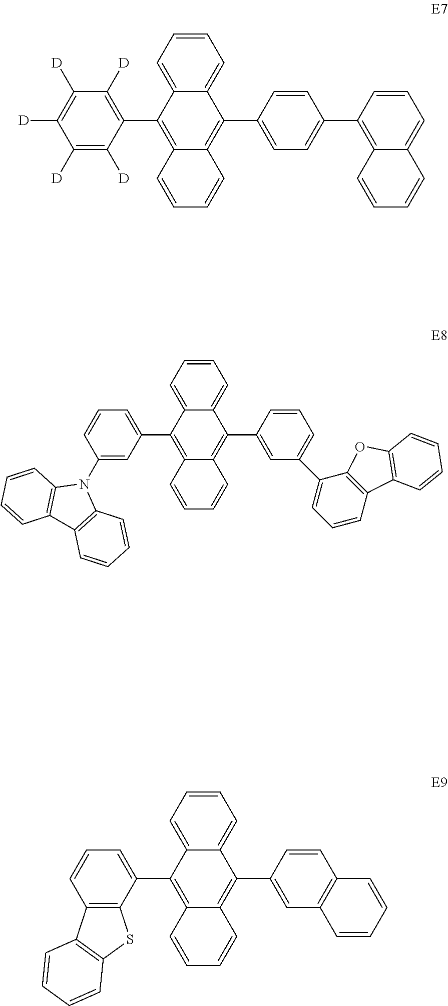 [SMILES: [2H]c1c([2H])c([2H])c(-c2c3ccccc3c(-c3ccc(-c4cccc5ccccc45)cc3)c3ccccc23)c([2H])c1[2H] ; c1cc(-c2c3ccccc3c(-c3cccc(-n4c5ccccc5c5ccccc54)c3)c3ccccc23)cc(-c2cccc3c2oc2ccccc23)c1 ; c1ccc2cc(-c3c4ccccc4c(-c4cccc5c4sc4ccccc45)c4ccccc34)ccc2c1]

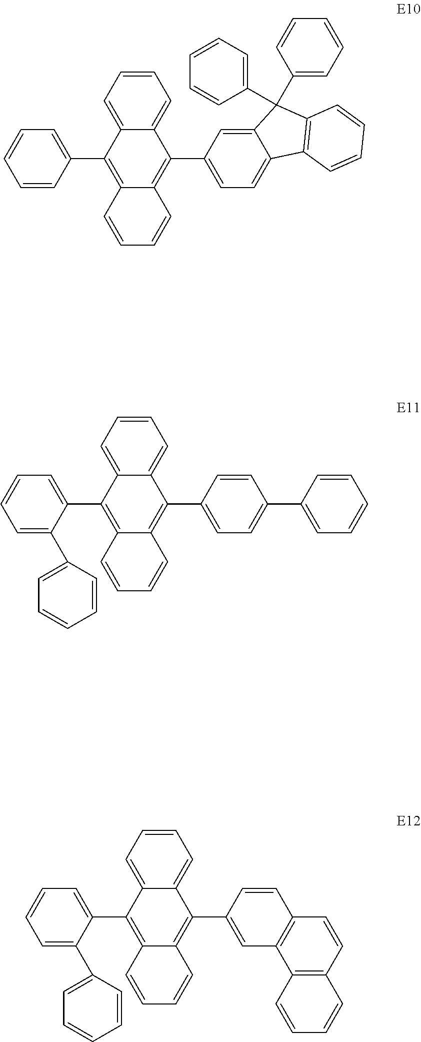

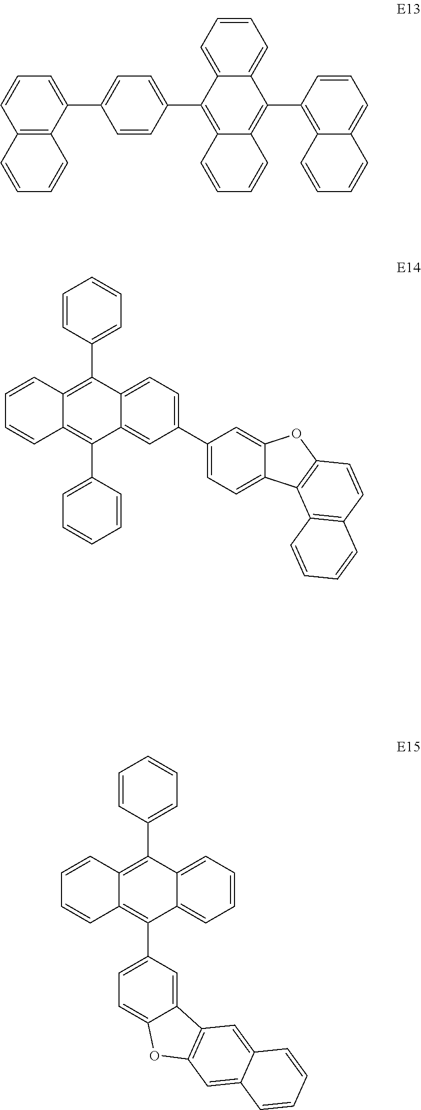

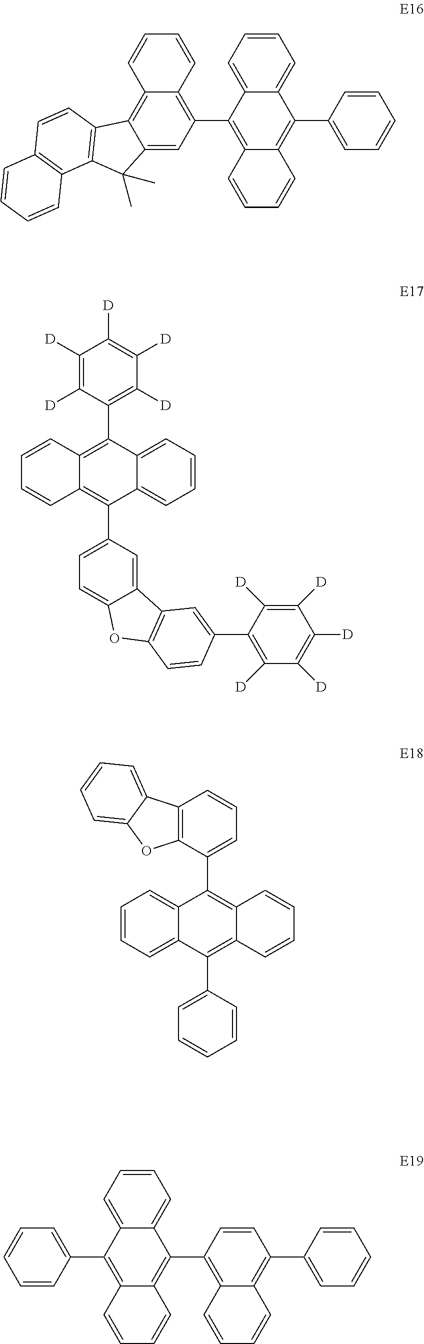

In one or more embodiments, the emission layer EML may include a compound represented by Formula E-2a or Formula E-2b. The compound represented by Formula E-2a or Formula E-2b may be utilized as a phosphorescence host material.

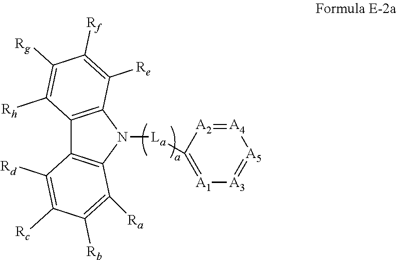

In Formula E-2a, “a” may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if “a” is an integer of 2 or more, multiple La's may each independently be a substituted or unsubstituted arylene group of 6 to 30 ring-forming carbon atoms, or a substituted or unsubstituted heteroarylene group of 2 to 30 ring-forming carbon atoms.

In one or more embodiments, in Formula E-2a, A₁ to A₅ may each independently be N or CRᵢ. Rₐ to Rᵢ may each independently be hydrogen, deuterium, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form (or provide) a ring. In some embodiments, one or more selected from among Rₐ to Rᵢ may be combined with an adjacent group to form (or provide) a hydrocarbon ring or a heterocycle including N, O, S, and/or the like, as a ring-forming atom.

In one or more embodiments, in Formula E-2a, two or three of (e.g., selected from among) A₁ to A₅ may be N, and the remainder may be CRᵢ.

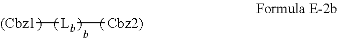

In Formula E-2b, Cbz1 and Cbz2 may each independently be an unsubstituted carbazole group or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and if “b” is an integer of 2 or more, multiple L_{b}'s may each independently be a substituted or unsubstituted arylene group of 6 to 30 ring-forming carbon atoms, or a substituted or unsubstituted heteroarylene group of 2 to 30 ring-forming carbon atoms. The compound represented by Formula E-2a or Formula E-2b may be any one selected from among compounds in Compound Group E-2. However, the compounds listed in Compound Group E-2 are only examples, and the compound represented by Formula E-2a or Formula E-2b is not limited to the compounds represented in Compound Group E-2.

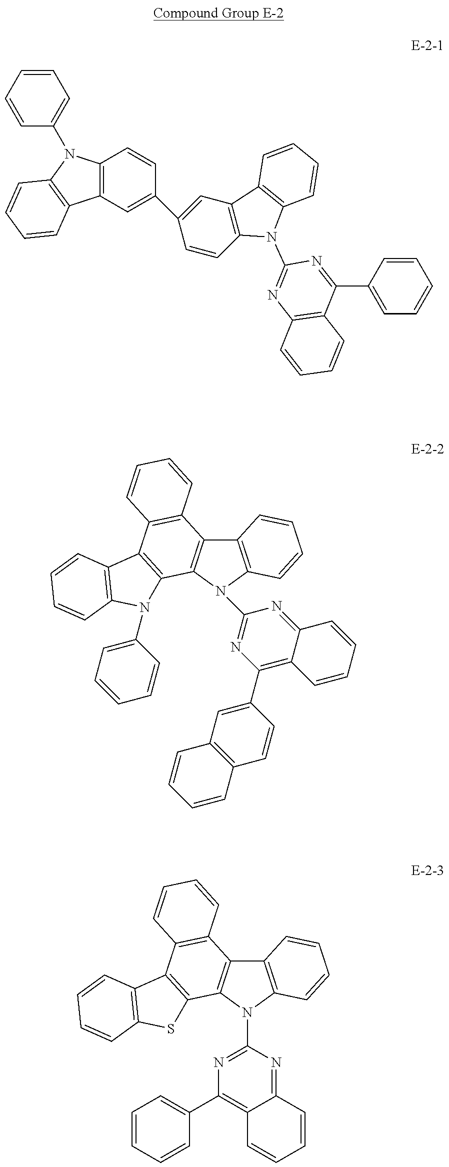 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

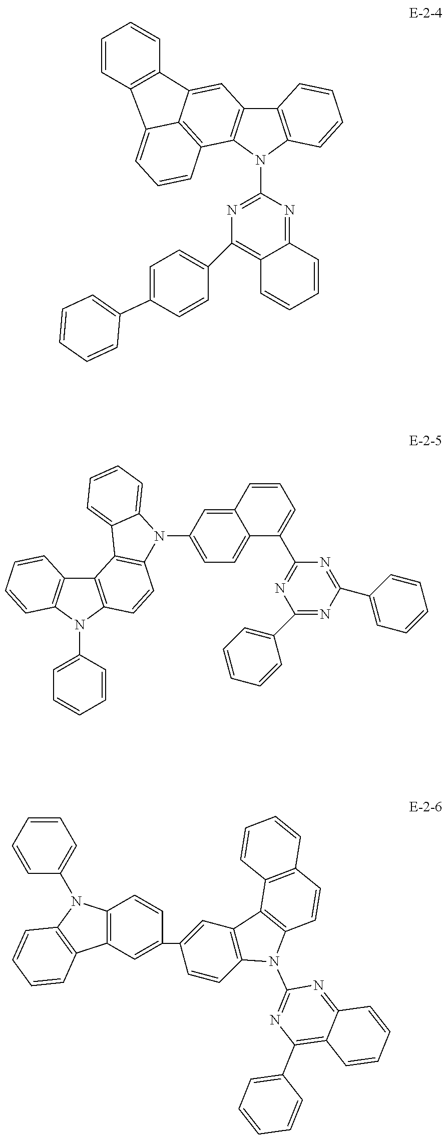 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

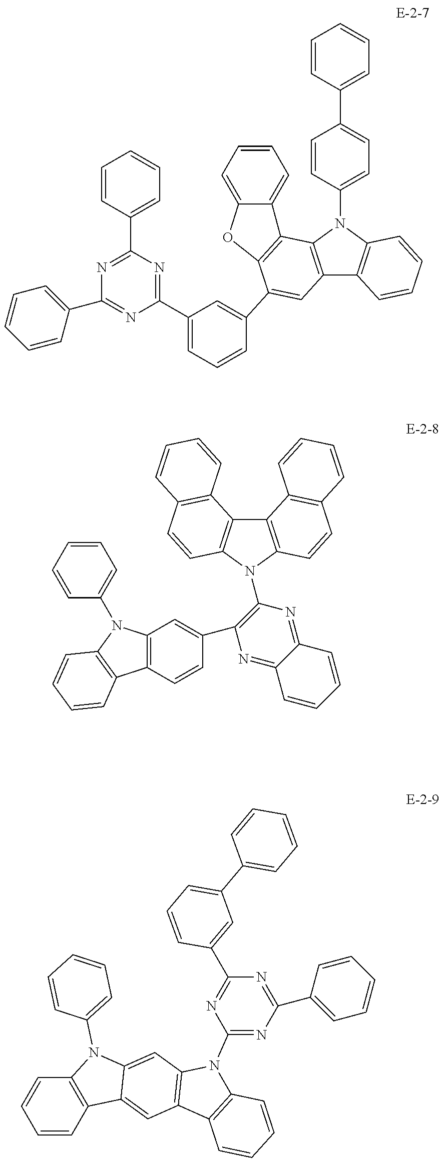 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

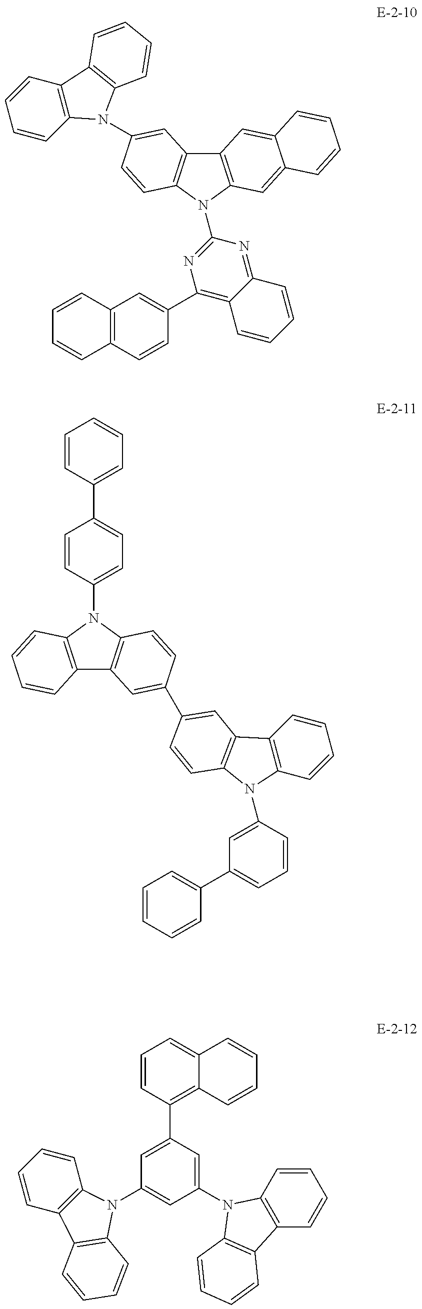 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

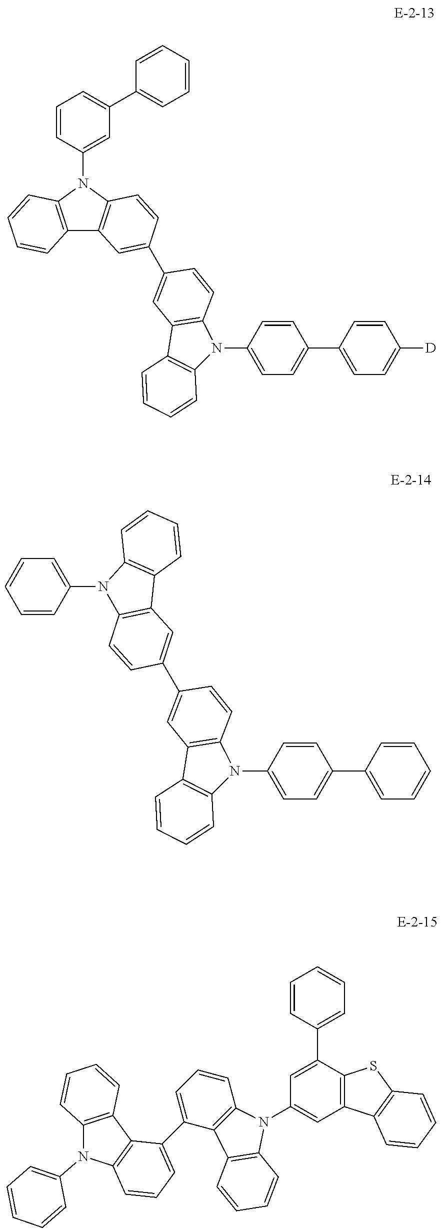 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

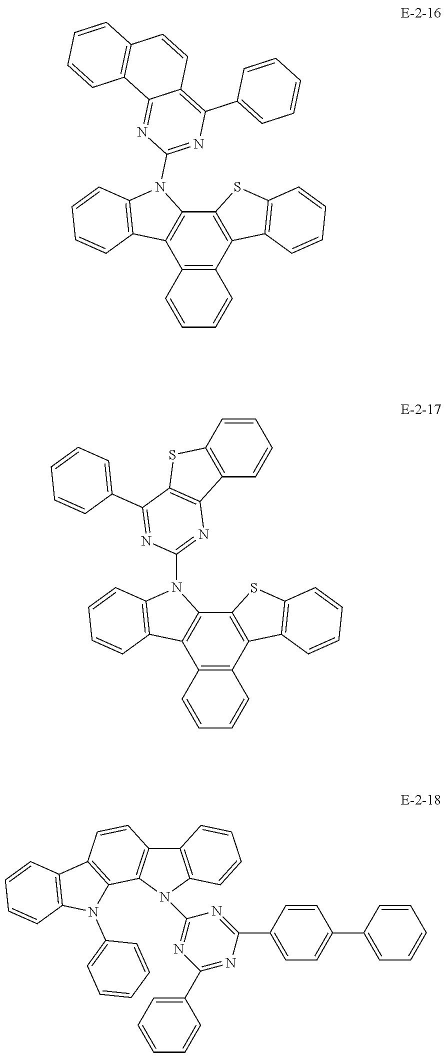 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

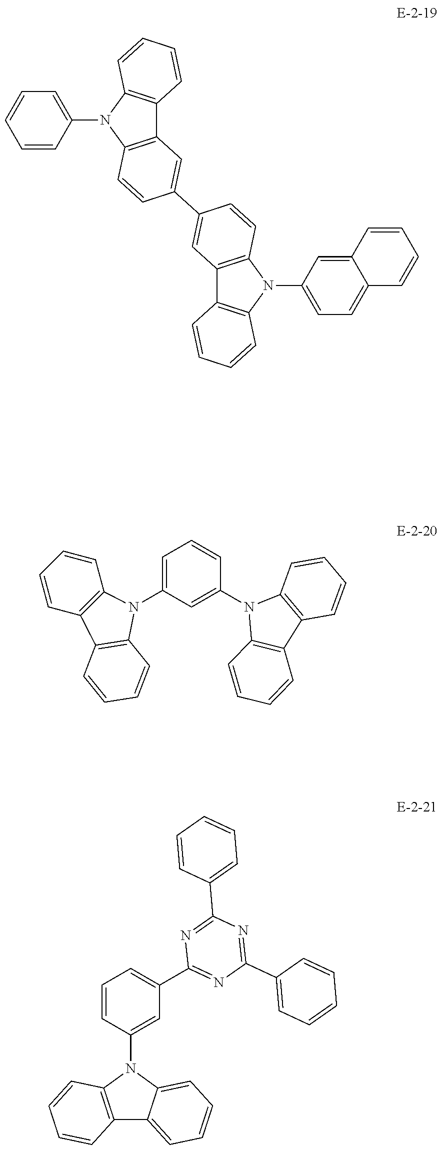 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

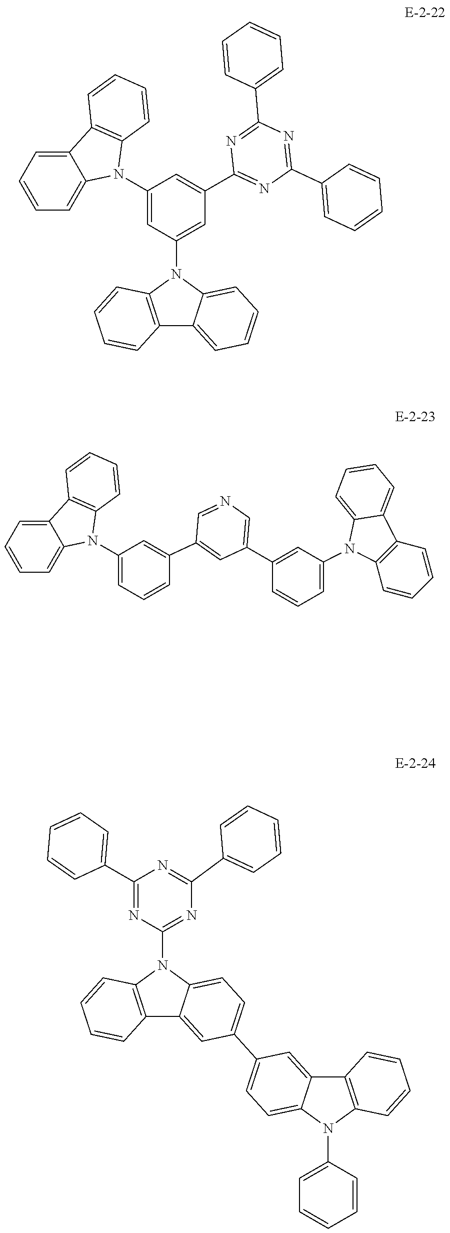 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

In one or more embodiments, the emission layer EML may further include a material well-suitable in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazol-2-yl)benzene (TPBi). However, embodiments of the present disclosure are not limited thereto. For example, tris(8-hydroxyquinolino)aluminum (Alq₃), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), and/or the like may be utilized as the host material.

In one or more embodiments, the emission layer EML may include a compound represented by Formula M-a or Formula M-b. The compound represented by Formula M-a or Formula M-b may be utilized as a phosphorescence dopant material.

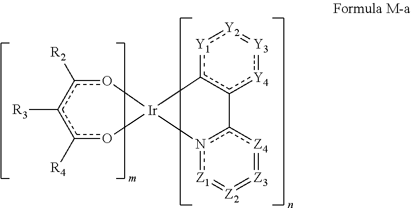

In Formula M-a, Y₁ to Y₄, and Z₁ to Z₄ may each independently be CR₁ or N, and R₁ to R₄ may each independently be hydrogen, deuterium, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form (or provide) a ring. In Formula M-a, “m” is 0 or 1, and “n” is 2 or 3. In Formula M-a, if “m” is 0, “n” is 3, and if “m” is 1, “n” is 2.

The compound represented by Formula M-a may be any one selected from among Compounds M-a1 to M-a25. However, Compounds M-a1 to M-a25 are mere examples, and the compound represented by Formula M-a is not limited to the compounds represented by Compounds M-a1 to M-a25.

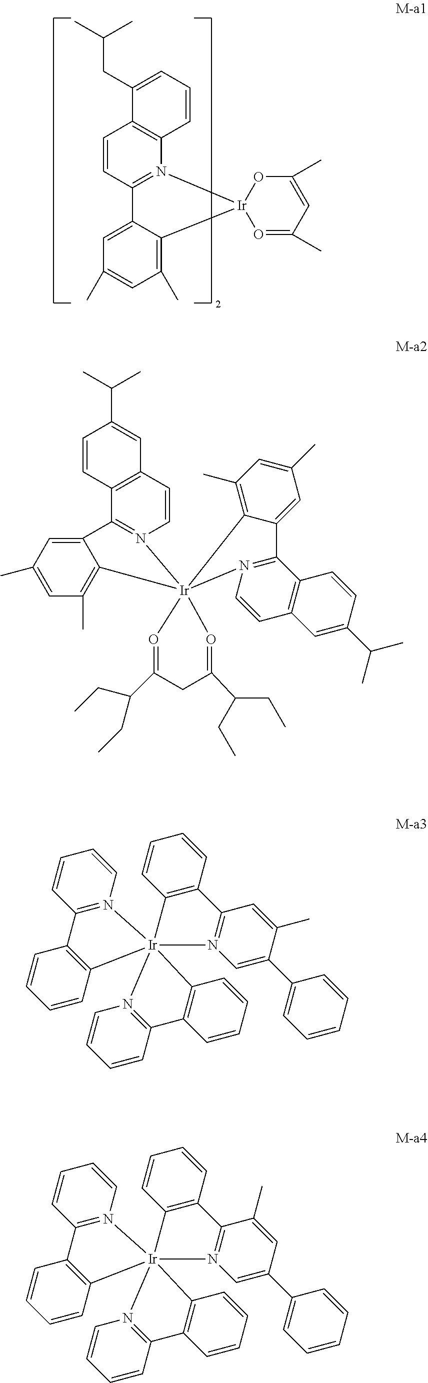

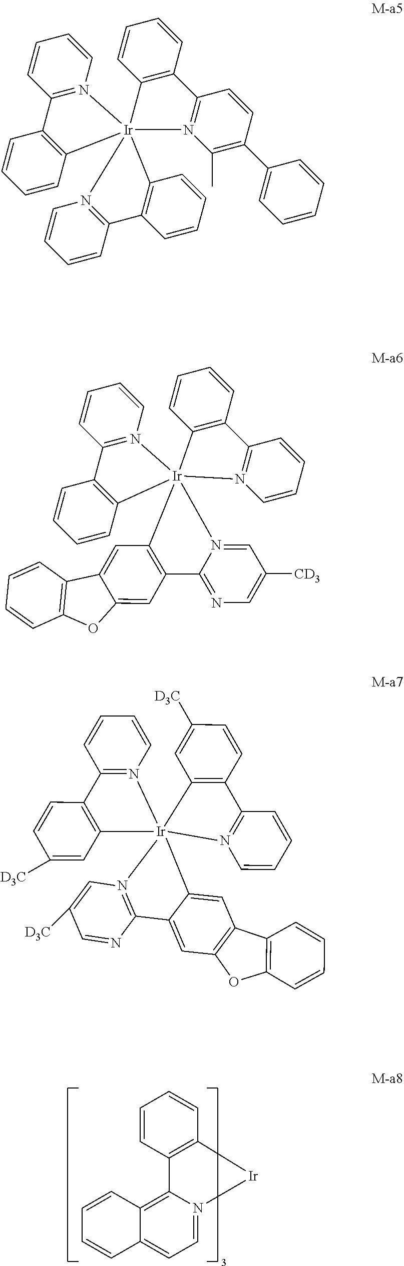

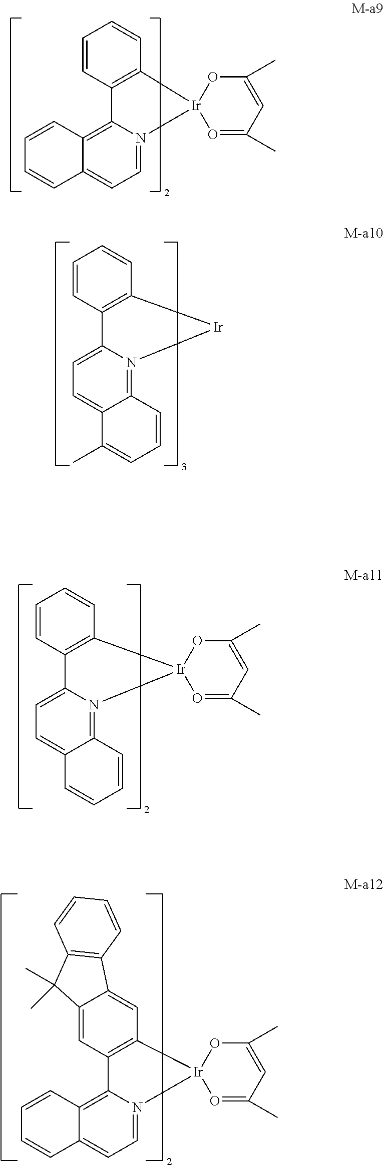

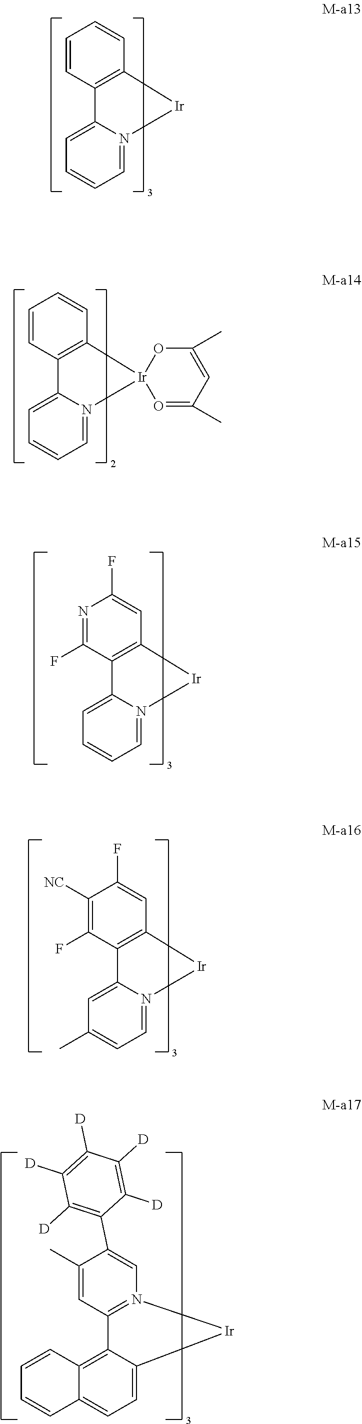

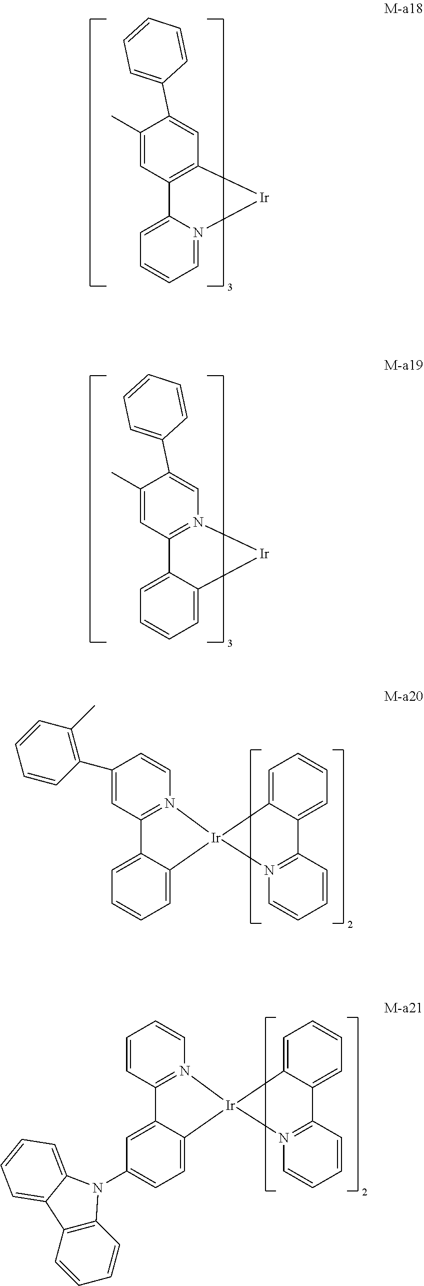

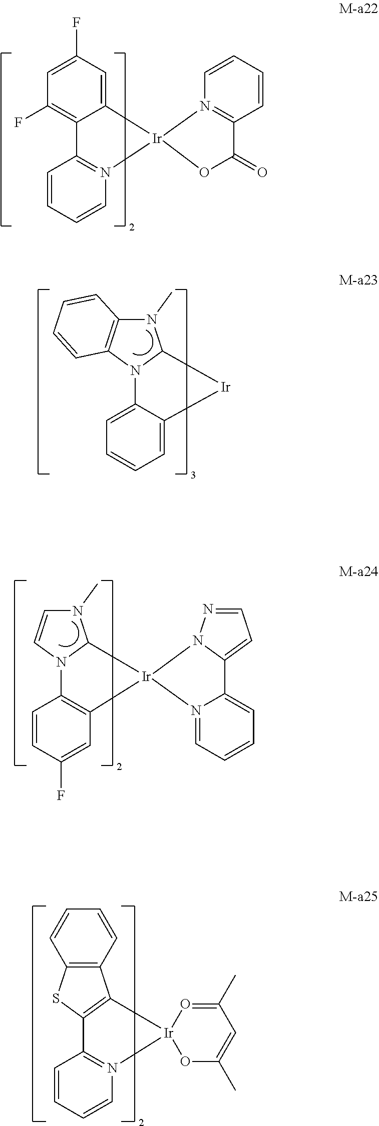

Compound M-a1 and Compound M-a2 may be utilized as red dopant materials, and Compound M-a3 to Compound M-a5 may be utilized as green dopant materials.

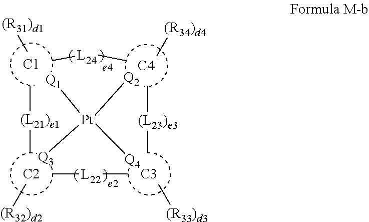

In Formula M-b, Q₁ to Q₄ may each independently be C or N, C1 to C4 may each independently be a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may each independently be a direct linkage,

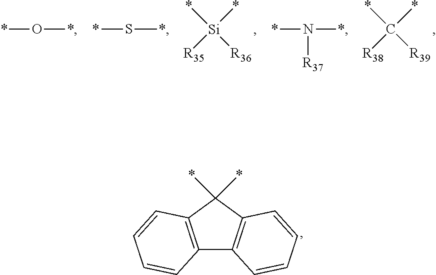

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R₃₁ to R₃₉ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form (or provide) a ring, and d1 to d4 may each independently be an integer of 0 to 4.

The compound represented by Formula M-b may be utilized as a blue phosphorescence dopant or a green phosphorescence dopant.

The compound represented by Formula M-b may be any one selected from among the compounds below. However, these compounds are mere example, and the compound represented by Formula M-b is not limited to the compounds represented below.

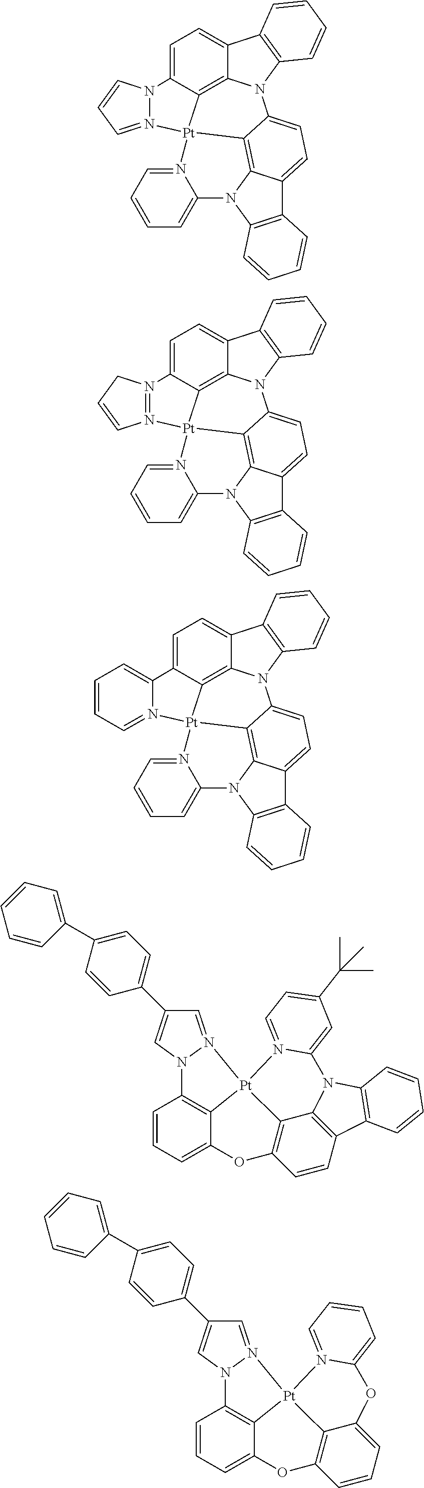

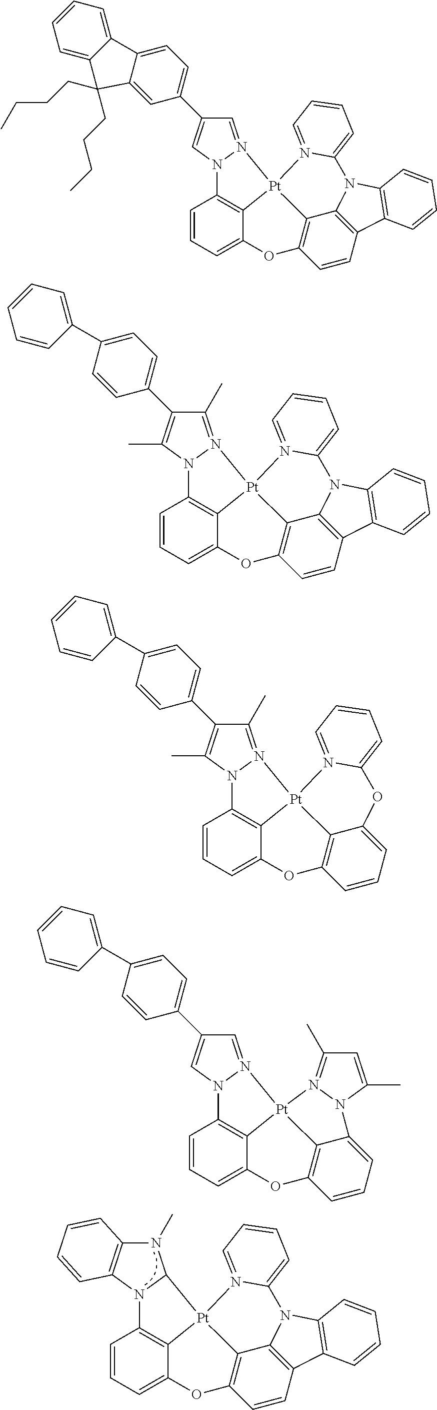

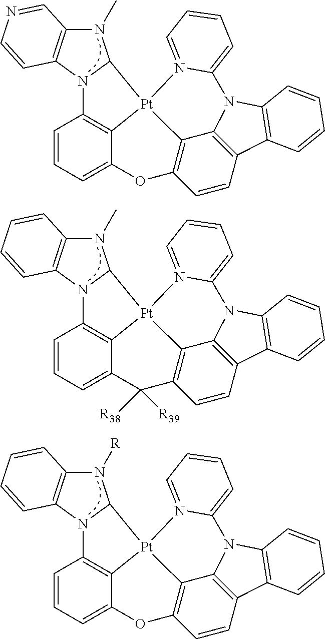

In the compounds above, R, R₃₈, and R₃₉ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In one or more embodiments, the emission layer EML may include a compound represented by any one selected from among Formula F-a to Formula F-c. The compounds represented by Formula F-a to Formula F-c may be utilized as fluorescence dopant materials.

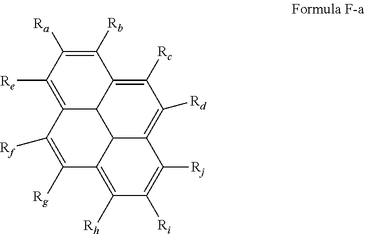

In Formula F-a, two of (e.g., selected from among) Rₐ to Rⱼ may each independently be substituted with

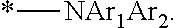

The remainder not substituted with

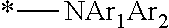

among Rₐ to Rⱼ may each independently by hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In

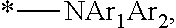

Ar₁ and Ar₂ may each independently be a substituted or unsubstituted aryl group of 6 to 30 ring-forming carbon atoms or a substituted or unsubstituted heteroaryl group of 2 to 30 ring-forming carbon atoms. For example, in some embodiments, at least one selected from among Ar₁ and Ar₂ may be a heteroaryl group including O or S as a ring-forming atom.

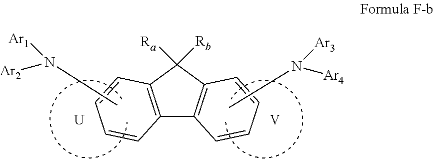

In Formula F-b, Rₐ and Rb may each independently be hydrogen, deuterium, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form (or provide) a ring. Ar₁ to Ar₄ may each independently be a substituted or unsubstituted aryl group of 6 to 30 ring-forming carbon atoms or a substituted or unsubstituted heteroaryl group of 2 to 30 ring-forming carbon atoms. In Formula F-b, U and V may each independently be a substituted or unsubstituted hydrocarbon ring of 5 to 30 ring-forming carbon atoms or a substituted or unsubstituted heterocycle of 2 to 30 ring-forming carbon atoms. In some embodiments, at least one selected from among Ar₁ to Ar₄ may be a heteroaryl group including O or S as a ring-forming atom.

In Formula F-b, the number of rings represented by U and V may each independently be 0 or 1. For example, in Formula F-b, if the number of U or V is 1, one ring forms a fused ring at the designated part by U or V, and if the number of U or Vis 0, a ring is not present at the designated part by U or V. For example, if the number of U is 0, and the number of V is 1, or if the number of U is 1, and the number of V is 0, a fused ring having the fluorene core of Formula F-b may be a ring compound with four rings. In one or more embodiments, if the number of both (e.g., simultaneously) U and V is 0, the fused ring of Formula F-b may be a ring compound with three rings. In one or more embodiments, if the number of both (e.g., simultaneously) U and V is 1, a fused ring having the fluorene core of Formula F-b may be a ring compound with five rings.

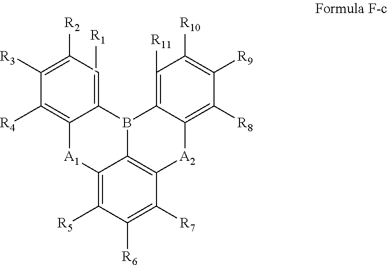

In Formula F-c, A₁ and A₂ may each independently be O, S, Se, or NRₘ, and Rₘ may be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be hydrogen, deuterium, a halogen, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form (or provide) a ring.

In some embodiments, in Formula F-c, A₁ and A₂ may each independently be combined with the substituents of an adjacent ring to form (or provide) a fused ring. For example, if A₁ and A₂ may each independently be NRₘ, in one or more embodiments, A₁ may be combined with R₄ or R₅ to form (or provide) a ring. In one or more embodiments, A₂ may be combined with R₇ or R₈ to form (or provide) a ring.

In one or more embodiments, the emission layer EML may include, as a suitable dopant material, one or more selected from styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and derivatives thereof (for example, 2,5,8,11-tetra-t-butylperylene (TBP)), pyrene and derivatives thereof (for example, 1,1-dipyrene, 1,4-dipyrenylbenzene, and 1,4-bis(N,N-diphenylamino) pyrene), and/or the like.

In one or more embodiments, if multiple emission layers EML are included, at least one emission layer EML may include a suitable phosphorescence dopant material. For example, the phosphorescence dopant may utilize a metal complex including iridium (Ir), platinum (Pt), osmium (Os), gold (Au), titanium (Ti), zirconium (Zr), hafnium (Hf), europium (Eu), terbium (Tb), or thulium (Tm). For example, in some embodiments, iridium (III)bis(4,6-difluorophenylpyridinato-N, C2′) picolinate (FIrpic), bis(2,4-difluorophenylpyridinato)-tetrakis(1-pyrazolyl) borate iridium (III) (Fir6), or platinum octaethyl porphyrin (PtOEP) may be utilized as the phosphorescence dopant. However, embodiments of the present disclosure are not limited thereto.

In one or more embodiments, in the light emitting element ED, the emission layer EML may be a delayed fluorescence emission layer including a host and a dopant. For example, the emission layer EML may be to emit thermally activated delayed fluorescence (TADF). In the light emitting element ED according to one or more embodiments, the emission layer EML may include a suitable thermally active delayed fluorescence dopant.

In one or more embodiments, the emission layer EML of the light emitting element ED may include a plurality of host materials, a thermally activated delayed fluorescent dopant, and a phosphorescent sensitizer.

In one or more embodiments, the emission layer EML may include a quantum dot material. In one or more embodiments, the quantum dot material may have a core/shell structure. The core of the quantum dots may be selected from among a Group II-VI compound, a Group I-II-VI compound, a Group II-IV-VI compound, a Group I-II-IV-VI compound, a Group II-IV-V compound, a Group III-VI compound, a Group I-III-VI compound, a Group III-V compound, a Group III-II-V compound, a Group IV-VI compound, a Group IV element, a Group IV compound, and/or a (e.g., any suitable) combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a mixture thereof.

The Group III-VI compound may include a binary compound such as In₂S₃ and/or In₂Se₃, a ternary compound such as InGaS₃ and/or InGaSe₃, or any combination thereof.

The Group I-III-VI compound may be selected from a ternary compound selected from the group consisting of AgInS, AgInS₂, CuInS, CuInS₂, AgGaS₂, CuGaS₂ CuGaO₂, AgGaO₂, AgAlO₂, and a mixture thereof, and/or a quaternary compound such as AgInGaS₂ and/or CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and a mixture thereof; a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof; and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. In one or more embodiments, the Group III-V compound may further include a Group II metal. For example, InZnP, and/or the like, may be selected as a Group III-II-V compound.

The Group IV-VI compound may be selected from the group consisting of a binary compound selected from the group consisting of SnS, SnSe, SnTe, PbS, PbSe, PbTe, and a mixture thereof; a ternary compound selected from the group consisting of SnSeS, SnSeTe, SnSTe, PbSeS, PbSeTe, PbSTe, SnPbS, SnPbSe, SnPbTe, and a mixture thereof; and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

Each element included in a polynary compound such as the binary compound, the ternary compound, or the quaternary compound may be present in a particle with a substantially uniform concentration distribution or a non-substantially uniform concentration distribution. For example, the formulae of the quantum dot material refer to the types (kinds) of elements included in the compounds, and the elemental ratio in the compound may be different. For example, AgInGaS₂ may refer to AgInₓGa₁₋ₓS₂ (where x is a real number of 0 to 1). In one or more embodiments, the quantum dot may have a single structure or a double structure of core-shell in which the concentration of each element included in the quantum dot is substantially uniform. For example, the material included in the core may be different from the material included in the shell.

The shell of the quantum dot may serve as a protection layer to prevent or reduce the chemical deformation of the core to maintain semiconductor properties, and/or a charging layer to impart electrophoresis properties to the quantum dot. The shell may be a single layer or a multilayer. An interface between the core and the shell may have a concentration gradient in which the concentration of an element present in the shell becomes lower towards the center. An example of the shell of the quantum dots may include a metal or non-metal oxide, a semiconductor compound, and/or a (e.g., any suitable) combination thereof. For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO, or a ternary compound such as MgAl₂O₄, CoFe₂O₄, NiFe₂O₄, or CoMn₂O₄, but embodiments of the present disclosure are not limited thereto.

Also, examples of the semiconductor compound suitable as a shell may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but embodiments of the present disclosure are not limited thereto.

Each element included in a polynary compound such as the binary compound, or the ternary compound may be present in a particle with a substantially uniform or non-substantially uniform concentration distribution. For example, the formulae the compound refer to the types (kinds) of elements included in the compounds, and the elemental ratio in the compound may be different.

The quantum dot may have a full width of half maximum (FWHM) of an emission spectrum of about 45 nm or less, about 40 nm or less, or about 30 nm or less, and color purity or color reproducibility thereof may be improved in the above range. In one or more embodiments, light emitted through such a quantum dot is emitted in all directions, and thus a wide viewing angle may be improved.

Although the form of the quantum dot is not particularly limited as long as it is a form commonly utilized in the art, for example, the quantum dot in the form of spherical nanoparticles, pyramidal nanoparticles, multi-arm nanoparticles, cubic nanoparticles, nanotubes, nanowires, nanofibers, nanoplate particles, and/or the like may be utilized.

As the size of the quantum dot is adjusted or the elemental ratio in the quantum dot compound is adjusted, it may control the energy band gap of the quantum dot, and thus light in one or more suitable wavelength ranges may be obtained in a quantum dot emission layer. Therefore, the quantum dot described above (for example, utilizing different sizes of quantum dots and/or different elemental ratios in the quantum dot compound) is utilized, a light emitting element, which emits light in one or more suitable wavelengths, may be implemented. For example, the adjustment of the size of the quantum dots and or the elemental ratio in the quantum dot compound may be selected to enable the quantum dots to emit red, green, and/or blue light. In one or more embodiments, the quantum dots may be configured to emit white light by combining one or more suitable colors of light.

In each of the light emitting elements ED of embodiments illustrated in FIGS. 3 to 7, the electron transport region ETR may be provided on the emission layer EML. The electron transport region ETR may include at least one of a hole blocking layer HBL, an electron transport layer ETL, or an electron injection layer EIL, but embodiments of the present disclosure are not limited thereto.

The electron transport region ETR may have a single layer formed of a single material, a single layer formed of a plurality of different materials, or a multilayer structure including a plurality of layers formed of a plurality of different materials.

For example, in one or more embodiments, the electron transport region ETR may have a single layer structure of an electron injection layer EIL or an electron transport layer ETL, and may have a single layer structure formed of an electron injection material and an electron transport material. In one or more embodiments, the electron transport region ETR may have a single layer structure formed of a plurality of different materials, or may have a structure in which electron transport layer ETL/electron injection layer EIL or hole blocking layer HBL/electron transport layer ETL/electron injection layer EIL are stacked in order (e.g., in the stated order) from the emission layer EML, but embodiments of the present disclosure are not limited thereto. The electron transport region ETR may have a thickness, for example, from about 1,000 Å to about 1,500 Å.

The electron transport region ETR may be formed utilizing one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, the electron transport region ETR may include a compound represented by Formula ET-2.

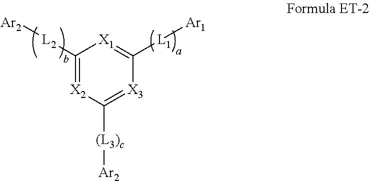

In Formula ET-2, at least one selected from among X₁ to X₃ is N, and the rest are CRₐ. Rₐ may be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer of 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, if (e.g., when) a to c are each independently an integer of 2 or more, L₁ to L₃ may each independently be a substituted or unsubstituted arylene group having 6 to 30 ring-forming carbon atoms or a substituted or unsubstituted heteroarylene group having 2 to 30 ring-forming carbon atoms.

In one or more embodiments, the electron transport region ETR may include an anthracene-based compound. However, embodiments of the present disclosure are not limited thereto, for example, in some embodiments, the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(biphenyl-4-yl)-4-phenyl-5-tert-butylphenyl-1,2,4-triazole (TAZ), 4-(naphthalen-1-yl)-3,5-diphenyl-4H-1,2,4-triazole (NTAZ), 2-(biphenyl-4-yl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), beryllium bis(benzoquinolin-10-olate) (Bebq₂), 9,10-di(naphthalen-2-yl)anthracene (ADN), 1,3-bis[3,5-di(pyridin-3-yl)phenyl]benzene (BmPyPhB), or a mixture thereof.

In one or more embodiments, the electron transport region ETR may include at least one selected from among Compound ET1 to Compound ET36.

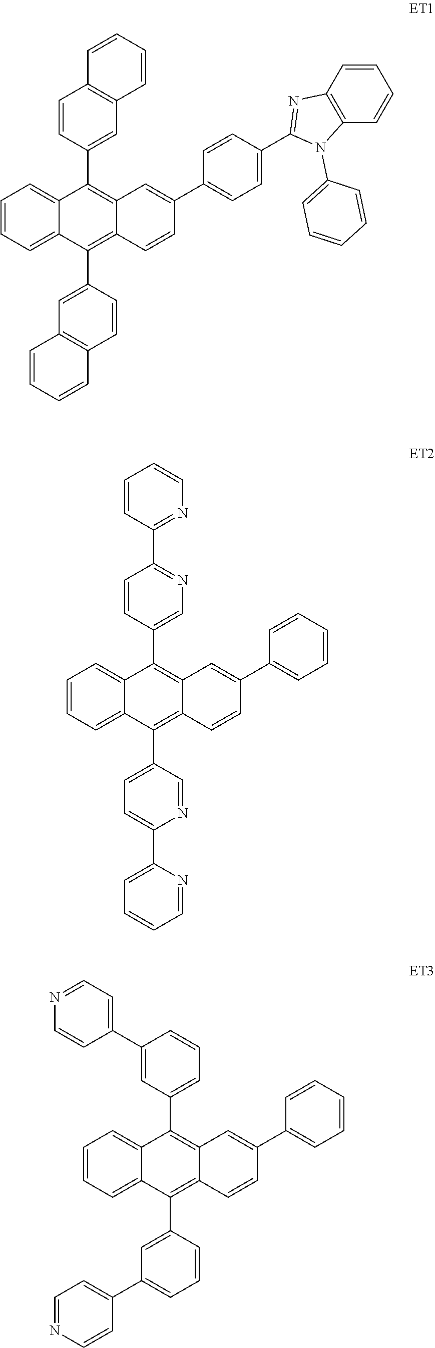 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

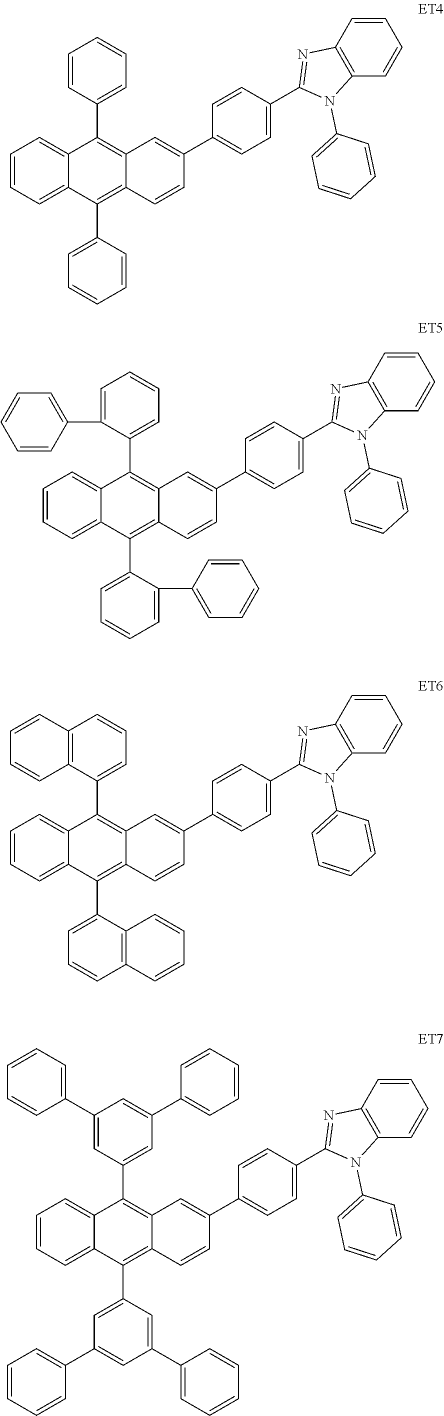 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

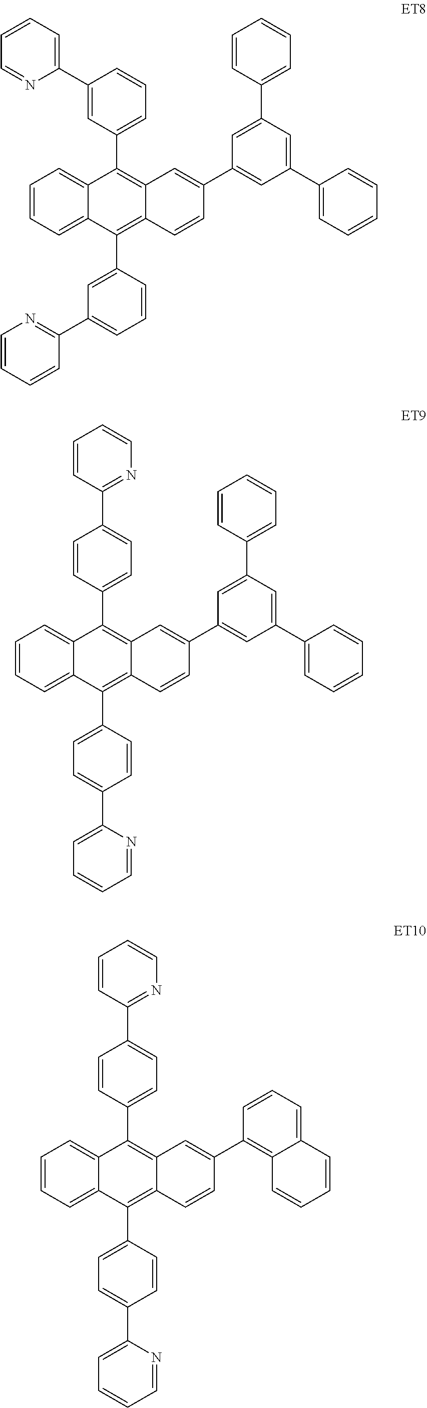 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

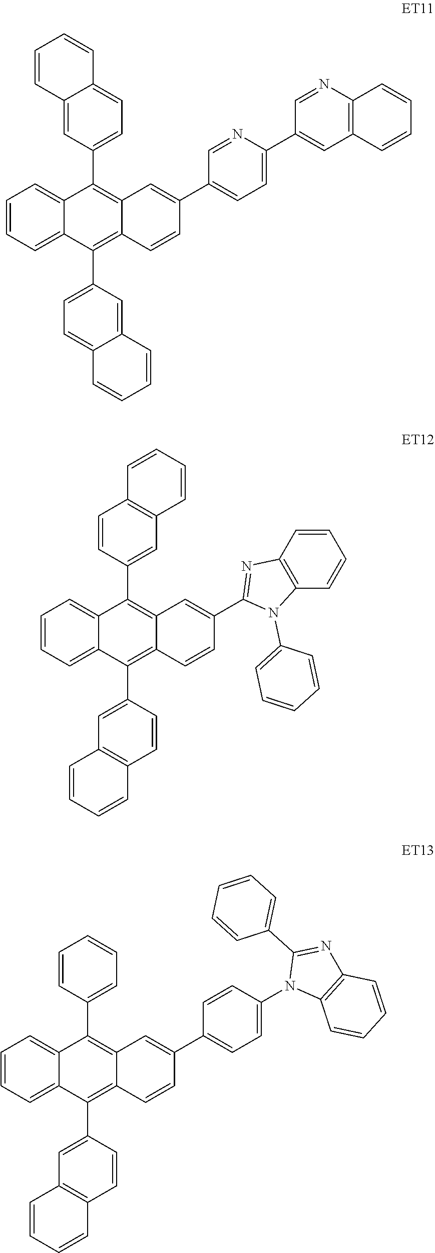 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

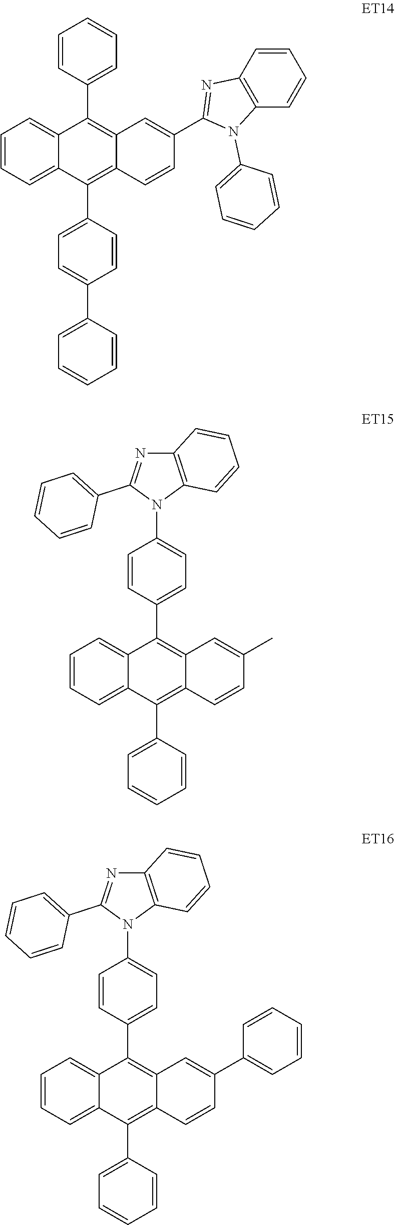

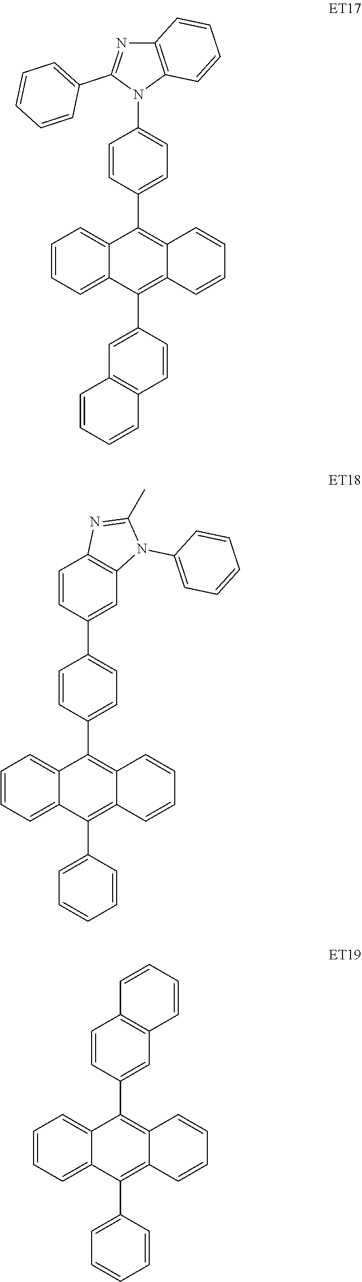

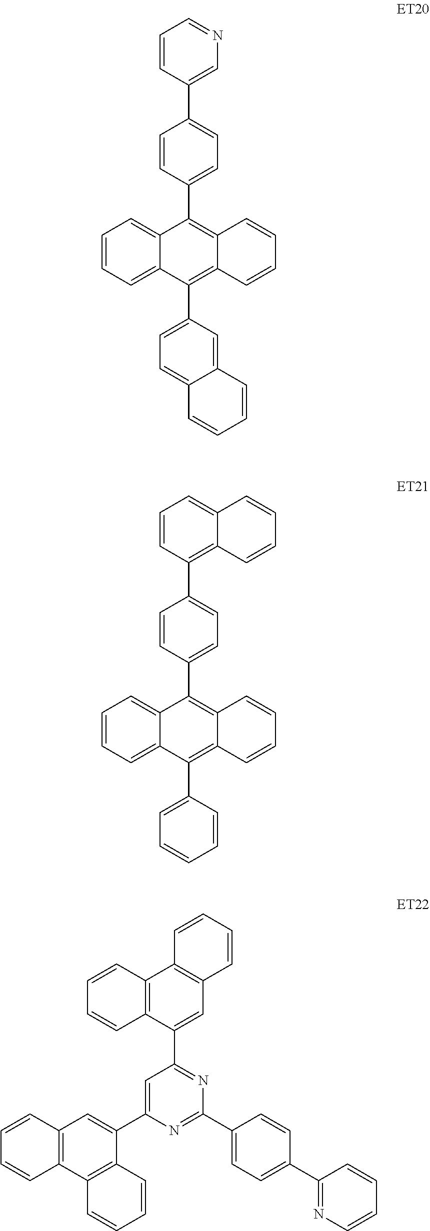

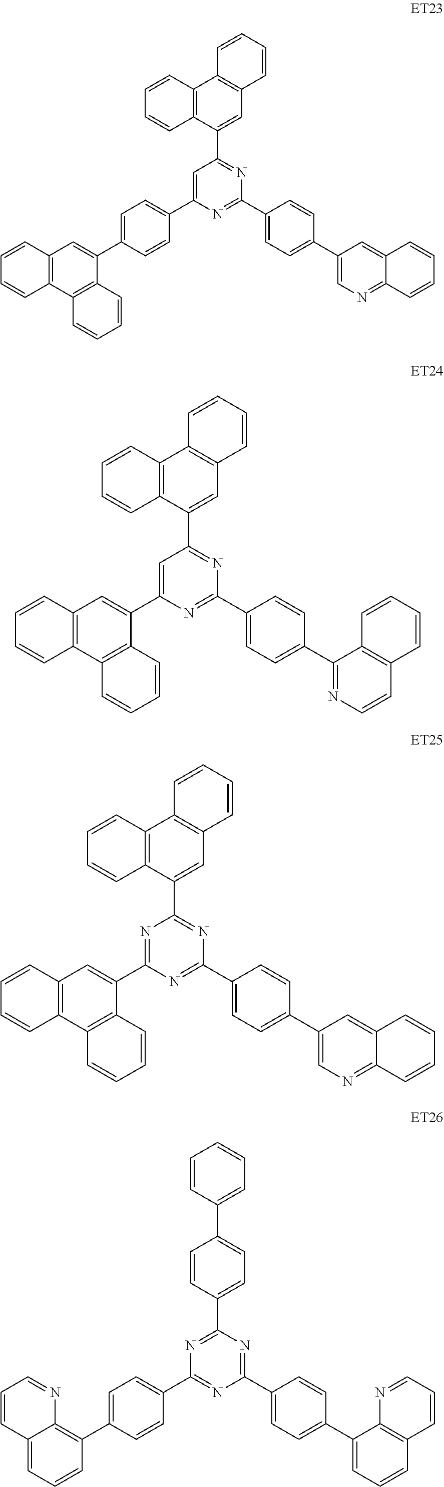 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1]

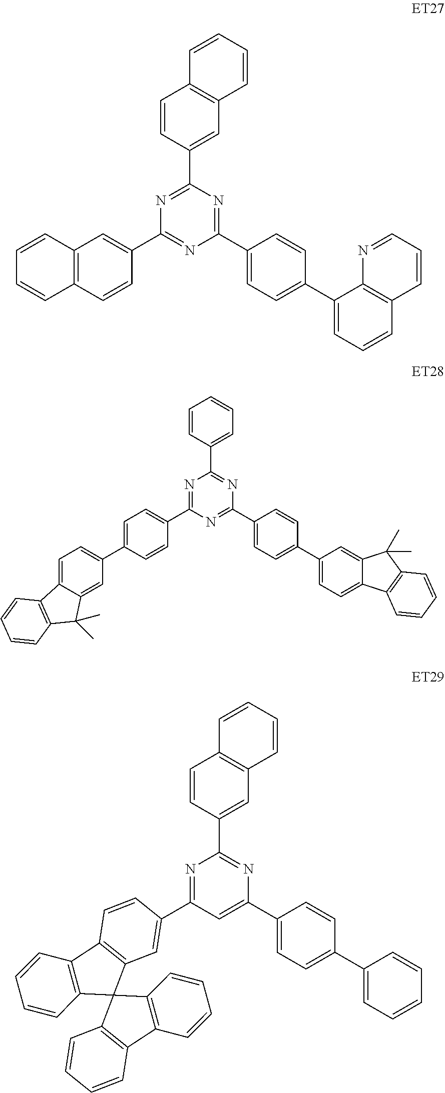 [SMILES: c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

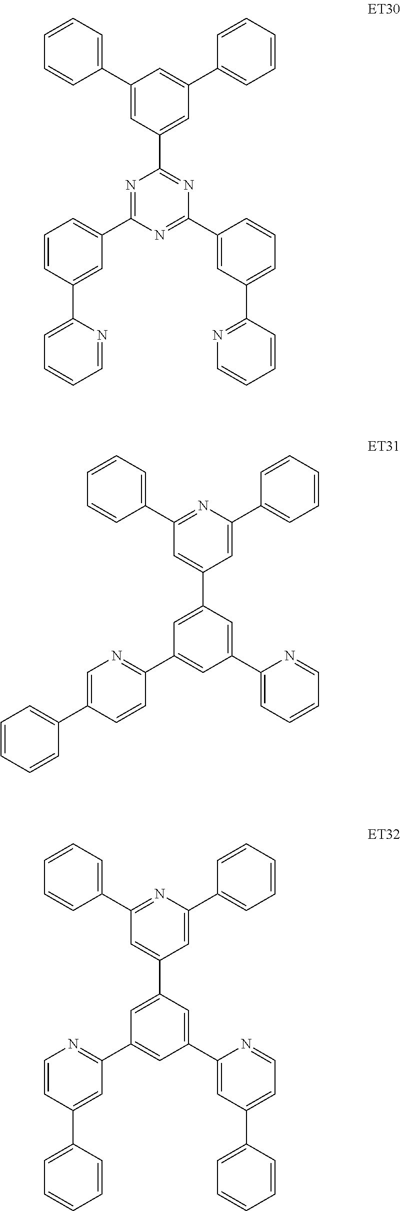 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

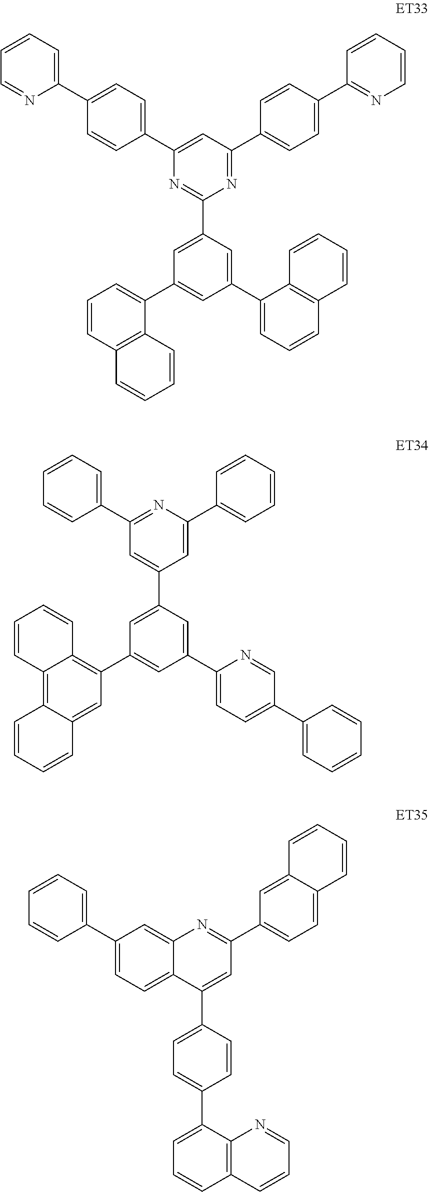 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1]

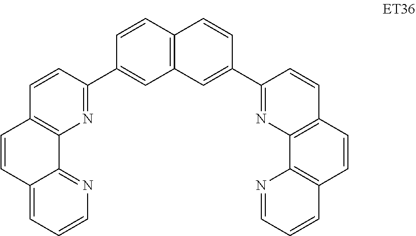 [SMILES: c1cnc2c(c1)ccc1ccc(-c3ccc4ccc(-c5ccc6ccc7cccnc7c6n5)cc4c3)nc12]

In one or more embodiments, the electron transport region ETR may include a metal halide such as LiF, NaCl, CsF, RbCl, RbI, CuI, and/or Kl, a lanthanide metal such as Yb, or a co-deposited material of the metal halide and the lanthanide metal. For example, in some embodiments, the electron transport region ETR may include Kl:Yb, RbI:Yb, LiF:Yb, and/or the like, as a co-deposited material. In one or more embodiments, the electron transport region ETR may be formed utilizing a metal oxide such as Li₂O and/or BaO, or 8-hydroxyl-lithium quinolate (Liq), and/or the like, but embodiments of the present disclosure are not limited thereto. The electron transport region ETR may also be formed of a mixture material of an electron transport material and an insulating organometallic salt. The organometallic salt may be a material having an energy band gap of about 4 eV or more. For example, the organometallic salt may include, for example, a metal acetate, a metal benzoate, a metal acetoacetate, a metal acetylacetonate, and/or a metal stearate.

In one or more embodiments, the electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the above-described materials, but embodiments of the present disclosure are not limited thereto.

The electron transport region ETR may include one or more of the above-described compounds of the electron transport region in at least one of the electron injection layer EIL, the electron transport layer ETL, or the hole blocking layer HBL.

When the electron transport region ETR includes an electron transport layer ETL, the electron transport layer ETL may have a thickness of about 100 angstrom (Å) to about 1,000 Å, for example, about 150 Å to about 500 Å. If the thickness of the electron transport layer ETL satisfies the aforementioned range, satisfactory electron transport characteristics may be obtained without a substantial increase in driving voltage. When the electron transport region ETR includes an electron injection layer EIL, the electron injection layer EIL may have a thickness of about 1 Å to about 100 Å, for example, about 3 Å to about 90 Å. If the thickness of the electron injection layer EIL satisfies the above-described range, satisfactory electron injection characteristics may be obtained without a substantial increase in driving voltage.

The second electrode EL2 may be provided on the electron transport region ETR. The second electrode EL2 may be a common electrode. The second electrode EL2 may be a cathode or an anode, but embodiments of the present disclosure are not limited thereto. For example, if (e.g., when) the first electrode EL1 is an anode, the second electrode EL2 may be a cathode, and if (e.g., when) the first electrode EL1 is a cathode, the second electrode EL2 may be an anode.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), and/or the like.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, or a mixture thereof (e.g., AgMg, AgYb, or MgAg). In one or more embodiments, the second electrode EL2 may have a multilayer structure including a reflective film or a transflective film formed of one or more of the above-described materials, and a transparent conductive film formed of ITO, IZO, ZnO, ITZO, and/or the like. For example, the second electrode EL2 may include one of the above-described metal materials, combinations of at least two metal materials of the above-described metal materials, oxides of the above-described metal materials, and/or the like.

In some embodiments, the second electrode EL2 may be connected with an auxiliary electrode. If the second electrode EL2 is connected with the auxiliary electrode, the resistance of the second electrode EL2 may be decreased.

In one or more embodiments, a capping layer CPL may further be on (e.g., arranged) on the second electrode EL2 of the light emitting element ED. The capping layer CPL may include a multilayer or a single layer.

In one or more embodiments, the capping layer CPL may be an organic layer or an inorganic layer. For example, if (e.g., when) the capping layer CPL contains an inorganic material, the inorganic material may include an alkali metal compound (e.g., LiF), an alkaline earth metal compound (e.g., MgF₂), SiON, SiNₓ, SiO_{y}, and/or the like.

For example, if (e.g., when) the capping layer CPL includes an organic material, the organic material may include a-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol-9-yl)triphenylamine (TCTA), and/or the like, or an epoxy resin, or acrylate such as methacrylate. However, embodiments of the present disclosure are not limited thereto, and the capping layer CPL may include at least one selected from among Compounds P1 to P5.

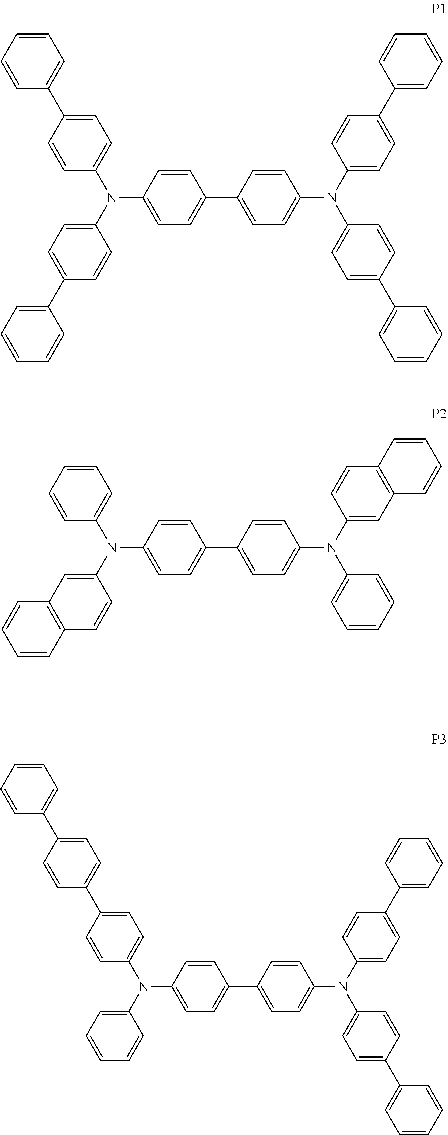 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

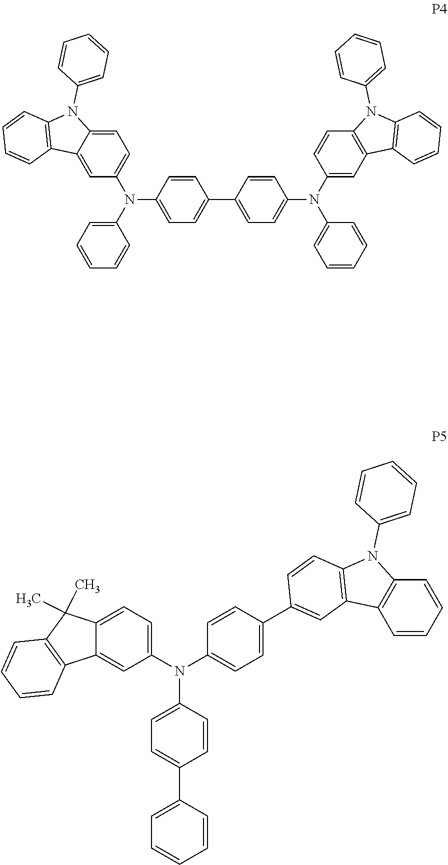 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the refractive index of the capping layer CPL may be about 1.6 or more. For example, in one or more embodiments, the refractive index of the capping layer CPL may be about 1.6 or more with respect to light in a wavelength range of about 550 nanometer (nm) to about 660 nm.

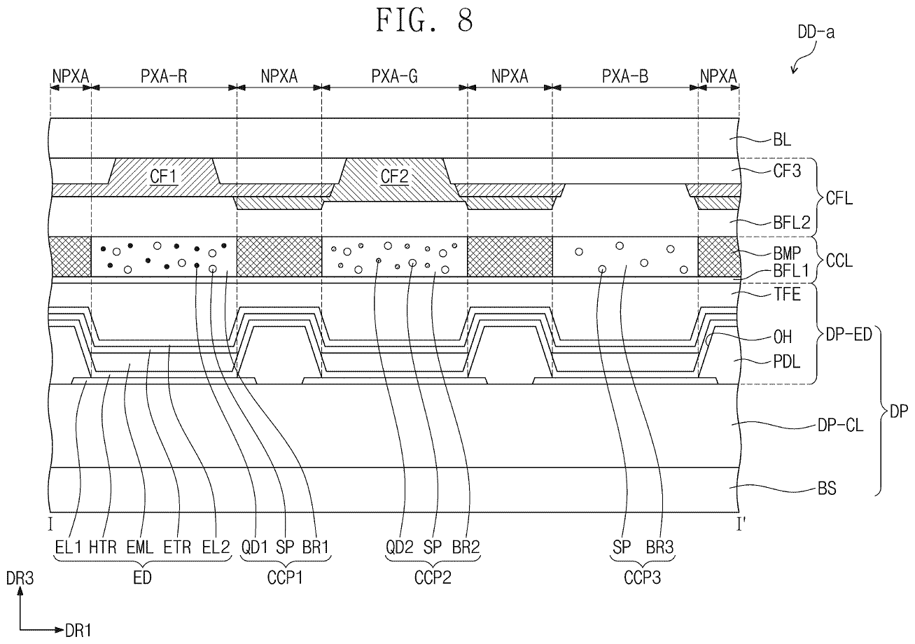

Each of FIGS. 8 to 11 is a cross-sectional view of a display device according to one or more embodiments of the present disclosure. Hereinafter, in describing the display devices of embodiments with reference to FIGS. 8 to 11, the duplicated features which have been described in FIGS. 1 to 7 are not described again for conciseness, but their differences will be mainly described.

Referring to FIG. 8, the display device DD-a according to one or more embodiments may include a display panel DP including a display element layer DP-ED, a light control layer CCL on (e.g., arranged on) the display panel DP, and a color filter layer CFL. In one or more embodiments illustrated in FIG. 8, the display panel DP may include a base layer BS, a circuit layer DP-CL provided on the base layer BS, and the display element layer DP-ED, and the display element layer DP-ED may include a light emitting element ED.

The light emitting element ED may include a first electrode EL1, a hole transport region HTR on (e.g., arranged on) the first electrode EL1, an emission layer EML on (e.g., arranged on) the hole transport region HTR, an electron transport region ETR on (e.g., arranged on) the emission layer EML, and a second electrode EL2 on (e.g., arranged on) the electron transport region ETR. In one or more embodiments, the structure of any one of the light emitting elements of FIG. 3 to FIG. 7 described above may be equally applied to the structure of the light emitting element ED illustrated in FIG. 8. The light emitting element ED illustrated in FIG. 8 may include the amine compound of one or more embodiments in the hole transport region HTR. Accordingly, the light emitting element ED may exhibit high efficiency and long service life characteristics. In one or more embodiments, the light emitting element ED of one or more embodiments may exhibit high light efficiency and improved service life characteristics in a blue light emitting region. The light emitting element ED according to one or more embodiments includes the amine compound of one or more embodiments in the hole transport region HTR to exhibit high efficiency and long service life characteristics, and thus the display device DD-a of one or more embodiments may exhibit excellent or suitable display quality. Referring to FIG. 8, the emission layer EML may be arranged in an opening OH defined in a pixel defining layer PDL. For example, the emission layer EML which is divided by the pixel defining layer PDL and provided corresponding to each light emitting regions PXA-R, PXA-G, and PXA-B may be to emit light in substantially the same wavelength range. In the display device DD-a of one or more embodiments, the emission layer EML may be to emit blue light. In one or more embodiments, the emission layer EML may be provided as a common layer in the entire light emitting regions PXA-R, PXA-G, and PXA-B.

The light control layer CCL may be arranged on the display panel DP. Although the light control layer CCL is shown as being arranged above the display element layer DP-ED, embodiments of the present disclosure are not limited to this, in some embodiments, the light control layer CCL may be arranged below the display element layer DP-ED. The light control layer CCL may include a light conversion body. The light conversion body may be a quantum dot, a phosphor, and/or the like. The light conversion body may be to emit provided light by converting the wavelength thereof. For example, the light control layer CCL may a layer containing a quantum dot or a layer containing a phosphor.

The light control layer CCL may include a plurality of light control parts CCP1, CCP2, and CCP3. The light control parts CCP1, CCP2, and CCP3 may be spaced and apart from each other.

Referring to FIG. 8, divided patterns BMP may be arranged between the light control parts CCP1, CCP2, and CCP3 which are spaced and apart from each other, but embodiments of the present disclosure are not limited thereto. FIG. 8 illustrates that the divided patterns BMP do not overlap the light control parts CCP1, CCP2, and

CCP3, but, in some embodiments, at least a portion of the edges of the light control parts CCP1, CCP2, and CCP3 may overlap the divided patterns BMP.

The light control layer CCL may include a first light control part CCP1 containing a first quantum dot QD1 which converts first color light provided from the light emitting element ED into second color light, a second light control part CCP2 containing a second quantum dot QD2 which converts the first color light into third color light, and a third light control part CCP3 which transmits the first color light.

In one or more embodiments, the first light control part CCP1 may provide red light that is the second color light, and the second light control part CCP2 may provide green light that is the third color light. The third light control part CCP3 may provide blue light by transmitting the blue light that is the first color light provided from the light emitting element ED. For example, in some embodiments, the first quantum dot QD1 may be a red quantum dot to emit red light, and the second quantum dot QD2 may be a green quantum dot to emit green light. The same as described above may be applied with respect to the quantum dots QD1 and QD2.

In one or more embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but may include the scatterer SP.

The scatterer SP may be inorganic particles. For example, the scatterer SP may include at least one of TiO₂, ZnO, Al₂O₃, SiO₂, or hollow sphere silica. In one or more embodiments, the scatterer SP may include any one selected from among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 may include base resins BR1, BR2, and BR3, respectively, in which the quantum dots QD1 and QD2 and the scatterer SP are dispersed. In one or more embodiments, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may each independently be acrylic-based resins, urethane-based resins, silicone-based resins, epoxy-based resins, and/or the like. The base resins BR1, BR2, and BR3 may be transparent resins. In one or more embodiments, the first base resin BR1, the second base resin BR2, and the third base resin BR3 may be the same as or different from each other.

In one or more embodiments, the light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may block or reduce the light control parts CCP1, CCP2, and CCP3 from being exposed to moisture/oxygen. In one or more embodiments, the barrier layer BFL1 may cover the light control parts CCP1, CCP2, and CCP3. In one or more embodiments, a barrier layer BFL2 may be provided between the light control parts CCP1, CCP2, and CCP3 and the color filter layer CFL.

The barrier layers BFL1 and BFL2 may include at least one inorganic layer. For example, in some embodiments, the barrier layers BFL1 and BFL2 may include an inorganic material. For example, the barrier layers BFL1 and BFL2 may include one or more selected from among a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, and/or the like. In one or more embodiments, the barrier layers BFL1 and BFL2 may further include an organic film. The barrier layers BFL1 and BFL2 may be formed of a single layer or a plurality of layers.

In the display device DD-a of one or more embodiments, the color filter layer CFL may be arranged on the light control layer CCL. For example, the color filter layer CFL may be directly arranged on the light control layer CCL. In these embodiments, the barrier layer BFL2 may not be provided.

The color filter layer CFL may include color filters CF1, CF2, and CF3. The color filter layer CFL may include a first filter CF1 configured to transmit the second color light, a second filter CF2 configured to transmit the third color light, and a third filter CF3 configured to transmit the first color light. For example, in some embodiments, the first filter CF1 may be a red filter, the second filter CF2 may be a green filter, and the third filter CF3 may be a blue filter. The filters CF1, CF2, and CF3 each may include a polymeric photosensitive resin and a pigment and/or dye. The first filter CF1 may include a red pigment and/or dye, the second filter CF2 may include a green pigment and/or dye, and the third filter CF3 may include a blue pigment and/or dye. Embodiments of the present disclosure are not limited thereto, for example, in one or more embodiments, the third filter CF3 may not include (e.g., may exclude) any pigment and/or dye. The third filter CF3 may include a polymeric photosensitive resin and may not include (e.g., may exclude) any pigment and/or dye. The third filter CF3 may be transparent. The third filter CF3 may be formed of a transparent photosensitive resin.

In some embodiments, the first filter CF1 and the second filter CF2 may each be a yellow filter. The first filter CF1 and the second filter CF2 may not be separated but be provided as one filter.

In one or more embodiments, the color filter layer CFL may further include a light shielding part. The light shielding part may be a black matrix. The light shielding part may include an organic light shielding material and/or an inorganic light shielding material containing a black pigment and/or dye. The light shielding part may prevent or reduce light leakage, and may separate boundaries between adjacent filters CF1, CF2, and CF3. Also, in some embodiments, the light shielding part may be formed of a blue filter.

In one or more embodiments, the first to third filters CF1, CF2, and CF3 may be arranged corresponding to the red light emitting region PXA-R, the green light emitting region PXA-G, and the blue light emitting region PXA-B, respectively.

A base substrate BL may be arranged on the color filter layer CFL. The base substrate BL may be a member which provides a base surface on which the color filter layer CFL, the light control layer CCL, and/or the like are arranged. The base substrate BL may be a glass substrate, a metal substrate, a plastic substrate, and/or the like. However, embodiments of the present disclosure are not limited thereto, and the base substrate BL may be an inorganic layer, an organic layer, or a composite material layer. In one or more embodiments, the base substrate BL may not be provided.

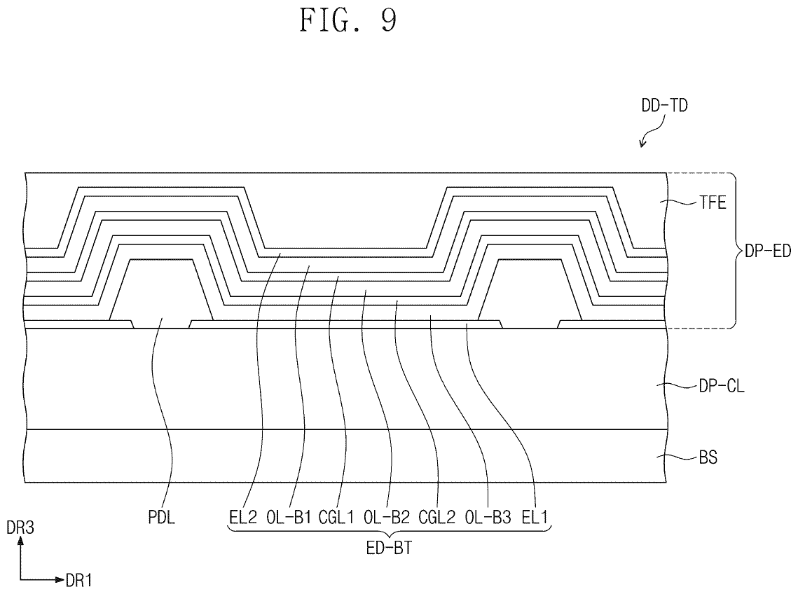

FIG. 9 is a cross-sectional view illustrating a portion of the display device according to one or more embodiments. In a display device DD-TD according to one or more embodiments, a light-emitting element ED-BT may include a plurality of light-emitting structures OL-B1, OL-B2, and OL-B3.

At least one selected from among the plurality of light emitting structures OL-B1, OL-B2, and OL-B3 may include the amine compound of one or more embodiments. Accordingly, the light emitting element ED-BT may exhibit high efficiency and long service life characteristics.

The light emitting element ED-BT may include a first electrode EL1 and a second electrode EL2 which face each other, and the plurality of light emitting structures OL-B1, OL-B2, and OL-B3 sequentially stacked in a thickness direction between the first electrode EL1 and the second electrode EL2. The light emitting structures OL-B1, OL-B2, and OL-B3 each may include an emission layer EML (FIG. 8) and a hole transport region HTR and an electron transport region ETR arranged with the emission layer EML (FIG. 8) located therebetween. For example, the light emitting element ED-BT included in the display device DD-TD of one or more embodiments may be a light emitting element having a tandem structure and including a plurality of emission layers.

In one or more embodiments illustrated in FIG. 9, all light beams respectively emitted from the light emitting structures OL-B1, OL-B2, and OL-B3 may be blue light. However, embodiments of the present disclosure are not limited thereto, and the light beams respectively emitted from the light emitting structures OL-B1, OL-B2, and OL-B3 may have wavelength ranges different from each other. For example, in one or more embodiments, the light emitting element ED-BT including the plurality of light emitting structures OL-B1, OL-B2, and OL-B3 which emit light beams having wavelength ranges different from each other may be to emit white light.

Charge generating layers CGL1 and CGL2 may be separately and respectively arranged between neighboring light-emitting structures OL-B1, OL-B2, and OL-B3. The charge generating layers CGL1 and CGL2 may include a p-type or kind charge (e.g., P-charge) generating layer and/or an n-type or kind charge (e.g., N-charge) generating layer.

In one or more embodiments illustrated in FIG. 9, at least one selected from among the light emitting structures OL-B1, OL-B2, and OL-B3 may include the amine compound of one or more embodiments in the hole transport region HTR (see FIG. 8). Thus, the at least one selected from among the light emitting structures OL-B1, OL-B2, and OL-B3 may exhibit high efficiency and long service life characteristics.

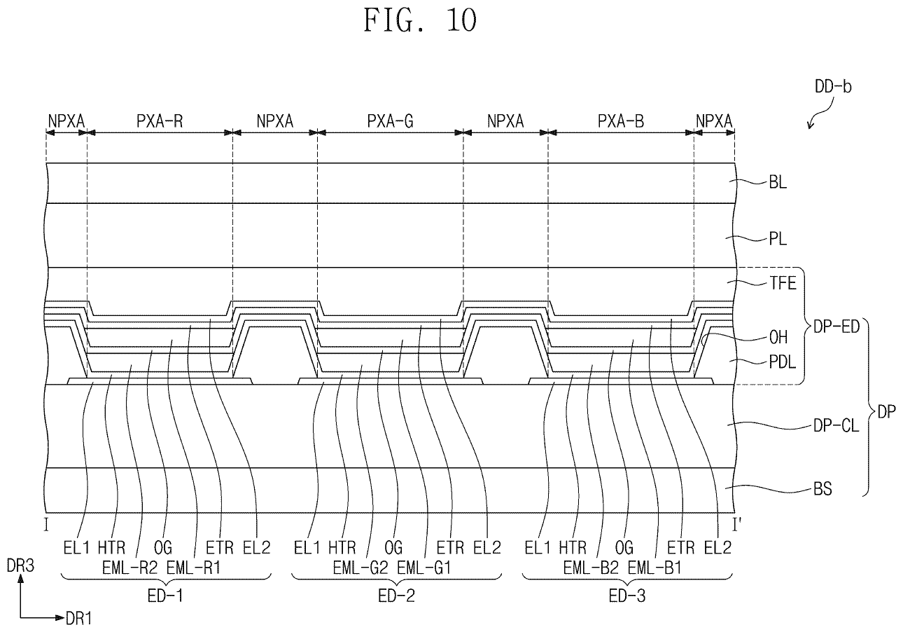

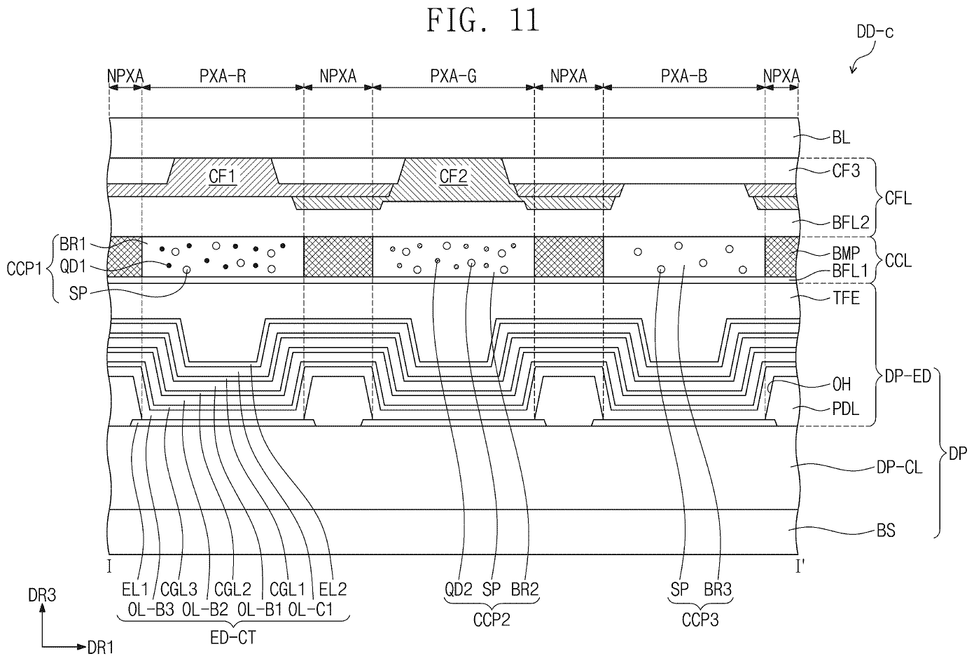

Referring to FIG. 10, the display device DD-b according to one or more embodiments may include light emitting elements ED-1, ED-2, and ED-3 in each of which two emission layers are stacked. At least one selected from among the light emitting elements ED-1, ED-2, and ED-3 may include the amine compound of one or more embodiments. Accordingly, the light emitting elements ED-1, ED-2, and ED-3 may exhibit high efficiency and long service life characteristics. In one or more embodiments, the light emitting element ED-3 of one or more embodiments may exhibit high efficiency and improved service life characteristics in a blue light emitting region.

Compared with the display device DD illustrated in FIG. 2, the display device DD-b illustrated in FIG. 10 has a difference in that the first to third light emitting elements ED-1, ED-2, and ED-3 each include two emission layers stacked in a thickness direction. In each of the first to third light emitting elements ED-1, ED-2, and ED-3, the two emission layers may be to emit light in substantially the same wavelength region.

In one or more embodiments, the first light emitting element ED-1 may include a first red emission layer EML-R1 and a second red emission layer EML-R2. The second light emitting element ED-2 may include a first green emission layer EML-G1 and a second green emission layer EML-G2. In addition, the third light emitting element ED-3 may include a first blue emission layer EML-B1 and a second blue emission layer EML-B2. An emission auxiliary part OG may be arranged between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. For example, the emission auxiliary part OG may include an electron transport region, a charge generation layer, and a hole transport region that are sequentially stacked (e.g., in the stated order). The emission auxiliary part OG may be provided as a common layer in the whole of the first to third light emitting elements ED-1, ED-2, and ED-3. However, embodiments of the present disclosure are not limited thereto, and the emission auxiliary part OG may be provided by being patterned within the openings OH defined in the pixel defining layer PDL.

The first red emission layer EML-R1, the first green emission layer EML-G1, and the first blue emission layer EML-B1 may be arranged between the electron transport region ETR and the emission auxiliary part OG. The second red emission layer EML-R2, the second green emission layer EML-G2, and the second blue emission layer EML-B2 may be arranged between the emission auxiliary part OG and the hole transport region HTR.

For example, in one or more embodiments, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2 that are sequentially stacked (e.g., in the stated order). The second light emitting element ED-2 may include a first electrode EL1, the hole transport region HTR, a second green emission layer EML-G2, the emission auxiliary part OG, a first green emission layer EML-G1, the electron transport region ETR, and the second electrode EL2 that are sequentially stacked (e.g., in the stated order). The third light emitting element ED-3 may include a first electrode EL1, the hole transport region HTR, a second blue emission layer EML-B2, the emission auxiliary part OG, a first blue emission layer EML-B1, the electron transport region ETR, and the second electrode EL2 that are sequentially stacked (e.g., in the stated order).

The light emitting elements ED-1, ED-2, and ED-3 according to one or more embodiments each include the amine compound of one or more embodiments in the hole transport region HTR to exhibit high efficiency and long service life characteristics, and thus the display device DD-b of one or more embodiments may exhibit excellent or suitable display quality.

In one or more embodiments, an optical auxiliary layer PL may be arranged on the display element layer DP-ED. The optical auxiliary layer PL may include a polarizing layer. The optical auxiliary layer PL may be arranged on the display panel DP and control reflected light in the display panel DP due to external light. In some embodiments, the optical auxiliary layer PL in the display device may not be provided.

Unlike FIGS. 9 and 10, a display device DD-c of FIG. 11 is illustrated to include four light emitting structures OL-B1, OL-B2, OL-B3, and OL-C1. A light emitting element ED-CT may include a first electrode EL1 and a second electrode EL2 facing each other, and the first to fourth light emitting structures OL-B1, OL-B2, OL-B3, and OL-C1 sequentially stacked in a thickness direction between the first electrode EL1 and the second electrode EL2.

At least one selected from among the first to fourth light emitting structures OL-B1, OL-B2, OL-B3, and OL-C1 may include the amine compound of one or more embodiments. Accordingly, the light emitting element ED-CT may exhibit high efficiency and long service life characteristics. In one or more embodiments, the display device DD-c of one or more embodiments may exhibit excellent or suitable display quality.

Charge generation layers CGL1, CGL2, and CGL3 may be arranged between the first to fourth light emitting structures OL-B1, OL-B2, OL-B3, and OL-C1. Among the four light emitting structures, the first to third light emitting structures OL-B1, OL-B2, and OL-B3 may be to emit blue light, and the fourth light emitting structure OL-C1 may be to emit green light. However, embodiments of the present disclosure are not limited thereto, and the first to fourth light emitting structures OL-B1, OL-B2, OL-B3, and OL-C1 may be to emit light beams in different wavelength regions. The charge generation layers CGL1, CGL2, and CGL3 arranged between adjacent light emitting structures OL-B1, OL-B2, OL-B3, and OL-C1 may include a p-type or kind charge generation layer and/or an n-type or kind charge generation layer.

In one or more embodiments, an electronic apparatus may include a display device including a plurality of light emitting elements, and a control part which controls the display device. The electronic apparatus of one or more embodiments may be a device that is activated according to an electrical signal. The electronic apparatus may include display devices of one or more suitable embodiments. For example, the electronic apparatus may include not only large-sized electronic apparatuses such as a television set, a monitor, or an outdoor billboard but also include small- and medium-sized electronic apparatuses such as a personal computer, a laptop computer, a personal digital terminal, a display device for a vehicle, a game console, a portable electronic device, or a camera.

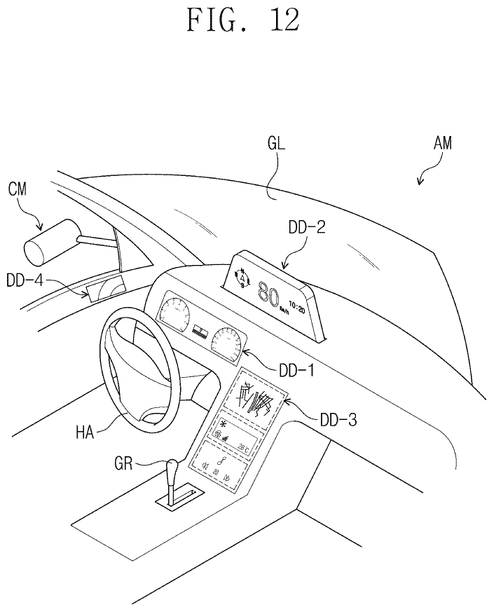

FIG. 12 is a view illustrating a vehicle AM in which first to fourth display devices DD-1, DD-2, DD-3, and DD-4 are arranged. At least one selected from among the first to fourth display devices DD-1, DD-2, DD-3, and DD-4 may include substantially the same configuration of display devices DD, DD-TD, DD-a, DD-b, or DD-c, according to one or more embodiments, described with reference to FIGS. 1, 2, and 8 to 11.

FIG. 12 illustrates a vehicle AM, but this is an example, and the first to fourth display devices DD-1, DD-2, DD-3, and DD-4 may be arranged in other transportation apparatuses such as bicycles, motorcycles, trains, ships, and/or airplanes. In one or more embodiments, at least one selected from among the first to fourth display devices DD-1, DD-2, DD-3, and DD-4 including substantially the same configuration as one of the display devices DD, DD-TD, DD-a, DD-b, and DD-c of one or more embodiments may be employed in a personal computer, a laptop computer, a personal digital terminal, a game console, a portable electronic device, a television, a monitor, an outdoor billboard, and/or the like. These are merely provided as embodiments, and the display devices may be employed in other electronic apparatuses unless departing from the present disclosure.

In one or more embodiments, at least one selected from among the first to fourth display devices DD-1, DD-2, DD-3, and DD-4 may include the light emitting element ED of one or more embodiments as described with reference to FIGS. 3 to 7. At least one selected from among the first to fourth display devices DD-1, DD-2, DD-3, and DD-4 may include the amine compound of one or more embodiments.

Accordingly, the first to fourth display devices DD-1, DD-2, DD-3, and DD-4 including the amine compound of one or more embodiments may have an improvement in display efficiency and a display service life. In one or more embodiments, the first to fourth display devices DD-1, DD-2, DD-3, and DD-4 including the amine compound of one or more embodiments may exhibit excellent or suitable display quality.

Referring to FIG. 12, the vehicle AM may include a steering wheel HA and a gear GR for driving the vehicle AM. In addition, the vehicle AM may include a front window GL arranged so as to face a driver.

The first display device DD-1 may be arranged in a first region overlapping the steering wheel HA. For example, the first display device DD-1 may be a digital cluster which displays first information of the vehicle AM. The first information may include a first scale which indicates a driving speed of the vehicle AM, a second scale which indicates an engine speed (that is, revolutions per minute (RPM)), an image which indicates a fuel state, and/or the like. The first scale and the second scale may be indicated as digital images.

The second display device DD-2 may be arranged in a second region facing a driver seat and overlapping the front window GL. The driver seat may be a seat in which the steering wheel HA faces. For example, the second display device DD-2 may be a head up display (HUD) which displays second information of the vehicle AM. The second display device DD-2 may be optically transparent. The second information may include digital numbers which indicate a driving speed, and may further include information such as the current time. In some embodiments, the second information of the second display device DD-2 may be projected to the front window GL to be displayed.

The third display device DD-3 may be arranged in a third region adjacent to the gear GR. For example, the third display device DD-3 may be arranged between the driver seat and the passenger seat and may be a center information display (CID) for a vehicle for displaying third information. The passenger seat may be a seat spaced apart from the driver seat with the gear GR arranged therebetween. The third information may include information about traffic (e.g., navigation information), playing music or radio or a video (or an image), temperatures inside the vehicle AM, and/or the like.

The fourth display device DD-4 may be spaced apart from the steering wheel HA and the gear GR, and may be arranged in a fourth region adjacent to a side of the vehicle AM. For example, the fourth display device DD-4 may be a digital side-view mirror which displays fourth information. The fourth display device DD-4 may display an image outside the vehicle AM taken by a camera module CM arranged outside the vehicle AM. The fourth information may include an image outside the vehicle AM.

The above-described first to fourth information may be examples, and the first to fourth display devices DD-1, DD-2, DD-3, and DD-4 may further display information about the inside and outside of the vehicle AM. The first to fourth information may include different information. However, embodiments of the present disclosure are not limited thereto, for example, in some embodiments, a part of the first to fourth information may include the same information as one another.

Hereinafter, referring to Examples and Comparative Examples, the amine compound according to one or more embodiments of the present disclosure and the light emitting element of one or more embodiments will be described in more detail. In addition, Examples shown are for illustrative purposes only to facilitate the understanding of the present disclosure, and thus, the scope of the present disclosure is not limited thereto.

### EXAMPLES

### 1. Synthesis of Amine Compound of Example

A synthetic method of the amine compound according to the example embodiments will be described in more detail by illustrating synthetic methods of Compounds 1, 4, 13, 26, 71, 74, 136, 174, 193, and 201. Also, in the following descriptions, the synthetic method of the amine compound is provided as an example, but the synthetic method according to one or more embodiments of the present disclosure is not limited to Examples.

### (1) Synthesis of Compound 1

Amine Compound 1 according to an example may be synthesized by, for example, the steps (e.g., acts or tasks) shown in Reaction Scheme 1:

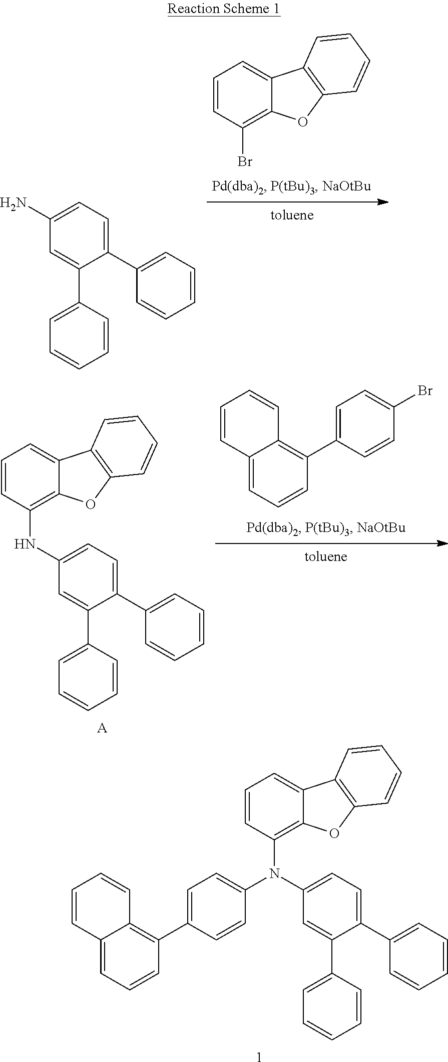

In an argon atmosphere, to a 1-L three-neck flask, [1,1′:2′,1″-terphenyl]-4′-amine (20.0 g), 4-bromodibenzo[b,d]furan (20.1 g), bis(dibenzylideneacetone) palladium (0) (Pd(dba)₂, 2.3 g), and sodium tert-butoxide (NaOtBu, 11.8 g) were added and dissolved in toluene (400 mL), tri-tert-butylphosphine (P(tBu)₃, 2.0 M in toluene, 4.0 mL) was added thereto, and the resultant mixture was stirred at room temperature for about 4 hours. The resultant mixture was extracted with CH₂Cl₂ by adding water to obtain organic layers. The obtained organic layers were collected and dried over MgSO₄, and then the solvent was removed by distillation under reduced pressure. The resulting crude product was purified by recrystallization in toluene to obtain Intermediate Compound A (24.1 g, yield: 72%). The molecular weight of Intermediate Compound A measured by fast atom bombardment mass spectrometry (FAB-MS) measurement was 411.

Thereafter, in an argon atmosphere, to 300-mL three-neck flask, Intermediate Compound A (5.0 g), 1-(4-bromophenyl)naphthalene (3.4 g), Pd(dba)₂ (0.35 g), and NaOtBu (1.8 g) were added and dissolved in toluene (100 mL), P(tBu)₃ (2.0 M in toluene, 0.6 mL) was added thereto, and the resultant mixture was heated and stirred at about 100° C. for about 4 hours. Then, the resultant mixture was extracted with CH₂Cl₂ by adding water to obtain organic layers. The obtained organic layers were collected and dried over MgSO₄, and then the solvent was removed by distillation under reduced pressure. The resulting crude product was purified by silica gel column chromatography to obtain Compound 1 (6.1 g, yield: 82%). The molecular weight of Compound 1 measured by FAB-MS measurement was 613.

### (2) Synthesis of Compound 4

Amine Compound 4 according to an example may be synthesized by, for example, the steps shown in Reaction Scheme 2:

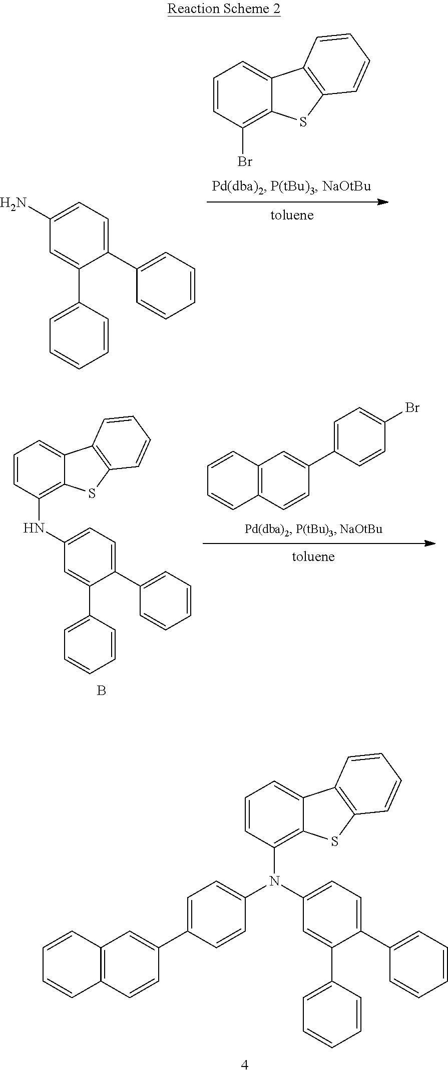

In the same manner as in the synthetic method of Intermediate Compound A above, Intermediate Compound B (22.6 g, yield: 65%) was obtained from [1,1′: 2′,1″-terphenyl]-4′-amine (20.0 g) and 4-bromodibenzo[b,d] thiophene (21.4 g). The molecular weight of Intermediate Compound B measured by FAB-MS measurement was 427.

In the same manner as in the synthetic method of Compound 1 above, Compound 4 (6.2 g, yield: 85%) was obtained from Intermediate Compound B (5.0 g) and 2-(4-bromophenyl)naphthalene (3.3 g). The molecular weight of Compound 4 measured by FAB-MS measurement was 629.

### (3) Synthesis of Compound 13

Amine Compound 13 according to an example may be synthesized by, for example, the steps shown in Reaction Scheme 3:

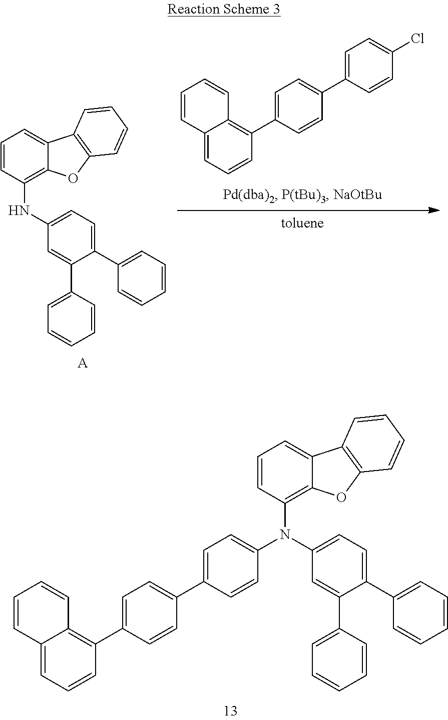

In the same manner as in the synthetic method of Compound 1 above, Compound 13 (6.3 g, yield: 76%) was obtained from Intermediate Compound A (5.0 g) and 1-(4′-chloro-[1,1′-biphenyl]-4-yl)naphthalene (3.8 g). The molecular weight of Compound 13 measured by FAB-MS measurement was 689.

### (4) Synthesis of Compound 26

Amine Compound 26 according to an example may be synthesized by, for example, the steps shown in Reaction Scheme 4:

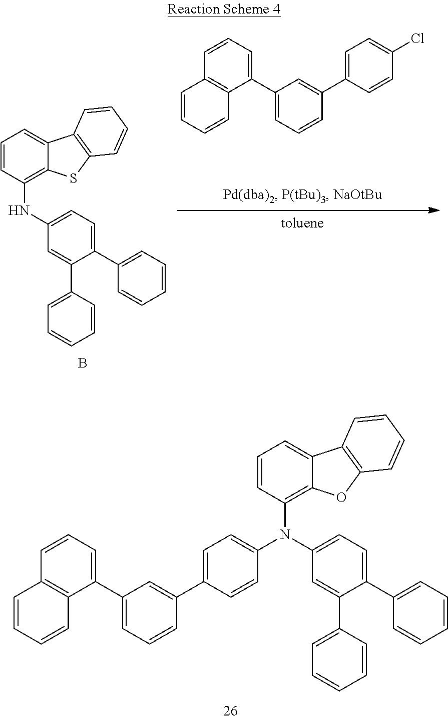

In the same manner as in the synthetic method of Compound 1 above, Compound 26 (6.2 g, yield: 75%) was obtained from Intermediate Compound B (5.0 g) and 1-(4′-chloro-[1,1′-biphenyl]-3-yl)naphthalene (3.7 g). The molecular weight of Compound 26 measured by FAB-MS measurement was 705.

### (5) Synthesis of Compound 71

Amine Compound 71 according to an example may be synthesized by, for example, the steps shown in Reaction Scheme 5:

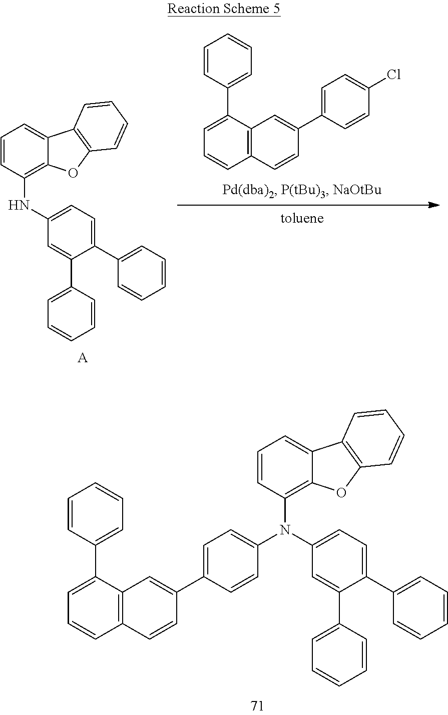

In the same manner as in the synthetic method of Compound 1 above, Compound 71 (6.8 g, yield: 82%) was obtained from Intermediate Compound A (5.0 g) and 7-(4-chlorophenyl)-1-phenylnaphthalene (3.8 g). The molecular weight of Compound 71 measured by FAB-MS measurement was 689.

### (6) Synthesis of Compound 74

Amine Compound 74 according to an example may be synthesized by, for example, the steps shown in Reaction Scheme 6:

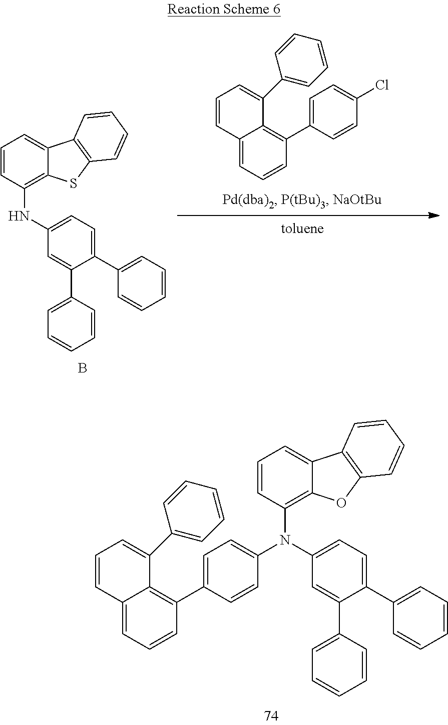

In the same manner as in the synthetic method of Compound 1 above, Compound 74 (5.7 g, yield: 69%) was obtained from Intermediate Compound B (5.0 g) and 1-(4-chlorophenyl)-8-phenylnaphthalene (3.7 g). The molecular weight of Compound 74 measured by FAB-MS measurement was 705.

### (7) Synthesis of Compound 136

Amine Compound 136 according to an example may be synthesized by, for example, the steps shown in Reaction Scheme 7:

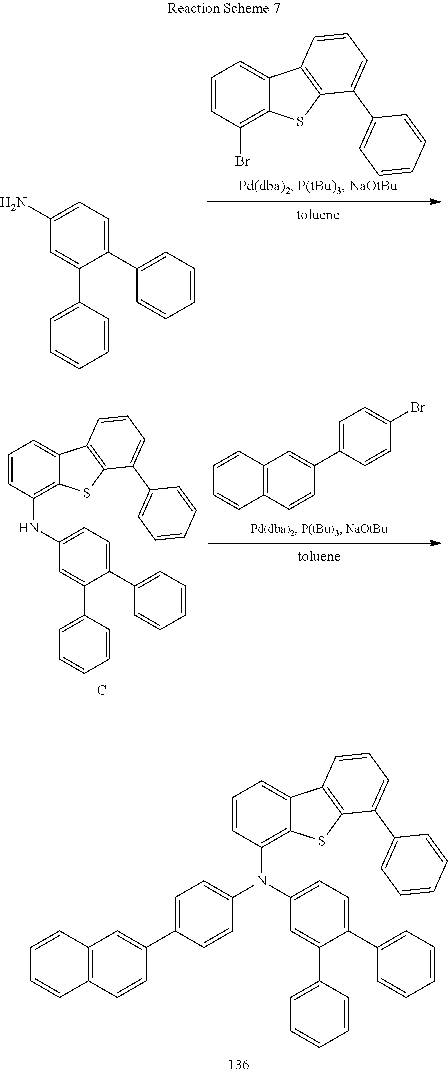

In the same manner as in the synthetic method of Intermediate Compound A above, Intermediate Compound C (23.8 g, yield: 58%) was obtained from [1,1′: 2′,1″-terphenyl]-4′-amine (20.0 g) and 4-bromo-6-phenyldibenzo[b,d] thiophene (27.6 g). The molecular weight of Intermediate Compound C measured by FAB-MS measurement was 503.

In the same manner as in the synthesis of Compound 1 above, Compound 136 (5.6 g, yield: 80%) was obtained from Intermediate Compound C (5.0 g) and 2-(4-bromophenyl)naphthalene (2.8 g). The molecular weight of Compound 136 measured by FAB-MS measurement was 705.

### (8) Synthesis of Compound 174

Amine Compound 174 according to an example may be synthesized by, for example, the steps shown in Reaction Scheme 8:

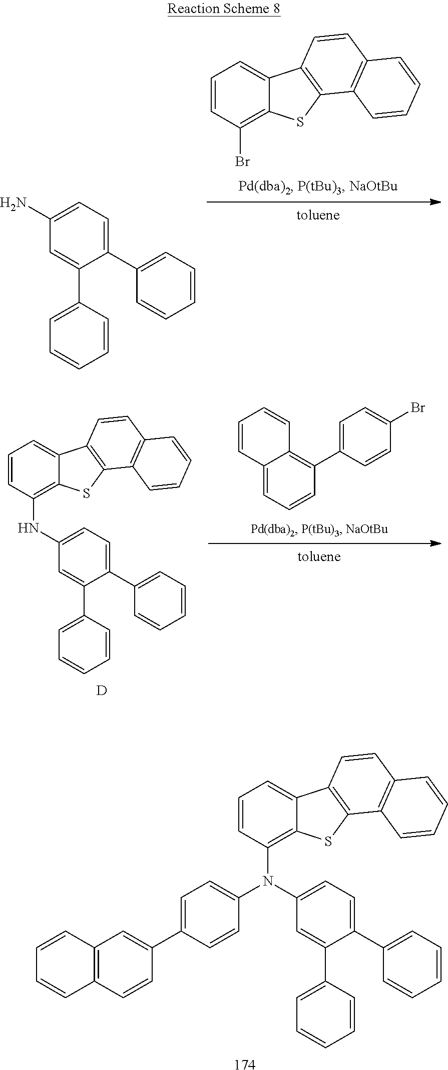

In the same manner as in the synthetic method of Intermediate Compound A above, Intermediate Compound D (23.7 g, yield: 61%) was obtained from [1,1′: 2′,1″-terphenyl]-4′-amine (20.0 g) and 10-bromobenzo[b]naphtho[2,1-d]thiophene (25.5 g).

The molecular weight of Intermediate Compound D measured by FAB-MS measurement was 477.

In the same manner as in the synthesis of Compound 1 above, Compound 174 (5.3 g, yield: 75%) was obtained from Intermediate Compound D (5.0 g) and 1-(4-bromophenyl)naphthalene (3.0 g). The molecular weight of Compound 174 measured by FAB-MS measurement was 679.

### (9) Synthesis of Compound 193

Amine Compound 193 according to an example may be synthesized by, for example, the steps shown in Reaction Scheme 9:

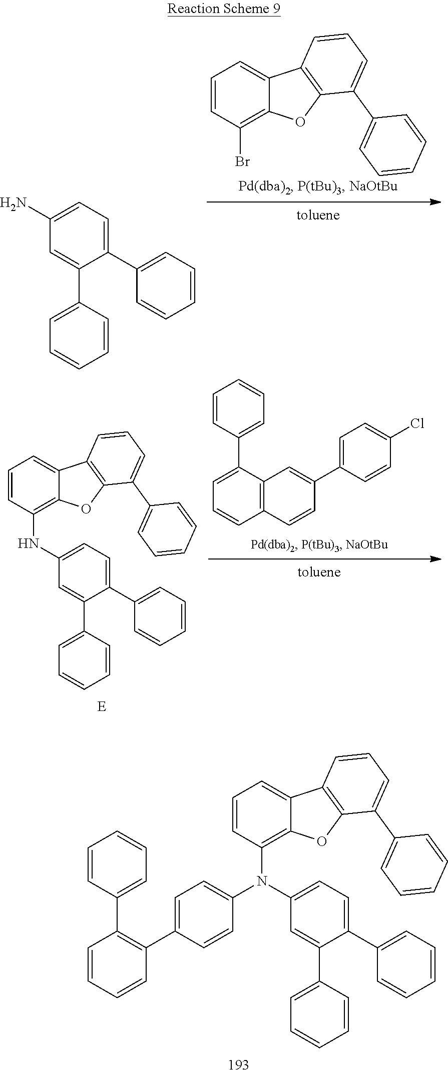

In the same manner as in the synthetic method of Intermediate Compound A above, Intermediate Compound E (21.0 g, yield: 53%) was obtained from [1,1′: 2′,1″-terphenyl]-4′-amine (20.0 g) and 4-bromo-6-phenyldibenzo[b,d]furan (26.3 g). The molecular weight of Intermediate Compound E measured by FAB-MS measurement was 487.

In the same manner as in the synthesis of Compound 1 above, Compound 193 (6.6 g, yield: 84%) was obtained from Intermediate Compound E (5.0 g) and 7-(4-chlorophenyl)-1-phenylnaphthalene (3.3 g). The molecular weight of Compound 193 measured by FAB-MS measurement was 765.

### (10) Synthesis of Compound 201

Amine Compound 201 according to an example may be synthesized by, for example, the steps shown in Reaction Scheme 10:

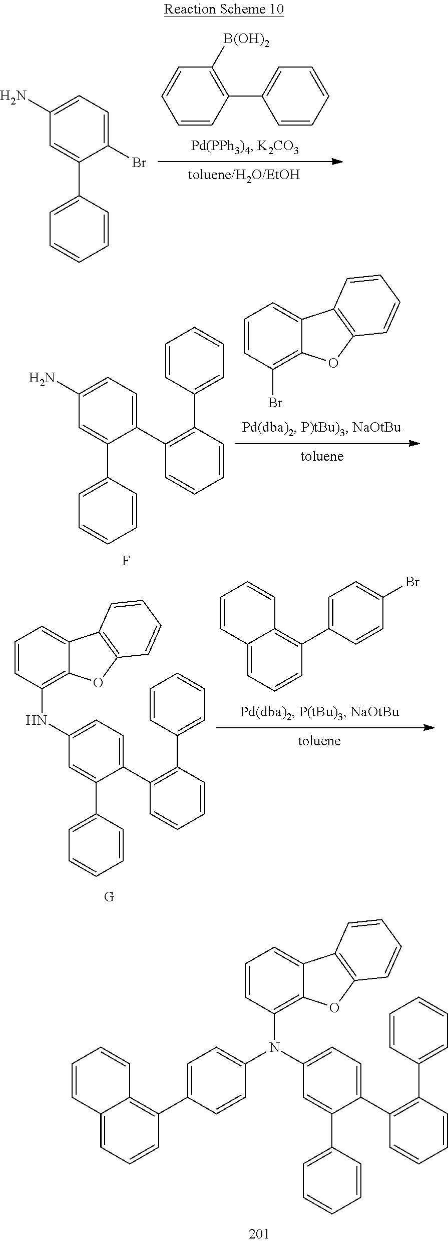

In an argon atmosphere, to a 1-L three-neck flask, 6-bromo-[1,1′-biphenyl]-3-amine (15.0 g), 2-biphenylboronic acid (11.9 g), tetrakis(triphenylphosphine) palladium (0) (Pd(PPh₃)₄, 3.5 g), and potassium carbonate (K₂CO₃, 16.7 g) were added, and dissolved in a mixed solvent of toluene, water, and ethanol (toluene:water:ethanol=10:2:1, 300 mL), and the resultant mixture was heated and stirred at about 80° C. for about 8 hours. The resultant mixture was extracted with CH₂Cl₂ by adding water to obtain organic layers. The obtained organic layers were collected and dried over MgSO₄, and then the solvent was removed by distillation under reduced pressure. The resulting crude product was purified by silica gel chromatography to obtain Intermediate Compound F (11.6 g, yield: 60%). The molecular weight of Intermediate Compound F measured by FAB-MS measurement was 321.

In the same manner as in the synthesis of Intermediate Compound A above, Intermediate Compound F (10.0 g) and 4-bromodibenzo[b,d]furan (7.6 g) were reacted to obtain a crude product. The resulting crude product was purified by silica gel chromatography to obtain Intermediate Compound G (11.8 g, yield: 78%). The molecular weight of Intermediate Compound G measured by FAB-MS measurement was 487.

In the same manner as in the synthesis of Compound 1 above, Compound 201 (5.3 g, yield: 75%) was obtained from the Intermediate Compound G (5.0 g) and 1-(4-bromophenyl)naphthalene (2.9 g). The molecular weight of Compound 201 measured by FAB-MS measurement was 689.

### 2. Manufacture and Evaluation of Light Emitting Elements

### (1) Manufacture of Light Emitting Elements

Light emitting elements including an amine compound of an example or Comparative Example Compound in a hole transport layer were manufactured as follows. The amine compounds of examples were utilized as a material for the hole transport layer to manufacture the light emitting elements of Examples 1 to 10, respectively. Comparative Example Compound X-1 to X-8 were respectively utilized as a material for the hole transport layer to manufacture the light emitting elements of Comparative Examples 1 to Comparative Example 8. Example Compounds utilized in Examples 1 to 10 and Comparative Example Compounds utilized in Comparative Examples 1 to 8 were as follows:

### Example Compounds

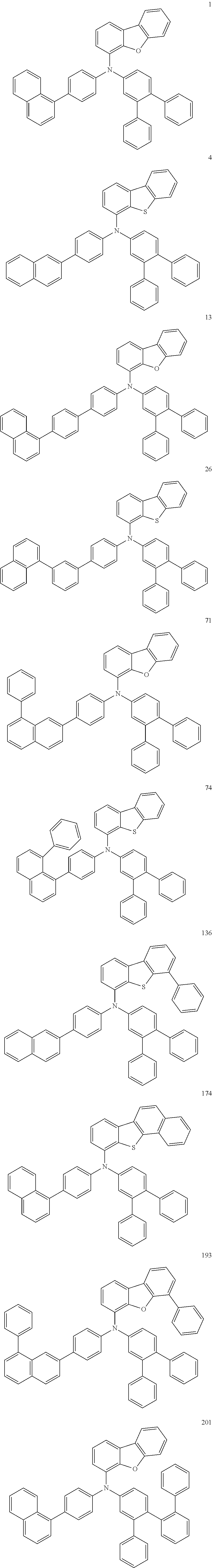

### Comparative Example Compounds

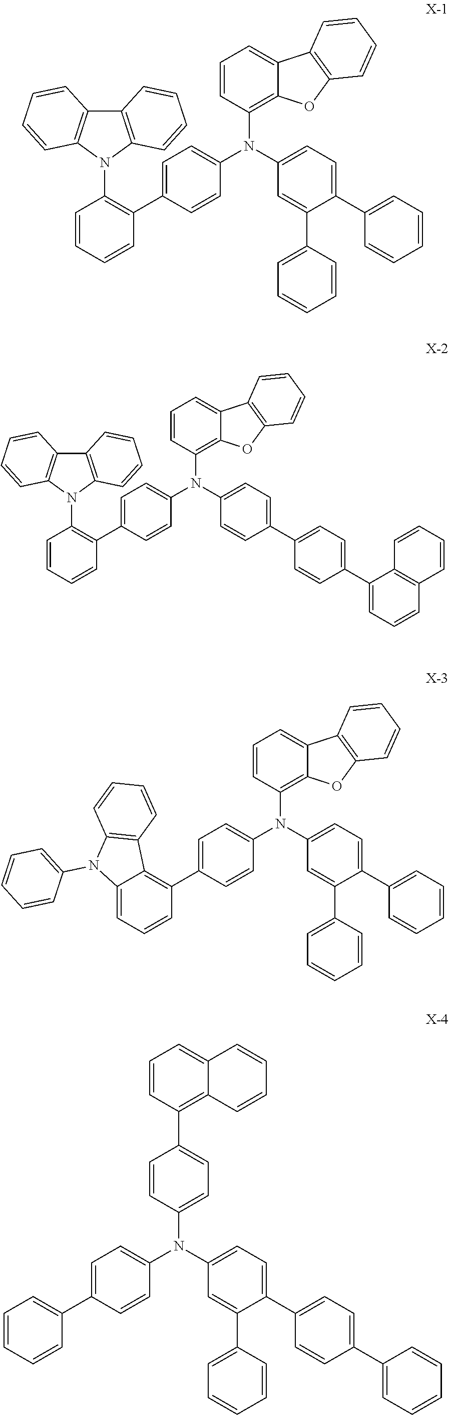 [SMILES: c1ccc(-c2ccc(N(c3ccc(-c4ccccc4-n4c5ccccc5c5ccccc54)cc3)c3cccc4c3oc3ccccc34)cc2-c2ccccc2)cc1 ; c1ccc(-n2c3ccccc3c3ccccc32)c(-c2ccc(N(c3ccc(-c4ccc(-c5cccc6ccccc56)cc4)cc3)c3cccc4c3oc3ccccc34)cc2)c1 ; c1ccc(-c2ccc(N(c3ccc(-c4cccc5c4c4ccccc4n5-c4ccccc4)cc3)c3cccc4c3oc3ccccc34)cc2-c2ccccc2)cc1 ; c1ccc(-c2ccc(-c3ccc(N(c4ccc(-c5ccccc5)cc4)c4ccc(-c5cccc6ccccc56)cc4)cc3-c3ccccc3)cc2)cc1]

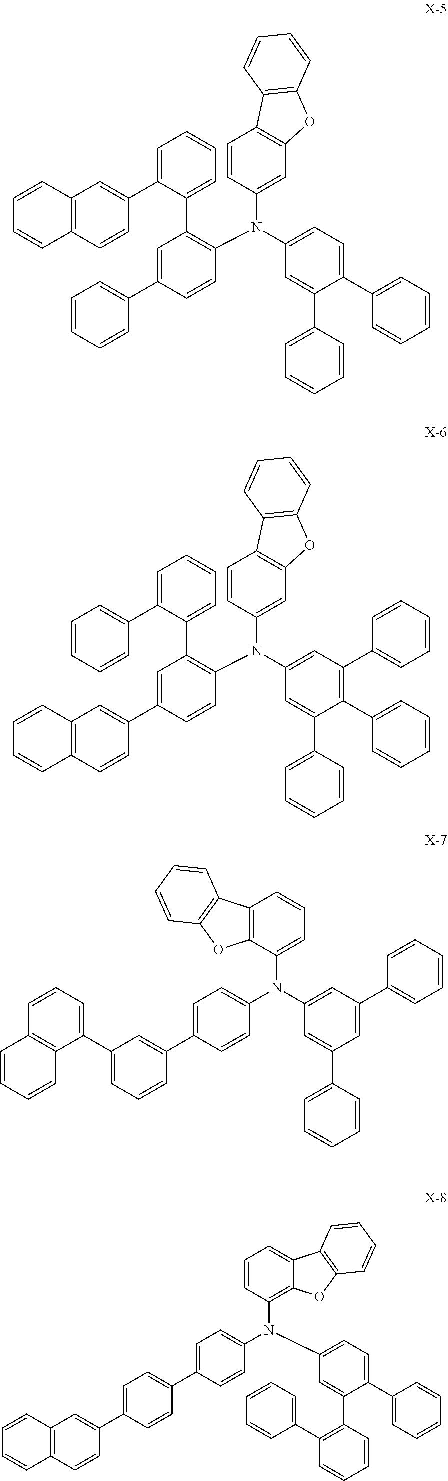 [SMILES: c1ccc(-c2ccc(N(c3ccc(-c4ccccc4)c(-c4ccccc4)c3)c3ccc4c(c3)oc3ccccc34)c(-c3ccccc3-c3ccc4ccccc4c3)c2)cc1 ; c1ccc(-c2ccccc2-c2cc(-c3ccc4ccccc4c3)ccc2N(c2cc(-c3ccccc3)c(-c3ccccc3)c(-c3ccccc3)c2)c2ccc3c(c2)oc2ccccc23)cc1 ; c1ccc(-c2cc(-c3ccccc3)cc(N(c3ccc(-c4cccc(-c5cccc6ccccc56)c4)cc3)c3cccc4c3oc3ccccc34)c2)cc1 ; c1ccc(-c2ccccc2-c2cc(N(c3ccc(-c4ccc(-c5ccc6ccccc6c5)cc4)cc3)c3cccc4c3oc3ccccc34)ccc2-c2ccccc2)cc1]

A glass substrate on which a 150 nm thick ITO had been patterned was ultrasonically washed and cleaned by utilizing isopropyl alcohol and pure water for about 5 minutes each. After ultrasonically washed and cleaned, the glass substrate was irradiated with UV rays for about 30 minutes and treated with ozone. Then, 2-TNATA was deposited to form (or provide) a 60 nm thick hole injection layer. On the hole injection layer, Example Compound or Comparative Example Compound was deposited to form (or provide) a 30 nm thick hole transport layer.

On the hole transport layer, TBP and ADN were co-deposited to form (or provide) a 25 nm thick emission layer. TBP and ADN were co-deposited in a weight ratio of about 3:97. Then, Alq₃ was deposited to be a thickness of about 25 nm and LiF was deposited to be a thickness of about 1 nm in this order to form (or provide) an electron transport region.

Next, Al was deposited to form (or provide) a 100 nm thick second electrode.

In the Examples, the hole transport region, the emission layer, the electron transport region, and the second electrode were formed utilizing a vacuum deposition apparatus.

The compounds utilized to manufacture the light emitting element are as follows:

### Materials Used to Manufacture Light Emitting Element

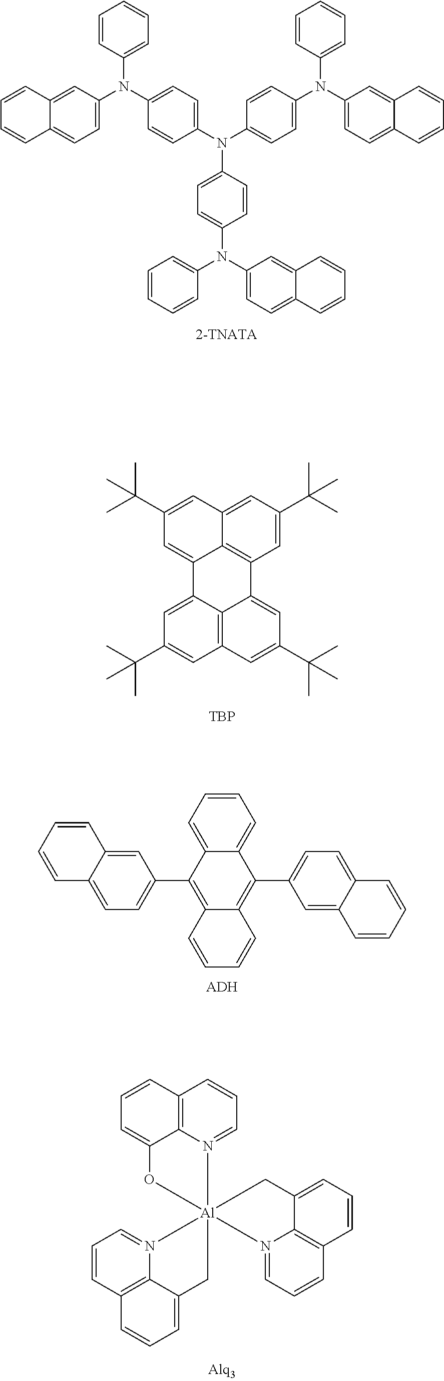

### (2) Evaluation of Light Emitting Elements

The light emitting elements of Examples and Comparative Examples were each evaluated and the results are shown in Table 1. Luminous efficiencies and element service life of each of the light emitting elements of Examples and Comparative Examples are shown in Table 1. The evaluation of the luminous efficiencies and element service life were performed by utilizing brightness light distribution characteristics measurement equipment, C9920-11 manufactured by Hamamatsu Photonics, Inc.

The luminous efficiency is relatively indicated based on 100% of the luminous efficiency of Comparative Example 1 at a current density of 10 mA/cm². For the relative element service life, a time taken to deteriorate the brightness to 50% of an initial brightness during the continuous operation of a light emitting element is relatively indicated based on 100% of the value of Comparative Example 1.

TABLE 1

| Element | | | |
|---|---|---|---|
| Manufacture | Hole transport layer | Luminous | Element service |
| Examples | material | efficiency (%) | life (LT50) (%) |
| Example 1 | Compound 1 | 110% | 110% |
| Example 2 | Compound 4 | 107% | 130% |
| Example 3 | Compound 13 | 111% | 110% |
| Example 4 | Compound 26 | 108% | 120% |
| Example 5 | Compound 71 | 106% | 130% |
| Example 6 | Compound 74 | 102% | 130% |
| Example 7 | Compound 136 | 107% | 120% |
| Example 8 | Compound 174 | 112% | 110% |
| Example 9 | Compound 193 | 107% | 130% |
| Example 10 | Compound 201 | 107% | 110% |
| Comparative | Comparative Example | 100% | 100% |
| Example 1 | Compound X-1 | | |
| Comparative | Comparative Example | 100% | 95% |
| Example 2 | Compound X-2 | | |
| Comparative | Comparative Example | 95% | 95% |
| Example 3 | Compound X-3 | | |
| Comparative | Comparative Example | 100% | 100% |
| Example 4 | Compound X-4 | | |
| Comparative | Comparative Example | 97% | 110% |
| Example 5 | Compound X-5 | | |
| Comparative | Comparative Example | 95% | 90% |
| Example 6 | Compound X-6 | | |
| Comparative | Comparative Example | 102% | 90% |
| Example 7 | Compound X-7 | | |
| Comparative | Comparative Example | 102% | 50% |
| Example 8 | Compound X-8 | | |

Referring to the results of Table 1, Examples 1 to 10 each exhibited high efficiency and long life element characteristics compared with Comparative Examples 1 to 8. Comparative Example Compounds X-1 to X-3 do not include a naphthyl group compared with Example Compounds, and Comparative Compound X-4 does not include a dibenzoheterole group compared with Example Compounds. For example, compared with Comparative Examples 1 to 4, respectively including Comparative Example Compounds X-1 to X-4, which do not include some of the dibenzoheterole group, the naphthyl group, and the ortho-terphenyl group included in the amine compound of an embodiment, the light emitting elements of embodiments including the amine compounds of embodiments exhibited relatively high luminous efficiency and relatively long service life characteristics.

Comparative Example Compounds X-5 and X-6 have a difference in the bonding position of dibenzoheterole group from Example Compounds. Compared with Comparative Examples 5 and 6 respectively including Comparative Example Compounds X-5 and X-6, the light emitting elements of Examples including the amine compounds of embodiments exhibited relatively high luminous efficiency and relatively long service life characteristics.

In addition, Comparative Example Compound X-7 does not include the ortho-terphenyl group compared with Example Compounds, and Comparative Compound X-8 has a difference in the binding position of the ortho-terphenyl group from Example Compounds. Compared with Comparative Examples 7 and 8 respectively including Comparative Example Compounds X-7 and X-8, the light emitting elements of Examples including the amine compounds of embodiments exhibited relatively high luminous efficiency and relatively long service life characteristics. In particular, Comparative Example 8 exhibits significantly low service life characteristics compared with Examples, and, without being bound by any particular theory, it is believed that the molecular stability of the compound is increased as the ortho-terphenyl group is bonded to the specified position of the present disclosure.

For example, it may be confirmed that Example Compounds have excellent or suitable charge transport properties and charge balance due to the molecular structural characteristics of Example Compounds distinguished from Comparative Examples, and accordingly, the light emitting elements of Examples including the amine compounds of embodiments in the hole transport layer exhibit relatively high efficiency and relatively long service life characteristics.

The amine compound of embodiments includes all of the dibenzoheterole group, the naphthyl group, and the ortho-terphenyl group, and has a structure in which the dibenzoheterole group and the ortho-terphenyl group are bonded to the nitrogen atom at a specific position. Accordingly, the amine compound of embodiments may exhibit excellent or suitable material stability and relatively high charge transport properties. The light emitting element of one or more embodiments including the amine compound of one or more embodiments in the hole transport region may exhibit excellent or suitable luminous efficiency and long service life characteristics. In one or more embodiments, the light emitting element of one or more embodiments which emits blue light includes the amine compound of one or more embodiments in the hole transport region, and thus may exhibit relatively high efficiency and relatively long service life characteristics.

The light emitting element of one or more embodiments may include the amine compound of one or more embodiments, thereby exhibiting relatively high efficiency and relatively long service life characteristics.

The amine compound of one or more embodiments may be utilized to achieve improved characteristics of the light emitting element having relatively high efficiency and relatively long service life.

The display device of one or more embodiments may exhibit excellent or suitable display quality.

In present disclosure, “not include a or any ‘component” “exclude a or any ‘component”, “component’-free”, and/or the like refers to that the “component” not being added, selected, or utilized as a component in a compound/composition, but the “component” of less than a suitable amount may still be included due to other impurities and/or external factors in a composition.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within +30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

In the present disclosure, when particles are spherical, “size” indicates a particle diameter or an average particle diameter, and when the particles are non-spherical, the “size” indicates a major axis length or an average major axis length. The diameter (or size) of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter (or size) is referred to as D50. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

The light-emitting element, the display device, the electronic apparatus, or any other relevant apparatuses/devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the present disclosure has been described with reference to example embodiments of the present disclosure, it will be understood that the present disclosure should not be limited to these embodiments but one or more suitable changes and modifications can be made by those skilled in the art without departing from the spirit and scope of the present disclosure.

Accordingly, the technical scope of the present disclosure is not intended to be limited to the contents set forth in the detailed description of the specification, but is intended to be defined by the appended claims and equivalents thereof.

What is claimed is:

1. A light emitting element, comprising:
a first electrode;
a second electrode on the first electrode;
an emission layer between the first electrode and the second electrode; and
a hole transport region between the first electrode and the emission layer and comprising an amine compound represented by Formula 1: 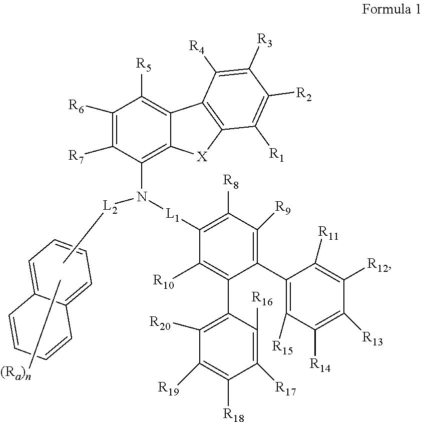
wherein, in Formula 1,
X is O or S,
R₁ to R₂₀ are each independently hydrogen, deuterium, a halogen, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or are independently bonded to an adjacent group to form a ring,
L₁ is a direct linkage, a substituted or unsubstituted arylene group having 6 to 30 ring-forming carbon atoms, or a substituted or unsubstituted heteroarylene group having 2 to 30 ring-forming carbon atoms,
L₂ is a substituted or unsubstituted arylene group having 6 to 30 ring-forming carbon atoms,
n is an integer of 1 to 7, and
Rₐ is hydrogen, deuterium, or a substituted or unsubstituted aryl group having 6 to 30 ring-forming carbon atoms.

2. The light emitting element of claim 1, wherein the hole transport region comprises at least one of a hole injection layer, a hole transport layer, an electron blocking layer, or an emission-auxiliary layer, and
at least one of the hole injection layer, the hole transport layer, the electron blocking layer, or the emission-auxiliary layer comprises the amine compound.

3. The light emitting element of claim 1, wherein the hole transport region comprises a hole injection layer on the first electrode and a hole transport layer on the hole injection layer, and
the hole transport layer comprises the amine compound.

4. The light emitting element of claim 1, wherein the amine compound represented by Formula 1 is represented by Formula 1A or Formula 1B: 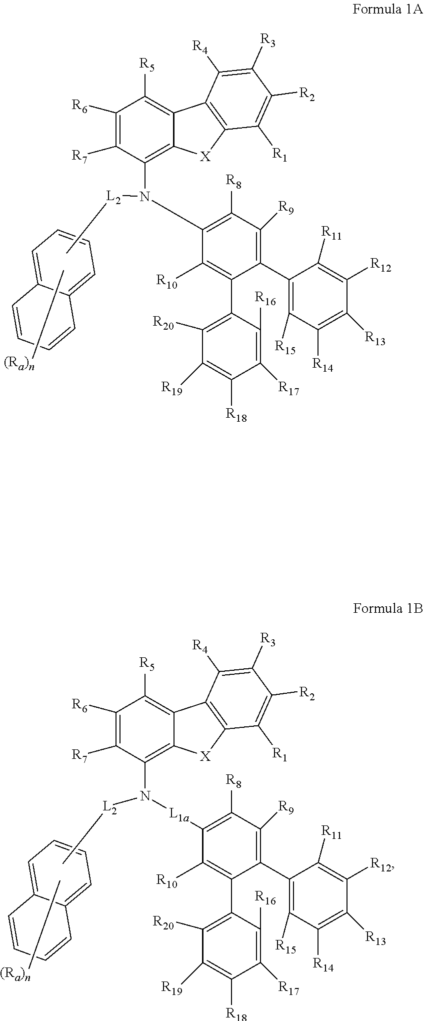
in Formula 1B, L₁ₐ being a substituted or unsubstituted arylene group having 6 to 30 ring-forming carbon atoms or a substituted or unsubstituted heteroarylene group having 2 to 30 ring-forming carbon atoms, and R₈ and R₉ being not bonded to an adjacent group to form a ring, and
in Formula 1A and Formula 1B, X, R₁ to R₂₀, n, Rₐ, and L₂ each being the same as defined in Formula 1.

5. The light emitting element of claim 1, wherein at least one hydrogen of the amine compound is substituted with deuterium.

6. The light emitting element of claim 1, wherein, in Formula 1, each of R₁ to R₂₀, Rₐ, L₁, and L₂ does not include a substituted or unsubstituted amine group.

7. The light emitting element of claim 1, wherein the emission layer comprises a compound represented by Formula E-1: 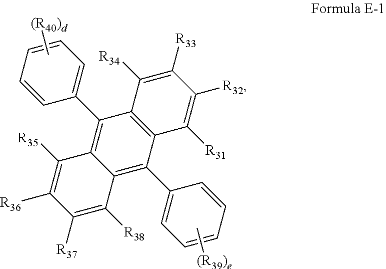
in Formula E-1,
c and d being each independently an integer of 0 to 5, and
R₃₁ to R₄₀ being each independently hydrogen, deuterium, a halogen, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or being bonded to an adjacent group to form a ring.

8. The light emitting element of claim 1, wherein the emission layer is to emit blue light.

9. The light emitting element of claim 1, wherein the amine compound is any one selected from among compounds of Compound Group 1: 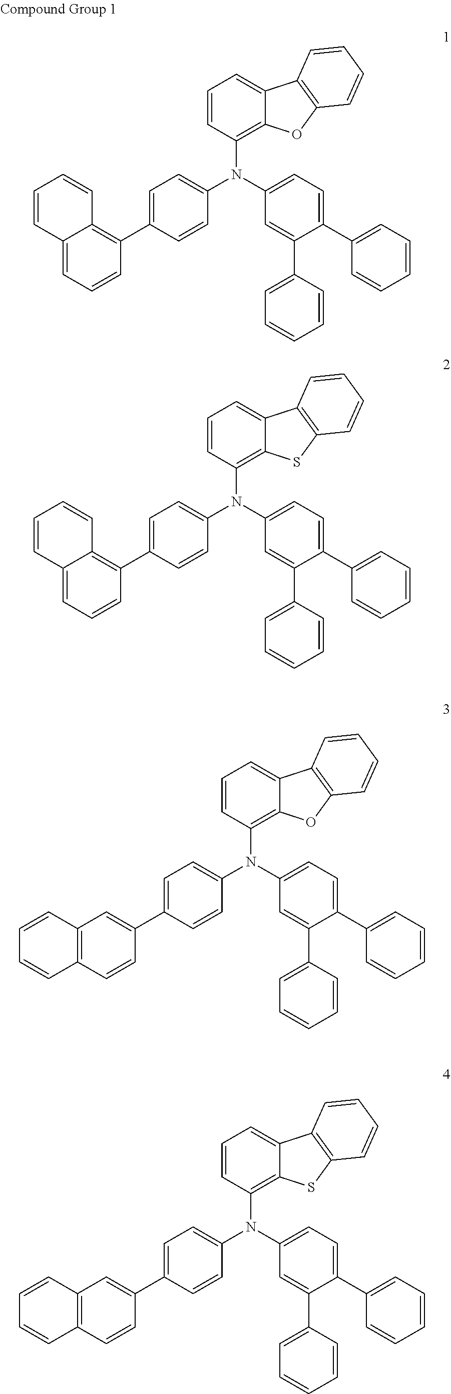 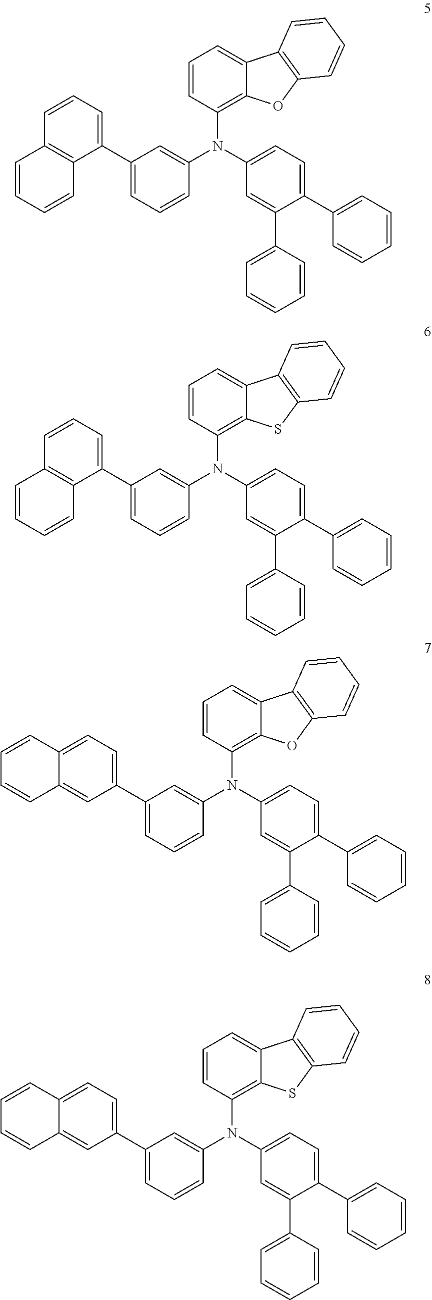 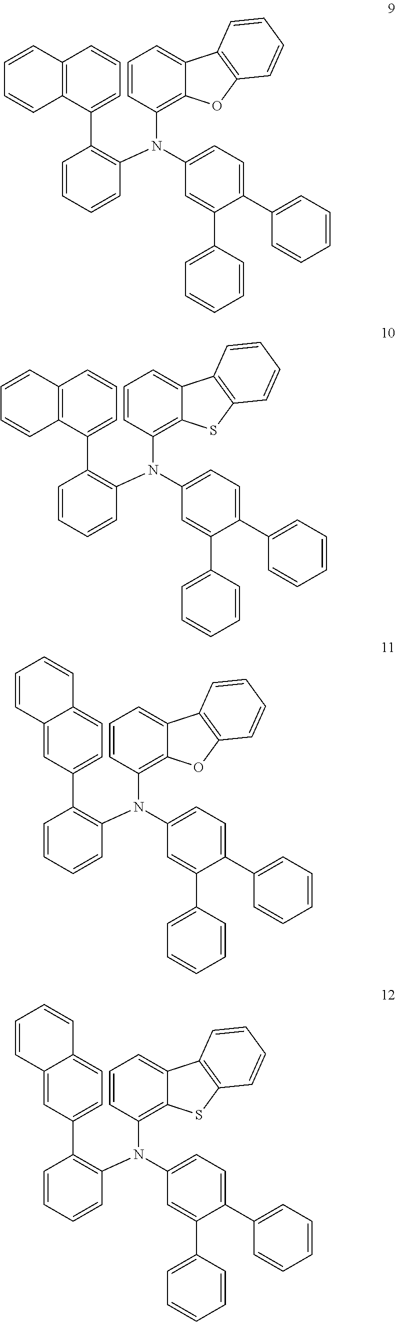 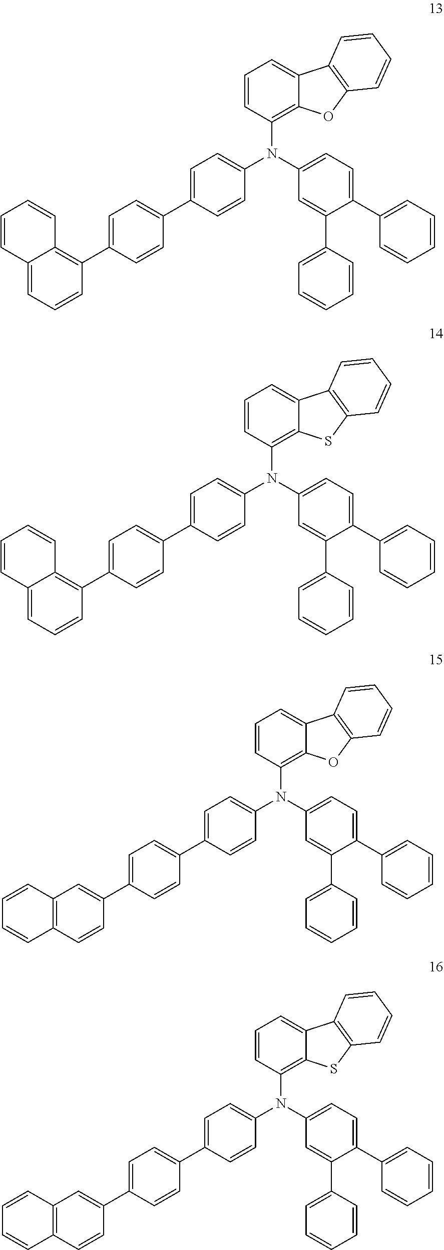 [SMILES: c1ccc(-c2ccc(N(c3ccc(-c4ccc(-c5cccc6ccccc56)cc4)cc3)c3cccc4c3oc3ccccc34)cc2-c2ccccc2)cc1 ; c1ccc(-c2ccc(N(c3ccc(-c4ccc(-c5cccc6ccccc56)cc4)cc3)c3cccc4c3sc3ccccc34)cc2-c2ccccc2)cc1 ; c1ccc(-c2ccc(N(c3ccc(-c4ccc(-c5ccc6ccccc6c5)cc4)cc3)c3cccc4c3oc3ccccc34)cc2-c2ccccc2)cc1 ; c1ccc(-c2ccc(N(c3ccc(-c4ccc(-c5ccc6ccccc6c5)cc4)cc3)c3cccc4c3sc3ccccc34)cc2-c2ccccc2)cc1] 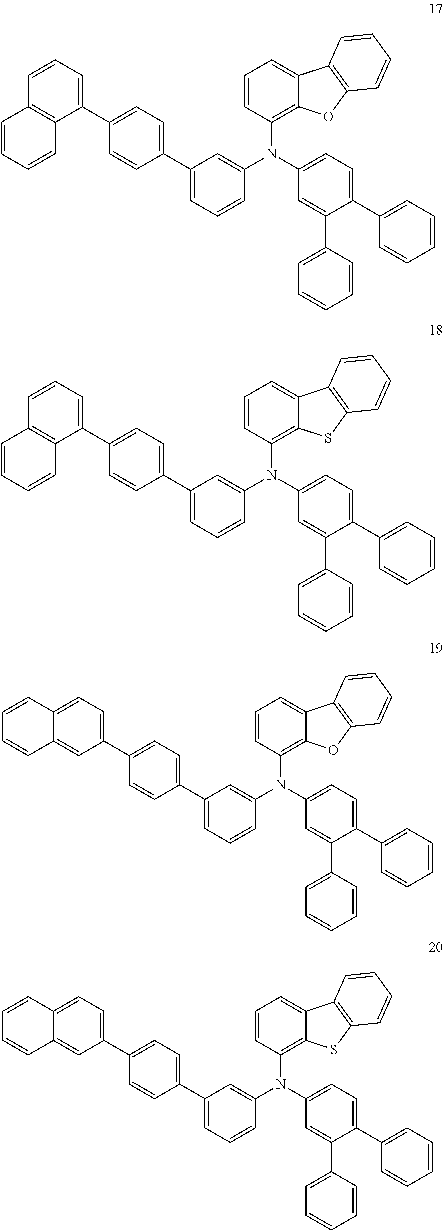 [SMILES: c1ccc(-c2ccc(N(c3cccc(-c4ccc(-c5cccc6ccccc56)cc4)c3)c3cccc4c3oc3ccccc34)cc2-c2ccccc2)cc1 ; c1ccc(-c2ccc(N(c3cccc(-c4ccc(-c5cccc6ccccc56)cc4)c3)c3cccc4c3sc3ccccc34)cc2-c2ccccc2)cc1 ; c1ccc(-c2ccc(N(c3cccc(-c4ccc(-c5ccc6ccccc6c5)cc4)c3)c3cccc4c3oc3ccccc34)cc2-c2ccccc2)cc1 ; c1ccc(-c2ccc(N(c3cccc(-c4ccc(-c5ccc6ccccc6c5)cc4)c3)c3cccc4c3sc3ccccc34)cc2-c2ccccc2)cc1] 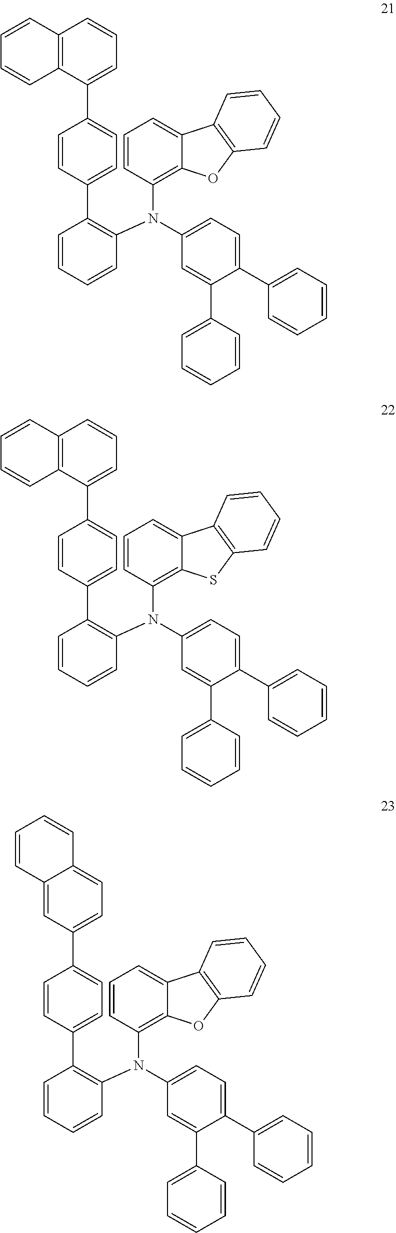 [SMILES: c1ccc(-c2ccc(N(c3ccccc3-c3ccc(-c4cccc5ccccc45)cc3)c3cccc4c3oc3ccccc34)cc2-c2ccccc2)cc1 ; c1ccc(-c2ccc(N(c3ccccc3-c3ccc(-c4cccc5ccccc45)cc3)c3cccc4c3sc3ccccc34)cc2-c2ccccc2)cc1 ; c1ccc(-c2ccc(N(c3ccccc3-c3ccc(-c4ccc5ccccc5c4)cc3)c3cccc4c3oc3ccccc34)cc2-c2ccccc2)cc1] 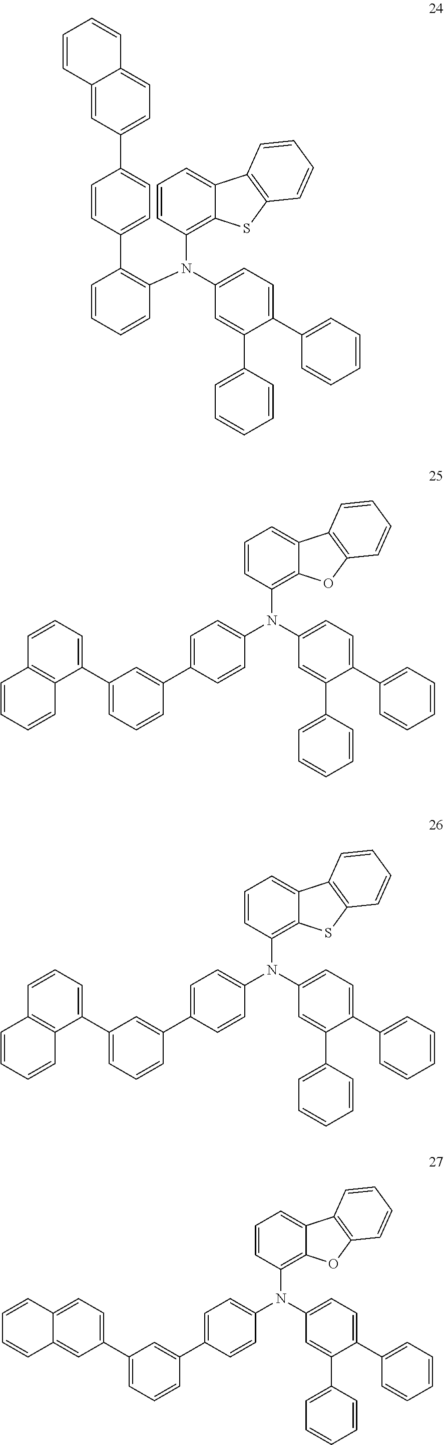 [SMILES: c1ccc(-c2ccc(N(c3ccccc3-c3ccc(-c4ccc5ccccc5c4)cc3)c3cccc4c3sc3ccccc34)cc2-c2ccccc2)cc1 ; c1ccc(-c2ccc(N(c3ccc(-c4cccc(-c5cccc6ccccc56)c4)cc3)c3cccc4c3oc3ccccc34)cc2-c2ccccc2)cc1 ; c1ccc(-c2ccc(N(c3ccc(-c4cccc(-c5cccc6ccccc56)c4)cc3)c3cccc4c3sc3ccccc34)cc2-c2ccccc2)cc1 ; c1ccc(-c2ccc(N(c3ccc(-c4cccc(-c5ccc6ccccc6c5)c4)cc3)c3cccc4c3oc3ccccc34)cc2-c2ccccc2)cc1] 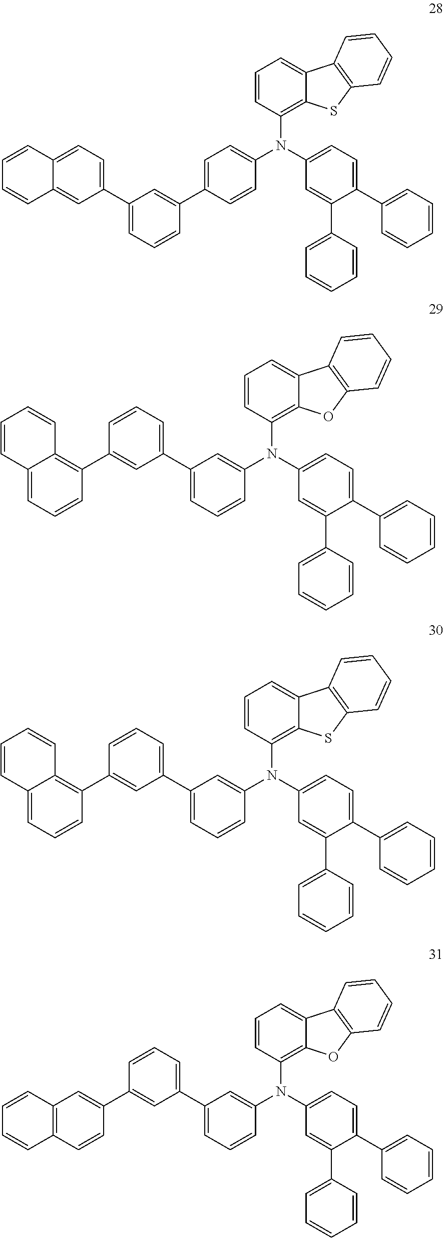 [SMILES: c1ccc(-c2ccc(N(c3ccc(-c4cccc(-c5ccc6ccccc6c5)c4)cc3)c3cccc4c3sc3ccccc34)cc2-c2ccccc2)cc1 ; c1ccc(-c2ccc(N(c3cccc(-c4cccc(-c5cccc6ccccc56)c4)c3)c3cccc4c3oc3ccccc34)cc2-c2ccccc2)cc1 ; c1ccc(-c2ccc(N(c3cccc(-c4cccc(-c5cccc6ccccc56)c4)c3)c3cccc4c3sc3ccccc34)cc2-c2ccccc2)cc1 ; c1ccc(-c2ccc(N(c3cccc(-c4cccc(-c5ccc6ccccc6c5)c4)c3)c3cccc4c3oc3ccccc34)cc2-c2ccccc2)cc1] 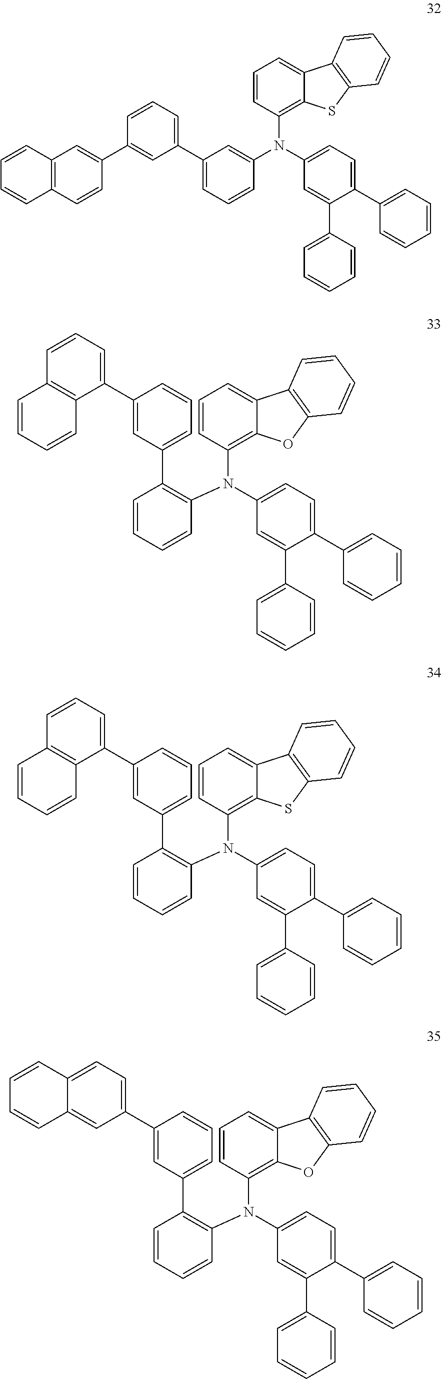 [SMILES: c1ccc(-c2ccc(N(c3cccc(-c4cccc(-c5ccc6ccccc6c5)c4)c3)c3cccc4c3sc3ccccc34)cc2-c2ccccc2)cc1 ; c1ccc(-c2ccc(N(c3ccccc3-c3cccc(-c4cccc5ccccc45)c3)c3cccc4c3oc3ccccc34)cc2-c2ccccc2)cc1 ; c1ccc(-c2ccc(N(c3ccccc3-c3cccc(-c4cccc5ccccc45)c3)c3cccc4c3sc3ccccc34)cc2-c2ccccc2)cc1 ; c1ccc(-c2ccc(N(c3ccccc3-c3cccc(-c4ccc5ccccc5c4)c3)c3cccc4c3oc3ccccc34)cc2-c2ccccc2)cc1] 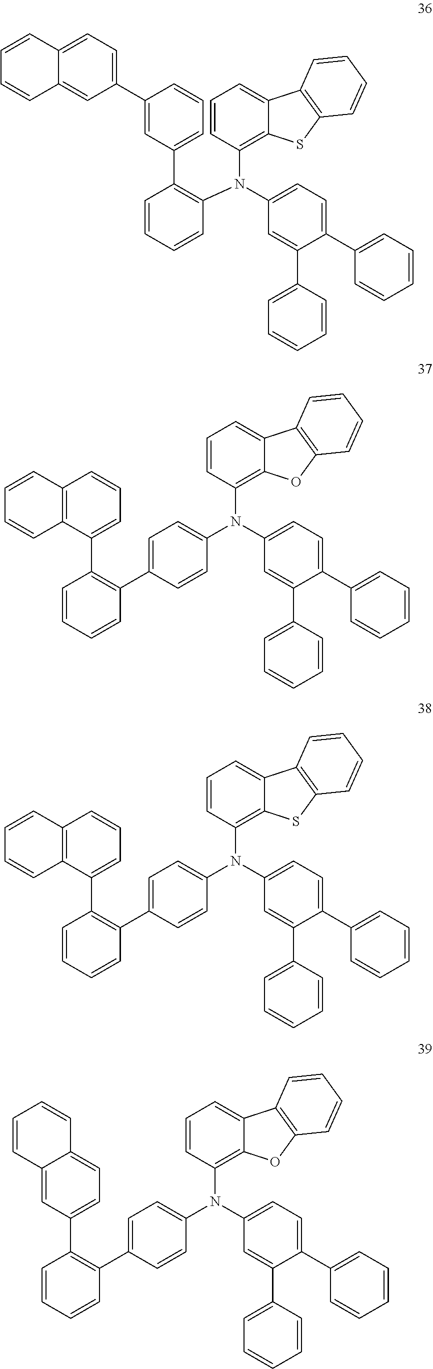 [SMILES: c1ccc(-c2ccc(N(c3ccccc3-c3cccc(-c4ccc5ccccc5c4)c3)c3cccc4c3sc3ccccc34)cc2-c2ccccc2)cc1 ; c1ccc(-c2ccc(N(c3ccc(-c4ccccc4-c4cccc5ccccc45)cc3)c3cccc4c3oc3ccccc34)cc2-c2ccccc2)cc1 ; c1ccc(-c2ccc(N(c3ccc(-c4ccccc4-c4cccc5ccccc45)cc3)c3cccc4c3sc3ccccc34)cc2-c2ccccc2)cc1 ; c1ccc(-c2ccc(N(c3ccc(-c4ccccc4-c4ccc5ccccc5c4)cc3)c3cccc4c3oc3ccccc34)cc2-c2ccccc2)cc1] 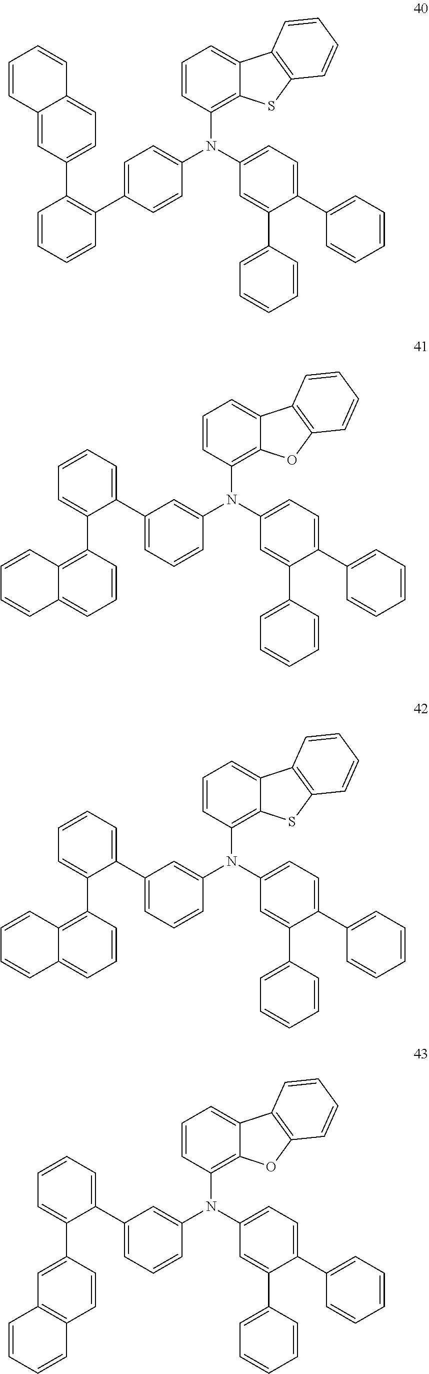 [SMILES: c1ccc(-c2ccc(N(c3ccc(-c4ccccc4-c4ccc5ccccc5c4)cc3)c3cccc4c3sc3ccccc34)cc2-c2ccccc2)cc1 ; c1ccc(-c2ccc(N(c3cccc(-c4ccccc4-c4cccc5ccccc45)c3)c3cccc4c3oc3ccccc34)cc2-c2ccccc2)cc1 ; c1ccc(-c2ccc(N(c3cccc(-c4ccccc4-c4cccc5ccccc45)c3)c3cccc4c3sc3ccccc34)cc2-c2ccccc2)cc1 ; c1ccc(-c2ccc(N(c3cccc(-c4ccccc4-c4ccc5ccccc5c4)c3)c3cccc4c3oc3ccccc34)cc2-c2ccccc2)cc1] 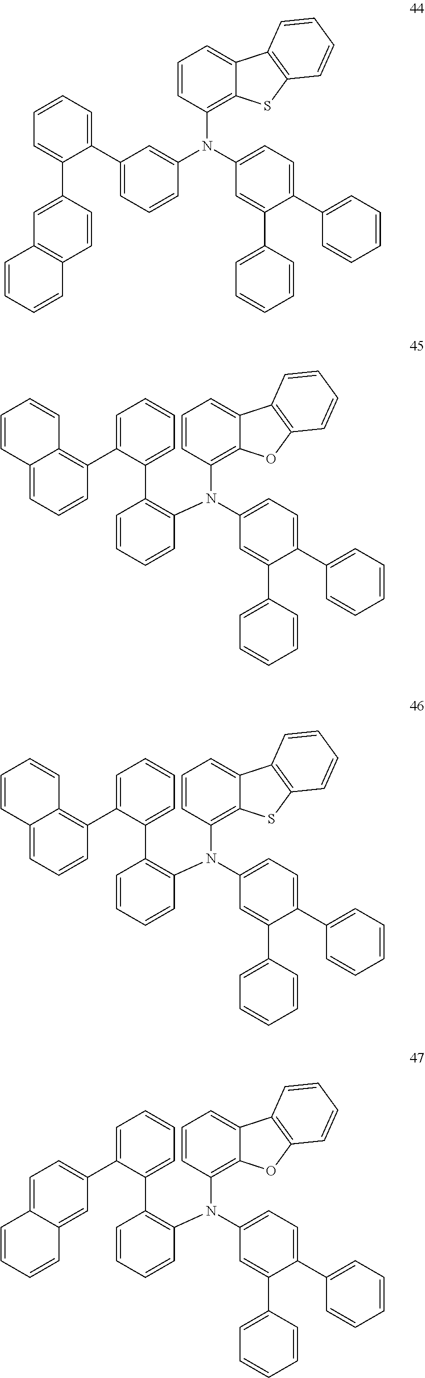 [SMILES: c1ccc(-c2ccc(N(c3cccc(-c4ccccc4-c4ccc5ccccc5c4)c3)c3cccc4c3sc3ccccc34)cc2-c2ccccc2)cc1 ; c1ccc(-c2ccc(N(c3ccccc3-c3ccccc3-c3cccc4ccccc34)c3cccc4c3oc3ccccc34)cc2-c2ccccc2)cc1 ; c1ccc(-c2ccc(N(c3ccccc3-c3ccccc3-c3cccc4ccccc34)c3cccc4c3sc3ccccc34)cc2-c2ccccc2)cc1 ; c1ccc(-c2ccc(N(c3ccccc3-c3ccccc3-c3ccc4ccccc4c3)c3cccc4c3oc3ccccc34)cc2-c2ccccc2)cc1] 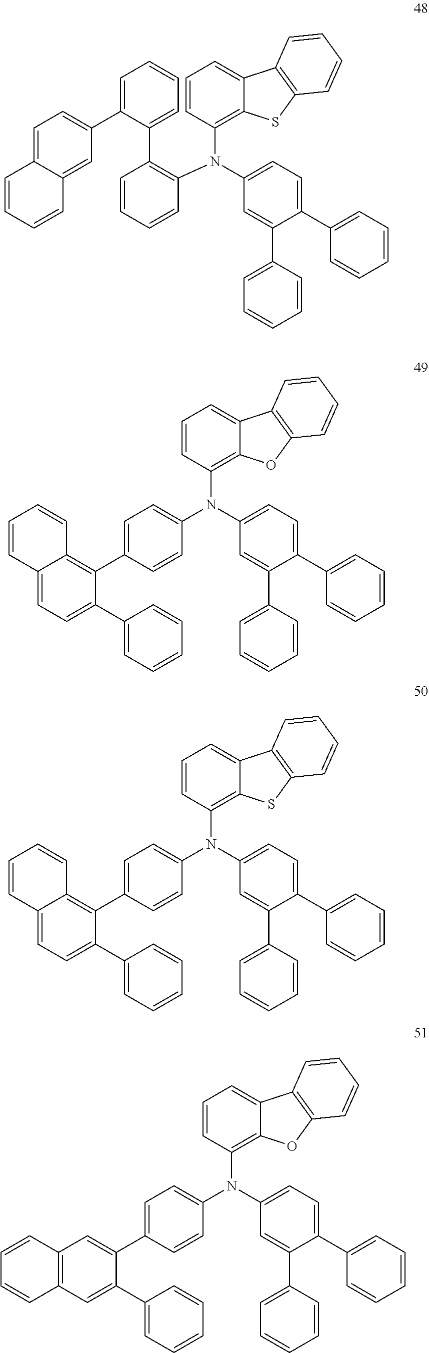 [SMILES: c1ccc(-c2ccc(N(c3ccccc3-c3ccccc3-c3ccc4ccccc4c3)c3cccc4c3sc3ccccc34)cc2-c2ccccc2)cc1 ; c1ccc(-c2ccc(N(c3ccc(-c4c(-c5ccccc5)ccc5ccccc45)cc3)c3cccc4c3oc3ccccc34)cc2-c2ccccc2)cc1 ; c1ccc(-c2ccc(N(c3ccc(-c4c(-c5ccccc5)ccc5ccccc45)cc3)c3cccc4c3sc3ccccc34)cc2-c2ccccc2)cc1 ; c1ccc(-c2ccc(N(c3ccc(-c4cc5ccccc5cc4-c4ccccc4)cc3)c3cccc4c3oc3ccccc34)cc2-c2ccccc2)cc1] 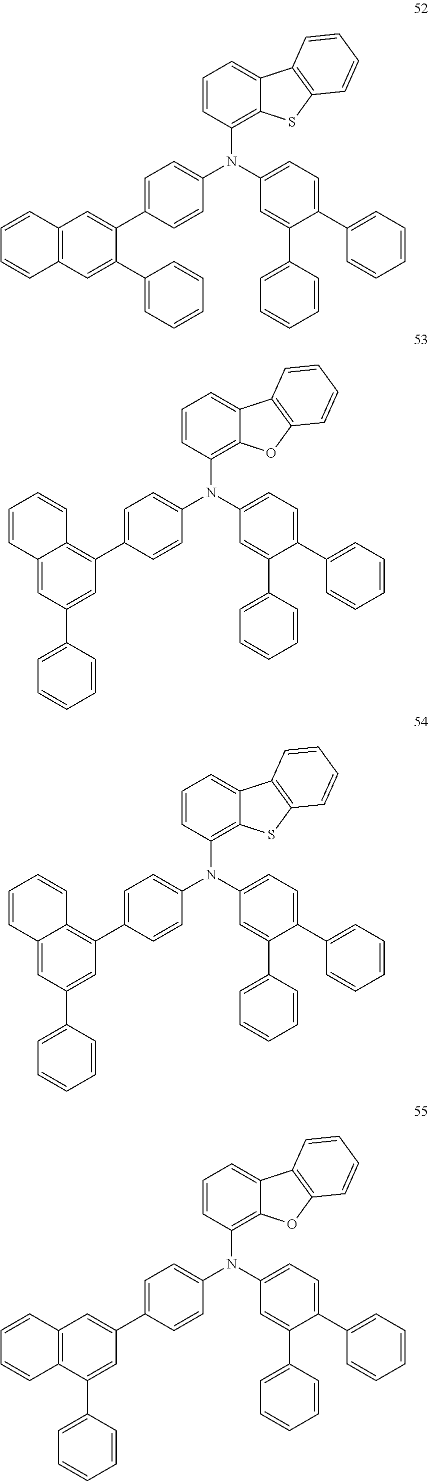 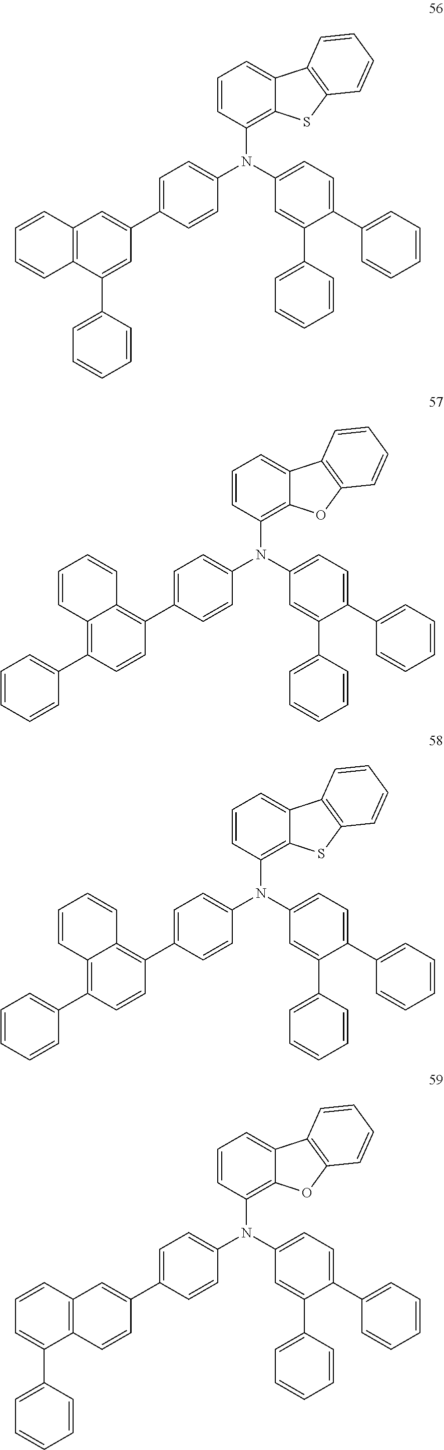 [SMILES: c1ccc(-c2ccc(N(c3ccc(-c4cc(-c5ccccc5)c5ccccc5c4)cc3)c3cccc4c3sc3ccccc34)cc2-c2ccccc2)cc1 ; c1ccc(-c2ccc(N(c3ccc(-c4ccc(-c5ccccc5)c5ccccc45)cc3)c3cccc4c3oc3ccccc34)cc2-c2ccccc2)cc1 ; c1ccc(-c2ccc(N(c3ccc(-c4ccc(-c5ccccc5)c5ccccc45)cc3)c3cccc4c3sc3ccccc34)cc2-c2ccccc2)cc1 ; c1ccc(-c2ccc(N(c3ccc(-c4ccc5c(-c6ccccc6)cccc5c4)cc3)c3cccc4c3oc3ccccc34)cc2-c2ccccc2)cc1] 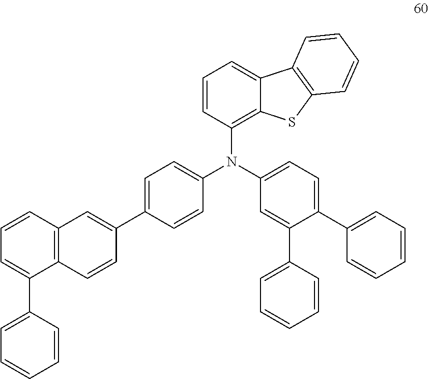 [SMILES: c1ccc(-c2ccc(N(c3ccc(-c4ccc5c(-c6ccccc6)cccc5c4)cc3)c3cccc4c3sc3ccccc34)cc2-c2ccccc2)cc1] 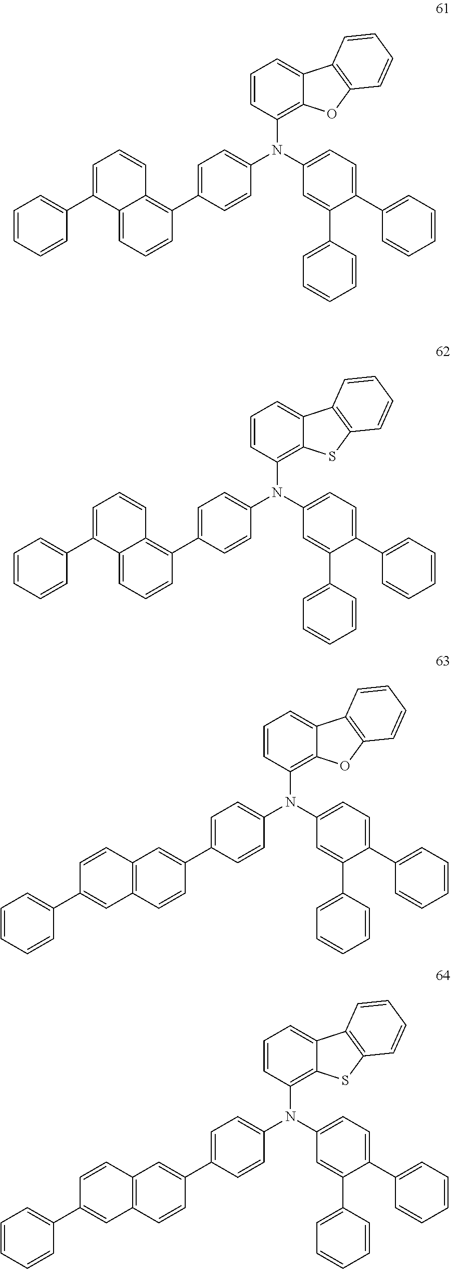 [SMILES: c1ccc(-c2ccc(N(c3ccc(-c4cccc5c(-c6ccccc6)cccc45)cc3)c3cccc4c3oc3ccccc34)cc2-c2ccccc2)cc1 ; c1ccc(-c2ccc(N(c3ccc(-c4cccc5c(-c6ccccc6)cccc45)cc3)c3cccc4c3sc3ccccc34)cc2-c2ccccc2)cc1 ; c1ccc(-c2ccc3cc(-c4ccc(N(c5ccc(-c6ccccc6)c(-c6ccccc6)c5)c5cccc6c5oc5ccccc56)cc4)ccc3c2)cc1 ; c1ccc(-c2ccc3cc(-c4ccc(N(c5ccc(-c6ccccc6)c(-c6ccccc6)c5)c5cccc6c5sc5ccccc56)cc4)ccc3c2)cc1] 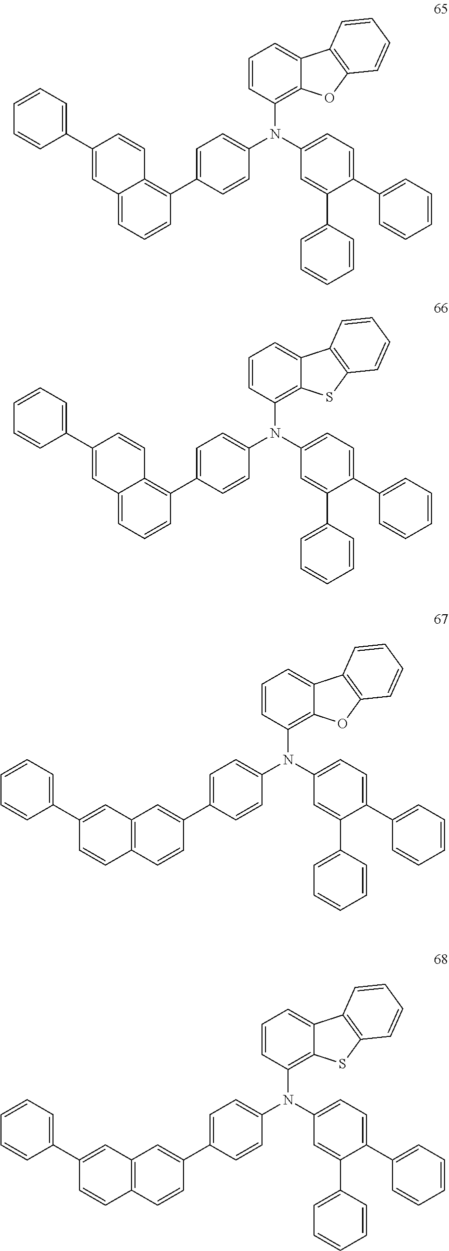 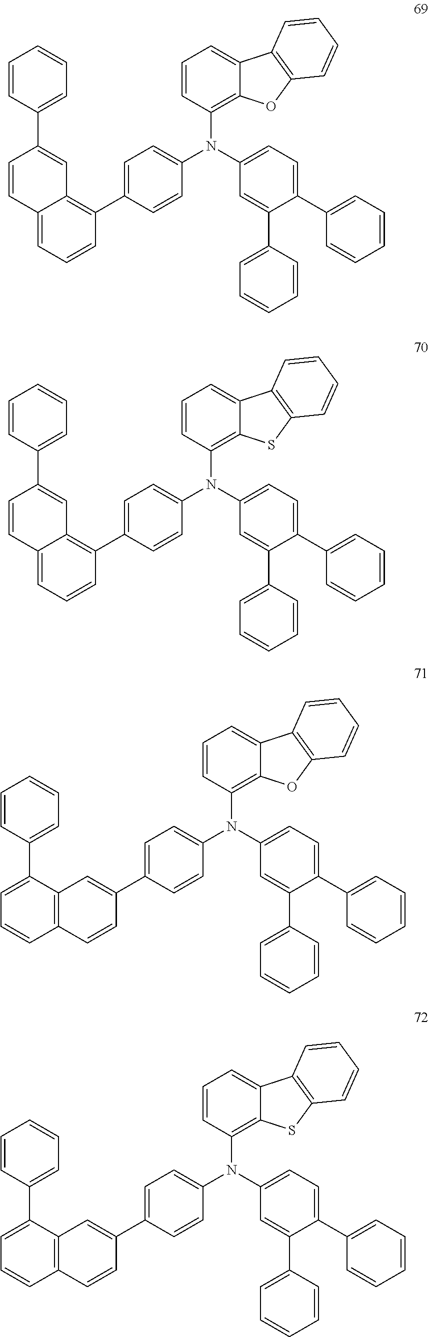 [SMILES: c1ccc(-c2ccc3cccc(-c4ccc(N(c5ccc(-c6ccccc6)c(-c6ccccc6)c5)c5cccc6c5oc5ccccc56)cc4)c3c2)cc1 ; c1ccc(-c2ccc3cccc(-c4ccc(N(c5ccc(-c6ccccc6)c(-c6ccccc6)c5)c5cccc6c5sc5ccccc56)cc4)c3c2)cc1 ; c1ccc(-c2ccc(N(c3ccc(-c4ccc5cccc(-c6ccccc6)c5c4)cc3)c3cccc4c3oc3ccccc34)cc2-c2ccccc2)cc1 ; c1ccc(-c2ccc(N(c3ccc(-c4ccc5cccc(-c6ccccc6)c5c4)cc3)c3cccc4c3sc3ccccc34)cc2-c2ccccc2)cc1] 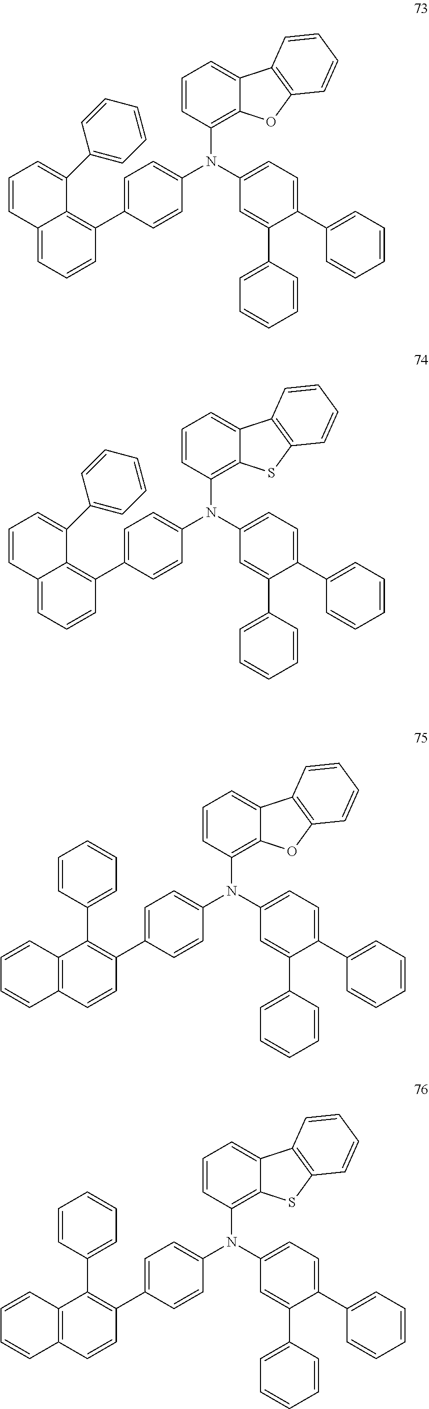 [SMILES: c1ccc(-c2ccc(N(c3ccc(-c4cccc5cccc(-c6ccccc6)c45)cc3)c3cccc4c3oc3ccccc34)cc2-c2ccccc2)cc1 ; c1ccc(-c2ccc(N(c3ccc(-c4cccc5cccc(-c6ccccc6)c45)cc3)c3cccc4c3sc3ccccc34)cc2-c2ccccc2)cc1 ; c1ccc(-c2ccc(N(c3ccc(-c4ccc5ccccc5c4-c4ccccc4)cc3)c3cccc4c3oc3ccccc34)cc2-c2ccccc2)cc1 ; c1ccc(-c2ccc(N(c3ccc(-c4ccc5ccccc5c4-c4ccccc4)cc3)c3cccc4c3sc3ccccc34)cc2-c2ccccc2)cc1] 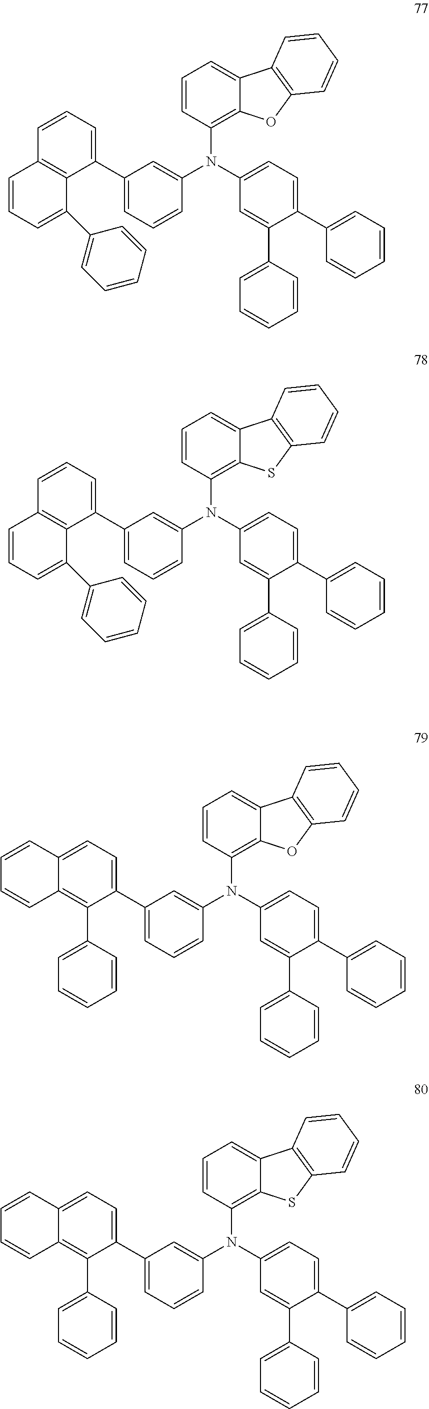 [SMILES: c1ccc(-c2ccc(N(c3cccc(-c4cccc5cccc(-c6ccccc6)c45)c3)c3cccc4c3oc3ccccc34)cc2-c2ccccc2)cc1 ; c1ccc(-c2ccc(N(c3cccc(-c4cccc5cccc(-c6ccccc6)c45)c3)c3cccc4c3sc3ccccc34)cc2-c2ccccc2)cc1 ; c1ccc(-c2ccc(N(c3cccc(-c4ccc5ccccc5c4-c4ccccc4)c3)c3cccc4c3oc3ccccc34)cc2-c2ccccc2)cc1 ; c1ccc(-c2ccc(N(c3cccc(-c4ccc5ccccc5c4-c4ccccc4)c3)c3cccc4c3sc3ccccc34)cc2-c2ccccc2)cc1] 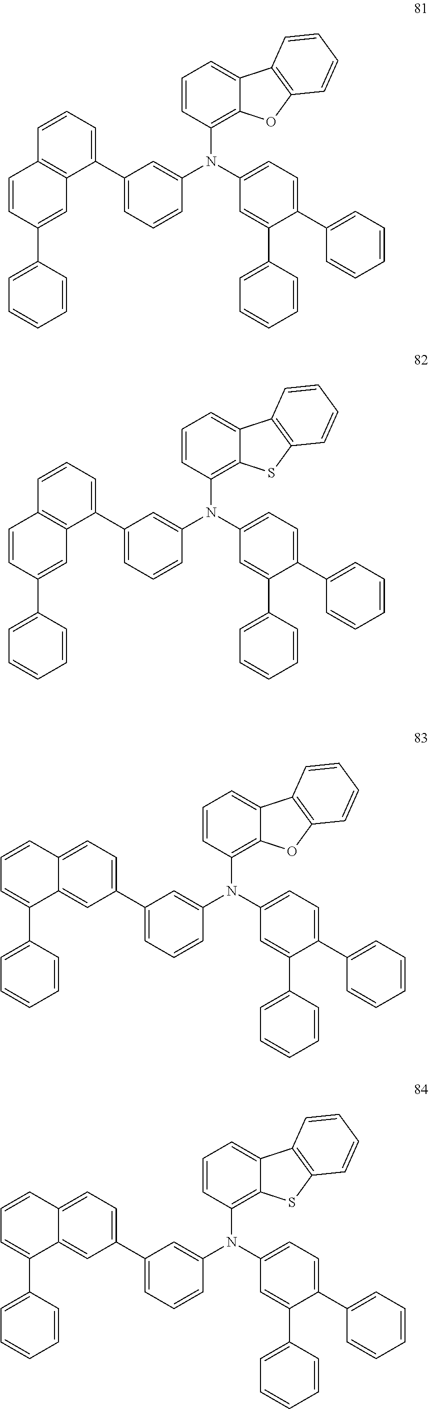 [SMILES: c1ccc(-c2ccc3cccc(-c4cccc(N(c5ccc(-c6ccccc6)c(-c6ccccc6)c5)c5cccc6c5oc5ccccc56)c4)c3c2)cc1 ; c1ccc(-c2ccc3cccc(-c4cccc(N(c5ccc(-c6ccccc6)c(-c6ccccc6)c5)c5cccc6c5sc5ccccc56)c4)c3c2)cc1 ; c1ccc(-c2ccc(N(c3cccc(-c4ccc5cccc(-c6ccccc6)c5c4)c3)c3cccc4c3oc3ccccc34)cc2-c2ccccc2)cc1 ; c1ccc(-c2ccc(N(c3cccc(-c4ccc5cccc(-c6ccccc6)c5c4)c3)c3cccc4c3sc3ccccc34)cc2-c2ccccc2)cc1] 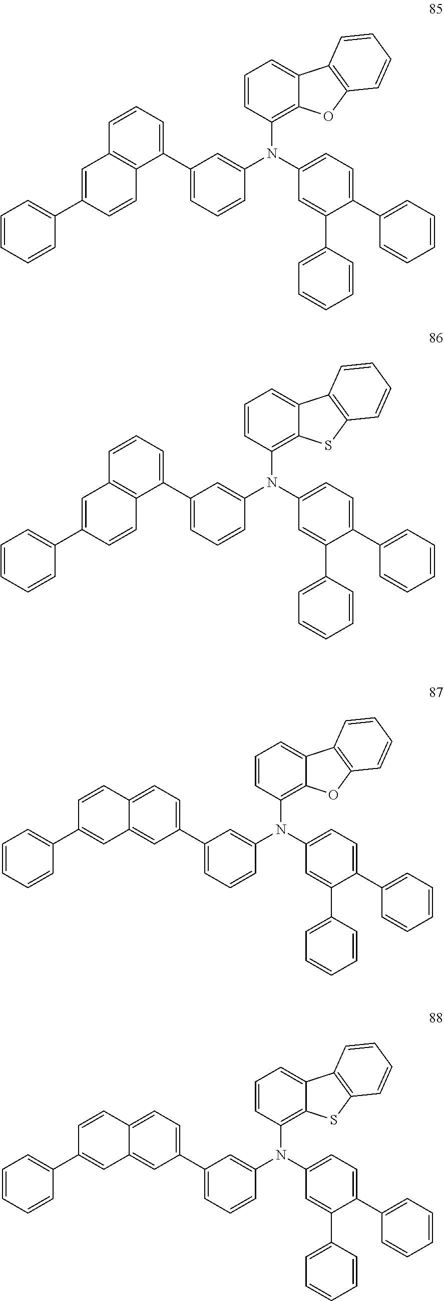 [SMILES: c1ccc(-c2ccc3c(-c4cccc(N(c5ccc(-c6ccccc6)c(-c6ccccc6)c5)c5cccc6c5oc5ccccc56)c4)cccc3c2)cc1 ; c1ccc(-c2ccc3c(-c4cccc(N(c5ccc(-c6ccccc6)c(-c6ccccc6)c5)c5cccc6c5sc5ccccc56)c4)cccc3c2)cc1 ; c1ccc(-c2ccc3ccc(-c4cccc(N(c5ccc(-c6ccccc6)c(-c6ccccc6)c5)c5cccc6c5oc5ccccc56)c4)cc3c2)cc1 ; c1ccc(-c2ccc3ccc(-c4cccc(N(c5ccc(-c6ccccc6)c(-c6ccccc6)c5)c5cccc6c5sc5ccccc56)c4)cc3c2)cc1] 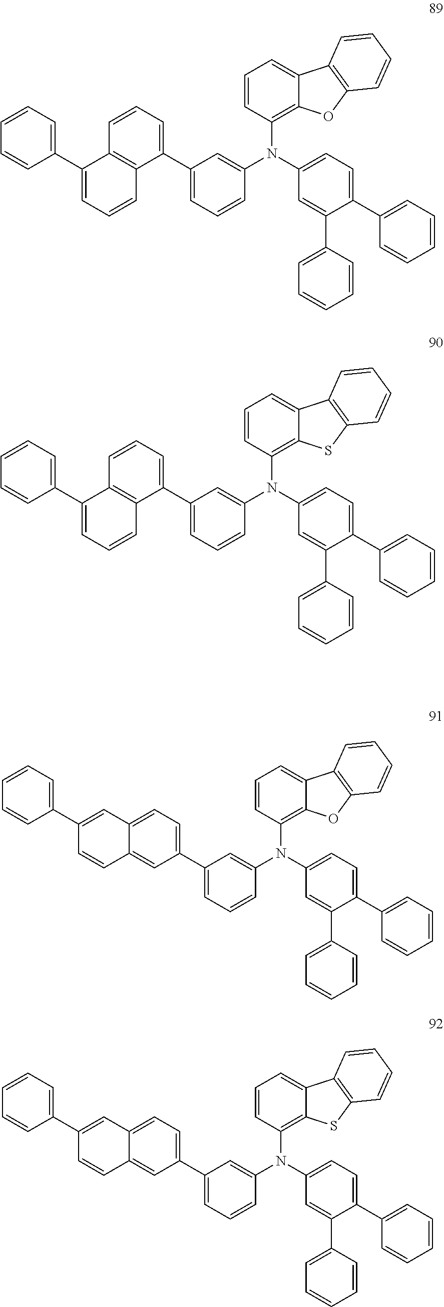 [SMILES: c1ccc(-c2ccc(N(c3cccc(-c4cccc5c(-c6ccccc6)cccc45)c3)c3cccc4c3oc3ccccc34)cc2-c2ccccc2)cc1 ; c1ccc(-c2ccc(N(c3cccc(-c4cccc5c(-c6ccccc6)cccc45)c3)c3cccc4c3sc3ccccc34)cc2-c2ccccc2)cc1 ; c1ccc(-c2ccc3cc(-c4cccc(N(c5ccc(-c6ccccc6)c(-c6ccccc6)c5)c5cccc6c5oc5ccccc56)c4)ccc3c2)cc1 ; c1ccc(-c2ccc3cc(-c4cccc(N(c5ccc(-c6ccccc6)c(-c6ccccc6)c5)c5cccc6c5sc5ccccc56)c4)ccc3c2)cc1] 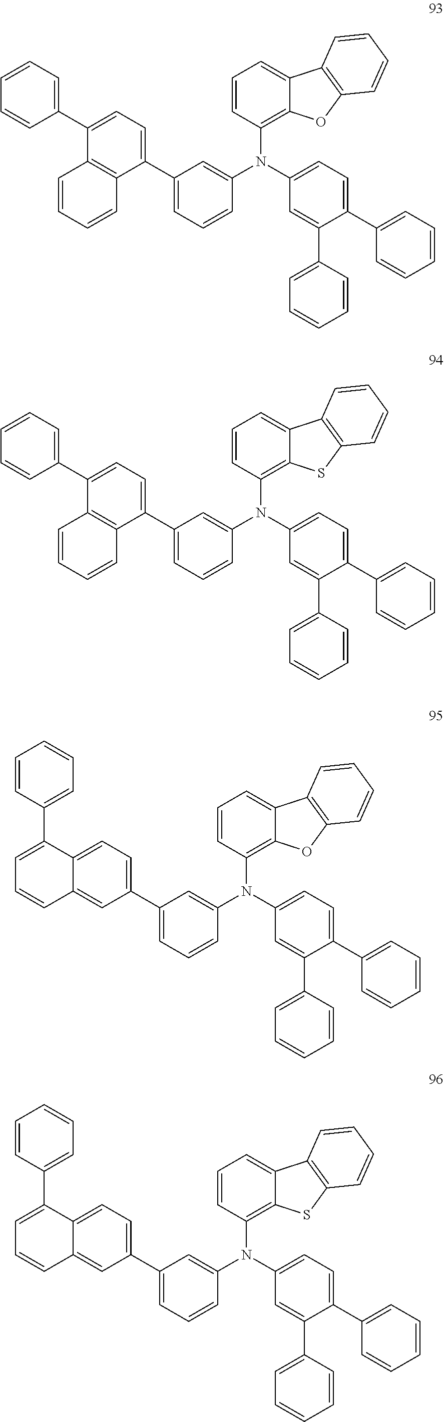 [SMILES: c1ccc(-c2ccc(N(c3cccc(-c4ccc(-c5ccccc5)c5ccccc45)c3)c3cccc4c3oc3ccccc34)cc2-c2ccccc2)cc1 ; c1ccc(-c2ccc(N(c3cccc(-c4ccc(-c5ccccc5)c5ccccc45)c3)c3cccc4c3sc3ccccc34)cc2-c2ccccc2)cc1 ; c1ccc(-c2ccc(N(c3cccc(-c4ccc5c(-c6ccccc6)cccc5c4)c3)c3cccc4c3oc3ccccc34)cc2-c2ccccc2)cc1 ; c1ccc(-c2ccc(N(c3cccc(-c4ccc5c(-c6ccccc6)cccc5c4)c3)c3cccc4c3sc3ccccc34)cc2-c2ccccc2)cc1] 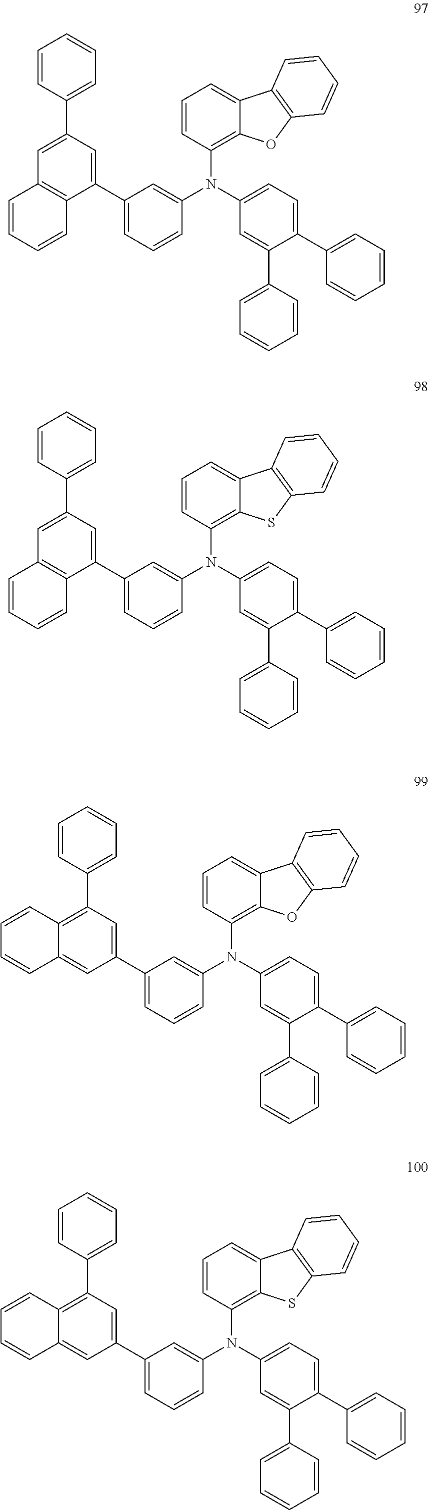 [SMILES: c1ccc(-c2cc(-c3cccc(N(c4ccc(-c5ccccc5)c(-c5ccccc5)c4)c4cccc5c4oc4ccccc45)c3)c3ccccc3c2)cc1 ; c1ccc(-c2cc(-c3cccc(N(c4ccc(-c5ccccc5)c(-c5ccccc5)c4)c4cccc5c4sc4ccccc45)c3)c3ccccc3c2)cc1 ; c1ccc(-c2ccc(N(c3cccc(-c4cc(-c5ccccc5)c5ccccc5c4)c3)c3cccc4c3oc3ccccc34)cc2-c2ccccc2)cc1 ; c1ccc(-c2ccc(N(c3cccc(-c4cc(-c5ccccc5)c5ccccc5c4)c3)c3cccc4c3sc3ccccc34)cc2-c2ccccc2)cc1] 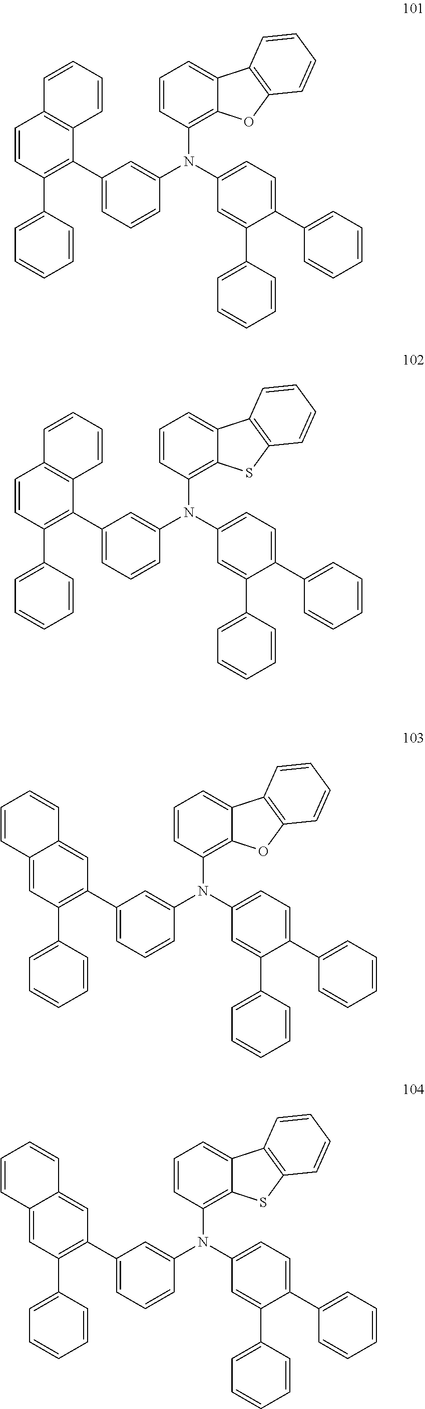 [SMILES: c1ccc(-c2ccc(N(c3cccc(-c4c(-c5ccccc5)ccc5ccccc45)c3)c3cccc4c3oc3ccccc34)cc2-c2ccccc2)cc1 ; c1ccc(-c2ccc(N(c3cccc(-c4c(-c5ccccc5)ccc5ccccc45)c3)c3cccc4c3sc3ccccc34)cc2-c2ccccc2)cc1 ; c1ccc(-c2ccc(N(c3cccc(-c4cc5ccccc5cc4-c4ccccc4)c3)c3cccc4c3oc3ccccc34)cc2-c2ccccc2)cc1 ; c1ccc(-c2ccc(N(c3cccc(-c4cc5ccccc5cc4-c4ccccc4)c3)c3cccc4c3sc3ccccc34)cc2-c2ccccc2)cc1] 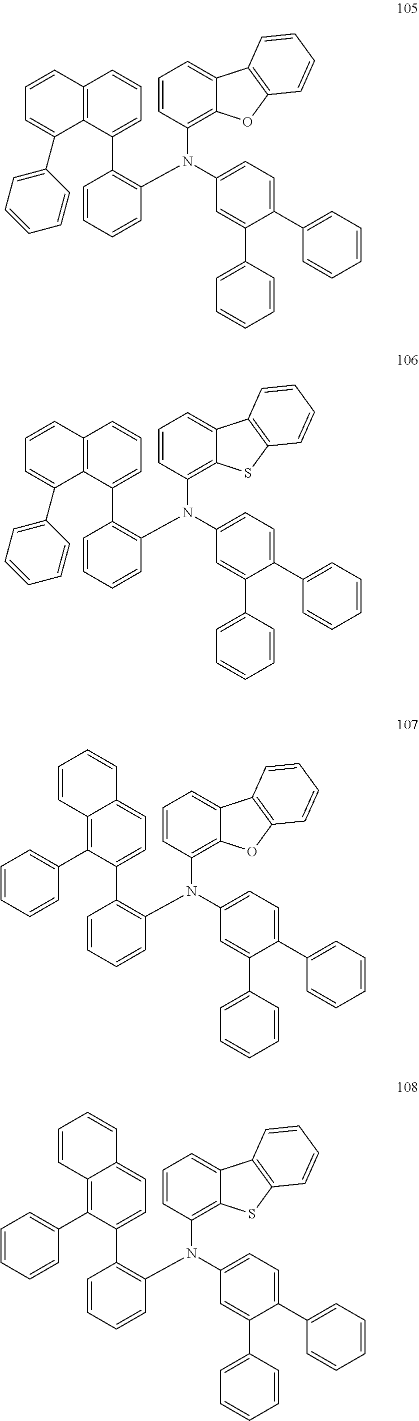 [SMILES: c1ccc(-c2ccc(N(c3ccccc3-c3cccc4cccc(-c5ccccc5)c34)c3cccc4c3oc3ccccc34)cc2-c2ccccc2)cc1 ; c1ccc(-c2ccc(N(c3ccccc3-c3cccc4cccc(-c5ccccc5)c34)c3cccc4c3sc3ccccc34)cc2-c2ccccc2)cc1 ; c1ccc(-c2ccc(N(c3ccccc3-c3ccc4ccccc4c3-c3ccccc3)c3cccc4c3oc3ccccc34)cc2-c2ccccc2)cc1 ; c1ccc(-c2ccc(N(c3ccccc3-c3ccc4ccccc4c3-c3ccccc3)c3cccc4c3sc3ccccc34)cc2-c2ccccc2)cc1] 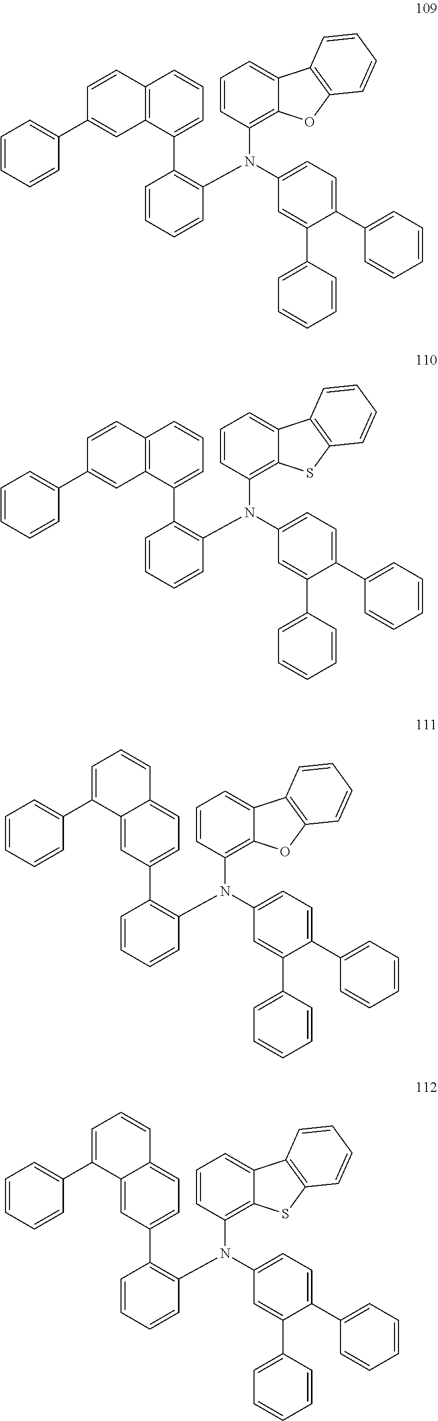 [SMILES: c1ccc(-c2ccc3cccc(-c4ccccc4N(c4ccc(-c5ccccc5)c(-c5ccccc5)c4)c4cccc5c4oc4ccccc45)c3c2)cc1 ; c1ccc(-c2ccc3cccc(-c4ccccc4N(c4ccc(-c5ccccc5)c(-c5ccccc5)c4)c4cccc5c4sc4ccccc45)c3c2)cc1 ; c1ccc(-c2ccc(N(c3ccccc3-c3ccc4cccc(-c5ccccc5)c4c3)c3cccc4c3oc3ccccc34)cc2-c2ccccc2)cc1 ; c1ccc(-c2ccc(N(c3ccccc3-c3ccc4cccc(-c5ccccc5)c4c3)c3cccc4c3sc3ccccc34)cc2-c2ccccc2)cc1] 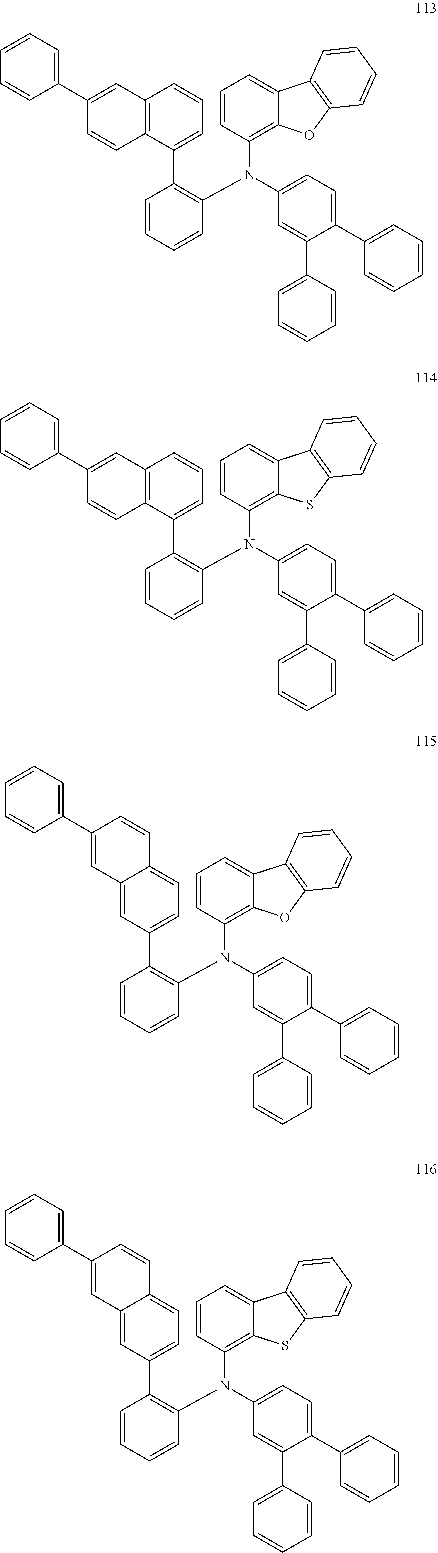 [SMILES: c1ccc(-c2ccc3c(-c4ccccc4N(c4ccc(-c5ccccc5)c(-c5ccccc5)c4)c4cccc5c4oc4ccccc45)cccc3c2)cc1 ; c1ccc(-c2ccc3c(-c4ccccc4N(c4ccc(-c5ccccc5)c(-c5ccccc5)c4)c4cccc5c4sc4ccccc45)cccc3c2)cc1 ; c1ccc(-c2ccc3ccc(-c4ccccc4N(c4ccc(-c5ccccc5)c(-c5ccccc5)c4)c4cccc5c4oc4ccccc45)cc3c2)cc1 ; c1ccc(-c2ccc3ccc(-c4ccccc4N(c4ccc(-c5ccccc5)c(-c5ccccc5)c4)c4cccc5c4sc4ccccc45)cc3c2)cc1] 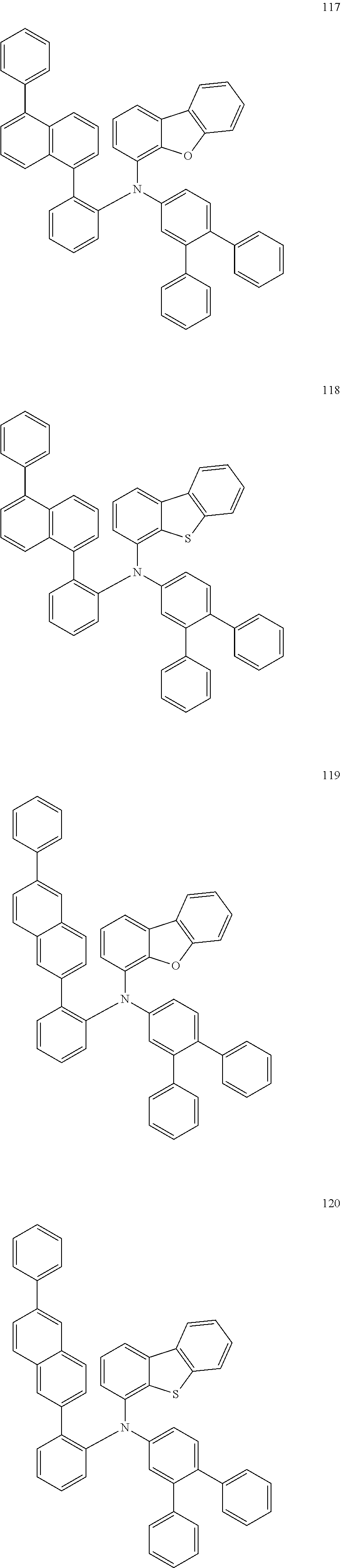 [SMILES: c1ccc(-c2ccc(N(c3ccccc3-c3cccc4c(-c5ccccc5)cccc34)c3cccc4c3oc3ccccc34)cc2-c2ccccc2)cc1 ; c1ccc(-c2ccc(N(c3ccccc3-c3cccc4c(-c5ccccc5)cccc34)c3cccc4c3sc3ccccc34)cc2-c2ccccc2)cc1 ; c1ccc(-c2ccc3cc(-c4ccccc4N(c4ccc(-c5ccccc5)c(-c5ccccc5)c4)c4cccc5c4oc4ccccc45)ccc3c2)cc1 ; c1ccc(-c2ccc3cc(-c4ccccc4N(c4ccc(-c5ccccc5)c(-c5ccccc5)c4)c4cccc5c4sc4ccccc45)ccc3c2)cc1] 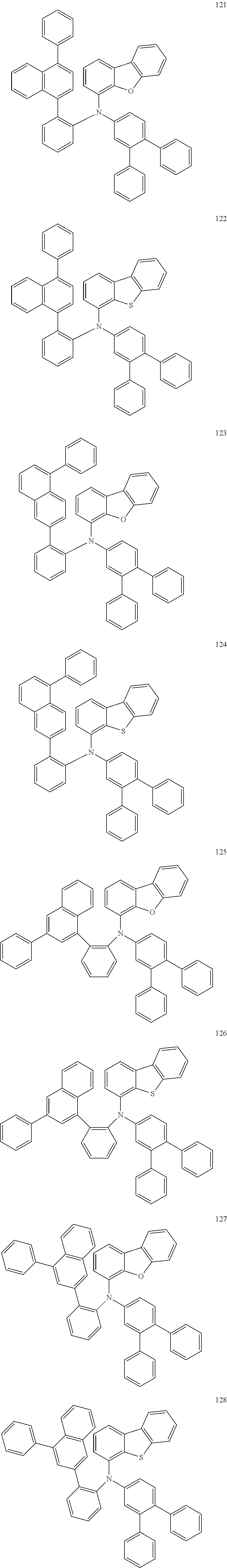 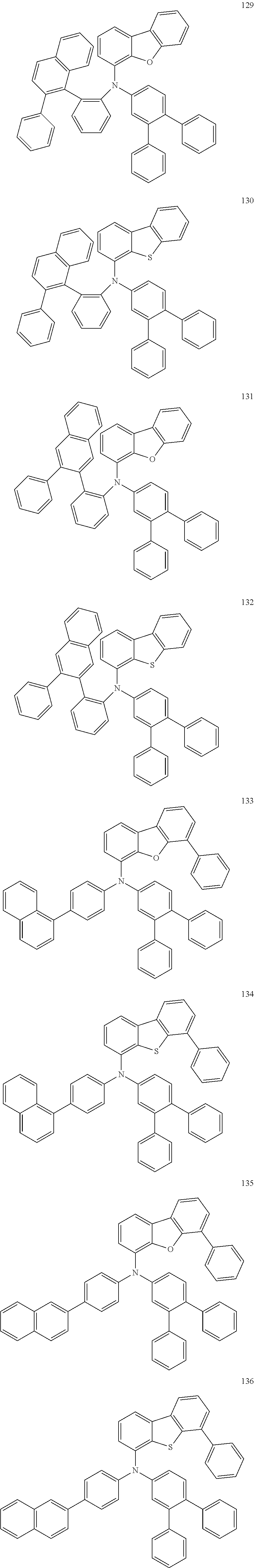 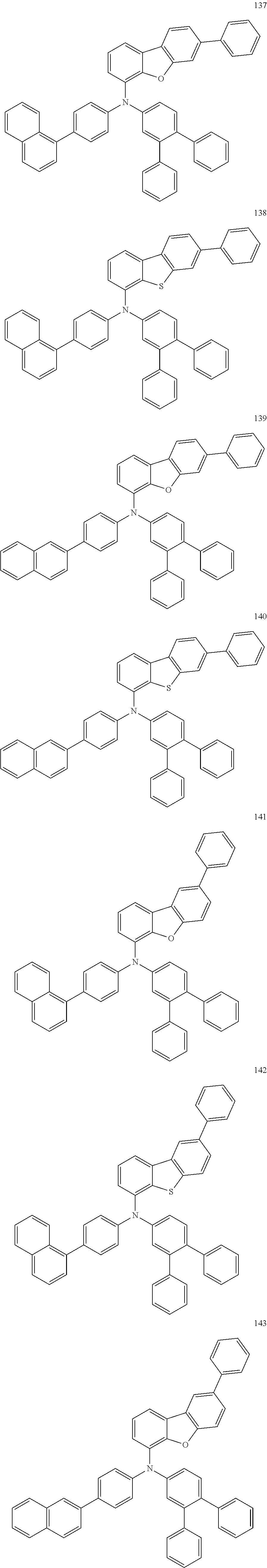 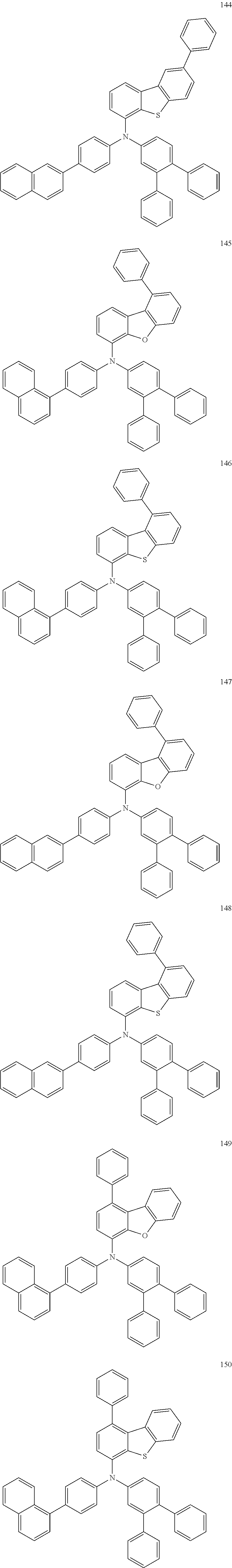 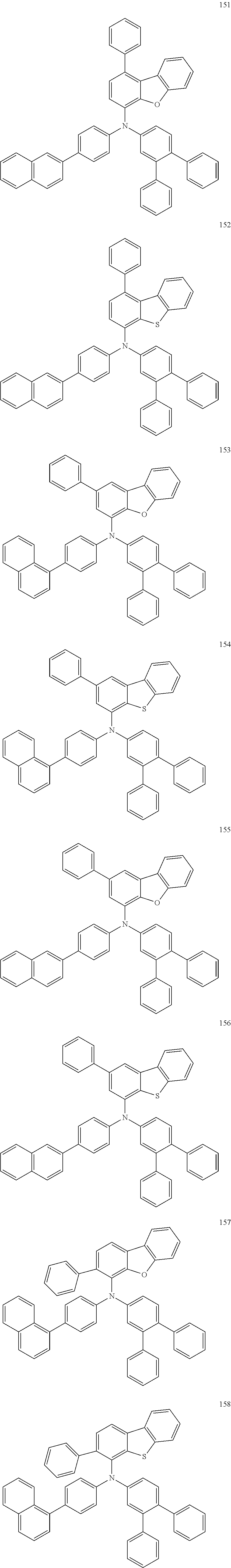 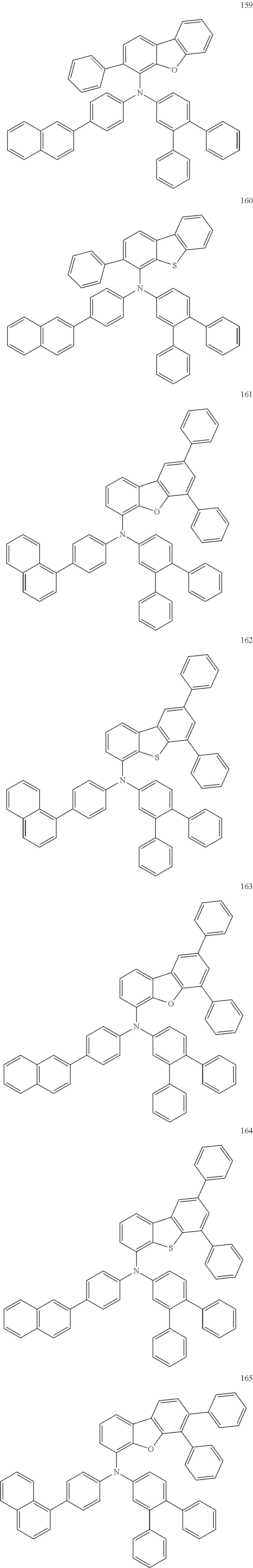 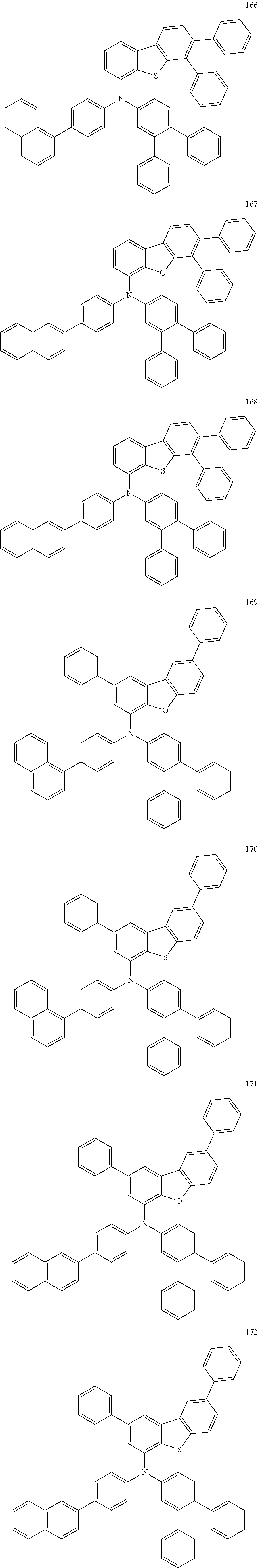 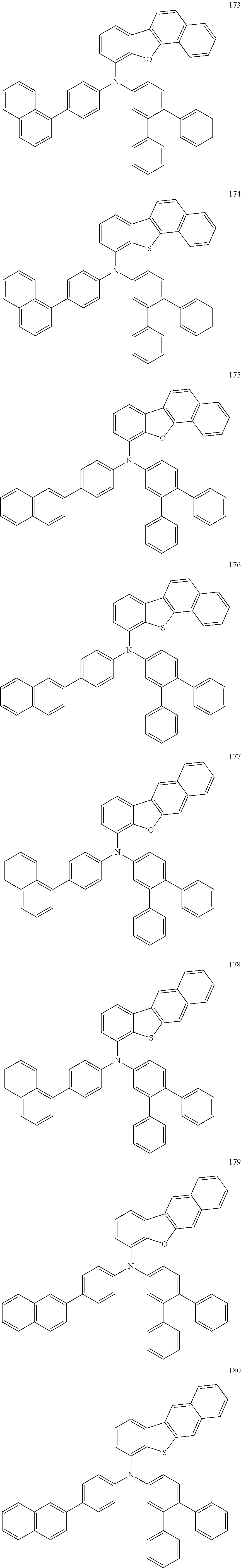 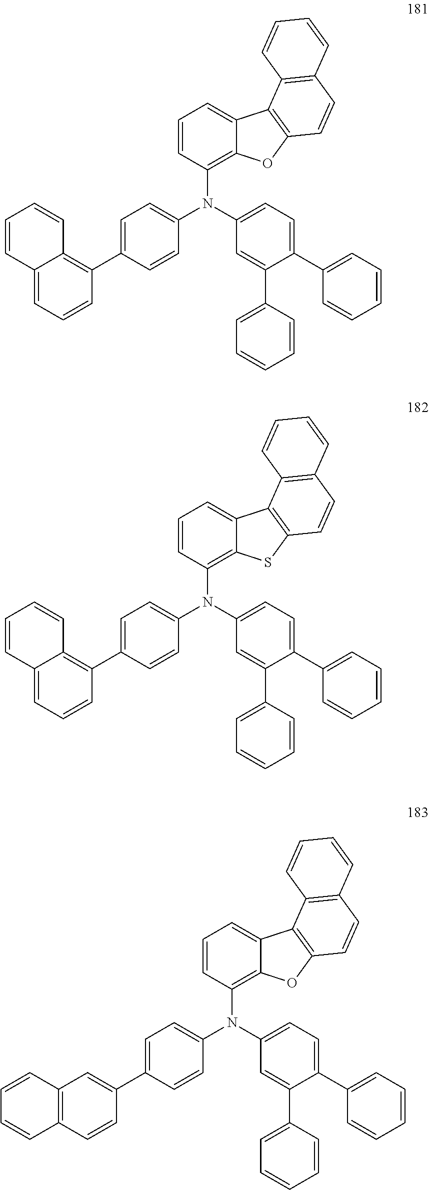 [SMILES: c1ccc(-c2ccc(N(c3ccc(-c4cccc5ccccc45)cc3)c3cccc4c3oc3ccc5ccccc5c34)cc2-c2ccccc2)cc1 ; c1ccc(-c2ccc(N(c3ccc(-c4cccc5ccccc45)cc3)c3cccc4c3sc3ccc5ccccc5c34)cc2-c2ccccc2)cc1 ; c1ccc(-c2ccc(N(c3ccc(-c4ccc5ccccc5c4)cc3)c3cccc4c3oc3ccc5ccccc5c34)cc2-c2ccccc2)cc1] 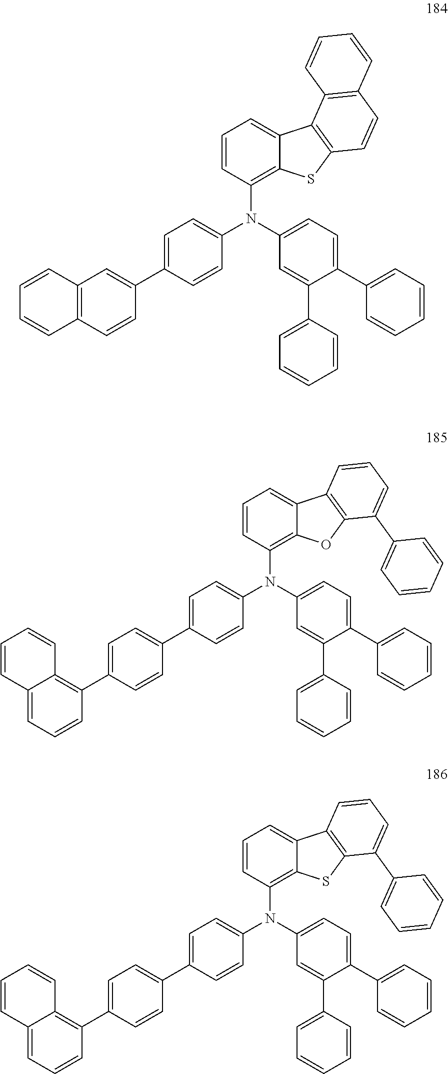 [SMILES: c1ccc(-c2ccc(N(c3ccc(-c4ccc5ccccc5c4)cc3)c3cccc4c3sc3ccc5ccccc5c34)cc2-c2ccccc2)cc1 ; c1ccc(-c2ccc(N(c3ccc(-c4ccc(-c5cccc6ccccc56)cc4)cc3)c3cccc4c3oc3c(-c5ccccc5)cccc34)cc2-c2ccccc2)cc1 ; c1ccc(-c2ccc(N(c3ccc(-c4ccc(-c5cccc6ccccc56)cc4)cc3)c3cccc4c3sc3c(-c5ccccc5)cccc34)cc2-c2ccccc2)cc1] 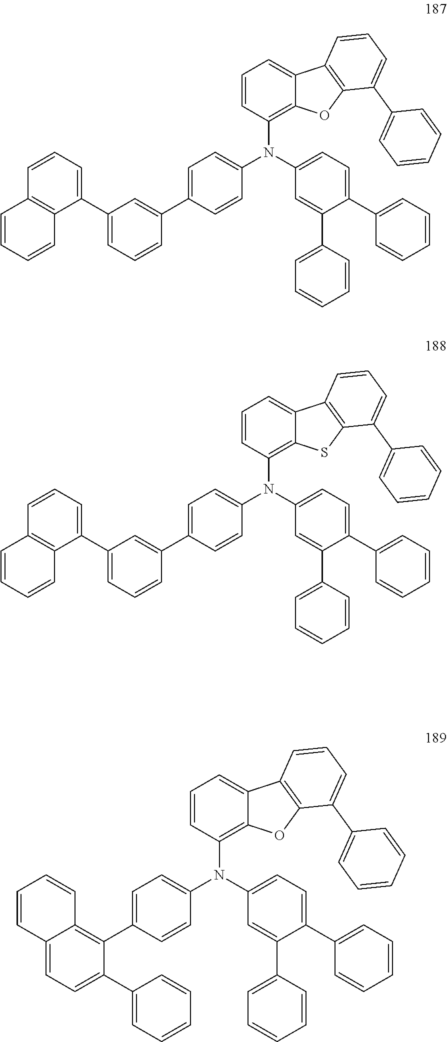 [SMILES: c1ccc(-c2ccc(N(c3ccc(-c4cccc(-c5cccc6ccccc56)c4)cc3)c3cccc4c3oc3c(-c5ccccc5)cccc34)cc2-c2ccccc2)cc1 ; c1ccc(-c2ccc(N(c3ccc(-c4cccc(-c5cccc6ccccc56)c4)cc3)c3cccc4c3sc3c(-c5ccccc5)cccc34)cc2-c2ccccc2)cc1 ; c1ccc(-c2ccc(N(c3ccc(-c4c(-c5ccccc5)ccc5ccccc45)cc3)c3cccc4c3oc3c(-c5ccccc5)cccc34)cc2-c2ccccc2)cc1] 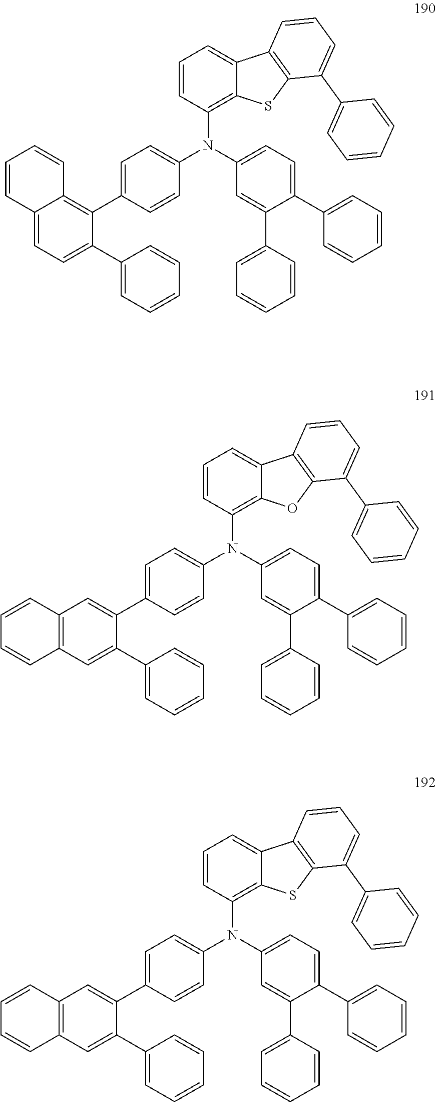 [SMILES: c1ccc(-c2ccc(N(c3ccc(-c4c(-c5ccccc5)ccc5ccccc45)cc3)c3cccc4c3sc3c(-c5ccccc5)cccc34)cc2-c2ccccc2)cc1 ; c1ccc(-c2ccc(N(c3ccc(-c4cc5ccccc5cc4-c4ccccc4)cc3)c3cccc4c3oc3c(-c5ccccc5)cccc34)cc2-c2ccccc2)cc1 ; c1ccc(-c2ccc(N(c3ccc(-c4cc5ccccc5cc4-c4ccccc4)cc3)c3cccc4c3sc3c(-c5ccccc5)cccc34)cc2-c2ccccc2)cc1] 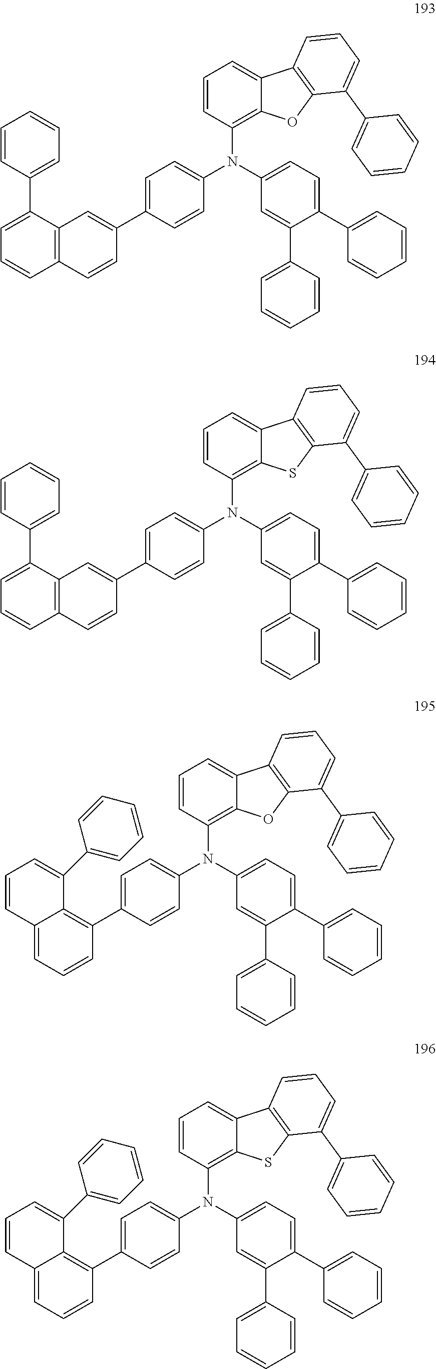 [SMILES: c1ccc(-c2ccc(N(c3ccc(-c4ccc5cccc(-c6ccccc6)c5c4)cc3)c3cccc4c3oc3c(-c5ccccc5)cccc34)cc2-c2ccccc2)cc1 ; c1ccc(-c2ccc(N(c3ccc(-c4ccc5cccc(-c6ccccc6)c5c4)cc3)c3cccc4c3sc3c(-c5ccccc5)cccc34)cc2-c2ccccc2)cc1 ; c1ccc(-c2ccc(N(c3ccc(-c4cccc5cccc(-c6ccccc6)c45)cc3)c3cccc4c3oc3c(-c5ccccc5)cccc34)cc2-c2ccccc2)cc1 ; c1ccc(-c2ccc(N(c3ccc(-c4cccc5cccc(-c6ccccc6)c45)cc3)c3cccc4c3sc3c(-c5ccccc5)cccc34)cc2-c2ccccc2)cc1] 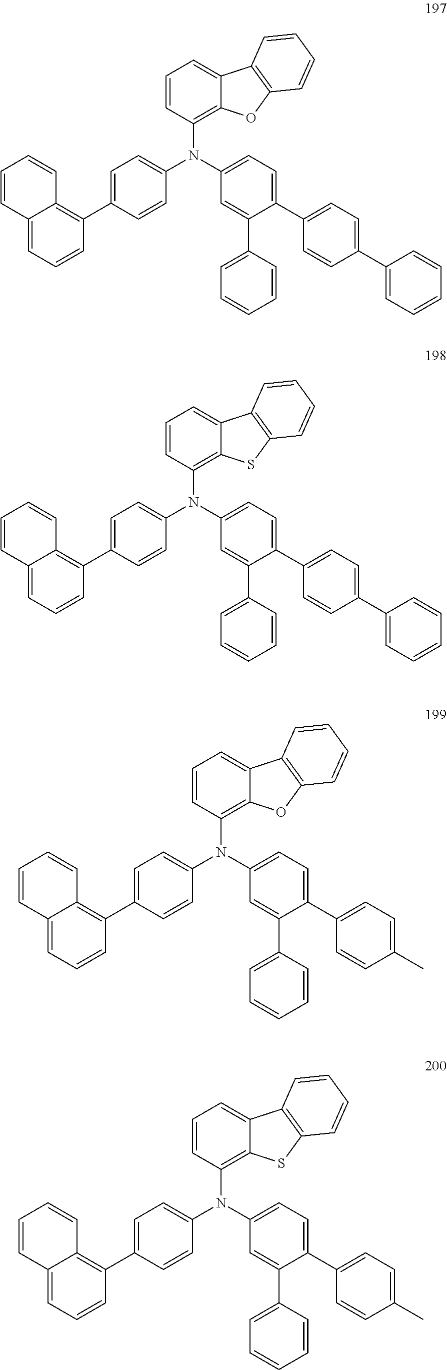 [SMILES: c1ccc(-c2ccc(-c3ccc(N(c4ccc(-c5cccc6ccccc56)cc4)c4cccc5c4oc4ccccc45)cc3-c3ccccc3)cc2)cc1 ; c1ccc(-c2ccc(-c3ccc(N(c4ccc(-c5cccc6ccccc56)cc4)c4cccc5c4sc4ccccc45)cc3-c3ccccc3)cc2)cc1 ; Cc1ccc(-c2ccc(N(c3ccc(-c4cccc5ccccc45)cc3)c3cccc4c3oc3ccccc34)cc2-c2ccccc2)cc1 ; Cc1ccc(-c2ccc(N(c3ccc(-c4cccc5ccccc45)cc3)c3cccc4c3sc3ccccc34)cc2-c2ccccc2)cc1] 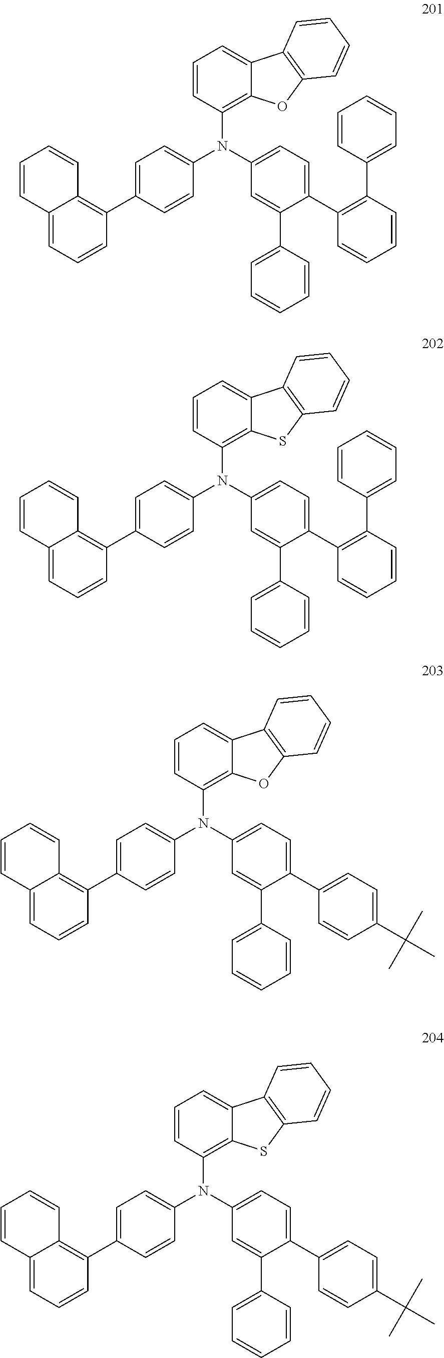 [SMILES: c1ccc(-c2ccccc2-c2ccc(N(c3ccc(-c4cccc5ccccc45)cc3)c3cccc4c3oc3ccccc34)cc2-c2ccccc2)cc1 ; c1ccc(-c2ccccc2-c2ccc(N(c3ccc(-c4cccc5ccccc45)cc3)c3cccc4c3sc3ccccc34)cc2-c2ccccc2)cc1 ; CC(C)(C)c1ccc(-c2ccc(N(c3ccc(-c4cccc5ccccc45)cc3)c3cccc4c3oc3ccccc34)cc2-c2ccccc2)cc1 ; CC(C)(C)c1ccc(-c2ccc(N(c3ccc(-c4cccc5ccccc45)cc3)c3cccc4c3sc3ccccc34)cc2-c2ccccc2)cc1] 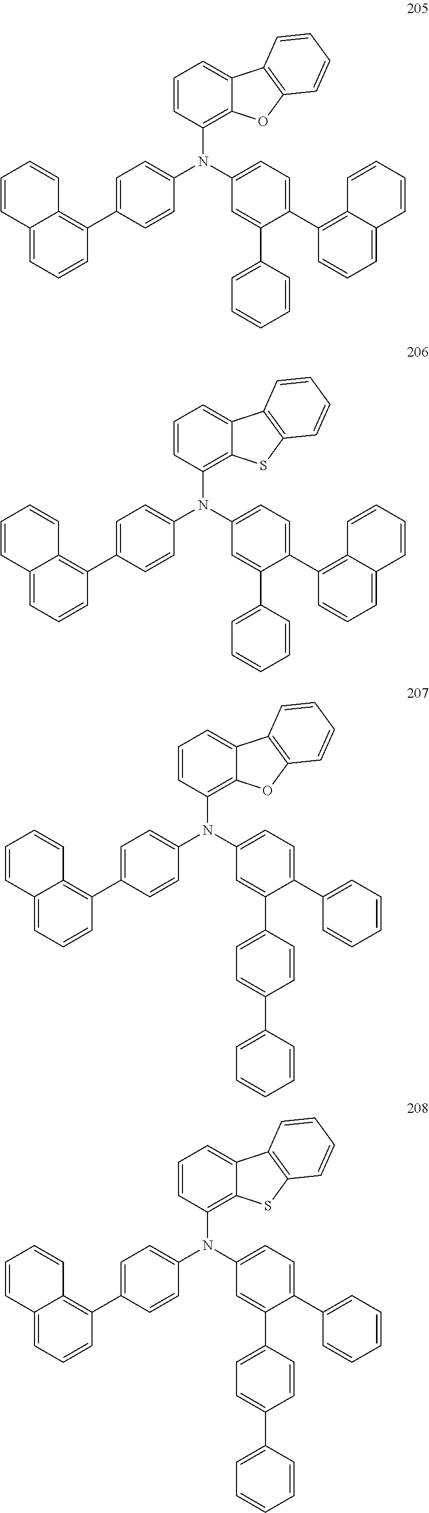 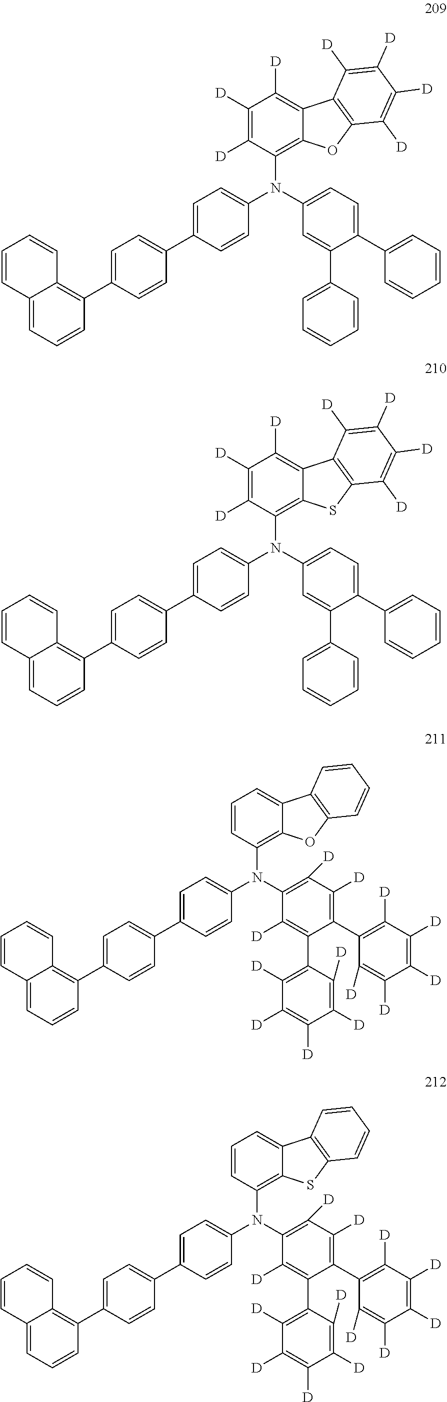 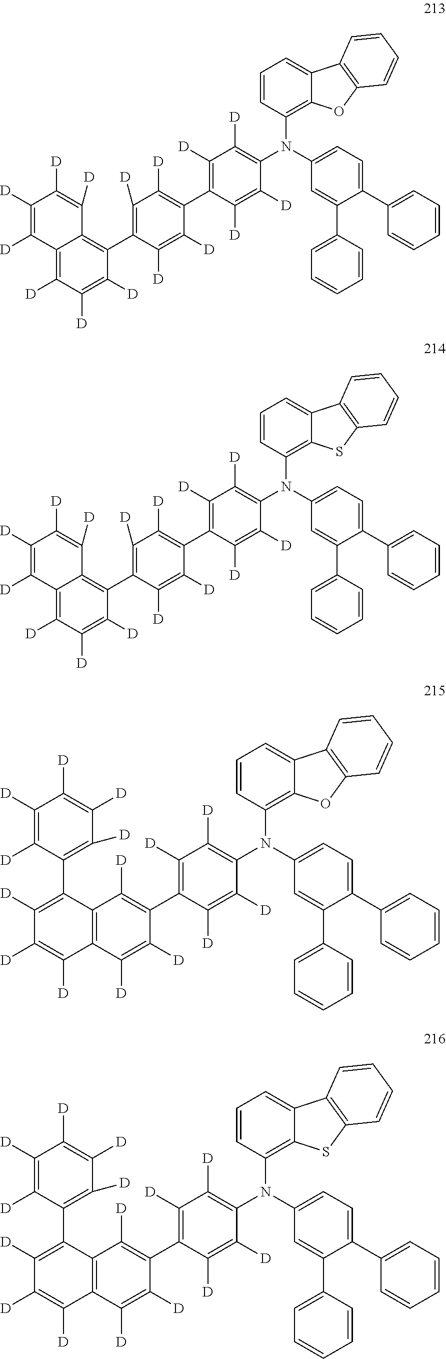 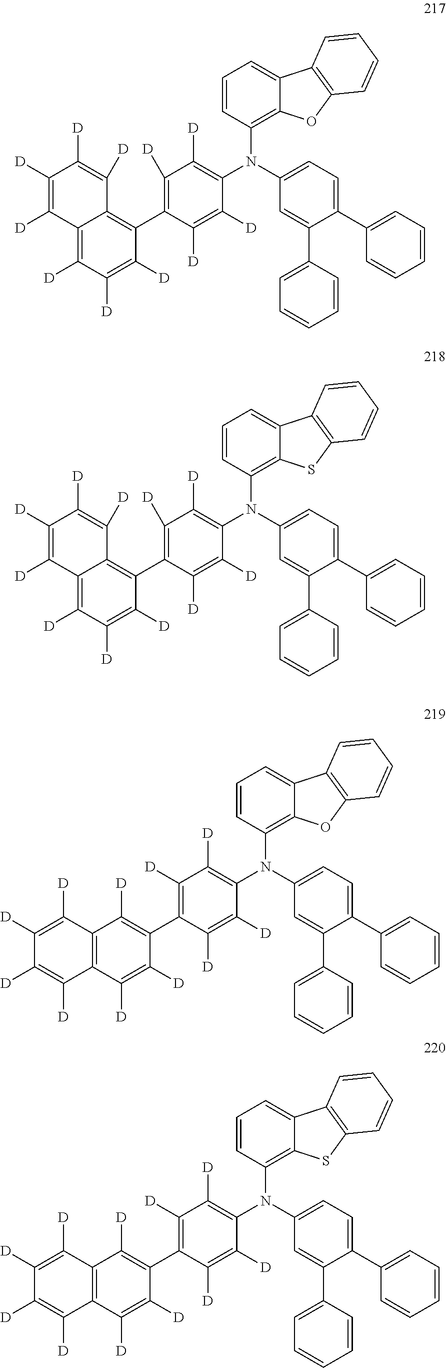 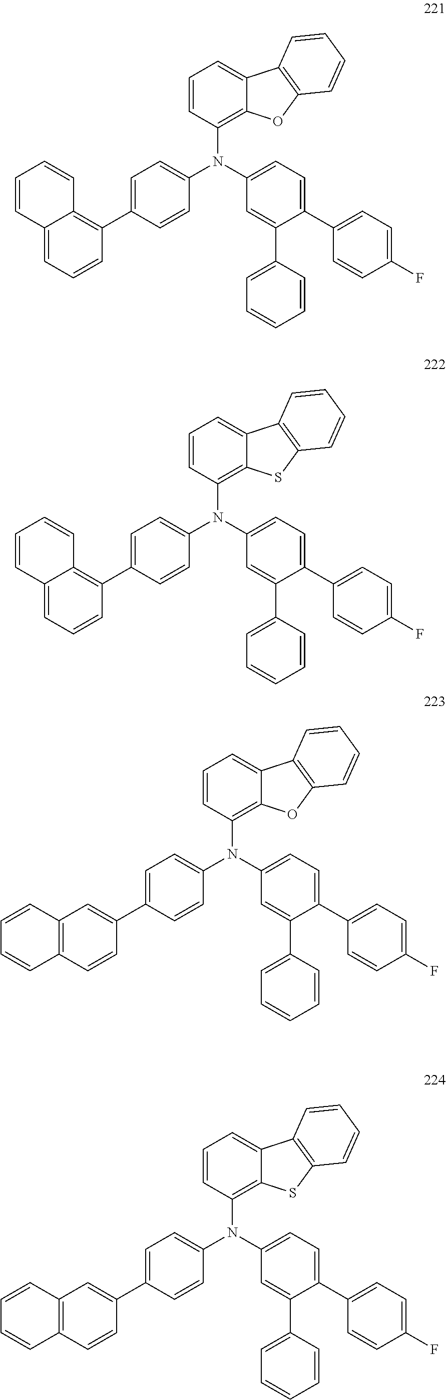 [SMILES: Fc1ccc(-c2ccc(N(c3ccc(-c4cccc5ccccc45)cc3)c3cccc4c3oc3ccccc34)cc2-c2ccccc2)cc1 ; Fc1ccc(-c2ccc(N(c3ccc(-c4cccc5ccccc45)cc3)c3cccc4c3sc3ccccc34)cc2-c2ccccc2)cc1 ; Fc1ccc(-c2ccc(N(c3ccc(-c4ccc5ccccc5c4)cc3)c3cccc4c3oc3ccccc34)cc2-c2ccccc2)cc1 ; Fc1ccc(-c2ccc(N(c3ccc(-c4ccc5ccccc5c4)cc3)c3cccc4c3sc3ccccc34)cc2-c2ccccc2)cc1] 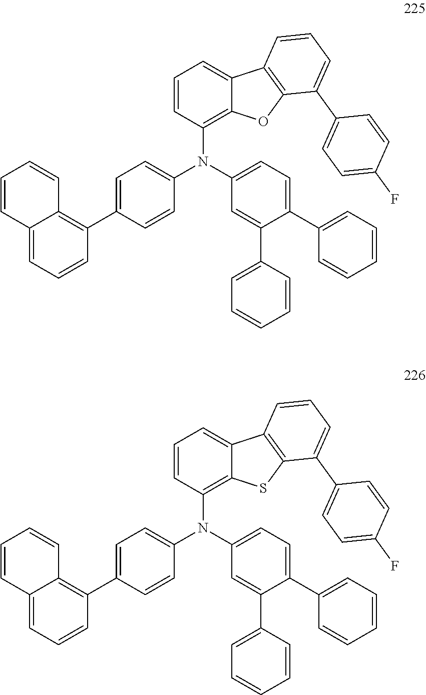 [SMILES: Fc1ccc(-c2cccc3c2oc2c(N(c4ccc(-c5cccc6ccccc56)cc4)c4ccc(-c5ccccc5)c(-c5ccccc5)c4)cccc23)cc1 ; Fc1ccc(-c2cccc3c2sc2c(N(c4ccc(-c5cccc6ccccc56)cc4)c4ccc(-c5ccccc5)c(-c5ccccc5)c4)cccc23)cc1] 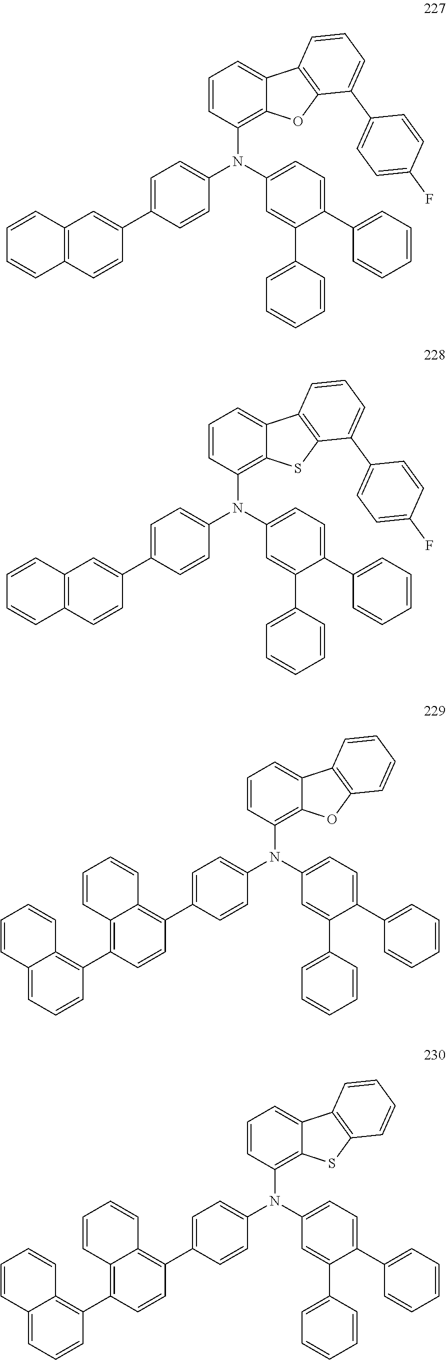 [SMILES: Fc1ccc(-c2cccc3c2oc2c(N(c4ccc(-c5ccc6ccccc6c5)cc4)c4ccc(-c5ccccc5)c(-c5ccccc5)c4)cccc23)cc1 ; Fc1ccc(-c2cccc3c2sc2c(N(c4ccc(-c5ccc6ccccc6c5)cc4)c4ccc(-c5ccccc5)c(-c5ccccc5)c4)cccc23)cc1 ; c1ccc(-c2ccc(N(c3ccc(-c4ccc(-c5cccc6ccccc56)c5ccccc45)cc3)c3cccc4c3oc3ccccc34)cc2-c2ccccc2)cc1 ; c1ccc(-c2ccc(N(c3ccc(-c4ccc(-c5cccc6ccccc56)c5ccccc45)cc3)c3cccc4c3sc3ccccc34)cc2-c2ccccc2)cc1] 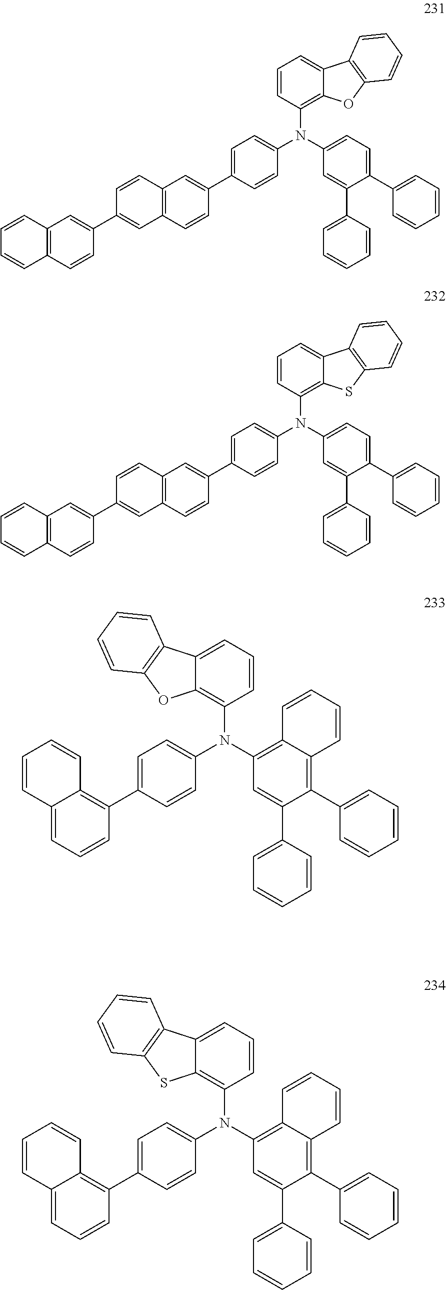 [SMILES: c1ccc(-c2ccc(N(c3ccc(-c4ccc5cc(-c6ccc7ccccc7c6)ccc5c4)cc3)c3cccc4c3oc3ccccc34)cc2-c2ccccc2)cc1 ; c1ccc(-c2ccc(N(c3ccc(-c4ccc5cc(-c6ccc7ccccc7c6)ccc5c4)cc3)c3cccc4c3sc3ccccc34)cc2-c2ccccc2)cc1 ; c1ccc(-c2cc(N(c3ccc(-c4cccc5ccccc45)cc3)c3cccc4c3oc3ccccc34)c3ccccc3c2-c2ccccc2)cc1 ; c1ccc(-c2cc(N(c3ccc(-c4cccc5ccccc45)cc3)c3cccc4c3sc3ccccc34)c3ccccc3c2-c2ccccc2)cc1] 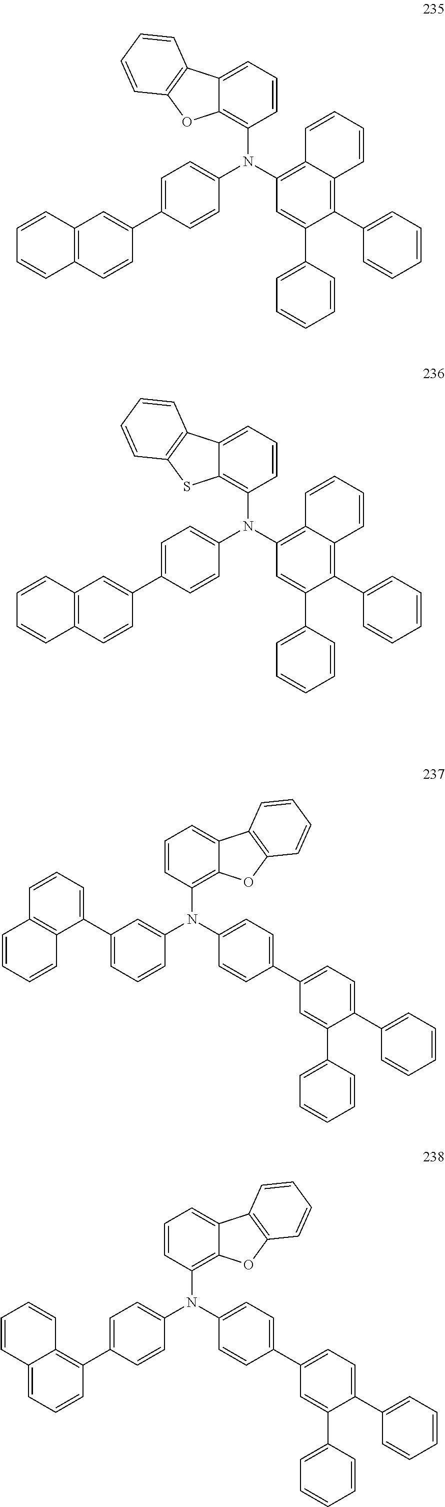 [SMILES: c1ccc(-c2cc(N(c3ccc(-c4ccc5ccccc5c4)cc3)c3cccc4c3oc3ccccc34)c3ccccc3c2-c2ccccc2)cc1 ; c1ccc(-c2cc(N(c3ccc(-c4ccc5ccccc5c4)cc3)c3cccc4c3sc3ccccc34)c3ccccc3c2-c2ccccc2)cc1 ; c1ccc(-c2ccc(-c3ccc(N(c4cccc(-c5cccc6ccccc56)c4)c4cccc5c4oc4ccccc45)cc3)cc2-c2ccccc2)cc1 ; c1ccc(-c2ccc(-c3ccc(N(c4ccc(-c5cccc6ccccc56)cc4)c4cccc5c4oc4ccccc45)cc3)cc2-c2ccccc2)cc1] 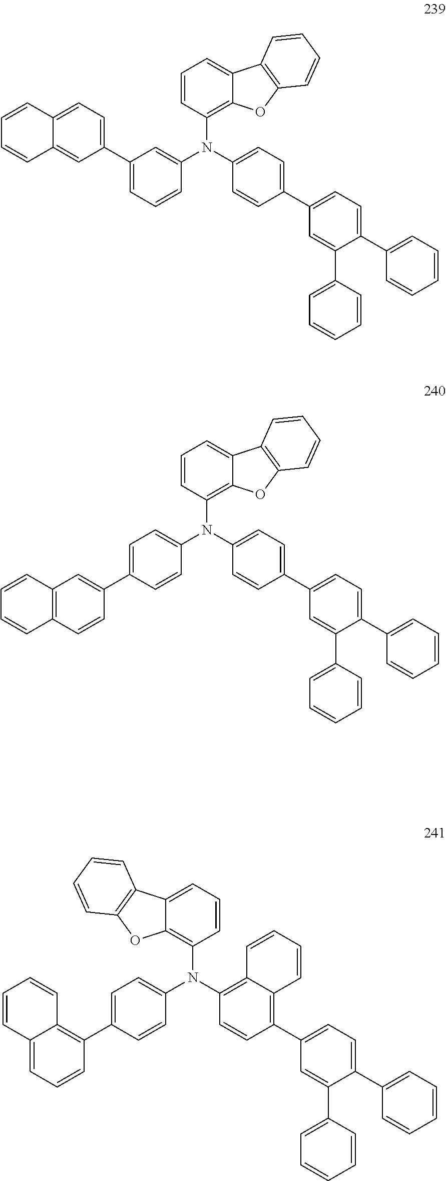 [SMILES: c1ccc(-c2ccc(-c3ccc(N(c4cccc(-c5ccc6ccccc6c5)c4)c4cccc5c4oc4ccccc45)cc3)cc2-c2ccccc2)cc1 ; c1ccc(-c2ccc(-c3ccc(N(c4ccc(-c5ccc6ccccc6c5)cc4)c4cccc5c4oc4ccccc45)cc3)cc2-c2ccccc2)cc1 ; c1ccc(-c2ccc(-c3ccc(N(c4ccc(-c5cccc6ccccc56)cc4)c4cccc5c4oc4ccccc45)c4ccccc34)cc2-c2ccccc2)cc1] 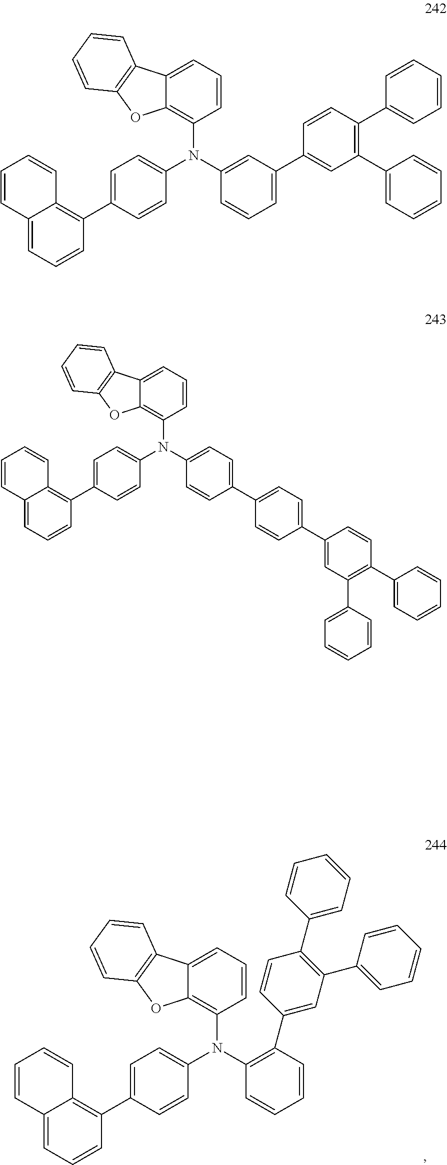 [SMILES: c1ccc(-c2ccc(-c3cccc(N(c4ccc(-c5cccc6ccccc56)cc4)c4cccc5c4oc4ccccc45)c3)cc2-c2ccccc2)cc1 ; c1ccc(-c2ccc(-c3ccc(-c4ccc(N(c5ccc(-c6cccc7ccccc67)cc5)c5cccc6c5oc5ccccc56)cc4)cc3)cc2-c2ccccc2)cc1 ; c1ccc(-c2ccc(-c3ccccc3N(c3ccc(-c4cccc5ccccc45)cc3)c3cccc4c3oc3ccccc34)cc2-c2ccccc2)cc1]
in Compound Group 1, D being deuterium.

10. An amine compound represented by Formula 1: 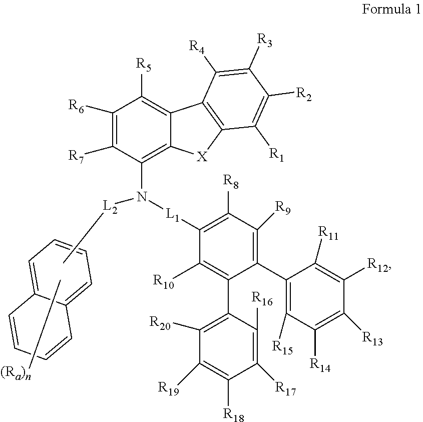
wherein, in Formula 1,
X is O or S,
R₁ to R₂₀ are each independently hydrogen, deuterium, a halogen, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or are independently bonded to an adjacent group to form a ring,
L₁ is a direct linkage, a substituted or unsubstituted arylene group having 6 to 30 ring-forming carbon atoms, or a substituted or unsubstituted heteroarylene group having 2 to 30 ring-forming carbon atoms,
L₂ is a substituted or unsubstituted arylene group having 6 to 30 ring-forming carbon atoms,
n is an integer of 1 to 7, and
Rₐ is hydrogen, deuterium, or a substituted or unsubstituted aryl group having 6 to 30 ring-forming carbon atoms.

11. The amine compound of claim 10, wherein the amine compound represented by Formula 1 is represented by Formula 1A or Formula 1B: 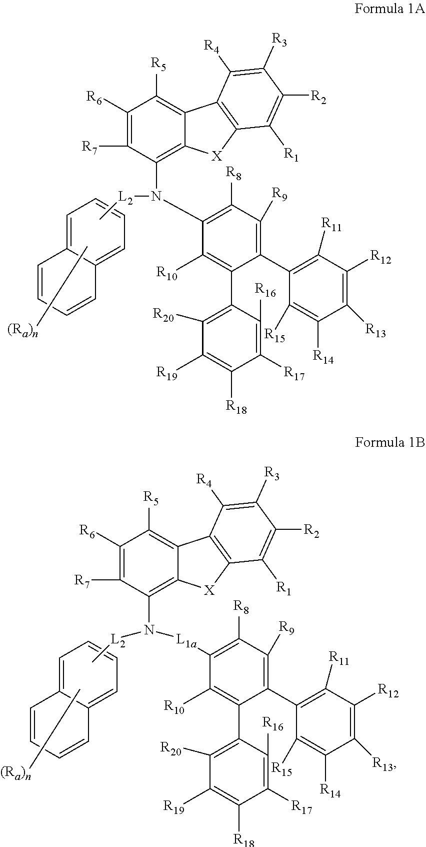
in Formula 1B, La being a substituted or unsubstituted arylene group having 6 to 30 ring-forming carbon atoms or a substituted or unsubstituted heteroarylene group having 2 to 30 ring-forming carbon atoms, and R₈ and R₉ being not bonded to an adjacent group to form a ring, and
in Formula 1A and Formula 1B, X, R₁ to R₂₀, n, Rₐ, and L₂ each being the same as defined in Formula 1.

12. The amine compound of claim 10, wherein the amine compound represented by Formula 1 is represented by Formula 1-1 or Formula 1-2: 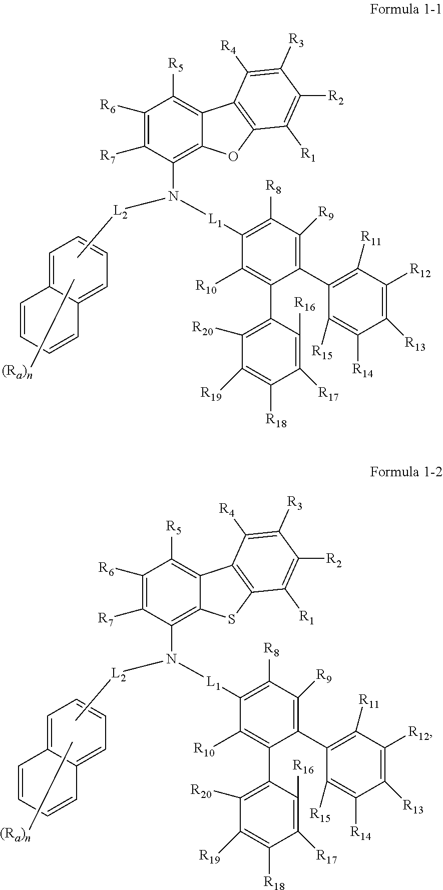
in Formula 1-1 and Formula 1-2, R₁ to R₂₀, L₁, L₂, Rₐ, and n each being the same as defined in Formula 1.

13. The amine compound of claim 10, wherein L₂ is a substituted or unsubstituted phenylene group or a substituted or unsubstituted divalent biphenyl group.

14. The amine compound of claim 10, wherein n is 1 and Rₐ is a substituted or unsubstituted phenyl group or a substituted or unsubstituted naphthyl group.

15. The amine compound of claim 10, wherein, in Formula 1, at least one hydrogen is substituted with deuterium.

16. The amine compound of claim 10, wherein, in Formula 1, each of R₁ to R₂₀, Rₐ, L₁, and L₂ does not include a substituted or unsubstituted amine group.

17. The amine compound of claim 10, wherein the amine compound represented by Formula 1 is any one selected from among compounds of Compound Group 1: 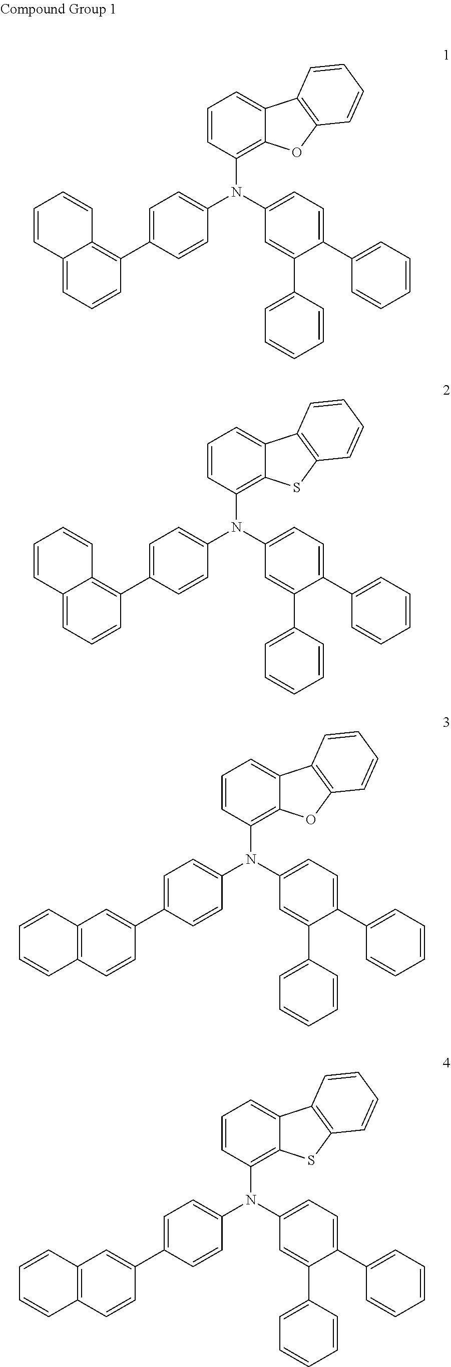 [SMILES: c1ccc(-c2ccc(N(c3ccc(-c4cccc5ccccc45)cc3)c3cccc4c3oc3ccccc34)cc2-c2ccccc2)cc1 ; c1ccc(-c2ccc(N(c3ccc(-c4cccc5ccccc45)cc3)c3cccc4c3sc3ccccc34)cc2-c2ccccc2)cc1 ; c1ccc(-c2ccc(N(c3ccc(-c4ccc5ccccc5c4)cc3)c3cccc4c3oc3ccccc34)cc2-c2ccccc2)cc1 ; c1ccc(-c2ccc(N(c3ccc(-c4ccc5ccccc5c4)cc3)c3cccc4c3sc3ccccc34)cc2-c2ccccc2)cc1] 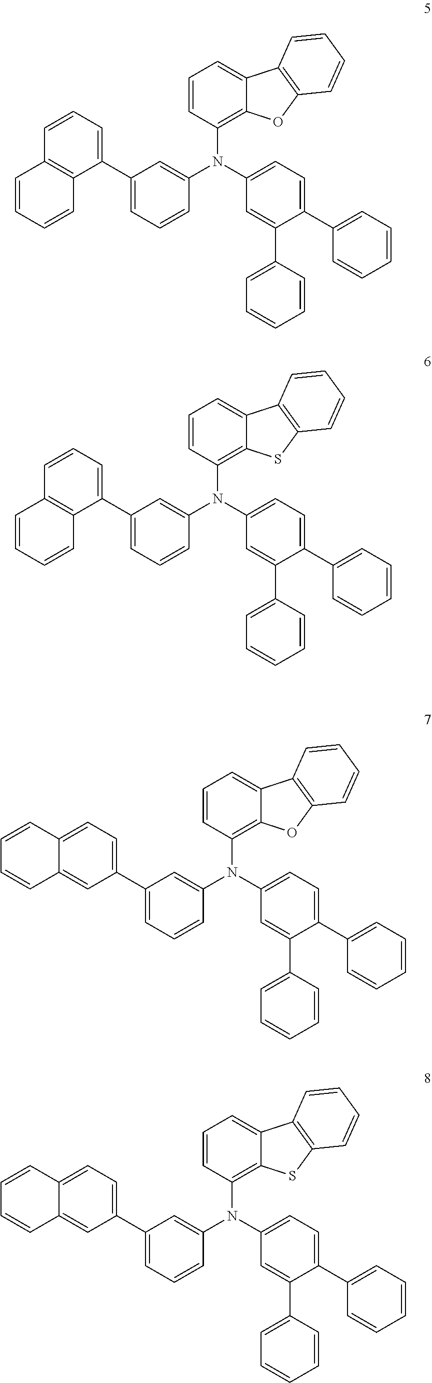 [SMILES: c1ccc(-c2ccc(N(c3cccc(-c4cccc5ccccc45)c3)c3cccc4c3oc3ccccc34)cc2-c2ccccc2)cc1 ; c1ccc(-c2ccc(N(c3cccc(-c4cccc5ccccc45)c3)c3cccc4c3sc3ccccc34)cc2-c2ccccc2)cc1 ; c1ccc(-c2ccc(N(c3cccc(-c4ccc5ccccc5c4)c3)c3cccc4c3oc3ccccc34)cc2-c2ccccc2)cc1 ; c1ccc(-c2ccc(N(c3cccc(-c4ccc5ccccc5c4)c3)c3cccc4c3sc3ccccc34)cc2-c2ccccc2)cc1] 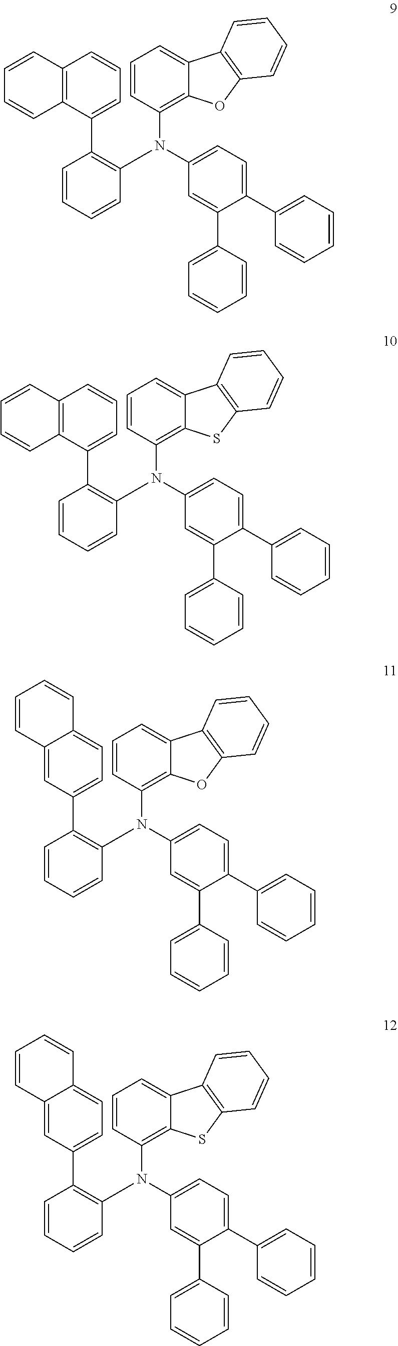 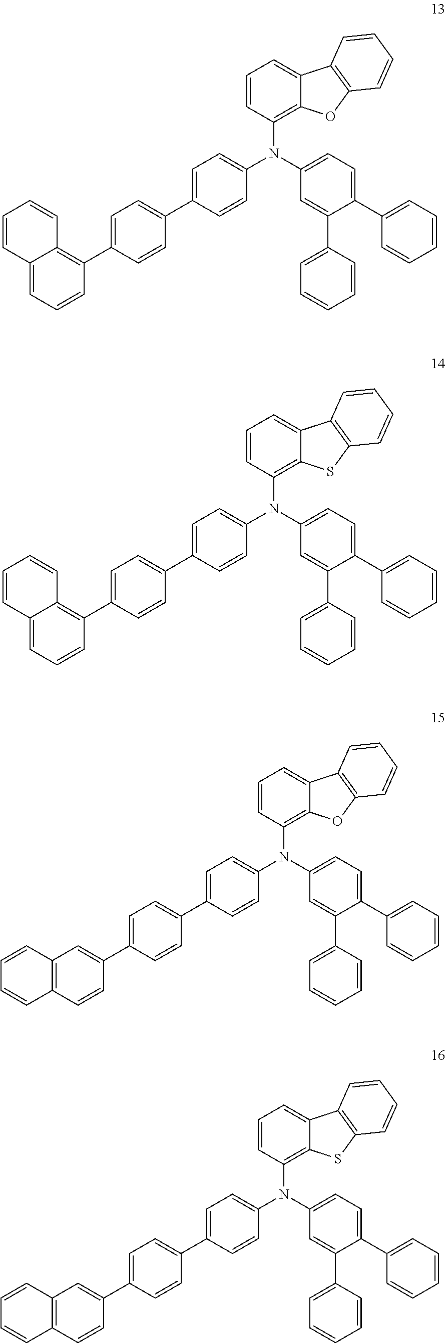 [SMILES: c1ccc(-c2ccc(N(c3ccc(-c4ccc(-c5cccc6ccccc56)cc4)cc3)c3cccc4c3oc3ccccc34)cc2-c2ccccc2)cc1 ; c1ccc(-c2ccc(N(c3ccc(-c4ccc(-c5cccc6ccccc56)cc4)cc3)c3cccc4c3sc3ccccc34)cc2-c2ccccc2)cc1 ; c1ccc(-c2ccc(N(c3ccc(-c4ccc(-c5ccc6ccccc6c5)cc4)cc3)c3cccc4c3oc3ccccc34)cc2-c2ccccc2)cc1 ; c1ccc(-c2ccc(N(c3ccc(-c4ccc(-c5ccc6ccccc6c5)cc4)cc3)c3cccc4c3sc3ccccc34)cc2-c2ccccc2)cc1] 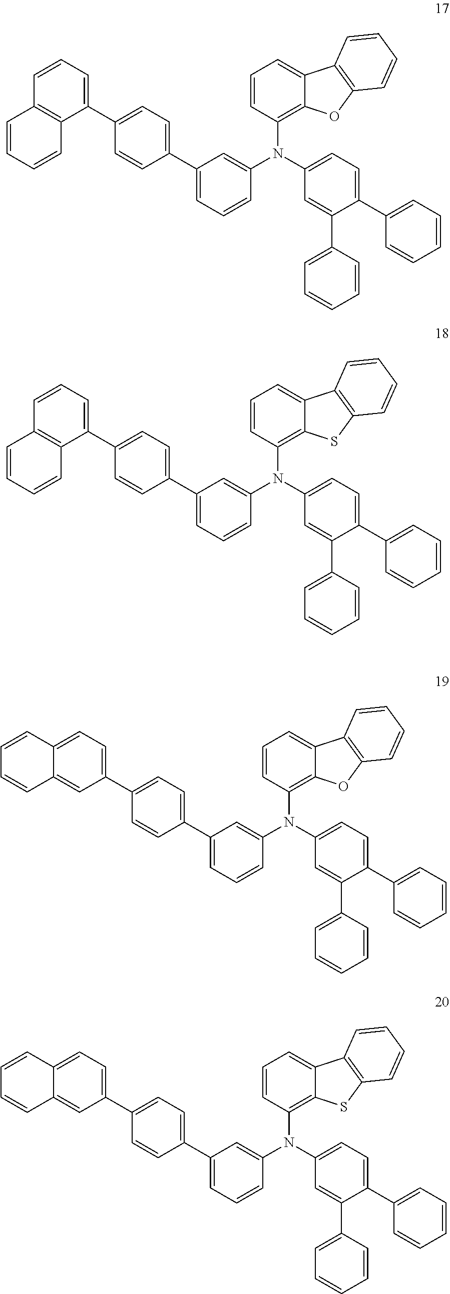 [SMILES: c1ccc(-c2ccc(N(c3cccc(-c4ccc(-c5cccc6ccccc56)cc4)c3)c3cccc4c3oc3ccccc34)cc2-c2ccccc2)cc1 ; c1ccc(-c2ccc(N(c3cccc(-c4ccc(-c5cccc6ccccc56)cc4)c3)c3cccc4c3sc3ccccc34)cc2-c2ccccc2)cc1 ; c1ccc(-c2ccc(N(c3cccc(-c4ccc(-c5ccc6ccccc6c5)cc4)c3)c3cccc4c3oc3ccccc34)cc2-c2ccccc2)cc1 ; c1ccc(-c2ccc(N(c3cccc(-c4ccc(-c5ccc6ccccc6c5)cc4)c3)c3cccc4c3sc3ccccc34)cc2-c2ccccc2)cc1] 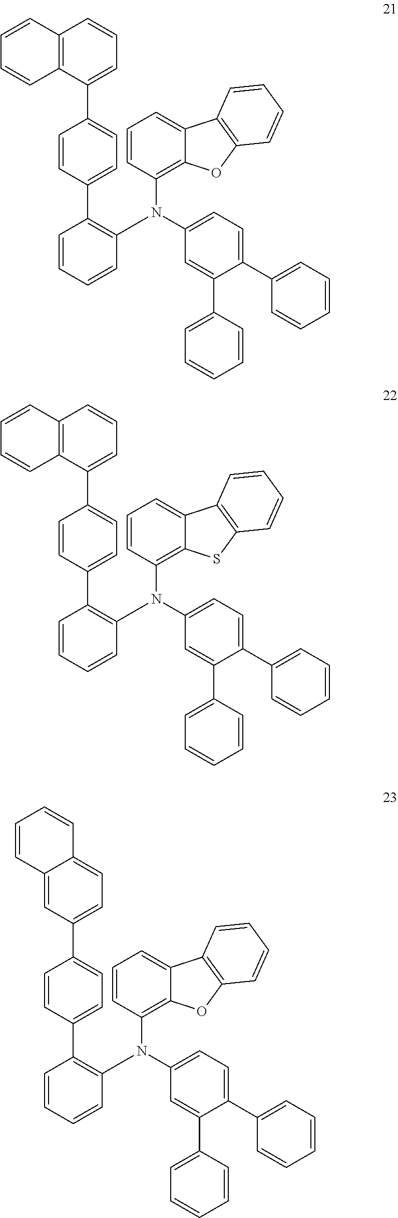 [SMILES: c1ccc(-c2ccc(N(c3ccccc3-c3ccc(-c4cccc5ccccc45)cc3)c3cccc4c3oc3ccccc34)cc2-c2ccccc2)cc1 ; c1ccc(-c2ccc(N(c3ccccc3-c3ccc(-c4cccc5ccccc45)cc3)c3cccc4c3sc3ccccc34)cc2-c2ccccc2)cc1 ; c1ccc(-c2ccc(N(c3ccccc3-c3ccc(-c4ccc5ccccc5c4)cc3)c3cccc4c3oc3ccccc34)cc2-c2ccccc2)cc1] 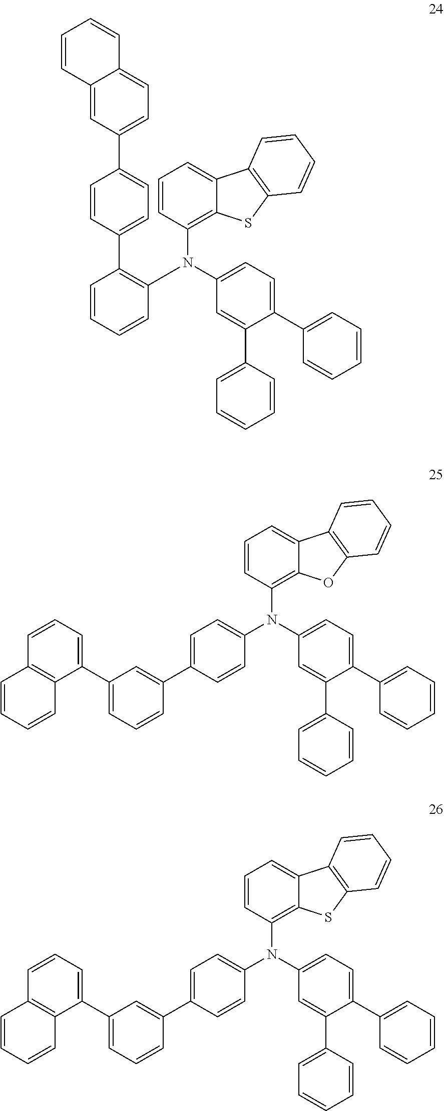 [SMILES: c1ccc(-c2ccc(N(c3ccccc3-c3ccc(-c4ccc5ccccc5c4)cc3)c3cccc4c3sc3ccccc34)cc2-c2ccccc2)cc1 ; c1ccc(-c2ccc(N(c3ccc(-c4cccc(-c5cccc6ccccc56)c4)cc3)c3cccc4c3oc3ccccc34)cc2-c2ccccc2)cc1 ; c1ccc(-c2ccc(N(c3ccc(-c4cccc(-c5cccc6ccccc56)c4)cc3)c3cccc4c3sc3ccccc34)cc2-c2ccccc2)cc1] 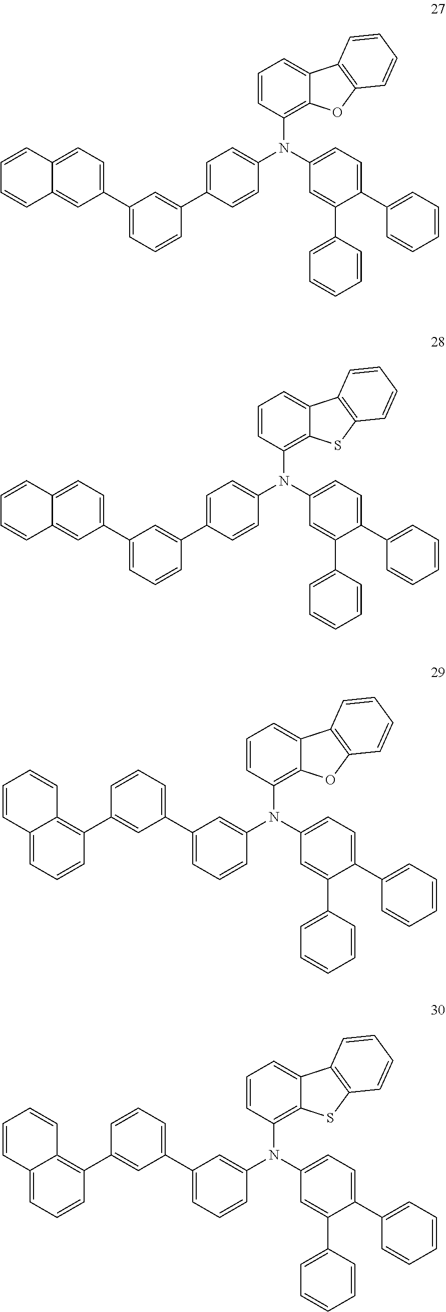 [SMILES: c1ccc(-c2ccc(N(c3ccc(-c4cccc(-c5ccc6ccccc6c5)c4)cc3)c3cccc4c3oc3ccccc34)cc2-c2ccccc2)cc1 ; c1ccc(-c2ccc(N(c3ccc(-c4cccc(-c5ccc6ccccc6c5)c4)cc3)c3cccc4c3sc3ccccc34)cc2-c2ccccc2)cc1 ; c1ccc(-c2ccc(N(c3cccc(-c4cccc(-c5cccc6ccccc56)c4)c3)c3cccc4c3oc3ccccc34)cc2-c2ccccc2)cc1 ; c1ccc(-c2ccc(N(c3cccc(-c4cccc(-c5cccc6ccccc56)c4)c3)c3cccc4c3sc3ccccc34)cc2-c2ccccc2)cc1] 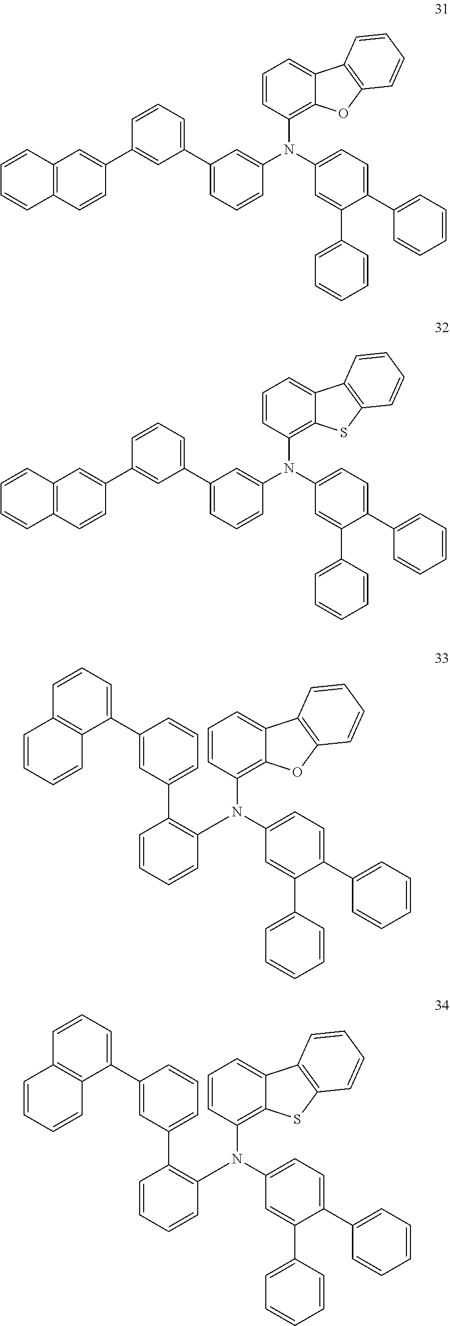 [SMILES: c1ccc(-c2ccc(N(c3cccc(-c4cccc(-c5ccc6ccccc6c5)c4)c3)c3cccc4c3oc3ccccc34)cc2-c2ccccc2)cc1 ; c1ccc(-c2ccc(N(c3cccc(-c4cccc(-c5ccc6ccccc6c5)c4)c3)c3cccc4c3sc3ccccc34)cc2-c2ccccc2)cc1 ; c1ccc(-c2ccc(N(c3ccccc3-c3cccc(-c4cccc5ccccc45)c3)c3cccc4c3oc3ccccc34)cc2-c2ccccc2)cc1 ; c1ccc(-c2ccc(N(c3ccccc3-c3cccc(-c4cccc5ccccc45)c3)c3cccc4c3sc3ccccc34)cc2-c2ccccc2)cc1] 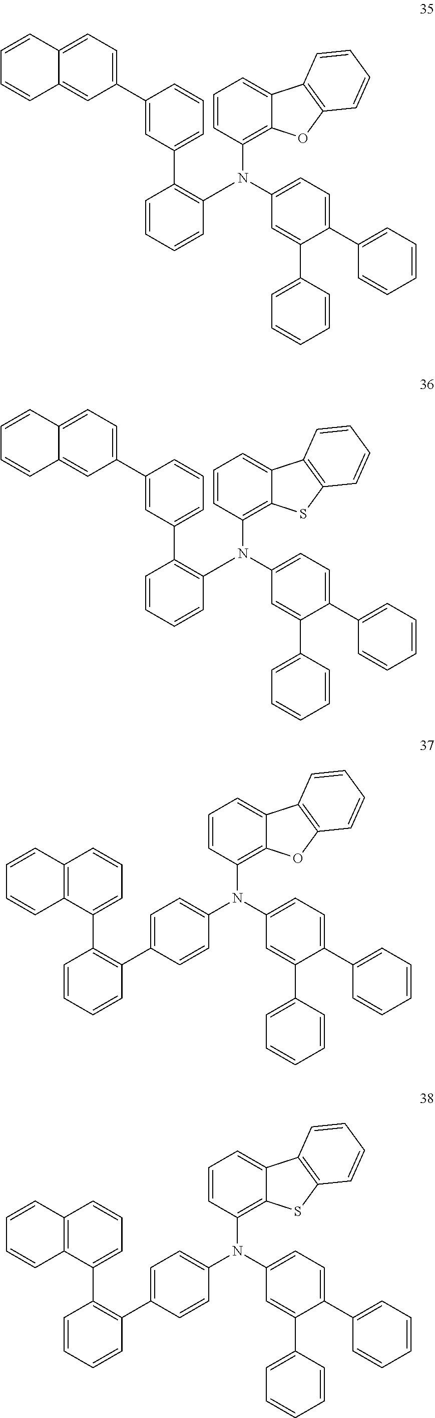 [SMILES: c1ccc(-c2ccc(N(c3ccccc3-c3cccc(-c4ccc5ccccc5c4)c3)c3cccc4c3oc3ccccc34)cc2-c2ccccc2)cc1 ; c1ccc(-c2ccc(N(c3ccccc3-c3cccc(-c4ccc5ccccc5c4)c3)c3cccc4c3sc3ccccc34)cc2-c2ccccc2)cc1 ; c1ccc(-c2ccc(N(c3ccc(-c4ccccc4-c4cccc5ccccc45)cc3)c3cccc4c3oc3ccccc34)cc2-c2ccccc2)cc1 ; c1ccc(-c2ccc(N(c3ccc(-c4ccccc4-c4cccc5ccccc45)cc3)c3cccc4c3sc3ccccc34)cc2-c2ccccc2)cc1] 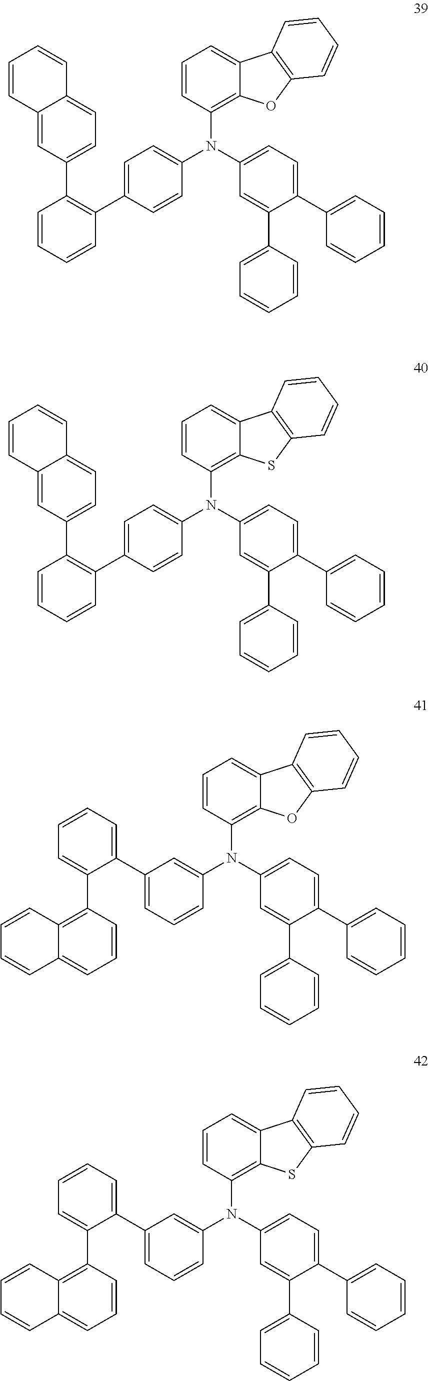 [SMILES: c1ccc(-c2ccc(N(c3ccc(-c4ccccc4-c4ccc5ccccc5c4)cc3)c3cccc4c3oc3ccccc34)cc2-c2ccccc2)cc1 ; c1ccc(-c2ccc(N(c3ccc(-c4ccccc4-c4ccc5ccccc5c4)cc3)c3cccc4c3sc3ccccc34)cc2-c2ccccc2)cc1 ; c1ccc(-c2ccc(N(c3cccc(-c4ccccc4-c4cccc5ccccc45)c3)c3cccc4c3oc3ccccc34)cc2-c2ccccc2)cc1 ; c1ccc(-c2ccc(N(c3cccc(-c4ccccc4-c4cccc5ccccc45)c3)c3cccc4c3sc3ccccc34)cc2-c2ccccc2)cc1] 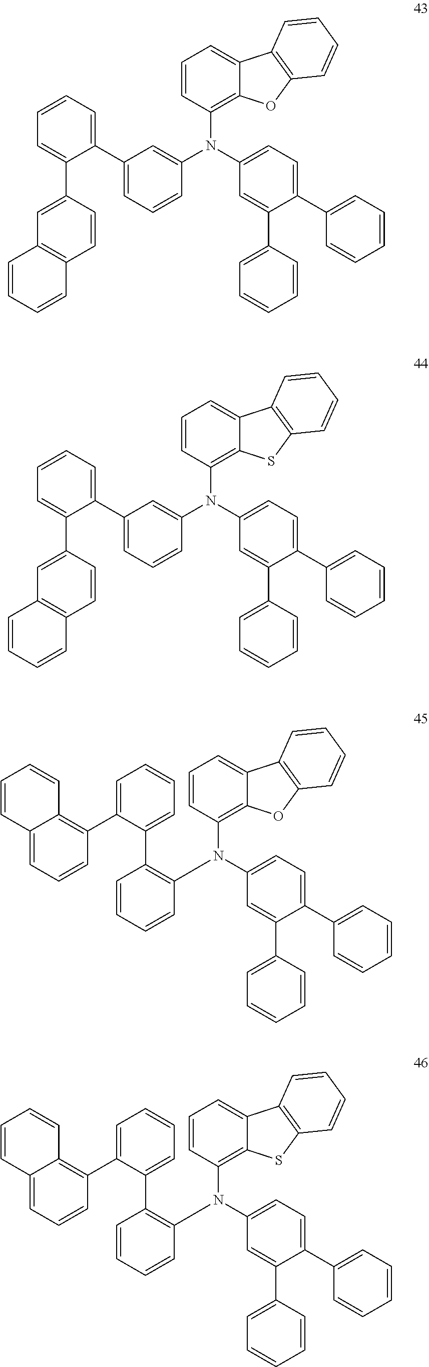 [SMILES: c1ccc(-c2ccc(N(c3cccc(-c4ccccc4-c4ccc5ccccc5c4)c3)c3cccc4c3oc3ccccc34)cc2-c2ccccc2)cc1 ; c1ccc(-c2ccc(N(c3cccc(-c4ccccc4-c4ccc5ccccc5c4)c3)c3cccc4c3sc3ccccc34)cc2-c2ccccc2)cc1 ; c1ccc(-c2ccc(N(c3ccccc3-c3ccccc3-c3cccc4ccccc34)c3cccc4c3oc3ccccc34)cc2-c2ccccc2)cc1 ; c1ccc(-c2ccc(N(c3ccccc3-c3ccccc3-c3cccc4ccccc34)c3cccc4c3sc3ccccc34)cc2-c2ccccc2)cc1] 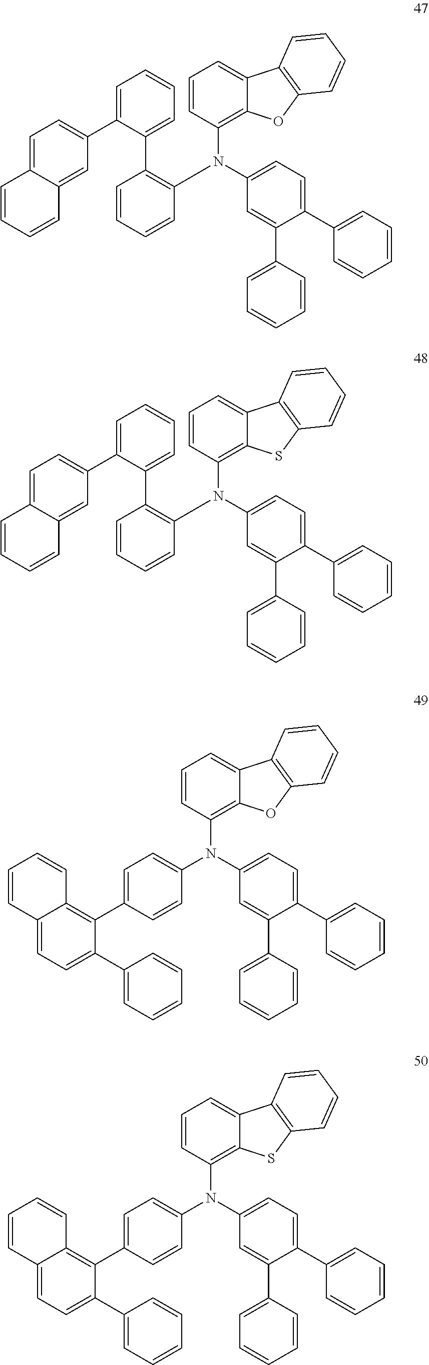 [SMILES: c1ccc(-c2ccc(N(c3ccccc3-c3ccccc3-c3ccc4ccccc4c3)c3cccc4c3oc3ccccc34)cc2-c2ccccc2)cc1 ; c1ccc(-c2ccc(N(c3ccccc3-c3ccccc3-c3ccc4ccccc4c3)c3cccc4c3sc3ccccc34)cc2-c2ccccc2)cc1 ; c1ccc(-c2ccc(N(c3ccc(-c4c(-c5ccccc5)ccc5ccccc45)cc3)c3cccc4c3oc3ccccc34)cc2-c2ccccc2)cc1 ; c1ccc(-c2ccc(N(c3ccc(-c4c(-c5ccccc5)ccc5ccccc45)cc3)c3cccc4c3sc3ccccc34)cc2-c2ccccc2)cc1] 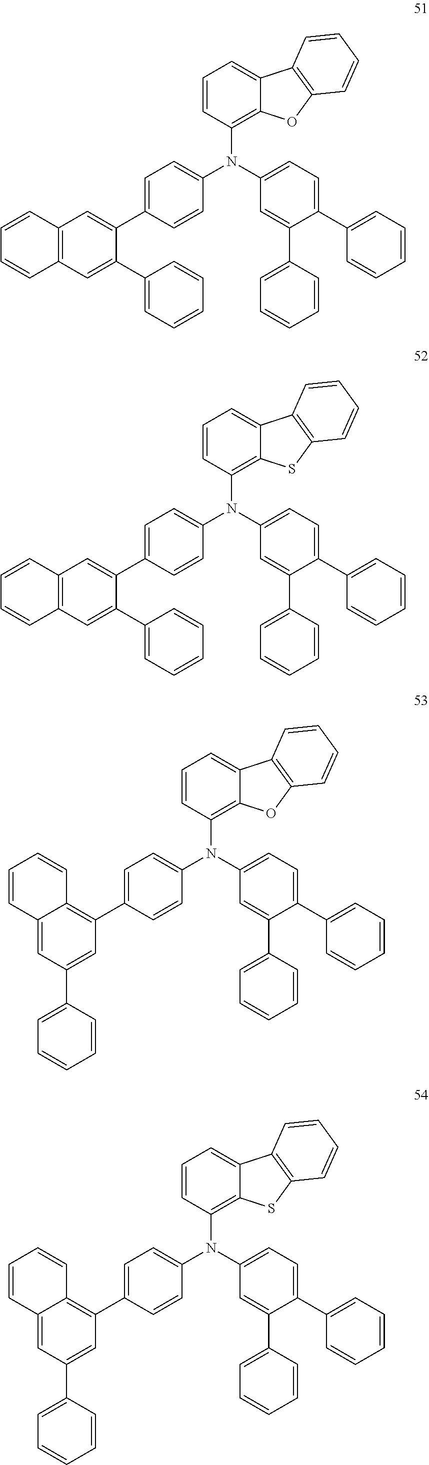 [SMILES: c1ccc(-c2ccc(N(c3ccc(-c4cc5ccccc5cc4-c4ccccc4)cc3)c3cccc4c3oc3ccccc34)cc2-c2ccccc2)cc1 ; c1ccc(-c2ccc(N(c3ccc(-c4cc5ccccc5cc4-c4ccccc4)cc3)c3cccc4c3sc3ccccc34)cc2-c2ccccc2)cc1 ; c1ccc(-c2cc(-c3ccc(N(c4ccc(-c5ccccc5)c(-c5ccccc5)c4)c4cccc5c4oc4ccccc45)cc3)c3ccccc3c2)cc1 ; c1ccc(-c2cc(-c3ccc(N(c4ccc(-c5ccccc5)c(-c5ccccc5)c4)c4cccc5c4sc4ccccc45)cc3)c3ccccc3c2)cc1] 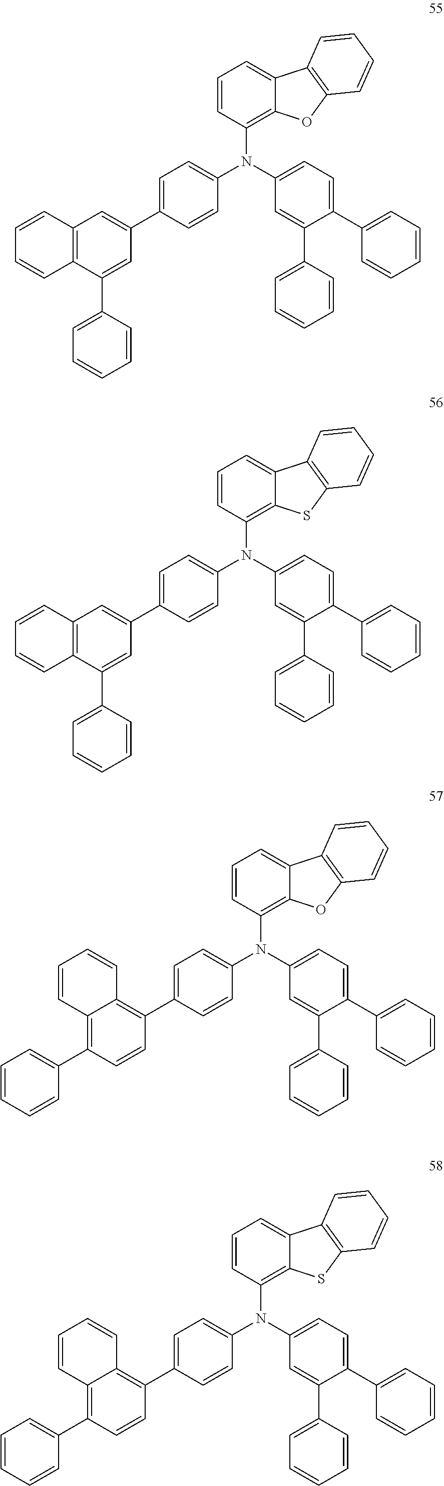 [SMILES: c1ccc(-c2ccc(N(c3ccc(-c4cc(-c5ccccc5)c5ccccc5c4)cc3)c3cccc4c3oc3ccccc34)cc2-c2ccccc2)cc1 ; c1ccc(-c2ccc(N(c3ccc(-c4cc(-c5ccccc5)c5ccccc5c4)cc3)c3cccc4c3sc3ccccc34)cc2-c2ccccc2)cc1 ; c1ccc(-c2ccc(N(c3ccc(-c4ccc(-c5ccccc5)c5ccccc45)cc3)c3cccc4c3oc3ccccc34)cc2-c2ccccc2)cc1 ; c1ccc(-c2ccc(N(c3ccc(-c4ccc(-c5ccccc5)c5ccccc45)cc3)c3cccc4c3sc3ccccc34)cc2-c2ccccc2)cc1] 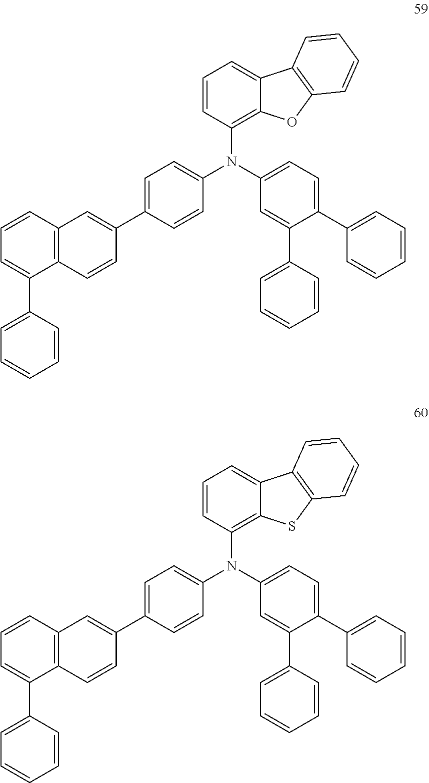 [SMILES: c1ccc(-c2ccc(N(c3ccc(-c4ccc5c(-c6ccccc6)cccc5c4)cc3)c3cccc4c3oc3ccccc34)cc2-c2ccccc2)cc1 ; c1ccc(-c2ccc(N(c3ccc(-c4ccc5c(-c6ccccc6)cccc5c4)cc3)c3cccc4c3sc3ccccc34)cc2-c2ccccc2)cc1] 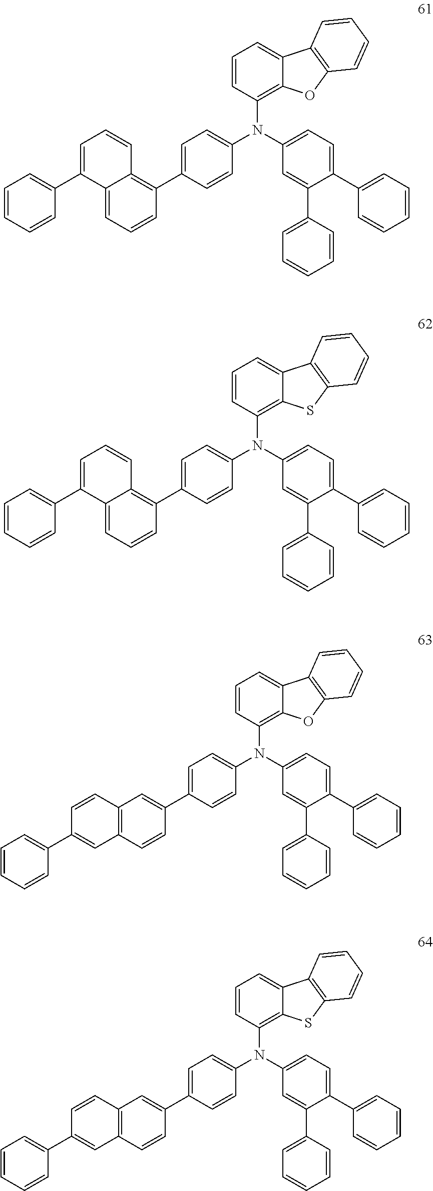 [SMILES: c1ccc(-c2ccc(N(c3ccc(-c4cccc5c(-c6ccccc6)cccc45)cc3)c3cccc4c3oc3ccccc34)cc2-c2ccccc2)cc1 ; c1ccc(-c2ccc(N(c3ccc(-c4cccc5c(-c6ccccc6)cccc45)cc3)c3cccc4c3sc3ccccc34)cc2-c2ccccc2)cc1 ; c1ccc(-c2ccc3cc(-c4ccc(N(c5ccc(-c6ccccc6)c(-c6ccccc6)c5)c5cccc6c5oc5ccccc56)cc4)ccc3c2)cc1 ; c1ccc(-c2ccc3cc(-c4ccc(N(c5ccc(-c6ccccc6)c(-c6ccccc6)c5)c5cccc6c5sc5ccccc56)cc4)ccc3c2)cc1] 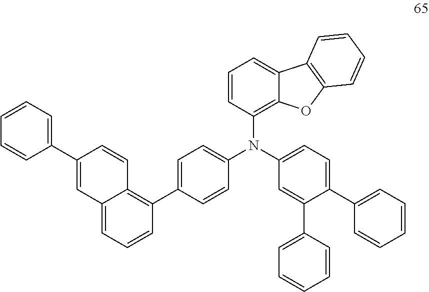 [SMILES: c1ccc(-c2ccc3c(-c4ccc(N(c5ccc(-c6ccccc6)c(-c6ccccc6)c5)c5cccc6c5oc5ccccc56)cc4)cccc3c2)cc1] 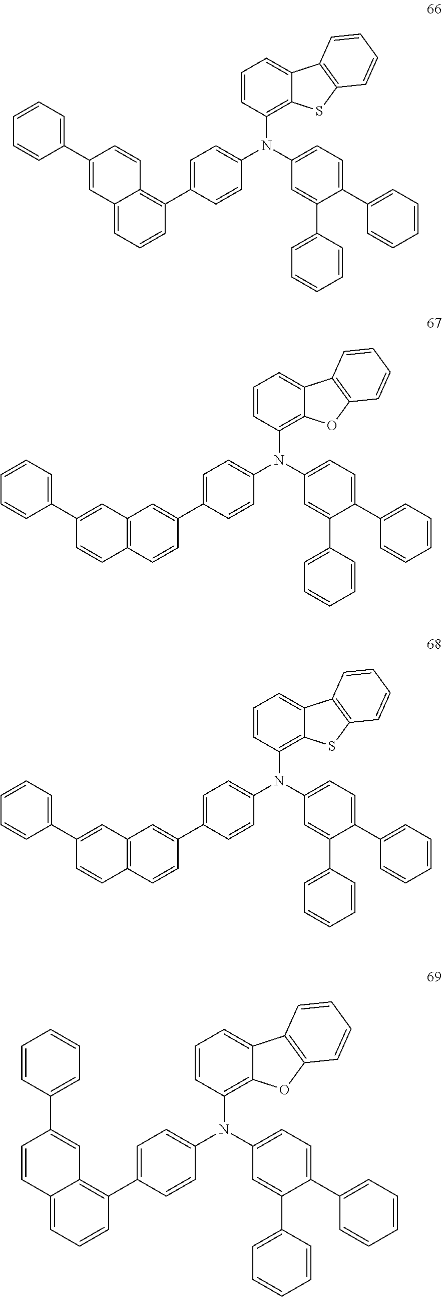 [SMILES: c1ccc(-c2ccc3c(-c4ccc(N(c5ccc(-c6ccccc6)c(-c6ccccc6)c5)c5cccc6c5sc5ccccc56)cc4)cccc3c2)cc1 ; c1ccc(-c2ccc3ccc(-c4ccc(N(c5ccc(-c6ccccc6)c(-c6ccccc6)c5)c5cccc6c5oc5ccccc56)cc4)cc3c2)cc1 ; c1ccc(-c2ccc3ccc(-c4ccc(N(c5ccc(-c6ccccc6)c(-c6ccccc6)c5)c5cccc6c5sc5ccccc56)cc4)cc3c2)cc1 ; c1ccc(-c2ccc3cccc(-c4ccc(N(c5ccc(-c6ccccc6)c(-c6ccccc6)c5)c5cccc6c5oc5ccccc56)cc4)c3c2)cc1] 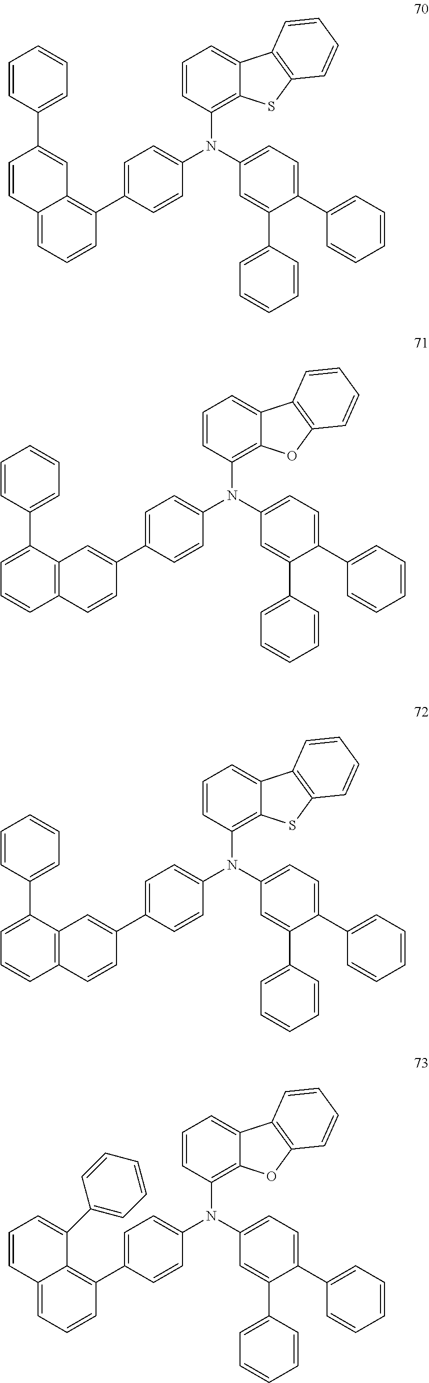 [SMILES: c1ccc(-c2ccc3cccc(-c4ccc(N(c5ccc(-c6ccccc6)c(-c6ccccc6)c5)c5cccc6c5sc5ccccc56)cc4)c3c2)cc1 ; c1ccc(-c2ccc(N(c3ccc(-c4ccc5cccc(-c6ccccc6)c5c4)cc3)c3cccc4c3oc3ccccc34)cc2-c2ccccc2)cc1 ; c1ccc(-c2ccc(N(c3ccc(-c4ccc5cccc(-c6ccccc6)c5c4)cc3)c3cccc4c3sc3ccccc34)cc2-c2ccccc2)cc1 ; c1ccc(-c2ccc(N(c3ccc(-c4cccc5cccc(-c6ccccc6)c45)cc3)c3cccc4c3oc3ccccc34)cc2-c2ccccc2)cc1] 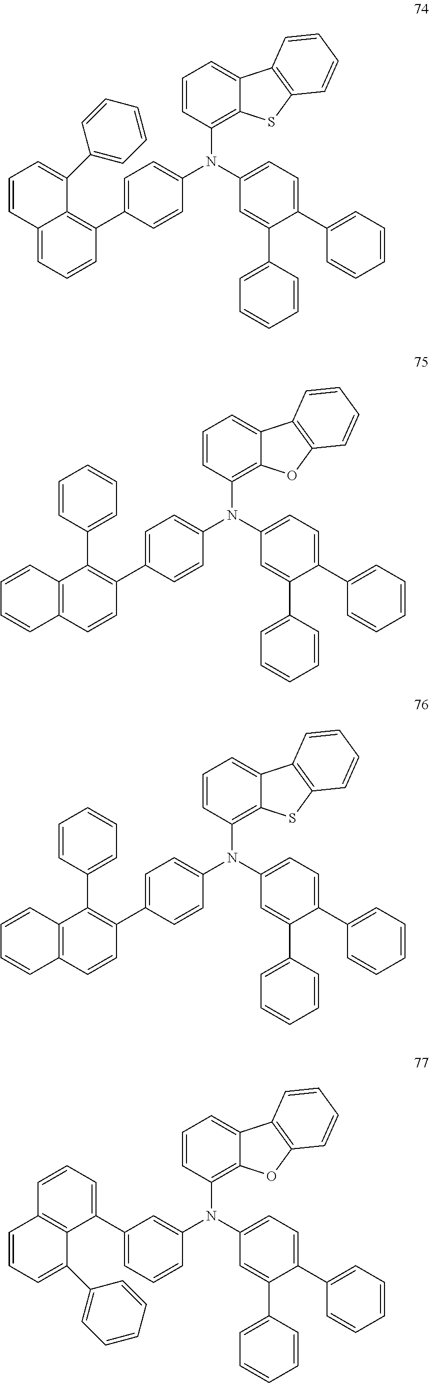 [SMILES: c1ccc(-c2ccc(N(c3ccc(-c4cccc5cccc(-c6ccccc6)c45)cc3)c3cccc4c3sc3ccccc34)cc2-c2ccccc2)cc1 ; c1ccc(-c2ccc(N(c3ccc(-c4ccc5ccccc5c4-c4ccccc4)cc3)c3cccc4c3oc3ccccc34)cc2-c2ccccc2)cc1 ; c1ccc(-c2ccc(N(c3ccc(-c4ccc5ccccc5c4-c4ccccc4)cc3)c3cccc4c3sc3ccccc34)cc2-c2ccccc2)cc1 ; c1ccc(-c2ccc(N(c3cccc(-c4cccc5cccc(-c6ccccc6)c45)c3)c3cccc4c3oc3ccccc34)cc2-c2ccccc2)cc1] 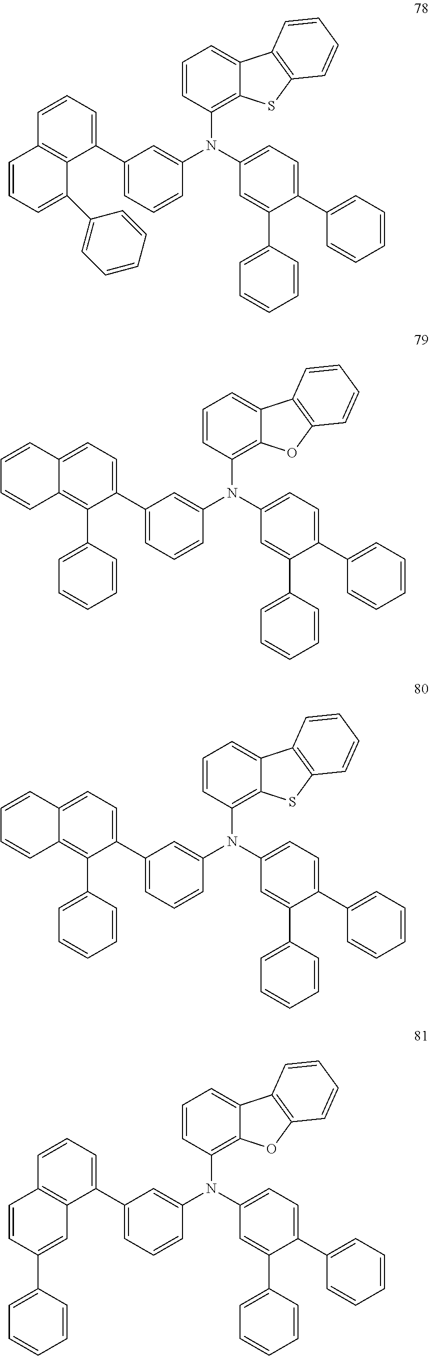 [SMILES: c1ccc(-c2ccc(N(c3cccc(-c4cccc5cccc(-c6ccccc6)c45)c3)c3cccc4c3sc3ccccc34)cc2-c2ccccc2)cc1 ; c1ccc(-c2ccc(N(c3cccc(-c4ccc5ccccc5c4-c4ccccc4)c3)c3cccc4c3oc3ccccc34)cc2-c2ccccc2)cc1 ; c1ccc(-c2ccc(N(c3cccc(-c4ccc5ccccc5c4-c4ccccc4)c3)c3cccc4c3sc3ccccc34)cc2-c2ccccc2)cc1 ; c1ccc(-c2ccc3cccc(-c4cccc(N(c5ccc(-c6ccccc6)c(-c6ccccc6)c5)c5cccc6c5oc5ccccc56)c4)c3c2)cc1] 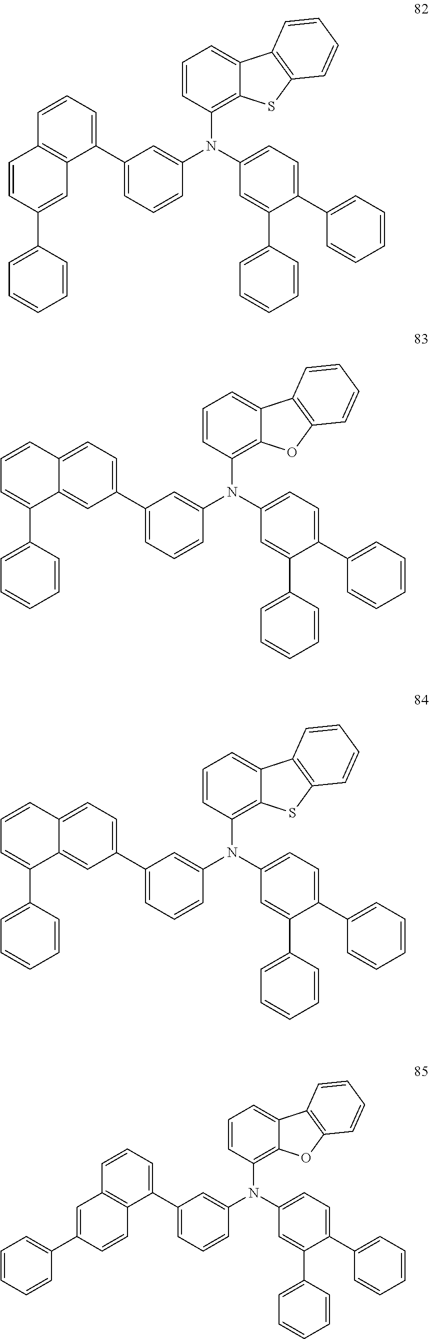 [SMILES: c1ccc(-c2ccc3cccc(-c4cccc(N(c5ccc(-c6ccccc6)c(-c6ccccc6)c5)c5cccc6c5sc5ccccc56)c4)c3c2)cc1 ; c1ccc(-c2ccc(N(c3cccc(-c4ccc5cccc(-c6ccccc6)c5c4)c3)c3cccc4c3oc3ccccc34)cc2-c2ccccc2)cc1 ; c1ccc(-c2ccc(N(c3cccc(-c4ccc5cccc(-c6ccccc6)c5c4)c3)c3cccc4c3sc3ccccc34)cc2-c2ccccc2)cc1 ; c1ccc(-c2ccc3c(-c4cccc(N(c5ccc(-c6ccccc6)c(-c6ccccc6)c5)c5cccc6c5oc5ccccc56)c4)cccc3c2)cc1] 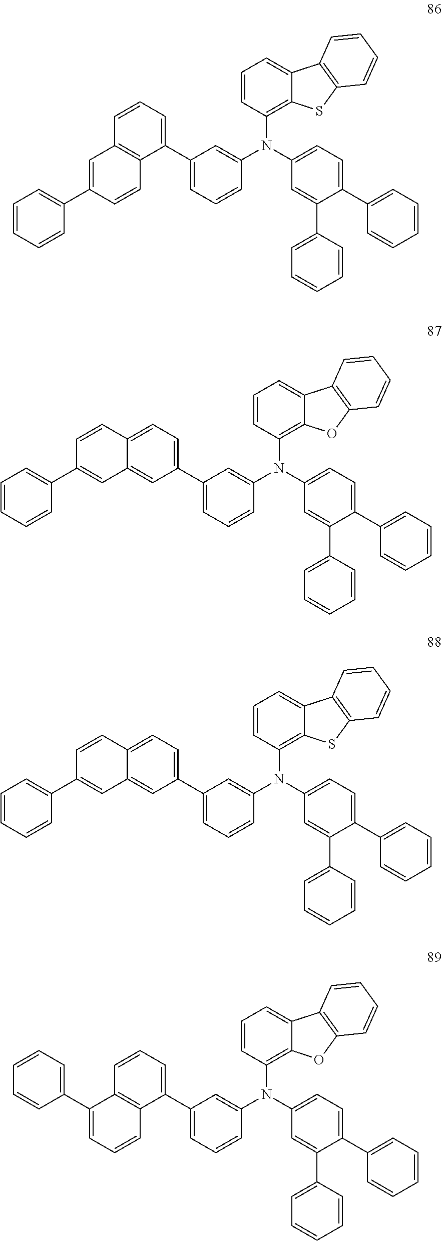 [SMILES: c1ccc(-c2ccc3c(-c4cccc(N(c5ccc(-c6ccccc6)c(-c6ccccc6)c5)c5cccc6c5sc5ccccc56)c4)cccc3c2)cc1 ; c1ccc(-c2ccc3ccc(-c4cccc(N(c5ccc(-c6ccccc6)c(-c6ccccc6)c5)c5cccc6c5oc5ccccc56)c4)cc3c2)cc1 ; c1ccc(-c2ccc3ccc(-c4cccc(N(c5ccc(-c6ccccc6)c(-c6ccccc6)c5)c5cccc6c5sc5ccccc56)c4)cc3c2)cc1 ; c1ccc(-c2ccc(N(c3cccc(-c4cccc5c(-c6ccccc6)cccc45)c3)c3cccc4c3oc3ccccc34)cc2-c2ccccc2)cc1] 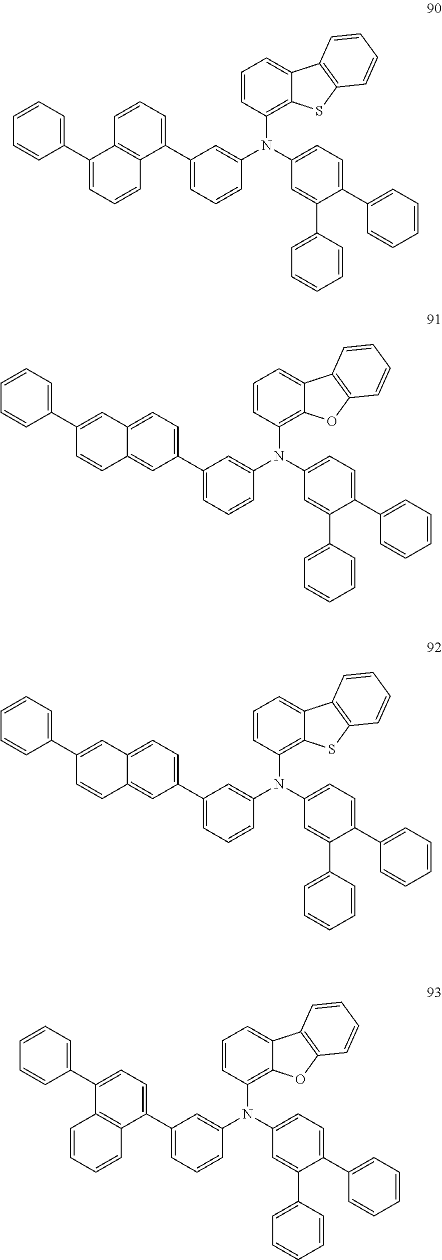 [SMILES: c1ccc(-c2ccc(N(c3cccc(-c4cccc5c(-c6ccccc6)cccc45)c3)c3cccc4c3sc3ccccc34)cc2-c2ccccc2)cc1 ; c1ccc(-c2ccc3cc(-c4cccc(N(c5ccc(-c6ccccc6)c(-c6ccccc6)c5)c5cccc6c5oc5ccccc56)c4)ccc3c2)cc1 ; c1ccc(-c2ccc3cc(-c4cccc(N(c5ccc(-c6ccccc6)c(-c6ccccc6)c5)c5cccc6c5sc5ccccc56)c4)ccc3c2)cc1 ; c1ccc(-c2ccc(N(c3cccc(-c4ccc(-c5ccccc5)c5ccccc45)c3)c3cccc4c3oc3ccccc34)cc2-c2ccccc2)cc1] 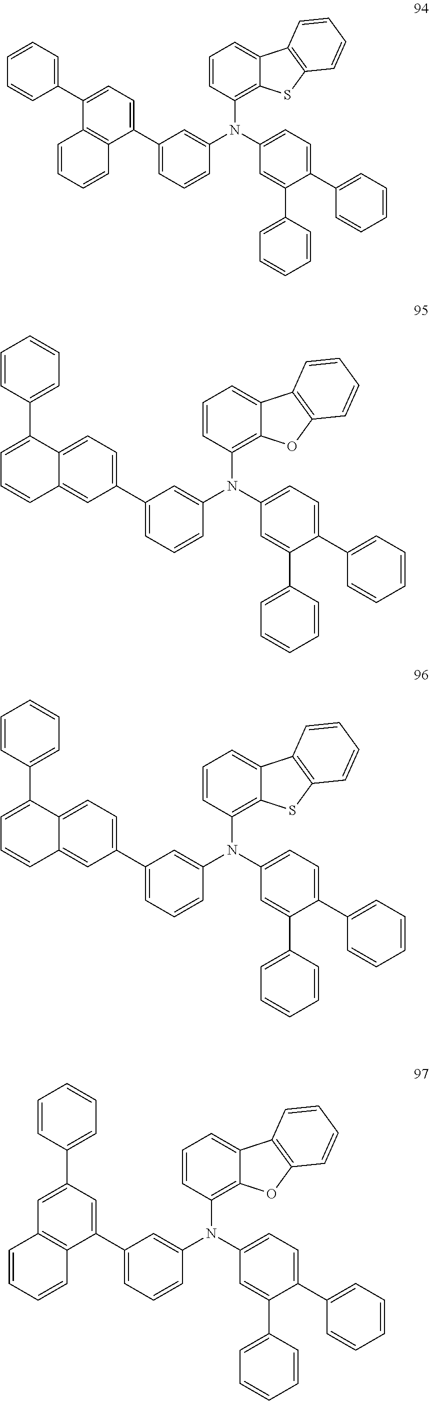 [SMILES: c1ccc(-c2ccc(N(c3cccc(-c4ccc(-c5ccccc5)c5ccccc45)c3)c3cccc4c3sc3ccccc34)cc2-c2ccccc2)cc1 ; c1ccc(-c2ccc(N(c3cccc(-c4ccc5c(-c6ccccc6)cccc5c4)c3)c3cccc4c3oc3ccccc34)cc2-c2ccccc2)cc1 ; c1ccc(-c2ccc(N(c3cccc(-c4ccc5c(-c6ccccc6)cccc5c4)c3)c3cccc4c3sc3ccccc34)cc2-c2ccccc2)cc1 ; c1ccc(-c2cc(-c3cccc(N(c4ccc(-c5ccccc5)c(-c5ccccc5)c4)c4cccc5c4oc4ccccc45)c3)c3ccccc3c2)cc1] 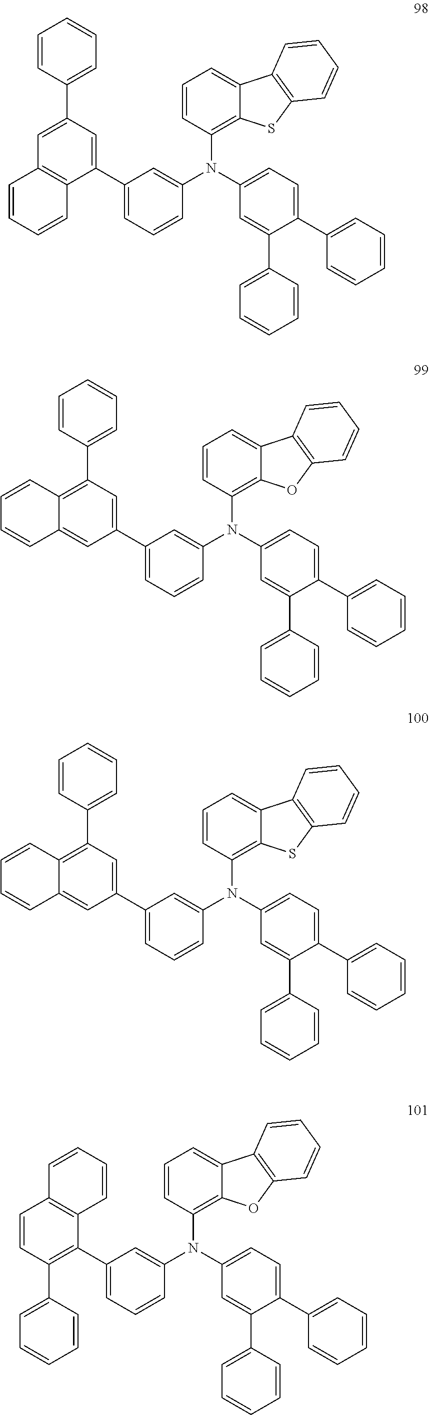 [SMILES: c1ccc(-c2cc(-c3cccc(N(c4ccc(-c5ccccc5)c(-c5ccccc5)c4)c4cccc5c4sc4ccccc45)c3)c3ccccc3c2)cc1 ; c1ccc(-c2ccc(N(c3cccc(-c4cc(-c5ccccc5)c5ccccc5c4)c3)c3cccc4c3oc3ccccc34)cc2-c2ccccc2)cc1 ; c1ccc(-c2ccc(N(c3cccc(-c4cc(-c5ccccc5)c5ccccc5c4)c3)c3cccc4c3sc3ccccc34)cc2-c2ccccc2)cc1 ; c1ccc(-c2ccc(N(c3cccc(-c4c(-c5ccccc5)ccc5ccccc45)c3)c3cccc4c3oc3ccccc34)cc2-c2ccccc2)cc1] 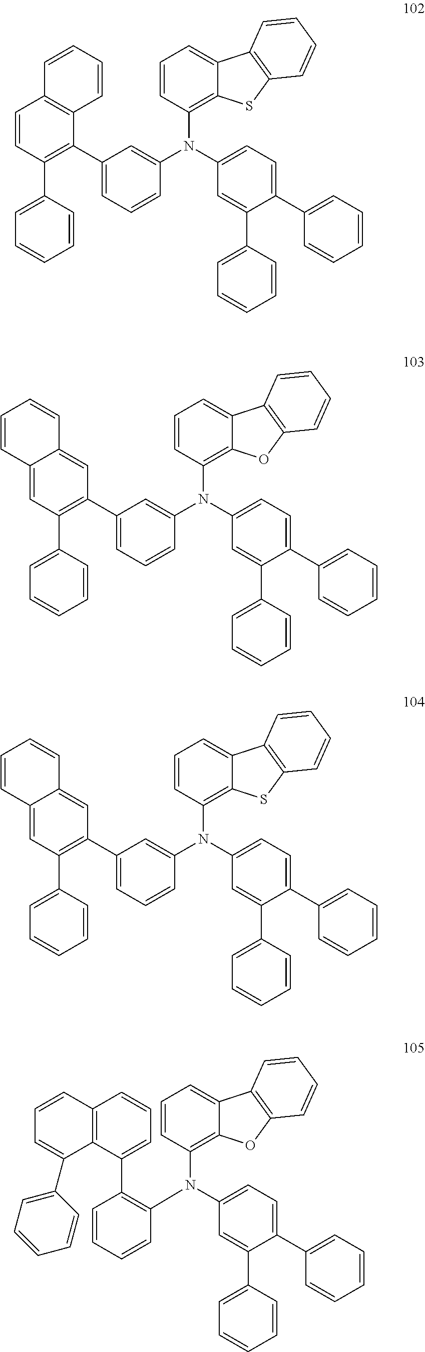 [SMILES: c1ccc(-c2ccc(N(c3cccc(-c4c(-c5ccccc5)ccc5ccccc45)c3)c3cccc4c3sc3ccccc34)cc2-c2ccccc2)cc1 ; c1ccc(-c2ccc(N(c3cccc(-c4cc5ccccc5cc4-c4ccccc4)c3)c3cccc4c3oc3ccccc34)cc2-c2ccccc2)cc1 ; c1ccc(-c2ccc(N(c3cccc(-c4cc5ccccc5cc4-c4ccccc4)c3)c3cccc4c3sc3ccccc34)cc2-c2ccccc2)cc1 ; c1ccc(-c2ccc(N(c3ccccc3-c3cccc4cccc(-c5ccccc5)c34)c3cccc4c3oc3ccccc34)cc2-c2ccccc2)cc1] 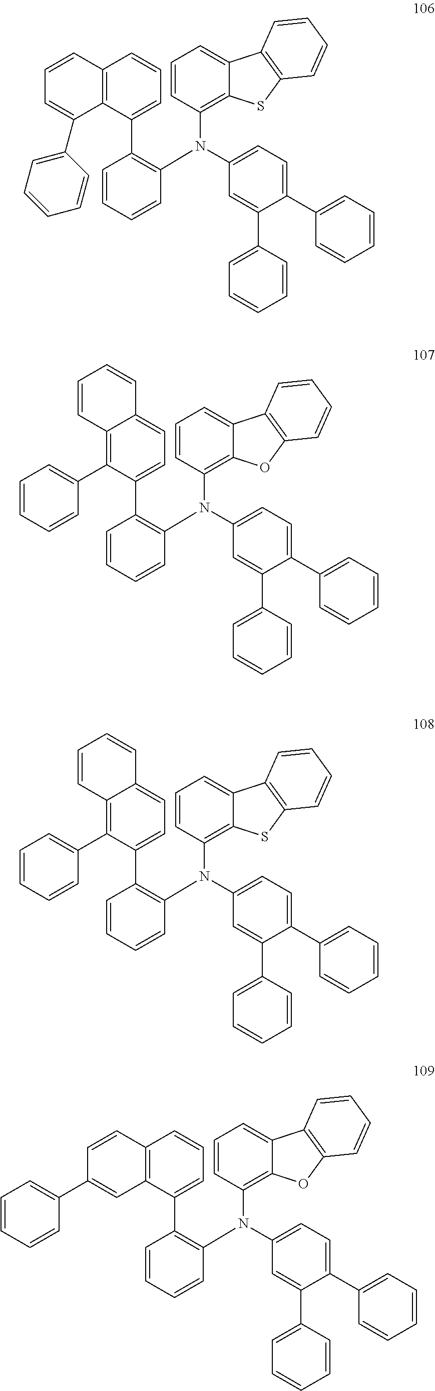 [SMILES: c1ccc(-c2ccc(N(c3ccccc3-c3cccc4cccc(-c5ccccc5)c34)c3cccc4c3sc3ccccc34)cc2-c2ccccc2)cc1 ; c1ccc(-c2ccc(N(c3ccccc3-c3ccc4ccccc4c3-c3ccccc3)c3cccc4c3oc3ccccc34)cc2-c2ccccc2)cc1 ; c1ccc(-c2ccc(N(c3ccccc3-c3ccc4ccccc4c3-c3ccccc3)c3cccc4c3sc3ccccc34)cc2-c2ccccc2)cc1 ; c1ccc(-c2ccc3cccc(-c4ccccc4N(c4ccc(-c5ccccc5)c(-c5ccccc5)c4)c4cccc5c4oc4ccccc45)c3c2)cc1] 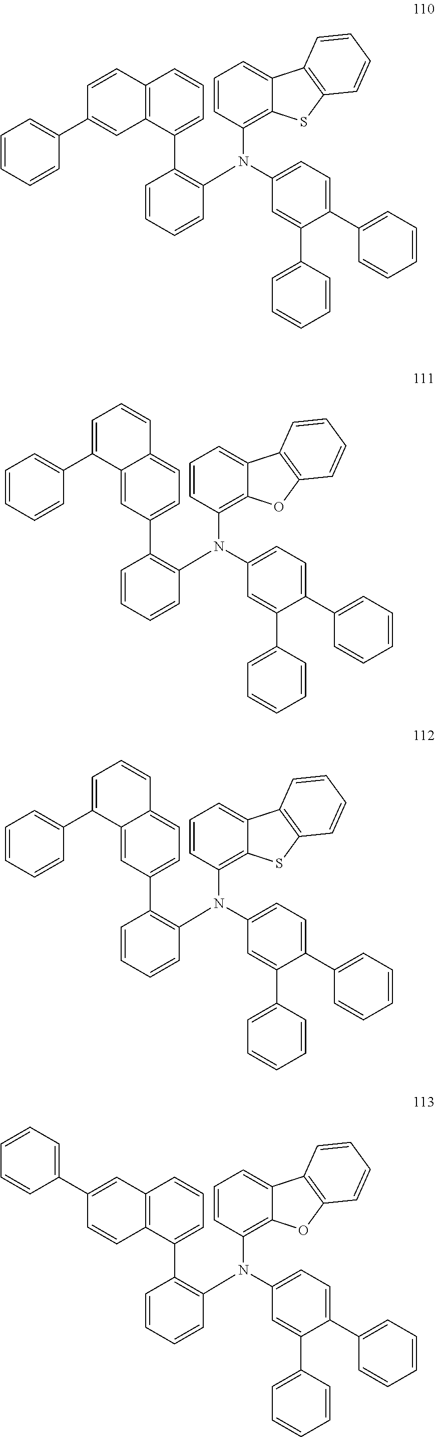 [SMILES: c1ccc(-c2ccc3cccc(-c4ccccc4N(c4ccc(-c5ccccc5)c(-c5ccccc5)c4)c4cccc5c4sc4ccccc45)c3c2)cc1 ; c1ccc(-c2ccc(N(c3ccccc3-c3ccc4cccc(-c5ccccc5)c4c3)c3cccc4c3oc3ccccc34)cc2-c2ccccc2)cc1 ; c1ccc(-c2ccc(N(c3ccccc3-c3ccc4cccc(-c5ccccc5)c4c3)c3cccc4c3sc3ccccc34)cc2-c2ccccc2)cc1 ; c1ccc(-c2ccc3c(-c4ccccc4N(c4ccc(-c5ccccc5)c(-c5ccccc5)c4)c4cccc5c4oc4ccccc45)cccc3c2)cc1] 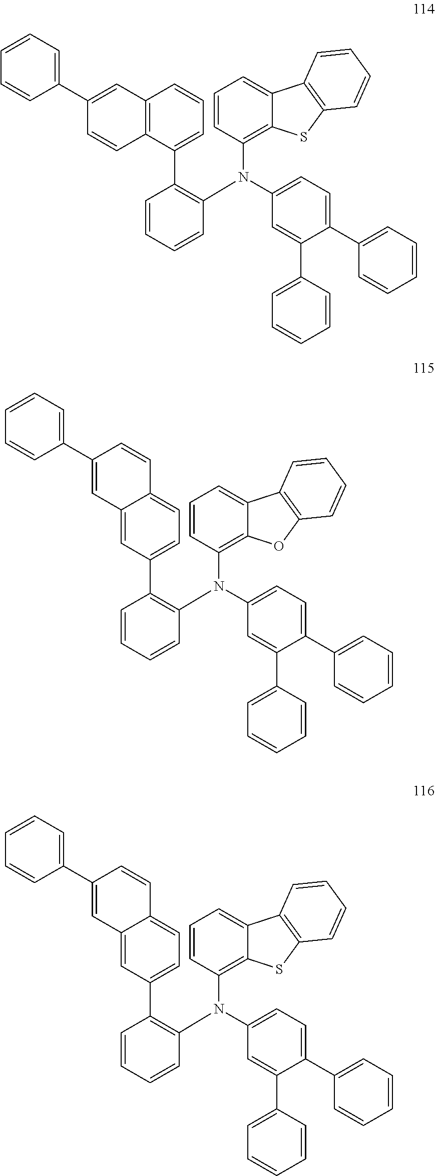 [SMILES: c1ccc(-c2ccc3c(-c4ccccc4N(c4ccc(-c5ccccc5)c(-c5ccccc5)c4)c4cccc5c4sc4ccccc45)cccc3c2)cc1 ; c1ccc(-c2ccc3ccc(-c4ccccc4N(c4ccc(-c5ccccc5)c(-c5ccccc5)c4)c4cccc5c4oc4ccccc45)cc3c2)cc1 ; c1ccc(-c2ccc3ccc(-c4ccccc4N(c4ccc(-c5ccccc5)c(-c5ccccc5)c4)c4cccc5c4sc4ccccc45)cc3c2)cc1] 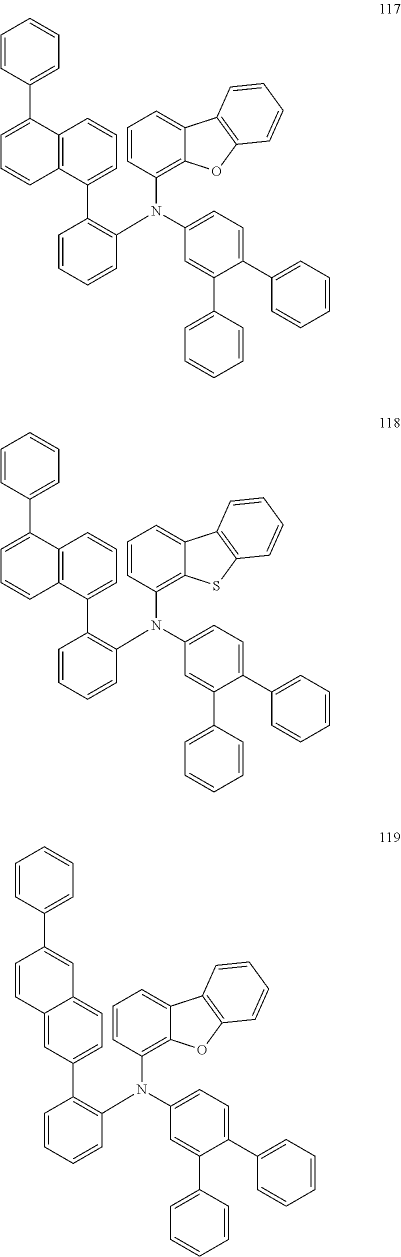 [SMILES: c1ccc(-c2ccc(N(c3ccccc3-c3cccc4c(-c5ccccc5)cccc34)c3cccc4c3oc3ccccc34)cc2-c2ccccc2)cc1 ; c1ccc(-c2ccc(N(c3ccccc3-c3cccc4c(-c5ccccc5)cccc34)c3cccc4c3sc3ccccc34)cc2-c2ccccc2)cc1 ; c1ccc(-c2ccc3cc(-c4ccccc4N(c4ccc(-c5ccccc5)c(-c5ccccc5)c4)c4cccc5c4oc4ccccc45)ccc3c2)cc1] 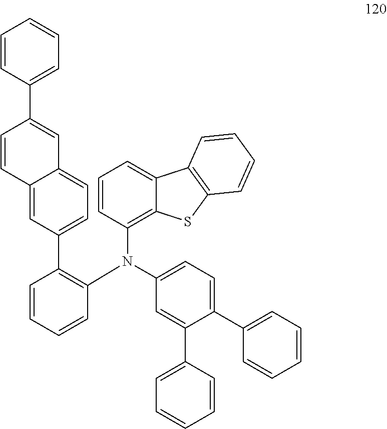 [SMILES: c1ccc(-c2ccc3cc(-c4ccccc4N(c4ccc(-c5ccccc5)c(-c5ccccc5)c4)c4cccc5c4sc4ccccc45)ccc3c2)cc1] 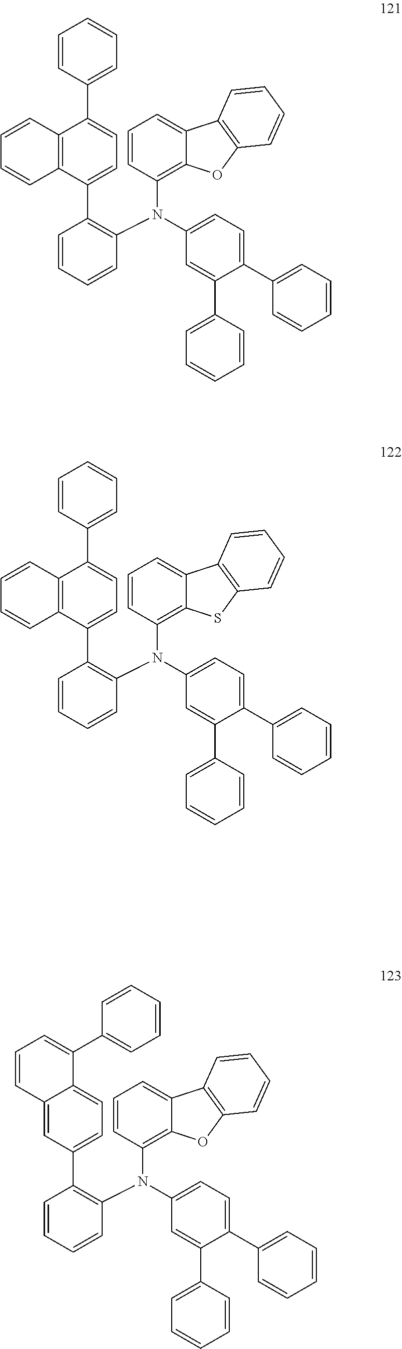 [SMILES: c1ccc(-c2ccc(N(c3ccccc3-c3ccc(-c4ccccc4)c4ccccc34)c3cccc4c3oc3ccccc34)cc2-c2ccccc2)cc1 ; c1ccc(-c2ccc(N(c3ccccc3-c3ccc(-c4ccccc4)c4ccccc34)c3cccc4c3sc3ccccc34)cc2-c2ccccc2)cc1 ; c1ccc(-c2ccc(N(c3ccccc3-c3ccc4c(-c5ccccc5)cccc4c3)c3cccc4c3oc3ccccc34)cc2-c2ccccc2)cc1] 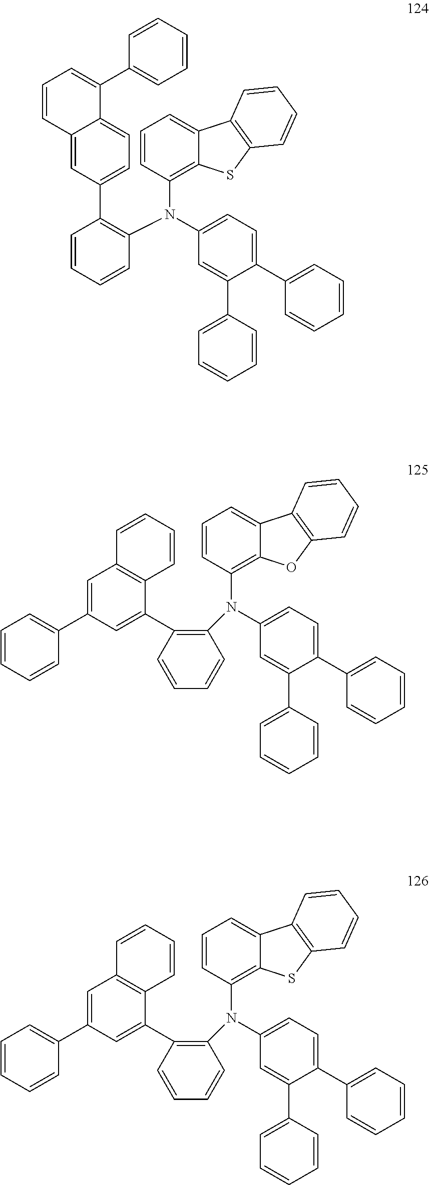 [SMILES: c1ccc(-c2ccc(N(c3ccccc3-c3ccc4c(-c5ccccc5)cccc4c3)c3cccc4c3sc3ccccc34)cc2-c2ccccc2)cc1 ; c1ccc(-c2cc(-c3ccccc3N(c3ccc(-c4ccccc4)c(-c4ccccc4)c3)c3cccc4c3oc3ccccc34)c3ccccc3c2)cc1 ; c1ccc(-c2cc(-c3ccccc3N(c3ccc(-c4ccccc4)c(-c4ccccc4)c3)c3cccc4c3sc3ccccc34)c3ccccc3c2)cc1] 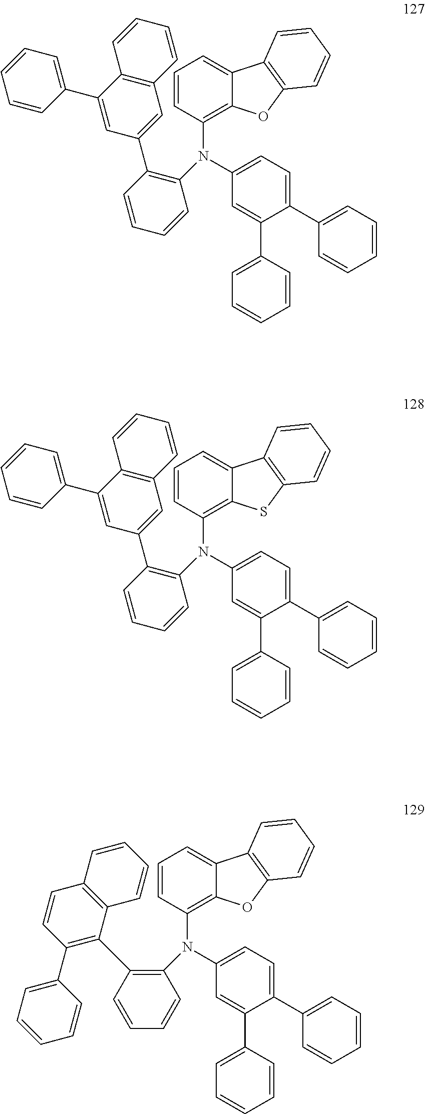 [SMILES: c1ccc(-c2ccc(N(c3ccccc3-c3cc(-c4ccccc4)c4ccccc4c3)c3cccc4c3oc3ccccc34)cc2-c2ccccc2)cc1 ; c1ccc(-c2ccc(N(c3ccccc3-c3cc(-c4ccccc4)c4ccccc4c3)c3cccc4c3sc3ccccc34)cc2-c2ccccc2)cc1 ; c1ccc(-c2ccc(N(c3ccccc3-c3c(-c4ccccc4)ccc4ccccc34)c3cccc4c3oc3ccccc34)cc2-c2ccccc2)cc1] 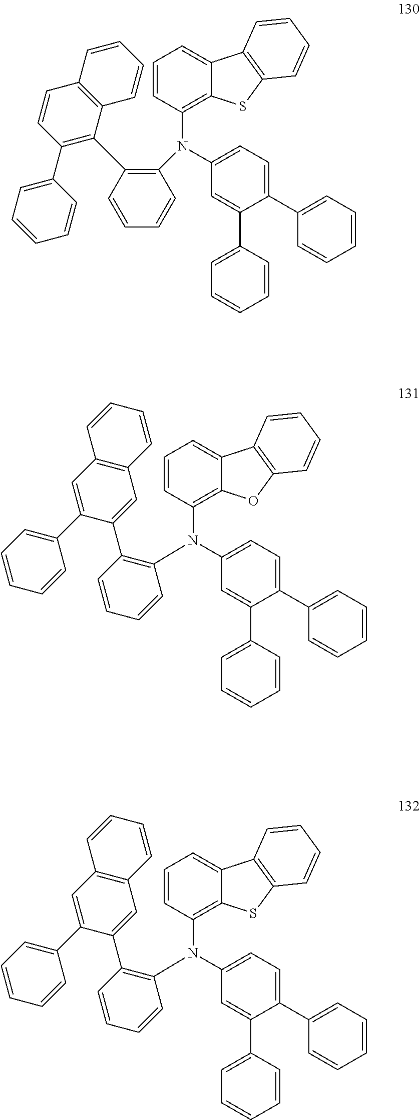 [SMILES: c1ccc(-c2ccc(N(c3ccccc3-c3c(-c4ccccc4)ccc4ccccc34)c3cccc4c3sc3ccccc34)cc2-c2ccccc2)cc1 ; c1ccc(-c2ccc(N(c3ccccc3-c3cc4ccccc4cc3-c3ccccc3)c3cccc4c3oc3ccccc34)cc2-c2ccccc2)cc1 ; c1ccc(-c2ccc(N(c3ccccc3-c3cc4ccccc4cc3-c3ccccc3)c3cccc4c3sc3ccccc34)cc2-c2ccccc2)cc1] 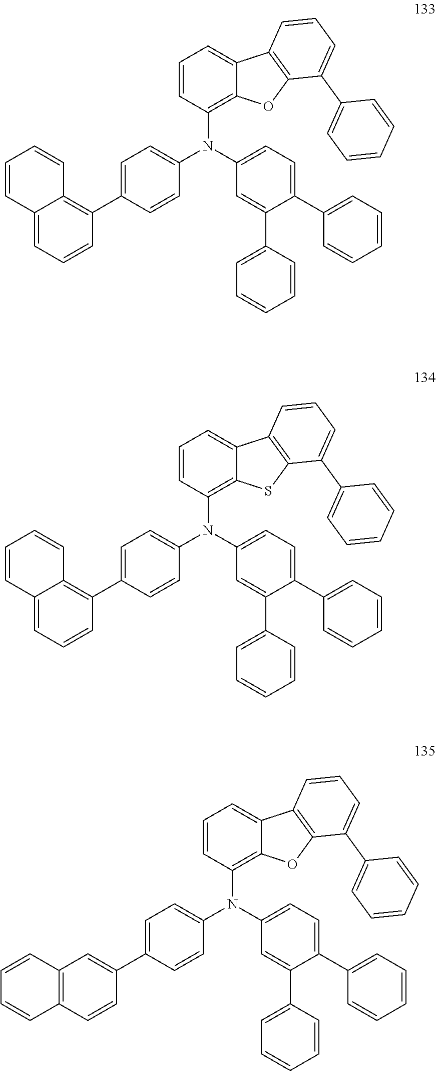 [SMILES: c1ccc(-c2ccc(N(c3ccc(-c4cccc5ccccc45)cc3)c3cccc4c3oc3c(-c5ccccc5)cccc34)cc2-c2ccccc2)cc1 ; c1ccc(-c2ccc(N(c3ccc(-c4cccc5ccccc45)cc3)c3cccc4c3sc3c(-c5ccccc5)cccc34)cc2-c2ccccc2)cc1 ; c1ccc(-c2ccc(N(c3ccc(-c4ccc5ccccc5c4)cc3)c3cccc4c3oc3c(-c5ccccc5)cccc34)cc2-c2ccccc2)cc1] 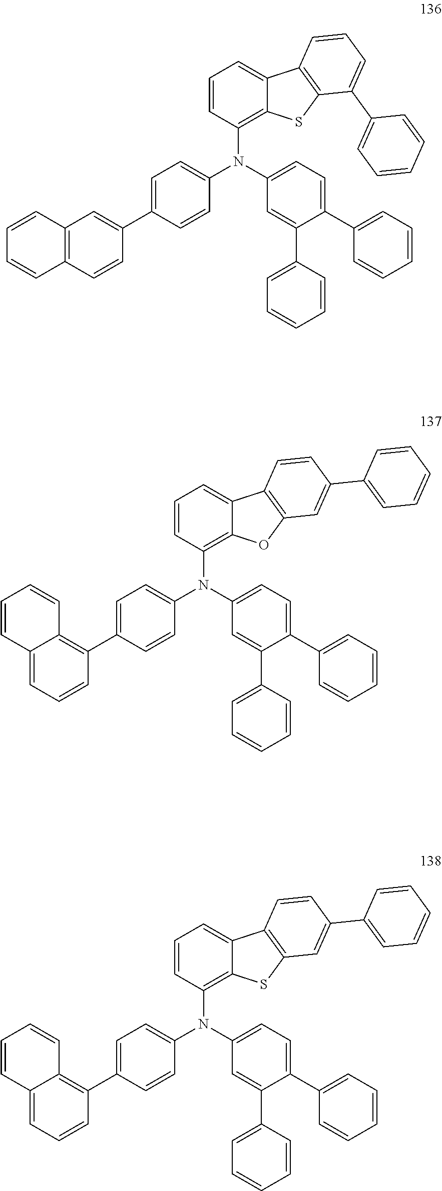 [SMILES: c1ccc(-c2ccc(N(c3ccc(-c4ccc5ccccc5c4)cc3)c3cccc4c3sc3c(-c5ccccc5)cccc34)cc2-c2ccccc2)cc1 ; c1ccc(-c2ccc3c(c2)oc2c(N(c4ccc(-c5cccc6ccccc56)cc4)c4ccc(-c5ccccc5)c(-c5ccccc5)c4)cccc23)cc1 ; c1ccc(-c2ccc3c(c2)sc2c(N(c4ccc(-c5cccc6ccccc56)cc4)c4ccc(-c5ccccc5)c(-c5ccccc5)c4)cccc23)cc1] 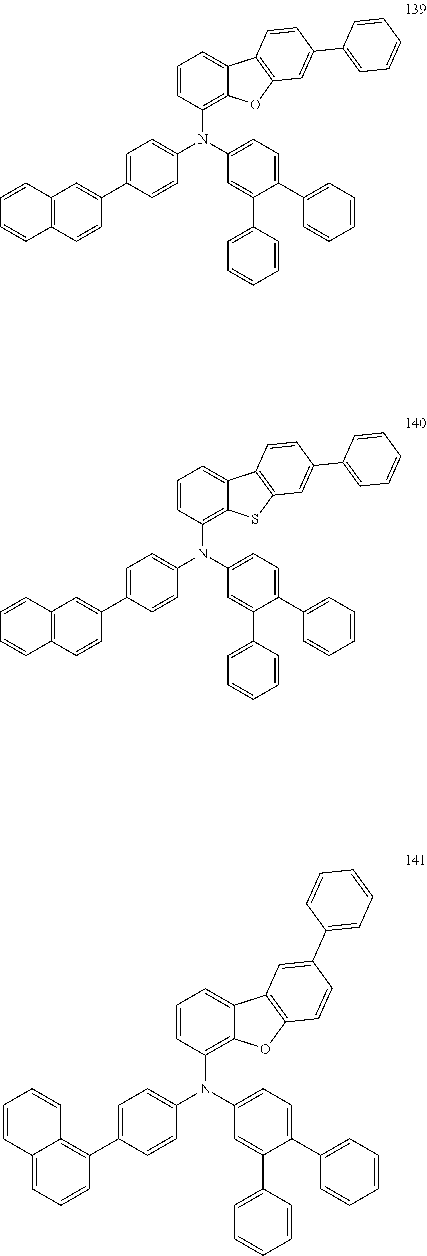 [SMILES: c1ccc(-c2ccc3c(c2)oc2c(N(c4ccc(-c5ccc6ccccc6c5)cc4)c4ccc(-c5ccccc5)c(-c5ccccc5)c4)cccc23)cc1 ; c1ccc(-c2ccc3c(c2)sc2c(N(c4ccc(-c5ccc6ccccc6c5)cc4)c4ccc(-c5ccccc5)c(-c5ccccc5)c4)cccc23)cc1 ; c1ccc(-c2ccc3oc4c(N(c5ccc(-c6cccc7ccccc67)cc5)c5ccc(-c6ccccc6)c(-c6ccccc6)c5)cccc4c3c2)cc1] 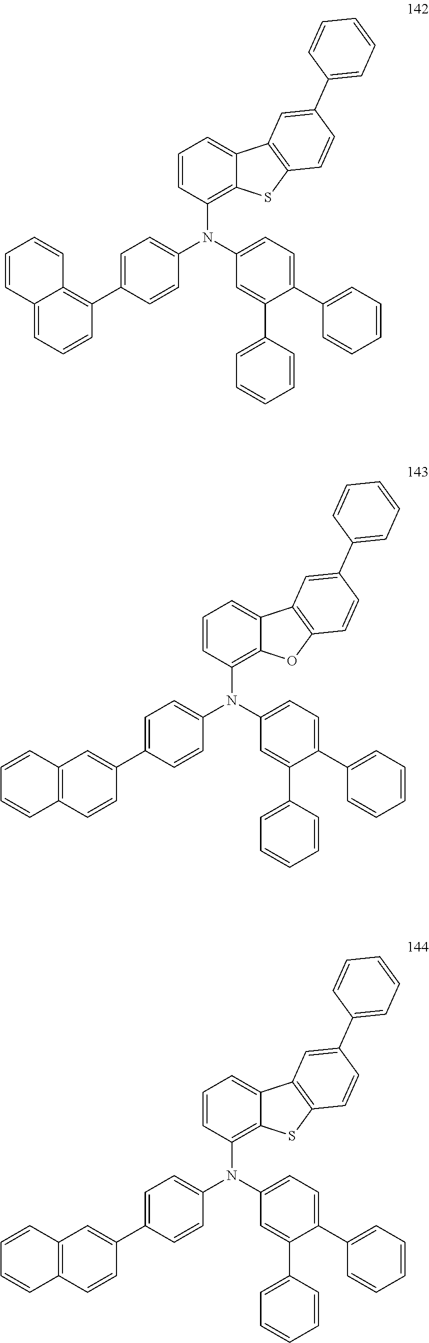 [SMILES: c1ccc(-c2ccc3sc4c(N(c5ccc(-c6cccc7ccccc67)cc5)c5ccc(-c6ccccc6)c(-c6ccccc6)c5)cccc4c3c2)cc1 ; c1ccc(-c2ccc3oc4c(N(c5ccc(-c6ccc7ccccc7c6)cc5)c5ccc(-c6ccccc6)c(-c6ccccc6)c5)cccc4c3c2)cc1 ; c1ccc(-c2ccc3sc4c(N(c5ccc(-c6ccc7ccccc7c6)cc5)c5ccc(-c6ccccc6)c(-c6ccccc6)c5)cccc4c3c2)cc1] 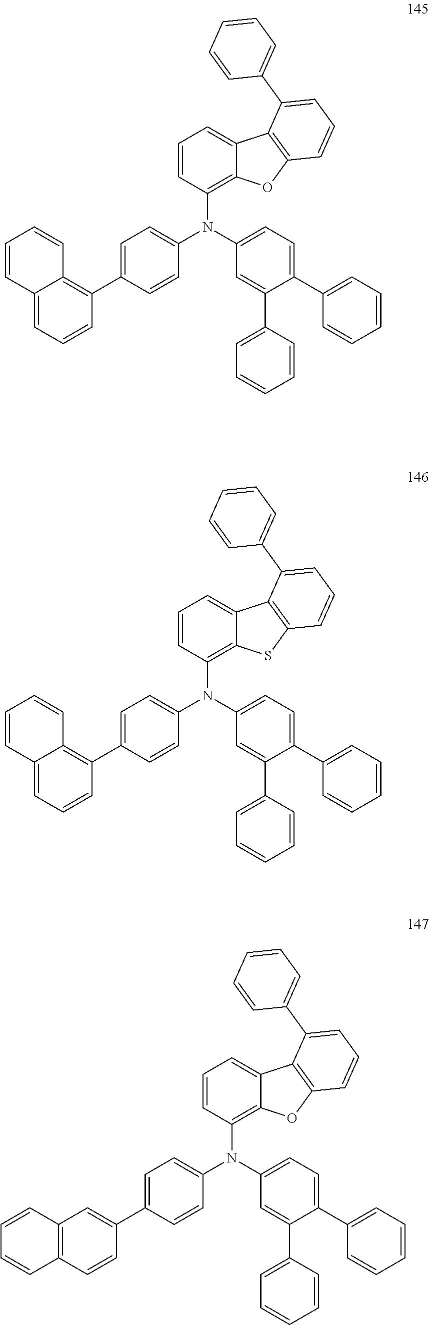 [SMILES: c1ccc(-c2ccc(N(c3ccc(-c4cccc5ccccc45)cc3)c3cccc4c3oc3cccc(-c5ccccc5)c34)cc2-c2ccccc2)cc1 ; c1ccc(-c2ccc(N(c3ccc(-c4cccc5ccccc45)cc3)c3cccc4c3sc3cccc(-c5ccccc5)c34)cc2-c2ccccc2)cc1 ; c1ccc(-c2ccc(N(c3ccc(-c4ccc5ccccc5c4)cc3)c3cccc4c3oc3cccc(-c5ccccc5)c34)cc2-c2ccccc2)cc1] 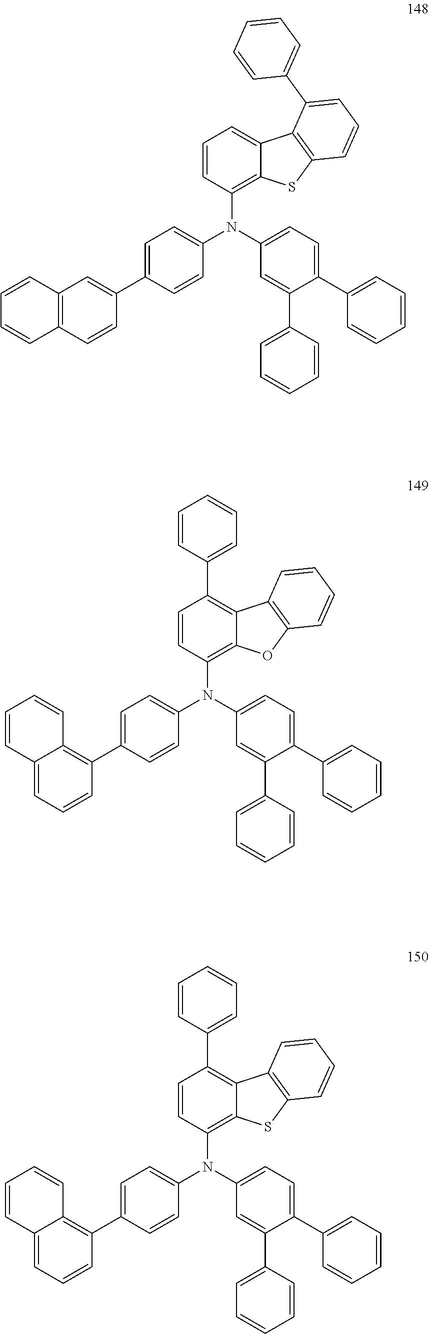 [SMILES: c1ccc(-c2ccc(N(c3ccc(-c4ccc5ccccc5c4)cc3)c3cccc4c3sc3cccc(-c5ccccc5)c34)cc2-c2ccccc2)cc1 ; c1ccc(-c2ccc(N(c3ccc(-c4cccc5ccccc45)cc3)c3ccc(-c4ccccc4)c4c3oc3ccccc34)cc2-c2ccccc2)cc1 ; c1ccc(-c2ccc(N(c3ccc(-c4cccc5ccccc45)cc3)c3ccc(-c4ccccc4)c4c3sc3ccccc34)cc2-c2ccccc2)cc1] 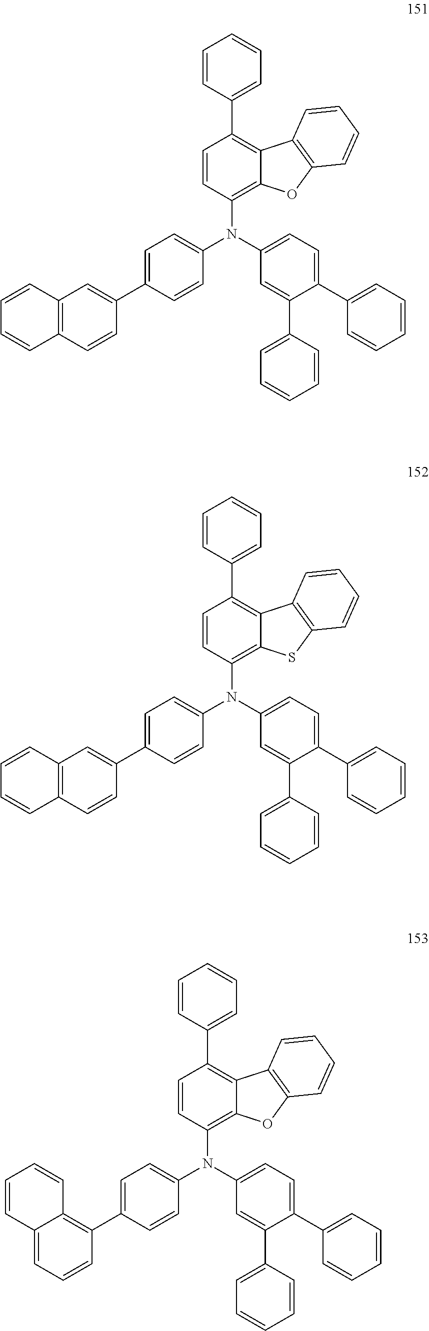 [SMILES: c1ccc(-c2ccc(N(c3ccc(-c4ccc5ccccc5c4)cc3)c3ccc(-c4ccccc4)c4c3oc3ccccc34)cc2-c2ccccc2)cc1 ; c1ccc(-c2ccc(N(c3ccc(-c4ccc5ccccc5c4)cc3)c3ccc(-c4ccccc4)c4c3sc3ccccc34)cc2-c2ccccc2)cc1 ; c1ccc(-c2ccc(N(c3ccc(-c4cccc5ccccc45)cc3)c3ccc(-c4ccccc4)c4c3oc3ccccc34)cc2-c2ccccc2)cc1] 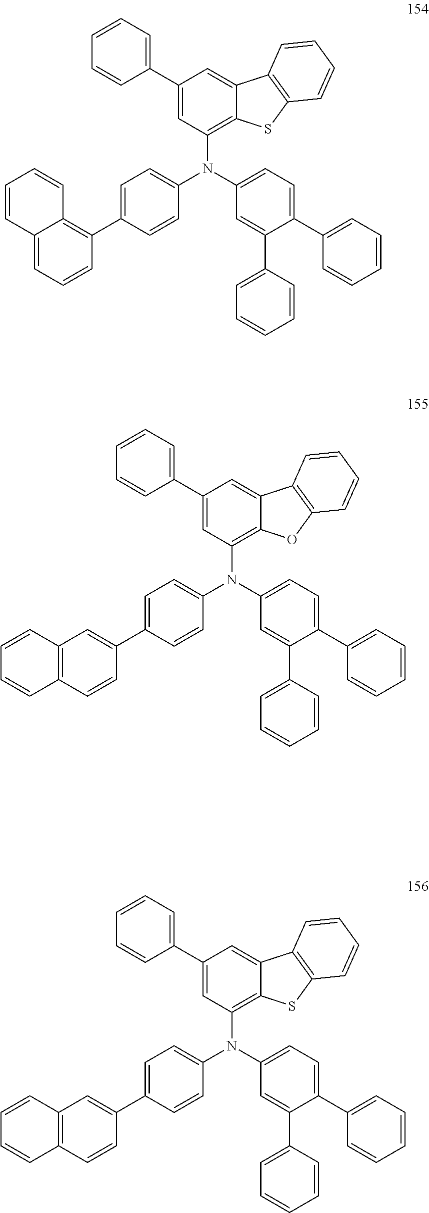 [SMILES: c1ccc(-c2cc(N(c3ccc(-c4cccc5ccccc45)cc3)c3ccc(-c4ccccc4)c(-c4ccccc4)c3)c3sc4ccccc4c3c2)cc1 ; c1ccc(-c2cc(N(c3ccc(-c4ccc5ccccc5c4)cc3)c3ccc(-c4ccccc4)c(-c4ccccc4)c3)c3oc4ccccc4c3c2)cc1 ; c1ccc(-c2cc(N(c3ccc(-c4ccc5ccccc5c4)cc3)c3ccc(-c4ccccc4)c(-c4ccccc4)c3)c3sc4ccccc4c3c2)cc1] 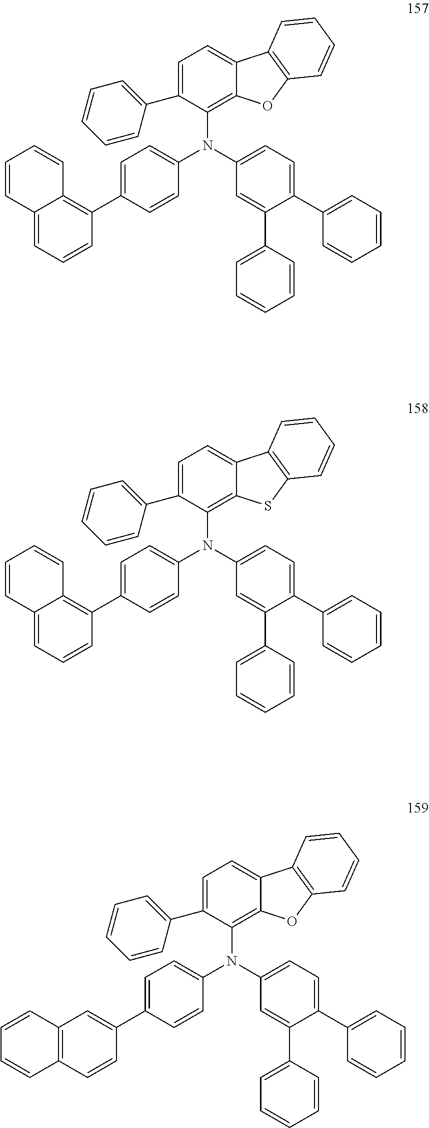 [SMILES: c1ccc(-c2ccc(N(c3ccc(-c4cccc5ccccc45)cc3)c3c(-c4ccccc4)ccc4c3oc3ccccc34)cc2-c2ccccc2)cc1 ; c1ccc(-c2ccc(N(c3ccc(-c4cccc5ccccc45)cc3)c3c(-c4ccccc4)ccc4c3sc3ccccc34)cc2-c2ccccc2)cc1 ; c1ccc(-c2ccc(N(c3ccc(-c4ccc5ccccc5c4)cc3)c3c(-c4ccccc4)ccc4c3oc3ccccc34)cc2-c2ccccc2)cc1] 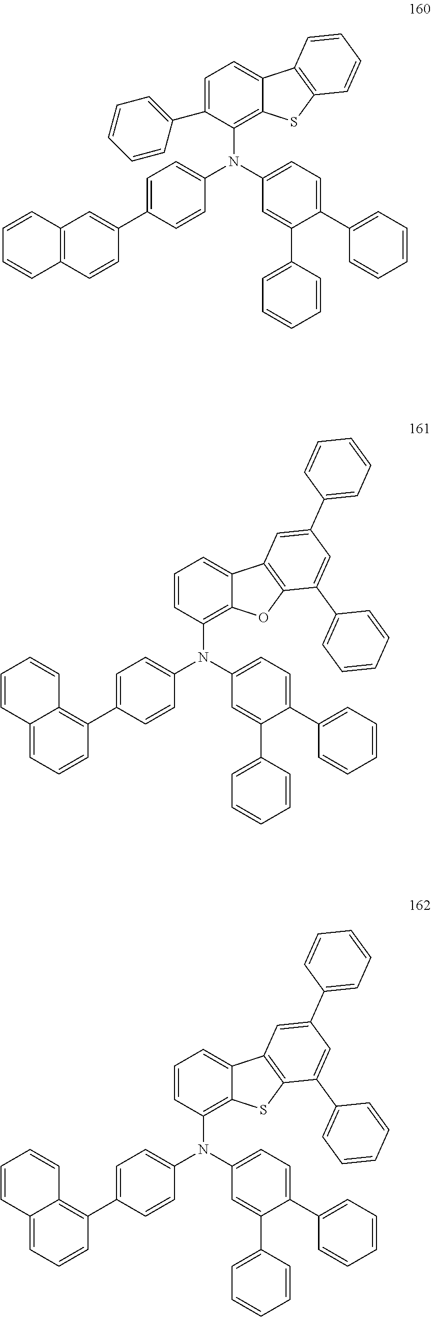 [SMILES: c1ccc(-c2ccc(N(c3ccc(-c4ccc5ccccc5c4)cc3)c3c(-c4ccccc4)ccc4c3sc3ccccc34)cc2-c2ccccc2)cc1 ; c1ccc(-c2cc(-c3ccccc3)c3oc4c(N(c5ccc(-c6cccc7ccccc67)cc5)c5ccc(-c6ccccc6)c(-c6ccccc6)c5)cccc4c3c2)cc1 ; c1ccc(-c2cc(-c3ccccc3)c3sc4c(N(c5ccc(-c6cccc7ccccc67)cc5)c5ccc(-c6ccccc6)c(-c6ccccc6)c5)cccc4c3c2)cc1] 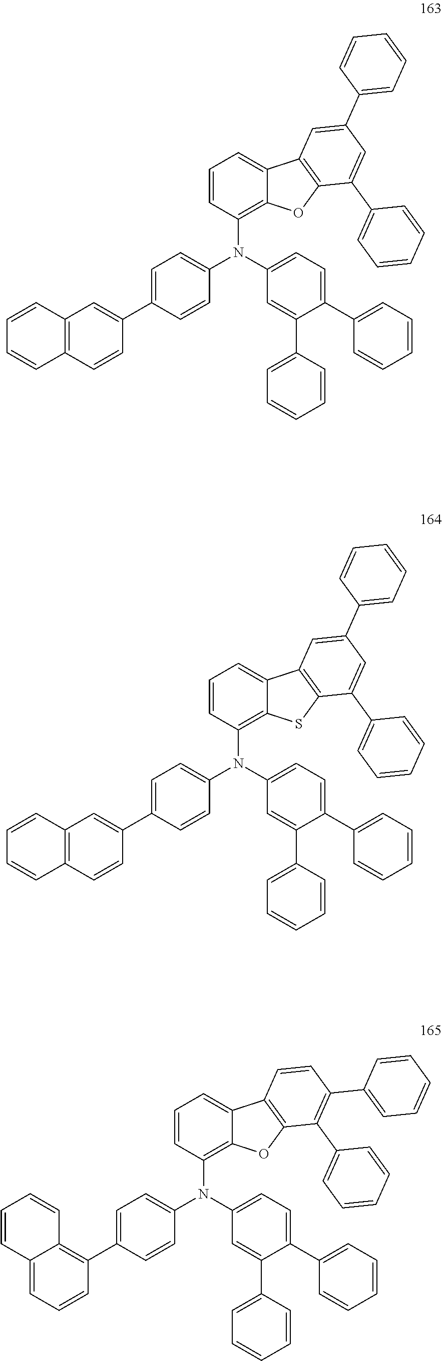 [SMILES: c1ccc(-c2cc(-c3ccccc3)c3oc4c(N(c5ccc(-c6ccc7ccccc7c6)cc5)c5ccc(-c6ccccc6)c(-c6ccccc6)c5)cccc4c3c2)cc1 ; c1ccc(-c2cc(-c3ccccc3)c3sc4c(N(c5ccc(-c6ccc7ccccc7c6)cc5)c5ccc(-c6ccccc6)c(-c6ccccc6)c5)cccc4c3c2)cc1 ; c1ccc(-c2ccc(N(c3ccc(-c4cccc5ccccc45)cc3)c3cccc4c3oc3c(-c5ccccc5)c(-c5ccccc5)ccc34)cc2-c2ccccc2)cc1] 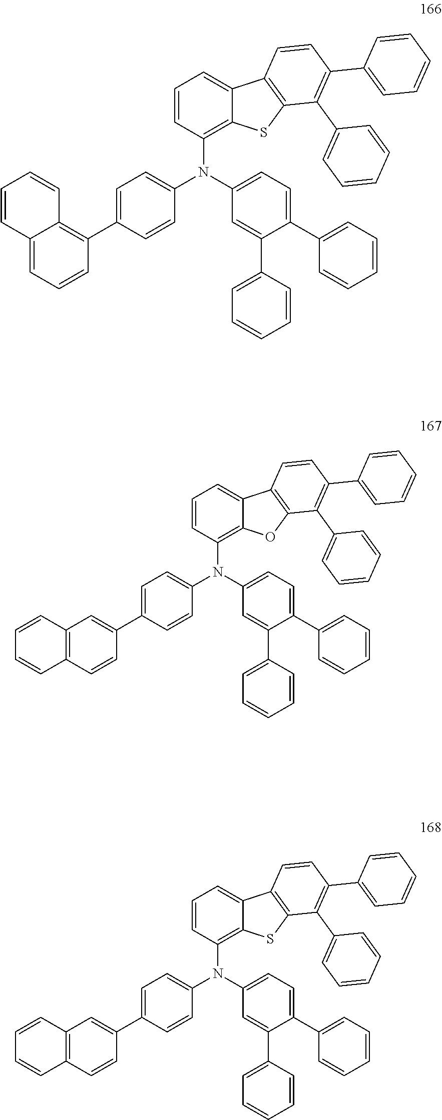 [SMILES: c1ccc(-c2ccc(N(c3ccc(-c4cccc5ccccc45)cc3)c3cccc4c3sc3c(-c5ccccc5)c(-c5ccccc5)ccc34)cc2-c2ccccc2)cc1 ; c1ccc(-c2ccc(N(c3ccc(-c4ccc5ccccc5c4)cc3)c3cccc4c3oc3c(-c5ccccc5)c(-c5ccccc5)ccc34)cc2-c2ccccc2)cc1 ; c1ccc(-c2ccc(N(c3ccc(-c4ccc5ccccc5c4)cc3)c3cccc4c3sc3c(-c5ccccc5)c(-c5ccccc5)ccc34)cc2-c2ccccc2)cc1] 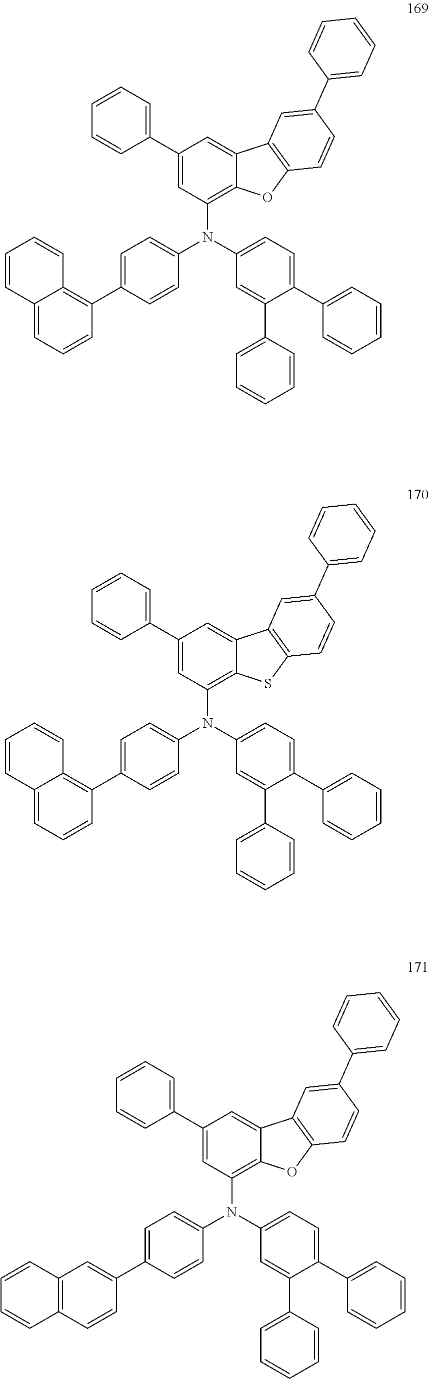 [SMILES: c1ccc(-c2ccc3oc4c(N(c5ccc(-c6cccc7ccccc67)cc5)c5ccc(-c6ccccc6)c(-c6ccccc6)c5)cc(-c5ccccc5)cc4c3c2)cc1 ; c1ccc(-c2ccc3sc4c(N(c5ccc(-c6cccc7ccccc67)cc5)c5ccc(-c6ccccc6)c(-c6ccccc6)c5)cc(-c5ccccc5)cc4c3c2)cc1 ; c1ccc(-c2ccc3oc4c(N(c5ccc(-c6ccc7ccccc7c6)cc5)c5ccc(-c6ccccc6)c(-c6ccccc6)c5)cc(-c5ccccc5)cc4c3c2)cc1] 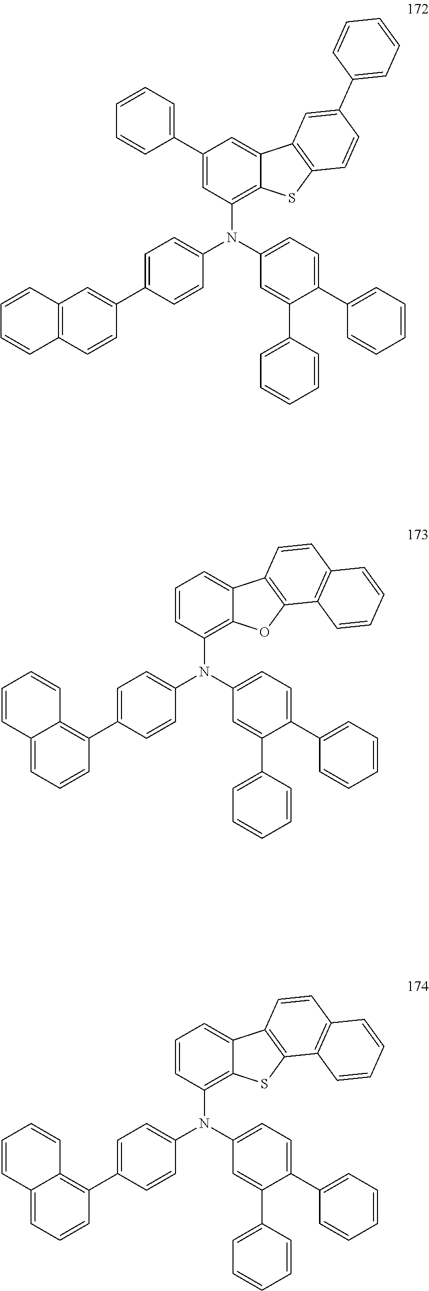 [SMILES: c1ccc(-c2ccc3sc4c(N(c5ccc(-c6ccc7ccccc7c6)cc5)c5ccc(-c6ccccc6)c(-c6ccccc6)c5)cc(-c5ccccc5)cc4c3c2)cc1 ; c1ccc(-c2ccc(N(c3ccc(-c4cccc5ccccc45)cc3)c3cccc4c3oc3c5ccccc5ccc43)cc2-c2ccccc2)cc1 ; c1ccc(-c2ccc(N(c3ccc(-c4cccc5ccccc45)cc3)c3cccc4c3sc3c5ccccc5ccc43)cc2-c2ccccc2)cc1] 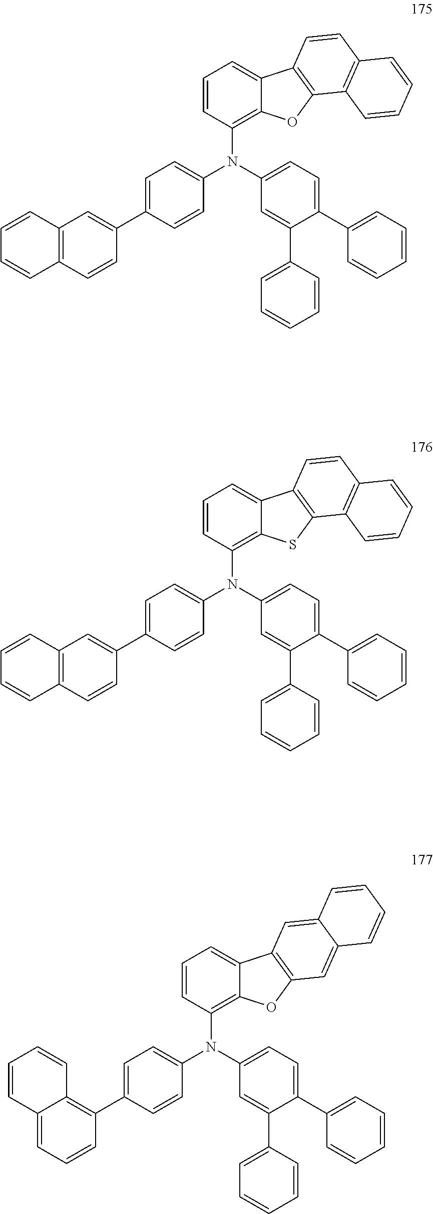 [SMILES: c1ccc(-c2ccc(N(c3ccc(-c4ccc5ccccc5c4)cc3)c3cccc4c3oc3c5ccccc5ccc43)cc2-c2ccccc2)cc1 ; c1ccc(-c2ccc(N(c3ccc(-c4ccc5ccccc5c4)cc3)c3cccc4c3sc3c5ccccc5ccc43)cc2-c2ccccc2)cc1 ; c1ccc(-c2ccc(N(c3ccc(-c4cccc5ccccc45)cc3)c3cccc4c3oc3cc5ccccc5cc34)cc2-c2ccccc2)cc1] 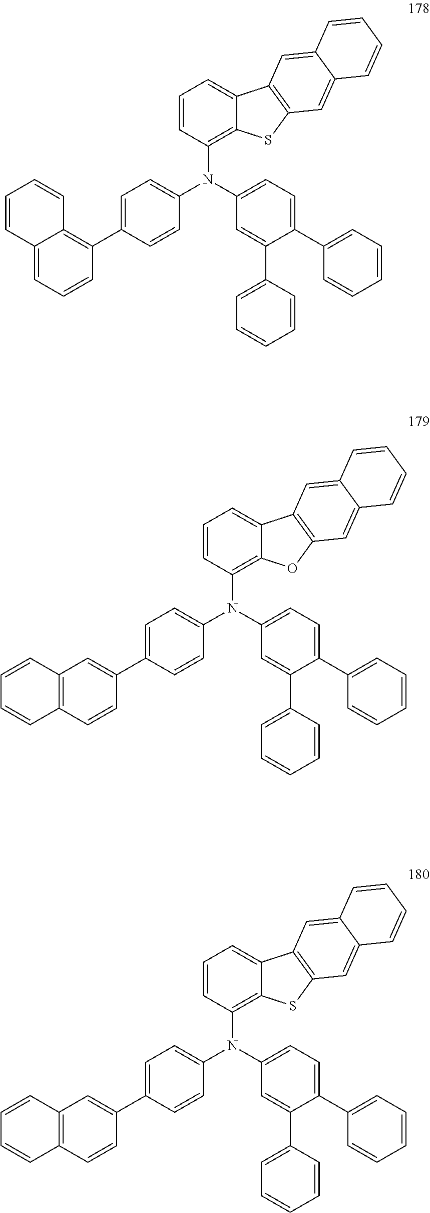 [SMILES: c1ccc(-c2ccc(N(c3ccc(-c4cccc5ccccc45)cc3)c3cccc4c3sc3cc5ccccc5cc34)cc2-c2ccccc2)cc1 ; c1ccc(-c2ccc(N(c3ccc(-c4ccc5ccccc5c4)cc3)c3cccc4c3oc3cc5ccccc5cc34)cc2-c2ccccc2)cc1 ; c1ccc(-c2ccc(N(c3ccc(-c4ccc5ccccc5c4)cc3)c3cccc4c3sc3cc5ccccc5cc34)cc2-c2ccccc2)cc1] 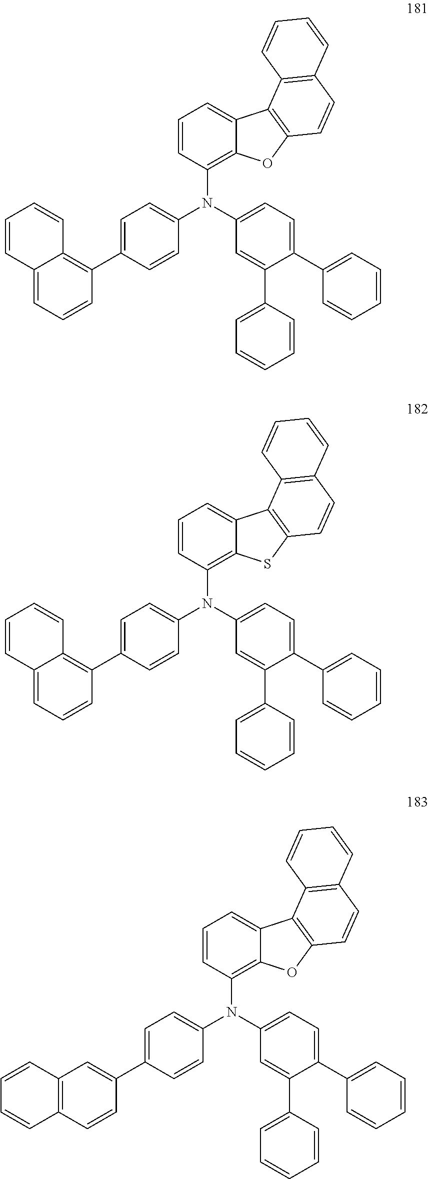 [SMILES: c1ccc(-c2ccc(N(c3ccc(-c4cccc5ccccc45)cc3)c3cccc4c3oc3ccc5ccccc5c34)cc2-c2ccccc2)cc1 ; c1ccc(-c2ccc(N(c3ccc(-c4cccc5ccccc45)cc3)c3cccc4c3sc3ccc5ccccc5c34)cc2-c2ccccc2)cc1 ; c1ccc(-c2ccc(N(c3ccc(-c4ccc5ccccc5c4)cc3)c3cccc4c3oc3ccc5ccccc5c34)cc2-c2ccccc2)cc1] 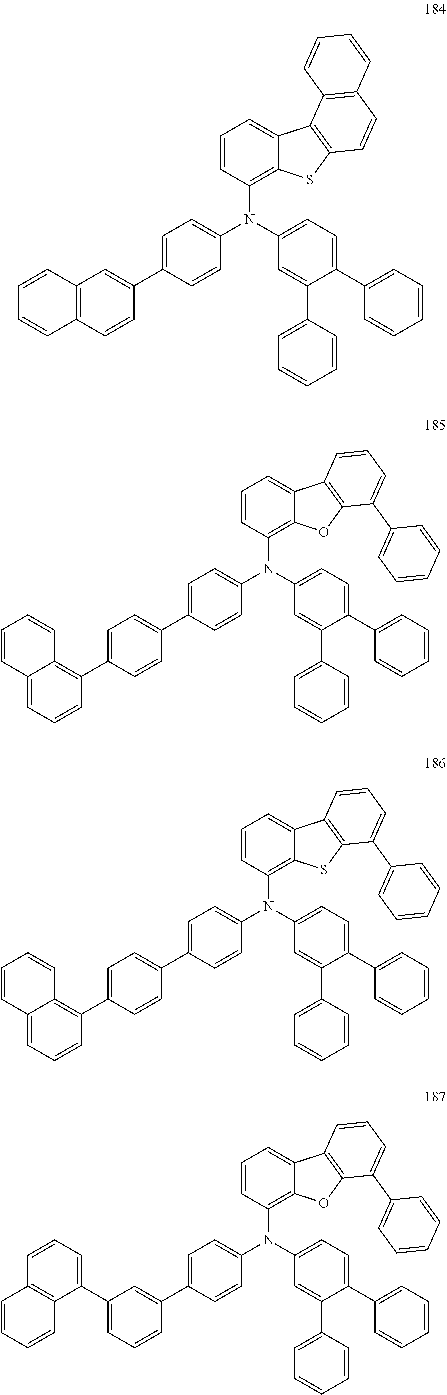 [SMILES: c1ccc(-c2ccc(N(c3ccc(-c4ccc5ccccc5c4)cc3)c3cccc4c3sc3ccc5ccccc5c34)cc2-c2ccccc2)cc1 ; c1ccc(-c2ccc(N(c3ccc(-c4ccc(-c5cccc6ccccc56)cc4)cc3)c3cccc4c3oc3c(-c5ccccc5)cccc34)cc2-c2ccccc2)cc1 ; c1ccc(-c2ccc(N(c3ccc(-c4ccc(-c5cccc6ccccc56)cc4)cc3)c3cccc4c3sc3c(-c5ccccc5)cccc34)cc2-c2ccccc2)cc1 ; c1ccc(-c2ccc(N(c3ccc(-c4cccc(-c5cccc6ccccc56)c4)cc3)c3cccc4c3oc3c(-c5ccccc5)cccc34)cc2-c2ccccc2)cc1] 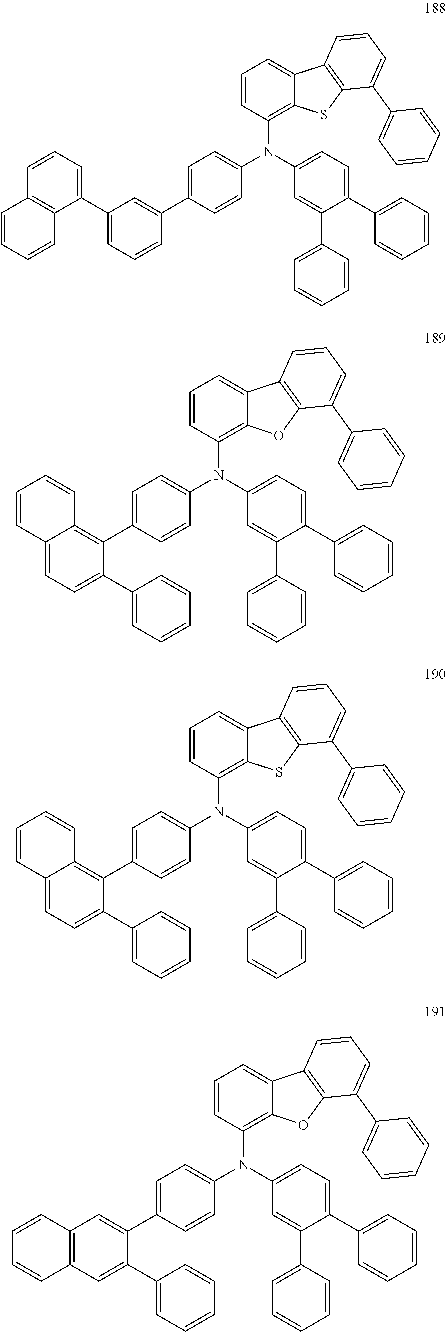 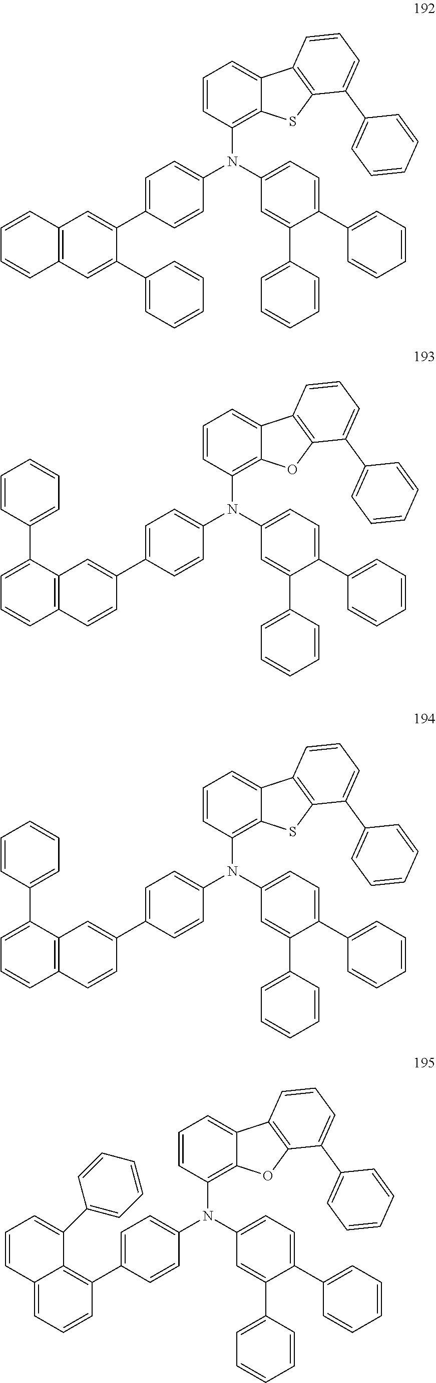 [SMILES: c1ccc(-c2ccc(N(c3ccc(-c4cc5ccccc5cc4-c4ccccc4)cc3)c3cccc4c3sc3c(-c5ccccc5)cccc34)cc2-c2ccccc2)cc1 ; c1ccc(-c2ccc(N(c3ccc(-c4ccc5cccc(-c6ccccc6)c5c4)cc3)c3cccc4c3oc3c(-c5ccccc5)cccc34)cc2-c2ccccc2)cc1 ; c1ccc(-c2ccc(N(c3ccc(-c4ccc5cccc(-c6ccccc6)c5c4)cc3)c3cccc4c3sc3c(-c5ccccc5)cccc34)cc2-c2ccccc2)cc1 ; c1ccc(-c2ccc(N(c3ccc(-c4cccc5cccc(-c6ccccc6)c45)cc3)c3cccc4c3oc3c(-c5ccccc5)cccc34)cc2-c2ccccc2)cc1] 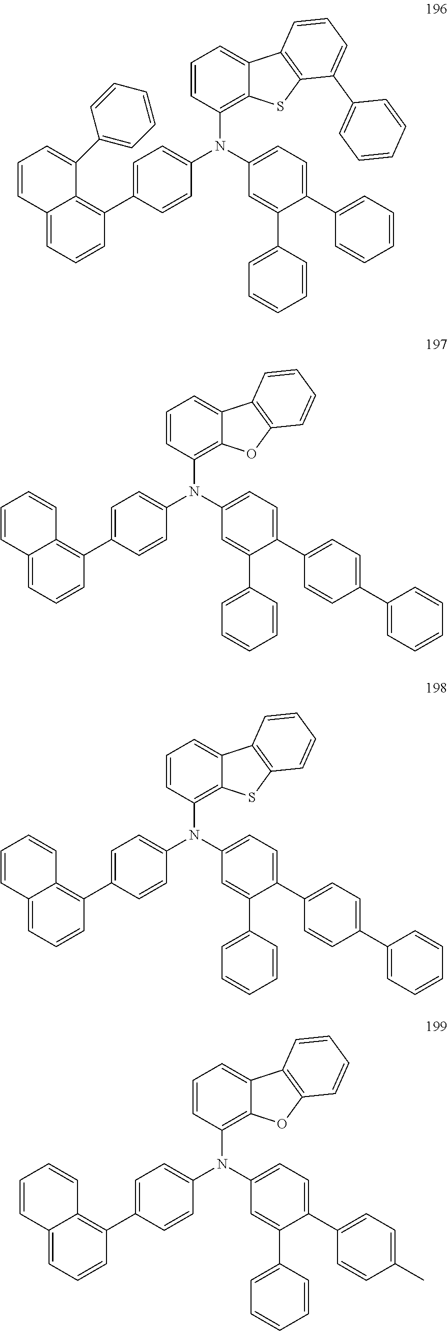 [SMILES: c1ccc(-c2ccc(N(c3ccc(-c4cccc5cccc(-c6ccccc6)c45)cc3)c3cccc4c3sc3c(-c5ccccc5)cccc34)cc2-c2ccccc2)cc1 ; c1ccc(-c2ccc(-c3ccc(N(c4ccc(-c5cccc6ccccc56)cc4)c4cccc5c4oc4ccccc45)cc3-c3ccccc3)cc2)cc1 ; c1ccc(-c2ccc(-c3ccc(N(c4ccc(-c5cccc6ccccc56)cc4)c4cccc5c4sc4ccccc45)cc3-c3ccccc3)cc2)cc1 ; Cc1ccc(-c2ccc(N(c3ccc(-c4cccc5ccccc45)cc3)c3cccc4c3oc3ccccc34)cc2-c2ccccc2)cc1] 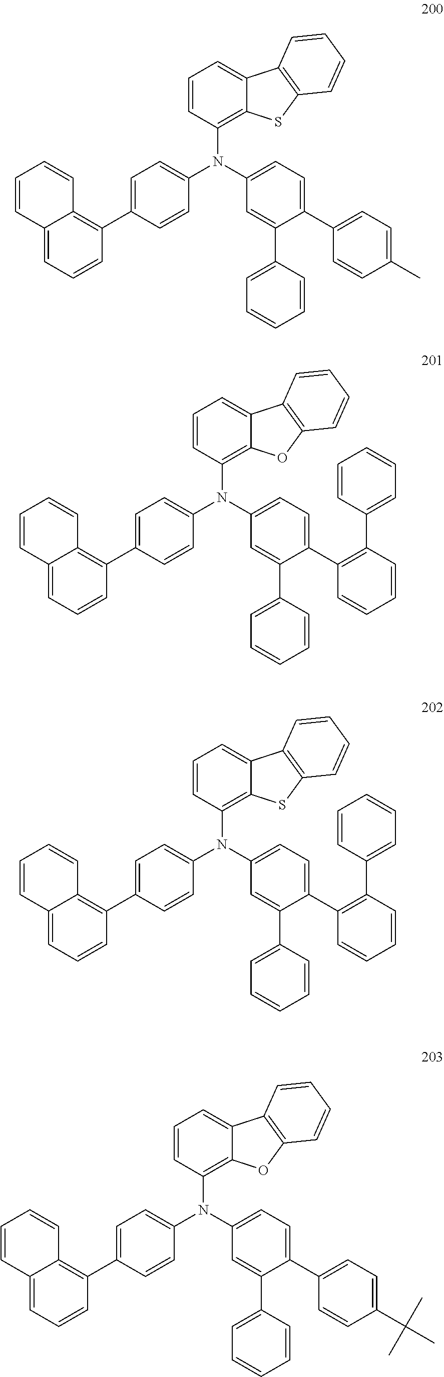 [SMILES: Cc1ccc(-c2ccc(N(c3ccc(-c4cccc5ccccc45)cc3)c3cccc4c3sc3ccccc34)cc2-c2ccccc2)cc1 ; c1ccc(-c2ccccc2-c2ccc(N(c3ccc(-c4cccc5ccccc45)cc3)c3cccc4c3oc3ccccc34)cc2-c2ccccc2)cc1 ; c1ccc(-c2ccccc2-c2ccc(N(c3ccc(-c4cccc5ccccc45)cc3)c3cccc4c3sc3ccccc34)cc2-c2ccccc2)cc1 ; CC(C)(C)c1ccc(-c2ccc(N(c3ccc(-c4cccc5ccccc45)cc3)c3cccc4c3oc3ccccc34)cc2-c2ccccc2)cc1] 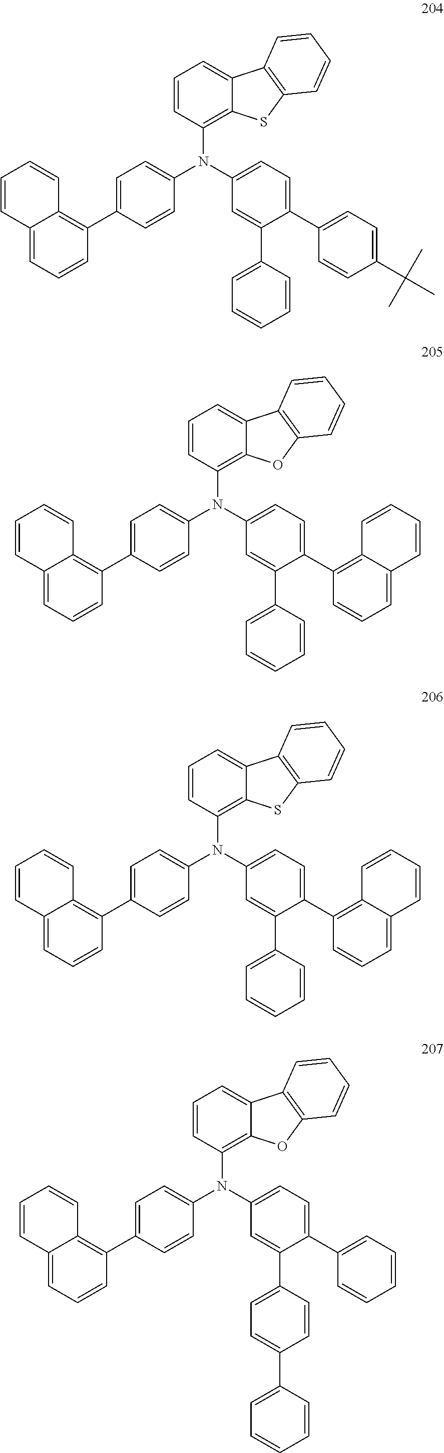 [SMILES: CC(C)(C)c1ccc(-c2ccc(N(c3ccc(-c4cccc5ccccc45)cc3)c3cccc4c3sc3ccccc34)cc2-c2ccccc2)cc1 ; c1ccc(-c2cc(N(c3ccc(-c4cccc5ccccc45)cc3)c3cccc4c3oc3ccccc34)ccc2-c2cccc3ccccc23)cc1 ; c1ccc(-c2cc(N(c3ccc(-c4cccc5ccccc45)cc3)c3cccc4c3sc3ccccc34)ccc2-c2cccc3ccccc23)cc1 ; c1ccc(-c2ccc(-c3cc(N(c4ccc(-c5cccc6ccccc56)cc4)c4cccc5c4oc4ccccc45)ccc3-c3ccccc3)cc2)cc1] 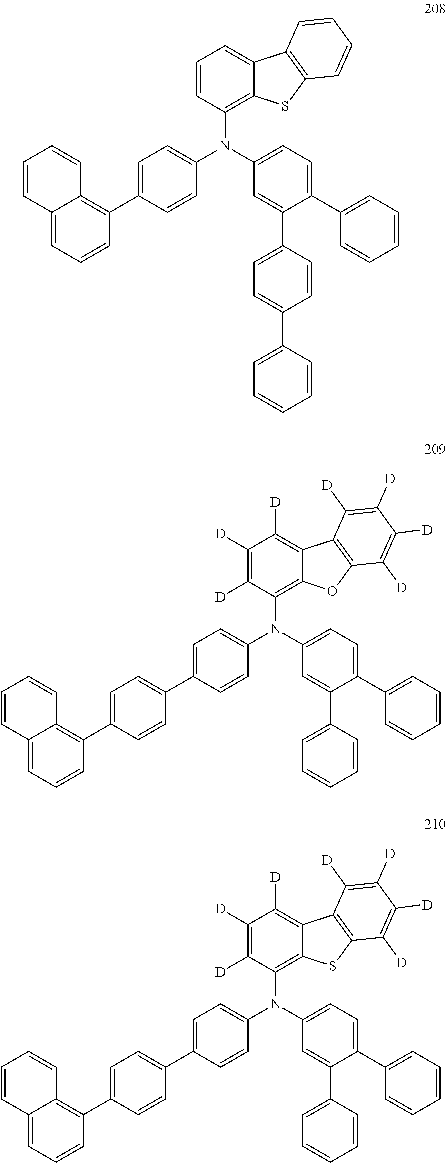 [SMILES: c1ccc(-c2ccc(-c3cc(N(c4ccc(-c5cccc6ccccc56)cc4)c4cccc5c4sc4ccccc45)ccc3-c3ccccc3)cc2)cc1 ; [2H]c1c([2H])c([2H])c2c(oc3c(N(c4ccc(-c5ccc(-c6cccc7ccccc67)cc5)cc4)c4ccc(-c5ccccc5)c(-c5ccccc5)c4)c([2H])c([2H])c([2H])c32)c1[2H] ; [2H]c1c([2H])c([2H])c2c(sc3c(N(c4ccc(-c5ccc(-c6cccc7ccccc67)cc5)cc4)c4ccc(-c5ccccc5)c(-c5ccccc5)c4)c([2H])c([2H])c([2H])c32)c1[2H]] 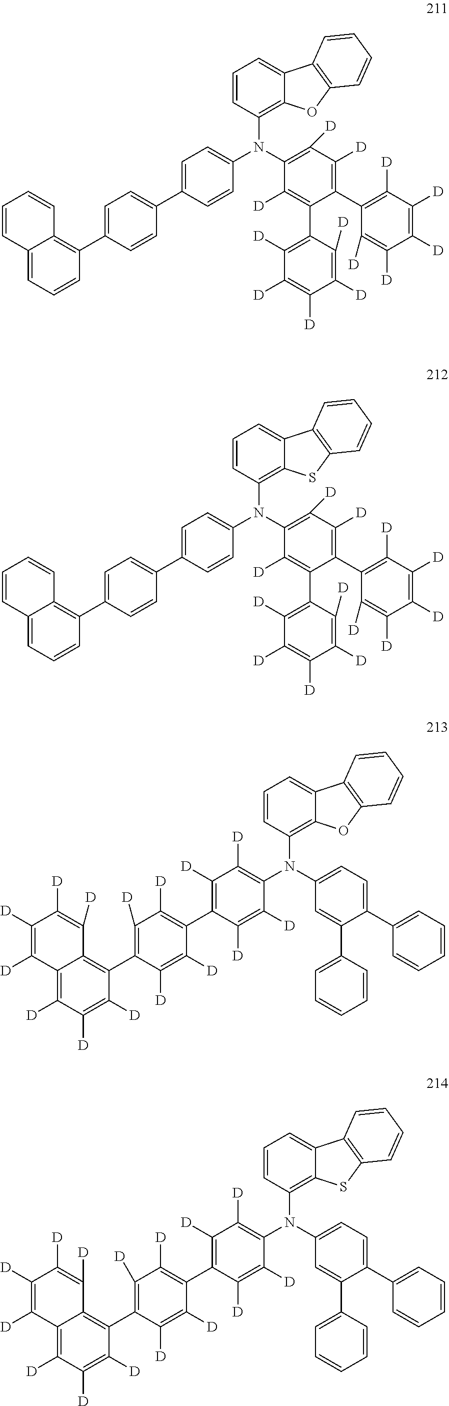 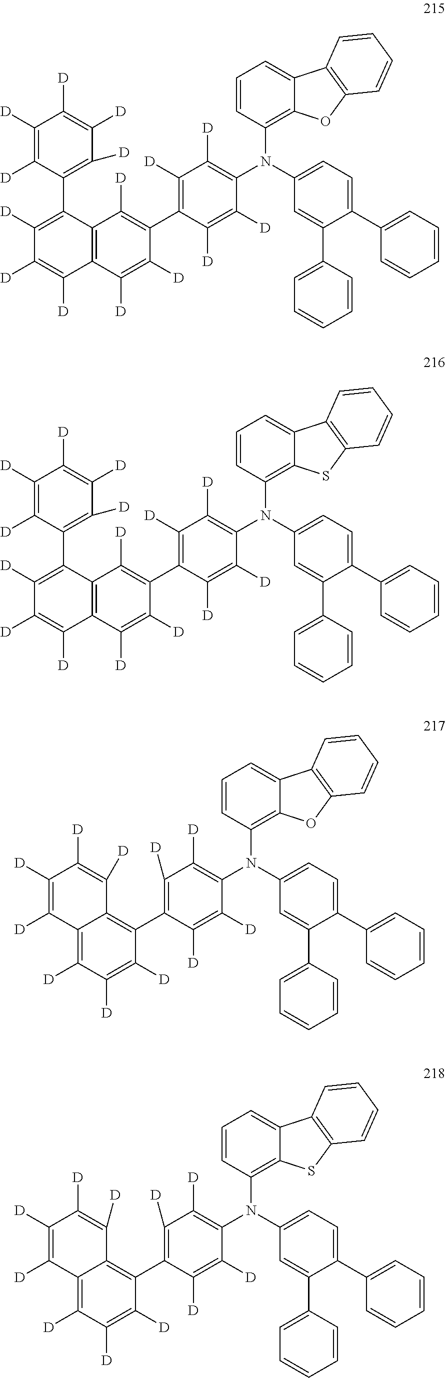 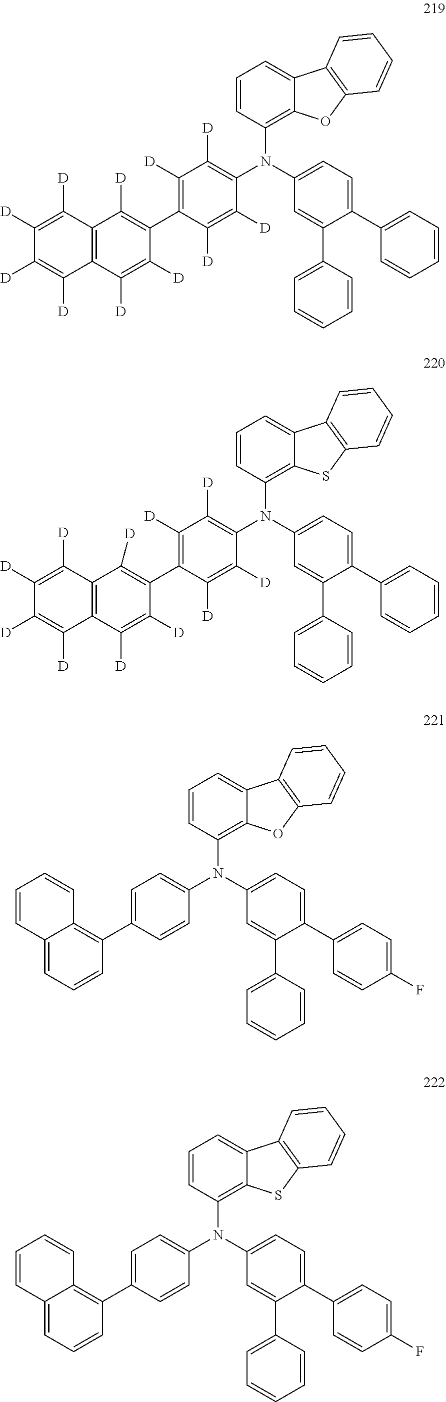 [SMILES: [2H]c1c([2H])c(N(c2ccc(-c3ccccc3)c(-c3ccccc3)c2)c2cccc3c2oc2ccccc23)c([2H])c([2H])c1-c1c([2H])c([2H])c2c([2H])c([2H])c([2H])c([2H])c2c1[2H] ; [2H]c1c([2H])c(N(c2ccc(-c3ccccc3)c(-c3ccccc3)c2)c2cccc3c2sc2ccccc23)c([2H])c([2H])c1-c1c([2H])c([2H])c2c([2H])c([2H])c([2H])c([2H])c2c1[2H] ; Fc1ccc(-c2ccc(N(c3ccc(-c4cccc5ccccc45)cc3)c3cccc4c3oc3ccccc34)cc2-c2ccccc2)cc1 ; Fc1ccc(-c2ccc(N(c3ccc(-c4cccc5ccccc45)cc3)c3cccc4c3sc3ccccc34)cc2-c2ccccc2)cc1] 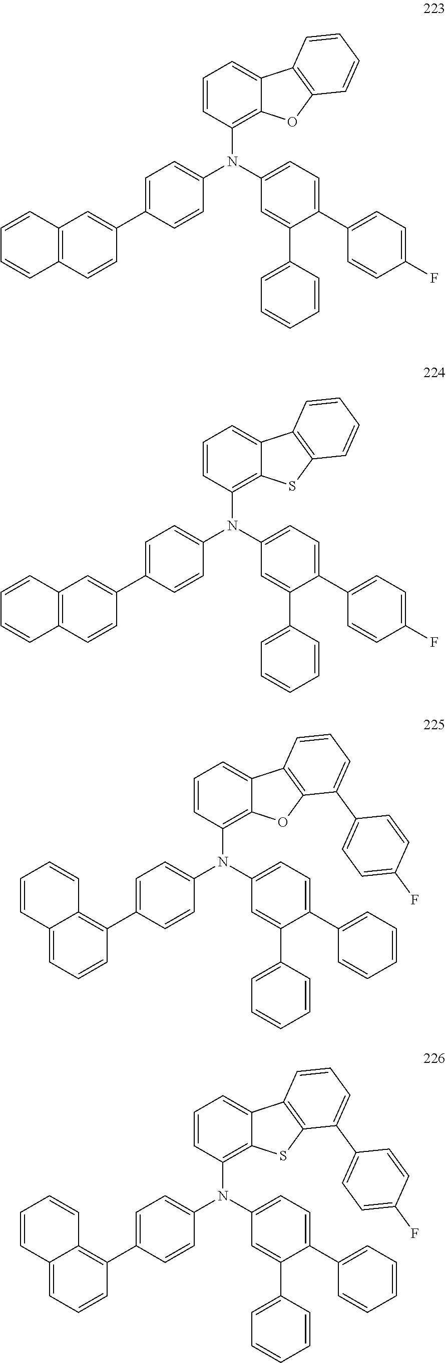 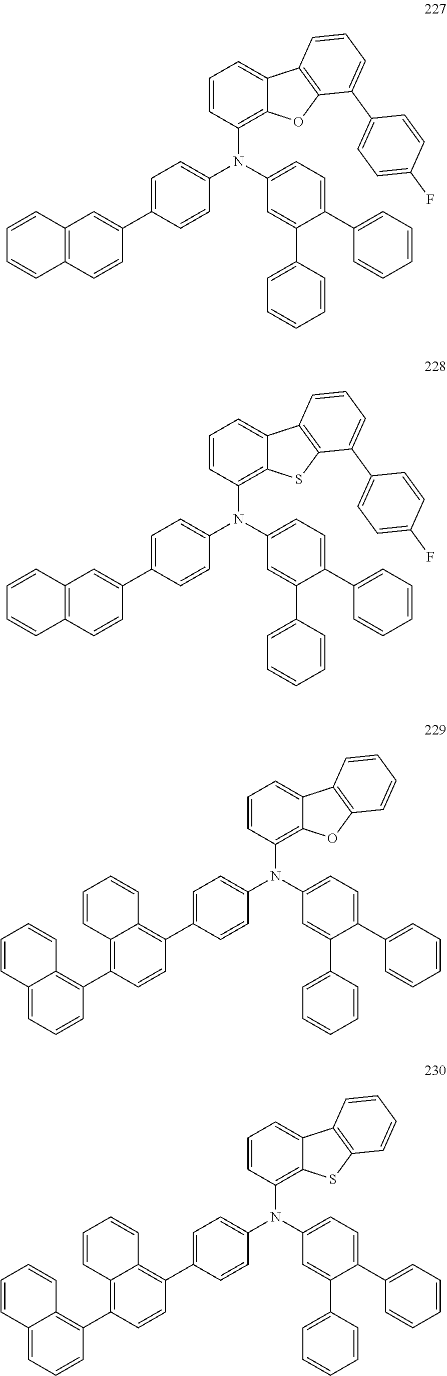 [SMILES: Fc1ccc(-c2cccc3c2oc2c(N(c4ccc(-c5ccc6ccccc6c5)cc4)c4ccc(-c5ccccc5)c(-c5ccccc5)c4)cccc23)cc1 ; Fc1ccc(-c2cccc3c2sc2c(N(c4ccc(-c5ccc6ccccc6c5)cc4)c4ccc(-c5ccccc5)c(-c5ccccc5)c4)cccc23)cc1 ; c1ccc(-c2ccc(N(c3ccc(-c4ccc(-c5cccc6ccccc56)c5ccccc45)cc3)c3cccc4c3oc3ccccc34)cc2-c2ccccc2)cc1 ; c1ccc(-c2ccc(N(c3ccc(-c4ccc(-c5cccc6ccccc56)c5ccccc45)cc3)c3cccc4c3sc3ccccc34)cc2-c2ccccc2)cc1] 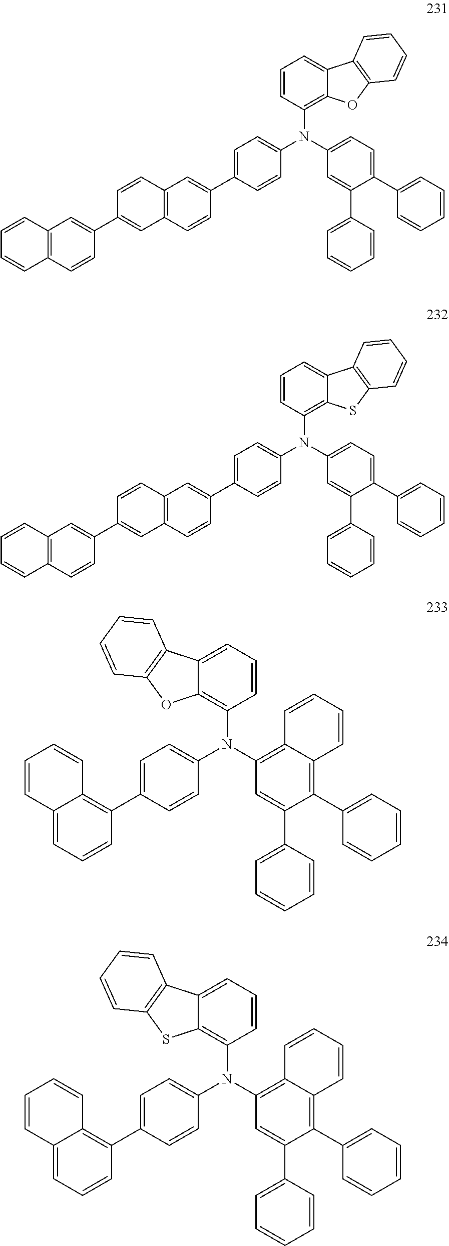 [SMILES: c1ccc(-c2ccc(N(c3ccc(-c4ccc5cc(-c6ccc7ccccc7c6)ccc5c4)cc3)c3cccc4c3oc3ccccc34)cc2-c2ccccc2)cc1 ; c1ccc(-c2ccc(N(c3ccc(-c4ccc5cc(-c6ccc7ccccc7c6)ccc5c4)cc3)c3cccc4c3sc3ccccc34)cc2-c2ccccc2)cc1 ; c1ccc(-c2cc(N(c3ccc(-c4cccc5ccccc45)cc3)c3cccc4c3oc3ccccc34)c3ccccc3c2-c2ccccc2)cc1 ; c1ccc(-c2cc(N(c3ccc(-c4cccc5ccccc45)cc3)c3cccc4c3sc3ccccc34)c3ccccc3c2-c2ccccc2)cc1] 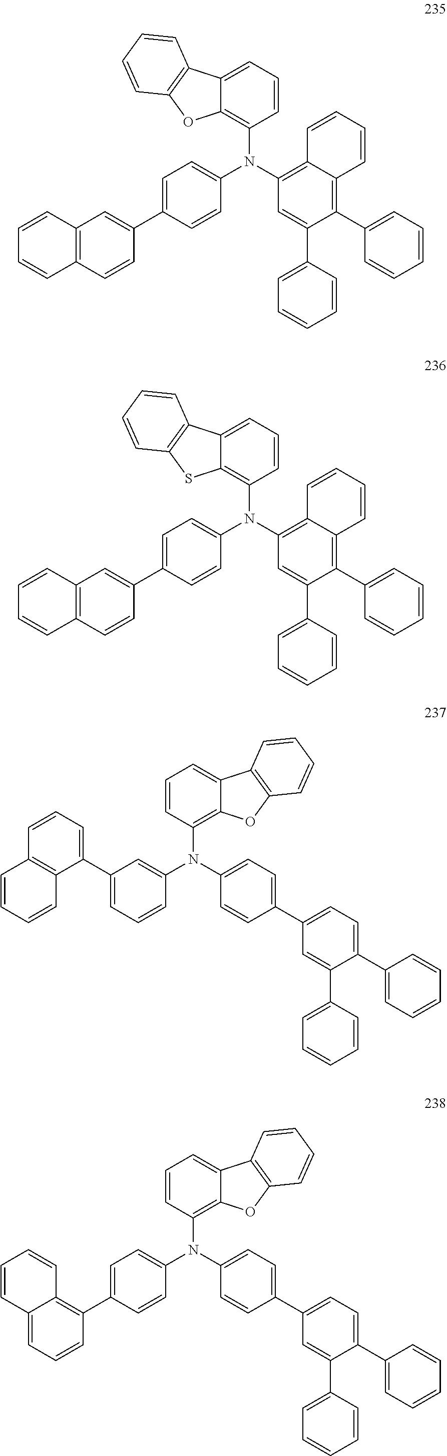 [SMILES: c1ccc(-c2cc(N(c3ccc(-c4ccc5ccccc5c4)cc3)c3cccc4c3oc3ccccc34)c3ccccc3c2-c2ccccc2)cc1 ; c1ccc(-c2cc(N(c3ccc(-c4ccc5ccccc5c4)cc3)c3cccc4c3sc3ccccc34)c3ccccc3c2-c2ccccc2)cc1 ; c1ccc(-c2ccc(-c3ccc(N(c4cccc(-c5cccc6ccccc56)c4)c4cccc5c4oc4ccccc45)cc3)cc2-c2ccccc2)cc1 ; c1ccc(-c2ccc(-c3ccc(N(c4ccc(-c5cccc6ccccc56)cc4)c4cccc5c4oc4ccccc45)cc3)cc2-c2ccccc2)cc1] 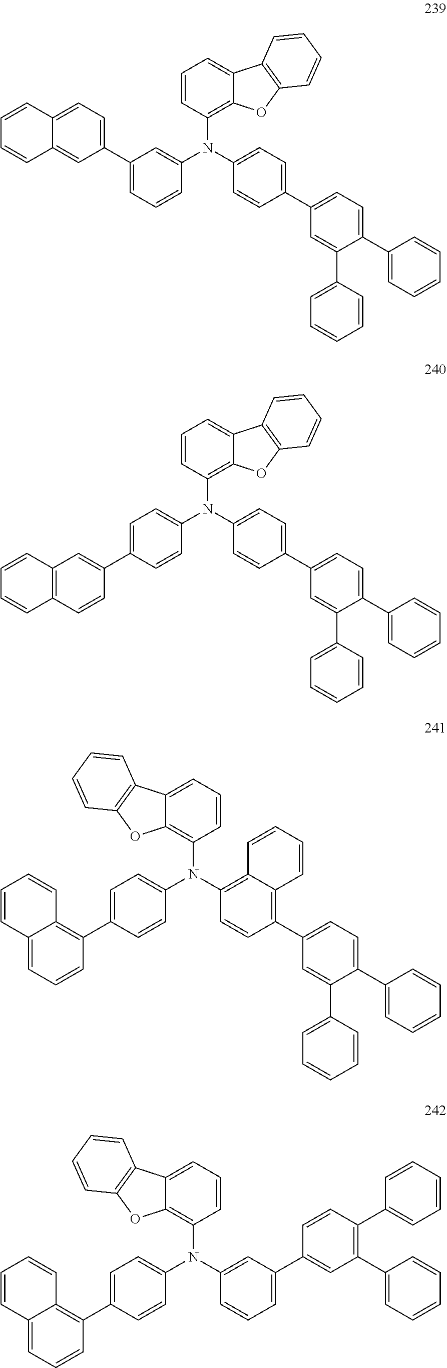 [SMILES: c1ccc(-c2ccc(-c3ccc(N(c4cccc(-c5ccc6ccccc6c5)c4)c4cccc5c4oc4ccccc45)cc3)cc2-c2ccccc2)cc1 ; c1ccc(-c2ccc(-c3ccc(N(c4ccc(-c5ccc6ccccc6c5)cc4)c4cccc5c4oc4ccccc45)cc3)cc2-c2ccccc2)cc1 ; c1ccc(-c2ccc(-c3ccc(N(c4ccc(-c5cccc6ccccc56)cc4)c4cccc5c4oc4ccccc45)c4ccccc34)cc2-c2ccccc2)cc1 ; c1ccc(-c2ccc(-c3cccc(N(c4ccc(-c5cccc6ccccc56)cc4)c4cccc5c4oc4ccccc45)c3)cc2-c2ccccc2)cc1] 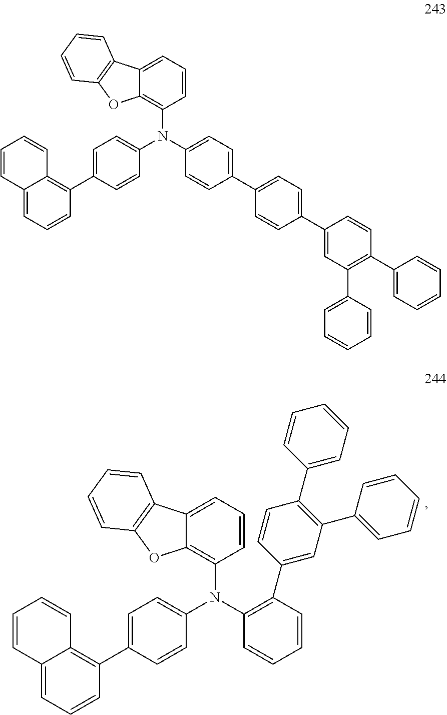 [SMILES: c1ccc(-c2ccc(-c3ccc(-c4ccc(N(c5ccc(-c6cccc7ccccc67)cc5)c5cccc6c5oc5ccccc56)cc4)cc3)cc2-c2ccccc2)cc1 ; c1ccc(-c2ccc(-c3ccccc3N(c3ccc(-c4cccc5ccccc45)cc3)c3cccc4c3oc3ccccc34)cc2-c2ccccc2)cc1]
in Compound Group 1, D being deuterium.

18. A display device, comprising:
a base layer;
a circuit layer on the base layer; and
a display element layer on the circuit layer and comprising a light emitting element,
wherein the light emitting element comprises a first electrode, a second electrode on the first electrode, an emission layer between the first electrode and the second electrode, and a hole transport region between the first electrode and the emission layer and comprising an amine compound represented by Formula 1: 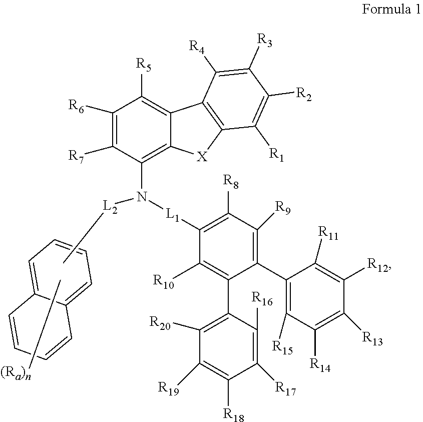
in Formula 1,
X being O or S,
R₁ to R₂₀ being each independently hydrogen, deuterium, a halogen, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or being independently bonded to an adjacent group to form a ring,
L₁ being a direct linkage, a substituted or unsubstituted arylene group having 6 to 30 ring-forming carbon atoms, or a substituted or unsubstituted heteroarylene group having 2 to 30 ring-forming carbon atoms,
L₂ being a substituted or unsubstituted arylene group having 6 to 30 ring-forming carbon atoms,
n being an integer of 1 to 7, and
Rₐ being hydrogen, deuterium, or a substituted or unsubstituted aryl group having 6 to 30 ring-forming carbon atoms.

19. The display device of claim 18, wherein the light emitting element is to emit blue light.

20. The display device of claim 18, further comprising a light control layer comprising a quantum dot.
